(19) United States
(12) Patent Application Publication
(10) Pub. No.: US 2009/0215075 A1
(43) Pub. Date: Aug. 27, 2009

(54) THREE-DIMENSIONAL STRUCTURE OF A DNAB-FAMILY REPLICATIVE HELICASE (G40P), USES THEREOF, AND METHODS FOR DEVELOPING ANTI-BACTERIAL PATHOGENS BY INHIBITING DNAB HELICASES AND THE INTERACTIONS OF DNAB HELICASE WITH PRIMASE

(76) Inventors: Xiaojiang CHEN, Los Angeles, CA (US); Ganggang WANG, Los Angeles, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 12/338,377

(22) Filed: Dec. 18, 2008

Related U.S. Application Data
(60) Provisional application No. 61/014,710, filed on Dec. 18, 2007.

(51) Int. Cl. G01N 33/53; G01N 33/566; G06G 7/48

(52) U.S. Cl. 435/7.1; 436/501; 703/11

(57) ABSTRACT

Structure and methods associated with the three-dimensional structure of G40P helicase and other structure models of any DnaB-like helicase obtained by computer modeling that bears similarity with a root-mean-square deviation (RMSD) of 2.0 with at least one of the three domain structures (N-globe, alpha-hairpin and the C-terminal ATPase domains). In one embodiment, a method for identifying a compound that binds to any fragment of a G40P protein is provided. The method including obtaining the three dimensional structure of the G40P hexamer whose sequence consists of SEQ ID NO:1 and identifying or designing one or more compounds that bind, mimic, enhance, disrupt, or compete with the G40P protein whose sequence consists of SEQ ID NO:1 or interactions of the G40P protein with its ligands based on the three dimensional structure of the G40P hexamer whose sequence consists of SEQ ID NO:1.

### RELATED APPLICATION

This application claims the benefit of and priority to U.S. Provisional Application Ser. No. 61/014,710, filed Dec. 18, 2007, the contents of which are incorporated by reference herein in its entirety.

### GOVERNMENT SUPPORT

This disclosure was made in part with government support under Grant No. NIH Al-055926 awarded by the National Institutes of Health. The government has certain rights to this disclosure.

### BACKGROUND

### Sequence Listing

This application contains a sequence listing, submitted in both paper and a Computer Readable Form (CRF) and filed electronically via EFS. The file is entitled “Helicase010201.txt”, is 26,589 bytes in size (measured in Windows XP) and was created on Dec. 16, 2008.

### Field of the Disclosure

The present disclosure relates generally to the information provided by the three-dimensional structure of G40P helicase and other structure models of any DnaB-like helicase obtained by computer modeling that bears similarity with a root-mean-square deviation (RMSD) of 2.0 with at least one of the three domain structures (N-globe, alpha-hairpin and the C-terminal ATPase domains). Additionally, the present disclosure relates to the uses of the three-dimensional structure of G40P and models of DnaB family helicases particularly for structure-based drug design of compounds designed to target the following interactions: G40P with primase, DNA or ATP; interactions between the domain structures (N-globe, alpha-hairpin and C-terminal ATPase domains) of G40P monomers and of other DnaB family helicase monomers. Lastly, the present disclosure relates generally to the use of the G40P structure and models of DnaB family helicases for structure-based methods designed to block DNA replication of bacterial pathogens and thereby serve as a novel antibiotic drug to inhibit bacterial pathogens that cause many different diseases in humans and animals.

### Background of the Disclosure

Helicases are essential enzymes for DNA replication, a fundamental process in all living organisms. Proteins in the DnaB family are hexameric replicative helicases that unwind duplex DNA and coordinate with RNA primase and other proteins at the replication fork in prokaryotes. Replication of cellular genomic DNA requires the highly coordinated activities of multiple factors. In E. coli cells, initiation of DNA replication occurs through the concerted actions of DnaA, the DnaB helicase, and the DnaG primase, which leads to the assembly of the replisome complex and establishment of two replication forks (reviewed in^{1,2}). For the replication fork to form, DnaB must be recruited to the melted origin of DNA by DnaC and DnaA³⁻⁵. The DnaB helicases also associate with DnaG primase and the polymerase loader DnaX to coordinate fork unwinding with RNA primase and DNA polymerase activities^{1,6-10}.

During elongation of DNA replication, DnaB helicase unwinds dsDNA to provide template for leading and lagging strand synthesis (reviewed in^{1,2,11}). Evidence indicates that the DnaB-family helicases encircle ssDNA near a DNA fork on the 5′ side, with the C-terminal domain facing the fork (reviewed in¹²). As DnaB unwinds the replication fork in a 5′-3′ direction, Okazaki fragments are primed by primase for lagging-strand synthesis using the ssDNA exiting the helicase channel. By recruiting DnaG to hexameric DnaB at the replication fork, DnaB regulates the priming activity and processivity of RNA primase^{7,8,13,14}. Conversely, primase stimulates the ATPase and helicase activities of DnaB^{15,16}. Although well documented, no mechanistic explanation is available to explain this cross-talk between primase and helicase.

The Bacillus subtilis bacteriophage SPP1 helicase, G40P, is a close homolog of bacterial DnaB helicase. G40P has the same domain structure as other bacterial DnaB homologs (FIG. 1a) and shares 35% and 45% sequence identity with the replicative helicases from E. coli and B. subtilis, respectively. In fact, G40P and the cellular helicase both interact with DnaG primase for DNA replication^{17,18}.

The low-resolution EM images obtained for DnaB and G40P revealed two main classes of double-tiered hexamers: 3-fold and 6-fold hexamers¹⁹⁻²³. Domain assignments were attempted by placing the N-terminal fragment of DnaB^{24,25} into the smaller ring and by positioning the T7 gp4 helicase domain^{26,27} into the larger ring, which, as discussed below and shown herein was incorrect. To advance the understanding of DnaB at the replication fork and its interactions with other replication proteins, such as DnaG primase, the crystal structure of the full-length G40P hexamer and other structure models of any DnaB-like helicase, along with their uses, and the methods of using G40P and related bacterial DnaB family helicases, need to be determined.

Therefore, there is a need in the art for a three-dimensional crystal structure of the full-length G40P hexamer and other structure models of any DnaB helicases obtained by computer modeling that bears similarity with a root-mean-square deviation (RMSD) of 2.0 with at least one of the three domain structures (N-globe, alpha-hairpin and the C-terminal ATPase domains) in order to: (i) better understand the molecular interactions of the DnaB family helicases with DnaG primase, (ii) enable the identification and/or design of compounds that mimic, enhance, disrupt or compete with the interactions of GP40 and related bacterial DnaB family helicases to inhibit the helicase function DnaB helicase, or to inhibit the interactions with primase and DNA, which are required for DNA replication, and (iii) to use G40P and related bacterial DnaB family helicases for their many uses.

Additionally, there is a need in the art for using G40P and related bacterial DnaB family helicases for structure based drug design of regulatory compounds that combat disease, especially bacterial pathogens. Furthermore, there exists a need in the art for methods using the G40P helicase and related bacterial DnaB family helicases to identify and develop drugs or compounds that inhibit helicases, including but not limited to bacterial DnaB helicases.

Furthermore, there exists a need in the art to be able to determine the regions of G40P and related DnaB helicase structures that are important for interactions with DnaG primase via structure-guided mutagenesis of G40P and related DnaB helicases designed to disrupt primase binding to the helicase and thereby inhibiting helicase activity or DNA replication.

There also exists a need in the art to be able to affect the following interactions of G40P and related DnaB helicases that are important for hexamerization, helicase activity and DNA replication: (i) interactions between the C-terminal ATPase domains and the N-globe domains of the monomers that comprise the hexameric helicase; (ii) interactions between the separate C-terminal ATPase domains of monomers that comprise the hexameric helicase; (iii) interactions between the individual N-globe domains of monomers that comprise the hexameric helicase; and (iv) interactions between the C-terminal ATPase domains and alpha-hairpin domains of the monomers that comprise the hexameric helicase.

There also exists a need in the art to be able to affect the regions of the ATPase binding pocket to prevent ATP hydrolysis leading to inhibition helicase activity or DNA replication.

Furthermore, there exists a need to alter the structural conformation of G40P or related DnaB helicases with a compound or peptide which can alter or disrupt helicase activity and inhibit DNA replication.

There also exists a need in the art to be able to affect binding of G40P or other related DnaB helicases with their respective ligands (e.g. DNA, ATP, DnaG primase, or DnaX) to prevent helicase activity or DNA replication.

There also exists a need in the art for methods for discovering or determining antibacterial drugs via structure-based drug design utilizing the information contained with the three-dimensional structure of G40P or other structure models of any related DnaB helicases obtained by computer modeling that bears similarity with a root-mean-square deviation (RMSD) of 2.0 with at least one of the three domain structures (N-globe, alpha-hairpin and the C-terminal ATPase domains).

Additionally, there exists a need in the art for methods of inhibiting DnaB helicase function and DnaG primase binding for bacterial strains, including but not limited to, strains that cause Tubercle bacillus (T.B.), Listeria monocytogenes (meningitis), Streptococcus pneumoniae (pneumonia) and related bacterial pathogens in an animal. The present disclosure provides these and other related benefits and advantages.

### SUMMARY

One embodiment of the present disclosure relates to the information derived from the three-dimensional structure of G40P helicase and other structure models of any related DnaB helicases obtained by computer modeling that bears similarity with at least one of the three domain structures (N-globe, the alpha-hairpin and C-terminal ATPase domains) with a root-mean-square deviation (RMSD) of 2.0. Another embodiment of the present disclosure relates to a method for the identification of compounds which inhibit helicase activity or DNA replication by affecting the proper function of G40P helicase or any other related DnaB helicases. These compounds may affect the hexameric or conformational structure of a helicase or affect the helicase binding to its ligand substrates.

This and other related methods include the steps of: (a) providing a three dimensional structure of a G40P or model of a related bacterial DnaB family helicase; and, (b) identifying a candidate compound that can affect helicase activity or DNA replication via structure based drug design utilizing structural information provided in (a). The three dimensional structure of G40P or models of related bacterial DnaB family helicases includes structures selected from: (i) a structure defined by atomic coordinates of a three dimensional structure of a crystalline G40P defined by the atomic coordinates represented in Tables 1 and 2, below and incorporated by reference, herein (atomic coordinates and related data of G40P truncated monomers and full-length hexamer, respectively); (ii) atomic coordinates that define a three dimensional structure, wherein at least 50% of the structure has an average root-mean-square deviation (RMSD) from backbone atoms in secondary structure elements in at least one domain of a three dimensional structure represented by the atomic coordinates of (i) of equal to or less than about 2.5 Å for main chain Ca carbon backbone; and (iii) a structure defined by atomic coordinates derived from G40P molecules arranged in a crystalline manner in a space group P2₁2₁2₁ so as to form a unit cell of dimensions a=114 Å, b=184 Å, c=184 Å.

In one aspect of this embodiment, the steps are included for identifying candidate compounds that potentially bind to and affect the proper function of G40P and related DnaB family helicases from bacterial pathogens, including but not limited to, Tubercle bacillus (T.B.), Listeria monocytogenes (meningitis), and Streptococcus pneumoniae (pneumonia).

In another aspect of this embodiment, the method further includes the step of: (c) selecting candidate compounds of (b) that inhibit the binding of G40P to its ligand. The step (c) of selecting can include: (i) contacting the candidate compound identified in step (b) with G40P or a fragment thereof or with a G40P ligand or a fragment thereof under conditions in which a G40P-G40P ligand complex can form in the absence of the candidate compound; and (ii) measuring the binding affinity of the G40P or fragment thereof to the G40P ligand or fragment thereof; wherein a candidate inhibitor compound is selected as a compound that inhibits the binding of G40P to its ligand when there is a decrease in the binding affinity of the G40P or fragment thereof for the G40P ligand or fragment thereof, as compared to in the absence of the candidate inhibitor compound. The G40P ligand can include, but is not limited to, double stranded DNA (dsDNA), single stranded DNA (ssDNA), primase, ATP or G40P-binding fragments of any of the ligands.

The method of selecting a candidate compound of (b) may also include identifying candidate compounds for binding to any one or all of the three domains (N-globe, the alpha-hairpin and C-terminal ATPase domains) of G40P or related DnaB helicases. In one aspect, the step of selecting a compound includes identifying candidate compounds that bind to the interface between the N-globe and the C-terminal ATPase domains of monomeric G40P or related DnaB helicases. In another aspect, the step of selecting a compound includes identifying candidate compounds that bind to one or more of the three domain structures (N-globe, C-terminal ATPase domain, and the alpha-hairpin) of G40P or related bacterial DnaB family helicases and affect helicase hexamerization or the structural conformation of the helicase. In another aspect, the step of selecting a compound includes identifying candidate compounds for binding to the interface between h7 at the N-terminus of the ATPase domain of G40P and the adjacent ATPase domain of another G40P monomer or a fragment thereof. In one aspect, the step of selecting a compound includes identifying candidate compounds that bind to the loop connecting h7 to the ATPase domain of G40P. In yet another aspect, the step of selecting a compound includes identifying candidate compounds that bind to any area of G40P or related DnaB helicases and affect helicase hexamerization or the structural conformation of the helicase.

The step of identifying a compound in the method of the present disclosure can include any suitable method of drug design, drug screening or identification, including, but not limited to: directed drug design, random drug design, grid-based drug design, and/or computational screening of one or more databases of chemical compounds.

Yet another embodiment of the present disclosure relates to a method to identify a compound that inhibits the G40P-dependent or related DnaB-dependent replication of bacteria. This method includes the steps of: (a) providing a three dimensional structure of G40P or one or more related bacterial DnaB family helicase models as described in detail above; (b) identifying a candidate compound for binding to G40P by performing structure based drug design with the information provided by the structure of (a) to identify a compound structure that binds to the three dimensional structure of the G40P or related DnaB helicases; (c) contacting the candidate compound identified in step (b) with a bacteria cell that expresses G40P or related DnaB helicases or a ligand binding fragment thereof under conditions in which G40P or related DnaB helicases can replicate in the absence of the candidate compound; and (d) measuring the DNA synthesis of the cell; wherein a candidate inhibitor compound is selected as a compound that inhibits the DNA synthesis, as compared to in the absence of the candidate inhibitor compound.

Yet another embodiment of the present disclosure relates to a method to identify a compound that inhibits the binding of G40P or related DnaB helicase ligand or fragment thereof as described previously to G40P or one or more related bacterial DnaB family helicases. This method includes the steps of: (a) providing a three dimensional structure of G40P or one or more related bacterial DnaB family helicase models as described in detail above; (b) identifying a candidate compound for binding to the G40P or one or more related bacterial DnaB family helicases by performing structure based drug design utilizing the information provided by the structure of (a) to identify a compound structure that binds to the three dimensional structure of the G40P or one or more related bacterial DnaB family helicases; (c) contacting the candidate compound identified in step (b) with a first cell expressing G40P, one or more related bacterial DnaB family helicases, or a fragment thereof of either and a second cell expressing a G40P ligand, related bacterial DnaB family helicase ligand or fragment thereof under conditions in which the G40P protein, related bacterial DnaB family helicases or fragment thereof and the G40P or related bacterial DnaB family helicases ligand binding fragment thereof can bind in the absence of the candidate compound; and (d) measuring a biological activity induced by the interaction of G40P, or related bacterial DnaB family helicases and the G40P ligand and related bacterial DnaB family helicase ligand, respectively, in the first or second cell; wherein a candidate inhibitor compound is selected as a compound that inhibits the biological activity as compared to in the absence of the candidate inhibitor compound. In a preferred embodiment, the biological activity is the creation of an unwounded DNA replication fork or DNA replication.

Another embodiment of the present disclosure is a therapeutic composition that, when administered to an animal, prevents replication of bacteria in the animal. The therapeutic composition comprises a compound that interacts with primase and/or helicase to prevent replication of the bacteria. The compound is identified by the method that includes the steps of: (a) providing a three dimensional structure of G40P or one or more related bacterial DnaB family helicase models as described in detail above; (b) identifying a candidate compound for binding to G40P or related bacterial DnaB family helicases by performing structure based drug design utilizing the information provided by the structure of (a) to identify a compound structure that binds to the three dimensional structure of G40P or related bacterial DnaB family helicases; (c) synthesizing the candidate compound; and (d) selecting candidate compounds that bind to and affect the proper functions of G40P or one or more related bacterial DnaB family helicases thereby preventing the replication of bacteria within the animal.

Yet another embodiment relates to a therapeutic composition that, when administered to an animal, inhibits the biological activity of G40P or related bacterial DnaB family helicases in the animal. The therapeutic composition includes a compound that inhibits the activity of G40P or related bacterial DnaB family helicases. The compound is identified by the method that includes the steps of: (a) providing a three dimensional structure of G40P or one or more related bacterial DnaB family helicases as described in detail above; (b) identifying a candidate compound for binding to the G40P or one or more related bacterial DnaB family helicases by performing structure based drug design utilizing the information provided by the structure of (a) to identify a compound structure that binds to the three dimensional structure of G40P or one or more related bacterial DnaB family helicase models; (c) synthesizing the candidate compound; and (d) selecting candidate compounds that inhibit the biological activity of G40P or one or more related bacterial DnaB family helicases. Preferably, the compounds inhibit the formation of a complex between G40P or one or more related bacterial DnaB family helicases, and their ligands. The ligand can include, but is not limited to: ssDNA, dsDNA, ATP, primase, G40P monomer, and G40P-binding fragments of any of the ligands. In one aspect, the compound inhibits the activation of G40P or one or more related bacterial DnaB family helicases.

Yet another embodiment of the present disclosure relates to a method of preparing G40P proteins or one or more related bacterial DnaB family helicases having modified biological activity. This method includes the steps of: (a) providing a three dimensional structure of a G40P or one or more related bacterial DnaB family helicases as described in detail herein; (b) utilizing the information provided by the three dimensional structure of G40P or one or more related bacterial DnaB family helicase models and performing structure based drug design with the structure of (a) to identify at least one or more sites in the structure contributing to the biological activity of G40P or one or more related bacterial DnaB family helicases; and (c) modifying at least one or more sites in a G40P protein to alter the biological activity of the G40P protein or one or more related bacterial DnaB family protein.

Yet another embodiment of the present disclosure relates to an isolated protein comprising a mutant G40P or one or more related mutant bacterial DnaB family helicases. The protein comprises an amino acid sequence that differs from the wildtype sequence via amino acid substitution. The G40P mutant protein or mutant bacterial DnaB family protein includes mutations that can reduce binding to the ATP in the ATPase binding pocket, as compared to a wildtype G40P or the wildtype related DnaB protein.

### DRAWINGS

The above-mentioned features and objects of the present disclosure will become more apparent with reference to the following description taken in conjunction with the accompanying drawings wherein like reference numerals denote like elements and in which:

The patent or application file contains at least one drawing executed in color. Copies of this patent or patent application with color drawing will be provided by the Office upon request and payment of the necessary fee.

FIG. 1 shows the overall features of the G40P hexamer structure. More specifically, FIG. 1a is a diagram showing the known domain organization of the full-length SPP1 G40P replicative helicase, a homolog of E. coli DnaB. FIG. 1b is a side-view of the full-length G40P hexamer structure. It should be appreciated that there is a distinct separation between the wider, thin top N-terminal tier (in green and cyan) and the narrower, thick bottom C-terminal tier (in purple). FIGS. 1c and 1d show a surface and ribbon representation of the G40P hexamer and demonstrate the wide-open, quasi 3-fold triangular ring on the top of the pseudo 6-fold C-terminal ring.

FIG. 2 shows two distinct monomeric structures of G40P. More specifically, FIG. 2a shows the cis-structure, in which the α-hairpin (helices h5, h6) points to the same side of the ATPase domain (in cyan). α-helices are labeled as h1-h15, and β strands from 1-9, both from N- to C-termini. In this conformation, the α-hairpin, but not the N-globe, contacts the ATPase domain. FIG. 2b shows the trans-structure, where the α-hairpin (in yellow) points away from the ATPase domain. In this conformation, neither the α-hairpin, nor the N-globe (in blue), contacts the ATPase domain. FIG. 2c shows the superposition of the two conformations based on the ATPase domains, which overlaps well (0.35 Å rmsd). However, from h7 toward the N-terminus, the α-hairpin and N-globe of the two conformers have dramatically different orientations and positions. FIG. 2d is a diagram depicting the actual domain boundaries of G40P structure. FIG. 2e shows the superposition of G40P ATPase domain (in red) with T7 gp4 helicase domain (in salmon) based on the β-sheet core. The superposition shows good overlaps in the core β-strands, but variability in a few loops, turns, and α-helices.

FIG. 3 shows assembly of the G40P hexamer. More specifically, FIG. 3a is a diagram showing subunit arrangement in a hexamer viewed from the N-tier. Trans-monomers are colored in blue (numbered 2, 4, 6) and cis-monomers in purple (numbered 1, 3, 5). Larger spheres represent the C-terminal ATPase domains (1C-6C), the smaller spheres represent the N-globe (1N-6N), and the α-helical structures are the α-hairpin (1t-6t). FIG. 3b shows the triangular shaped N-tier, showing two types of dimer interfaces: three N-globe-to-N-globe (head-head) and three α-hairpin-to-hairpin interfaces. The three cis-monomers (in purple) form the inner triangle, and the three trans-monomers (in blue) form the outer triangle. FIG. 3c shows the dimer formed by packing between the cis (purple) and trans (blue) α-hairpins. FIG. 3d shows the dimer formed between cis and trans N-globe. FIG. 3e shows the side-view of the G40P hexamer showing the inter-subunit arrangement of three monomers. The h7 of the red or cyan monomer reaches into the ATPase domain of the next monomer, and the N-globe of the cyan monomer (cis) packs with the h13/h14 of the ATPase domain of the red monomer (trans). FIG. 3f shows the G40P hexamer related by a 60-degree vertical rotation from the view in panel-e. The α-hairpin of the trans-monomer in blue reaches over to interact with h13/h14 of the ATPase domain of a neighboring cis-monomer in cyan, and packs with the α-hairpin of the cis-monomer to form a 4-helix bundle.

FIG. 4 shows G40P helicase activity and helicase-primase interactions. FIG. 4a shows the helicase activity of G40P N-terminal deletion mutants, showing the importance of the N-globe and α-hairpin for helicase function. The quantified helicase activity of the mutants was expressed as the % of the full-length (FL) activity. FIGS. 4b and 4c show the location of the residues on G40P investigated by mutational analysis to define the primase binding site. Residues mutated in each construct are represented in colored sphere (See FIG. 11 for additional details). FIG. 4d depicts a native gel shift assay of primase binding to the two N-terminal fragments of G40P, N149 and N171. FIG. 4e depicts a native gel shift assay of primase binding to wt and mutant G40P. FIG. 4f shows the primase-mediated helicase stimulation of G40P wt and mutants, expressed as fold of helicase stimulation by primase (compared to the activity in the absence of primase). A ratio of three primase molecules to one G40P hexamer was used in the helicase stimulation assay. Only those G40P mutants with detectable helicase activity were tested for primase-mediated stimulation. The mt3 mutant was used as a negative control for the primase-mediated stimulation as it had no detectable primase-binding. Error bars in panels a and f are representative of standard error as calculated from a minimum of three independent experiments.

FIG. 5 shows a model of helicase-primase complex at a DNA fork, with the 5′-end ssDNA (colored in orange) exiting the helicase channel for lagging strand synthesis. G40P helicase is in surface representation, with its C-terminal ATPase end facing toward the dsDNA fork. DnaG primase has three domains, the C-terminal P16 domain (shown as spheres labeled as prim. 1, 2, 3), the RNA polymerase domain (RPD), and the Zn domain (Zn)^{32,33,35,48}. Each of the three DnaG (colored in yellow, cyan, or salmon) uses its C-terminal P16 domain to contact the N-terminal tier of G40P. For primer synthesis, the Zn domain of primase 2 (prim. 2) interact with the RPD of primase 1 (prim. 1) to bind the ssDNA coming out of the channel of G40P/DnaB to initiate lagging strand primer synthesis.

FIG. 6, depicts the crystal structures of G40P deletion mutant and sequence alignment of DnaB from several organisms. FIG. 6a shows the filament arrangement of 2.35 Å crystal structure of six monomers of G40P deletion mutants ΔN129 from space group p6₁, viewing along the 6-fold screw-axis from the N-terminal end. FIG. 6b displays the conformations of the key residues around the ATP pocket in the ΔN129 structure that was crystallized in the presence of ATPγS. ATP was modeled into the electron density that has only strong triphosphate electron density across the p-loop, but not well-defined density corresponding to the base. The residues are in bonding distance with the ATP, including the Arg finger R414, and a nearby K412. FIG. 6c provides the amino acid sequence alignment of DnaB-homologs. The SPP1 G40P helicase polypeptide is aligned with its homologs from B. subtilis (Bsub), E. coli (Ecol, residue 18-471), and T7 gp4 (residue 35-503). Numbering and secondary structure elements depict the structure of G40P as shown in FIG. 2. Symbols are as follows: black rods=(α-helices, blue arrows=β-strands, blue diamonds=residues mutated in Bacillus stearothermophilus₁₆, green diamonds=mutated residues in the temperature-sensitive mutants of the Staphyloococcus aureus₃₀, stars=residues mutated in our study of G40P. Residues that lie in the conserved catalytic Walker A and Walker B motifs, as well as loop 2 are indicated.

FIG. 7, shows the mapping of the residues affecting DnaG binding onto the surface of G40P N-tier. The residues were identified from literature describing mutational data_{13,16,29} (blue) and from temperature sensitive (ts) genetic screens 30 (in green). The residues are located on the exposed side of the 3-fold N-tier of G40P. Sequence alignment of DnaB family members with G40P showed that four of these mutated residues are absolutely conserved (indicated by blue diamonds in FIG. 6c). Despite the distant positions between these four residues on the primary sequence, these residues are found clustered together on the exposed surface of the N-terminal tier of G40P (blue spots). Additionally, four temperature sensitive (ts) mutants of the DnaB-like helicase from S. aureus that affect DNA replication and cell growth, also cluster to the same surface (green spots) on the N-terminal ring. This co-localization of ts mutants with the residues affecting DnaG binding suggests that this exposed surface on the N-terminal tier may be involved in DnaG primase. Alternatively these residues may play an important structural role to support the scaffold of the N-terminal tier of G40P for primase binding.

FIG. 8 displays the experimental electron density map resulted from the 6-fold multidomain averaging at 4.5 Å, showing the N-terminal tier viewing along the hexameric channel (or quasi 3-fold axis) (FIG. 8a), and from the side (FIG. 8b). The boundaries for the six monomers are clear, and the connectivity of the main-chain density is obvious. The density corresponding to the N-globe and α-hairpin regions can be recognized in both panels. FIG. 8c shows examples of two different regions of the model-phased electron density map (2FoFc map to 3.9 Å). In addition to the excellent connectivity. of the map, the densities for some of the bulky aromatic side chains (Trp, His, Tyr, Phe, etc.) are well defined, and serve as the landmarks for registry during construction of the final refined model of G40P hexamer structure.

FIG. 9 depicts the anomalous map showing the Se peaks (in red mesh) located on the α-hairpins and N-terminal globes of one dimer of cis- and trans-monomers as found in the G40P hexamer. This map shows that Met143 from two α-hairpins of adjacent molecules pack side-side to each other, and Met side chains from the model fits nicely into the anomalous Se peaks. Similarly, Met52 side chain in the N-globe also fits right into the Se-peak. These and other Se peaks (a total of 58 Se peaks in one hexamer), together with the defined large side chain density were critical road marks for the construction of the initial model and verification of the registry of the polypeptide.

FIG. 10, is a table summarizing the crystallographic data collected and refinement statistics obtained by performing the experiments discussed herein.

FIG. 11, is a table summarizing the results of the G40P mutagenesis study discussed herein. FIG. 8 demonstrates the functional interactions of B. subtilis DnaG primase with wt and mutant G40P helicase proteins. The wt and all mutant proteins of G40P isolated from the hexamer peak in gel filtration were used for the primase-binding and functional assays. The location of G40P mutants on the hexamer structure are as follows: mt1 and mt2 on α-hairpin surface (αHp surface); mt3 at the interface between two N-globes (Ngb-to-Ngb) for trans monomers, or on the exposed surface for cis monomers; mt4 at the interface between N-globe and C-tier (Ngb-to-C); and mt5 at the interface between the α-hairpin and the C-tier (αHp-to-C). Note:^{1,2} Values for the ATPase and helicase activity are expressed as percentage of those of wt G40P in the absence of DnaG primase (set as 100%), with a standard deviation indicated by “±”. *^{,#}Values are given relative to full-length activity in the absence of DnaG.

FIG. 12 shows the atomic coordinates of G40P Truncated Monomers as represented by SEQ ID NO:2.

FIG. 13 shows the atomic coordinates and related data of G40P Full-Length Hexamer as represented by SEQ ID NO:1.

### DETAILED DESCRIPTION

The present disclosure relates to the discovery of the three-dimensional full-length crystal structure of a DnaB family helicase, the G40P from the B. subtilis phage SPP1, its various uses and methods for drug discovery related to the information provided by the G40P structure and related DnaB helicase model structures. G40P is a homolog of bacterial DnaB helicase and has the same domain structure as other bacterial DnaB homologs. Since G40P shares sufficient sequence and structural similarities to DnaB helicases from bacteria, it can be considered the same family of DnaB helicases from any bacterial strains. As a result, G40P structure can be used for homology modeling to obtain models of other DnaB helicases from any bacterial pathogens. The present disclosure provides these and other additional advantages described herein.

The hexamer structure of G40P reveals a unique architectural feature and a novel assembly mechanism. The hexamer has two-tiers: a 3-fold N-terminal tier and a 6-fold C-terminal tier. Monomers with two drastically different conformations, termed cis and trans, come together to provide a topological solution for the unusual dual symmetry within a hexamer. A structure-guided mutational study suggests an important role for the 3-fold N-terminal tier in binding primase and regulating primase-mediated stimulation of helicase activity.

Additionally, to advance the understanding of DnaB at the replication fork and its interactions with other replication proteins, such as DnaG primase, the crystal structure of the full-length G40P hexamer was determined, as disclosed herein. The structure shows a double-tiered architecture that has an unexpected dual symmetry: a 3-fold N-terminal tier and a near 6-fold C-terminal tier. Assembly of the two distinct tiers in a single hexamer is achieved by using two monomer conformations. Monomers with cis and trans conformations interact alternately, like a right hand holding the left hand in a circle to form a hexameric ring. The G40P structure guided mutagenesis has provided insights into the structural and functional interplay with DnaG primase.

The present disclosure also relates to the structural and functional interplay between G40P helicase and DnaG primase, to crystalline G40P complexes and related DnaB helicase structures, to models of such three-dimensional structures, to a method of structure-based drug design using G40P and related DnaB helicase structures, to the compounds identified by such methods and to the use of such compounds in therapeutic compositions and methods.

The results of the experiments, methods and structures disclosed herein provide the first detailed understanding of receptor-ligand interactions in this protein family and reveal potential target sites for molecular drug design.

Results

Overall Structure of the Hexameric Helicase

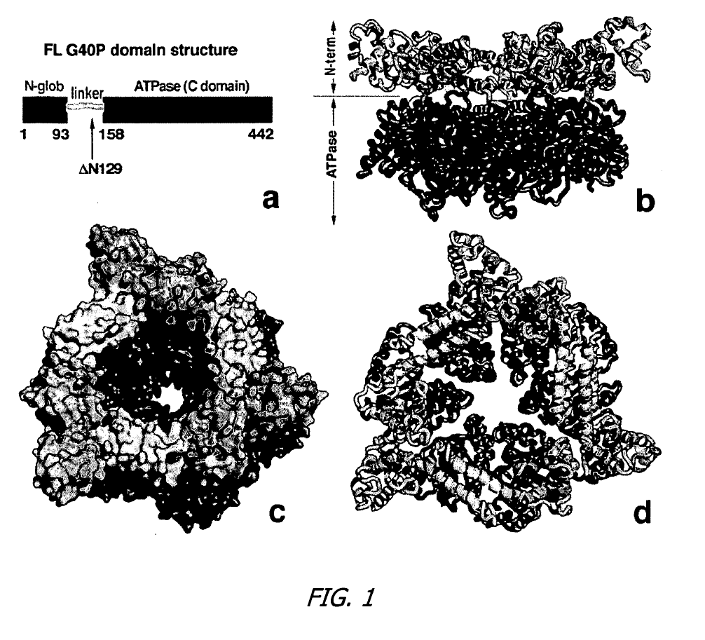

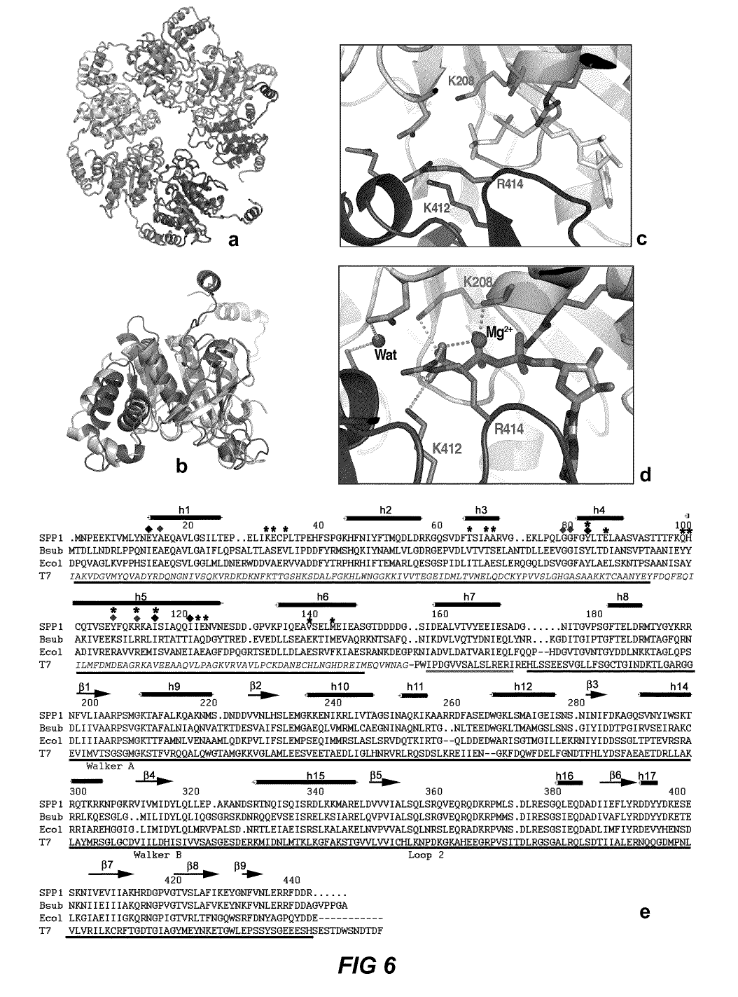

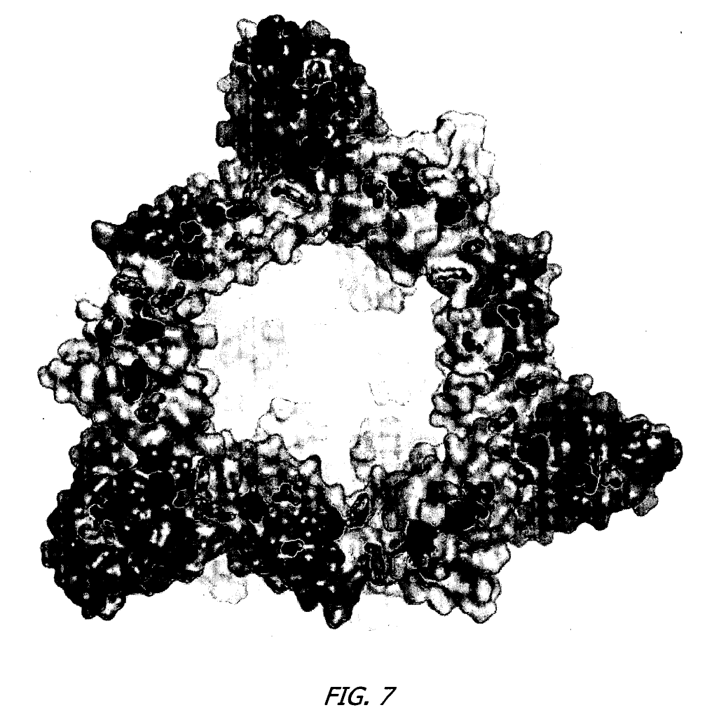

The present disclosure discloses two novel crystal structures of the DnaB homolog from B. subtilis bacteriophage SPP1, the full-length and a deletion mutant of the G40P (FIGS. 7 & 10). Truncation of the N-terminal 129 residues (ΔN129) (FIG. 1a) yielded a crystal form containing one molecule per asymmetric unit (asu). The structure of ΔN129 (FIG. 6a) was used to help determine the full-length G40P structure. The full-length G40P crystallized with one complete hexamer in one asu.

The G40P hexamer resembles a two-tiered ring (FIG. 1b-d). A very unusual feature of this double-tiered ring is the presence of two distinctive symmetry patterns (FIG. 1c-d). The top tier (in green/cyan) containing the N-terminal domains displays a near 3-fold symmetry. In contrast, the bottom tier (in purple) composed of the C-terminal ATPase domains has a quasi 6-fold symmetry. Unexpectedly, the top N-terminal tier (N-tier) is wider than the C-terminal tier (C-tier). This is in contrast to the EM reports of DnaB and G40P, which assigned the C-terminal ATPase ring as the wider of the two tiers^{22,23}. The top N-tier has a much larger channel diameter than the bottom C-tier, 42 Å vs. 17 Å, respectively, as measured between the nearest Cα carbons. Another unexpected result was that the linker region that was previously assumed to be flexible (FIG. 1a) is actually well structured in the full-length hexamer (the cyan part in FIGS. 1c-d).

Monomer Structure

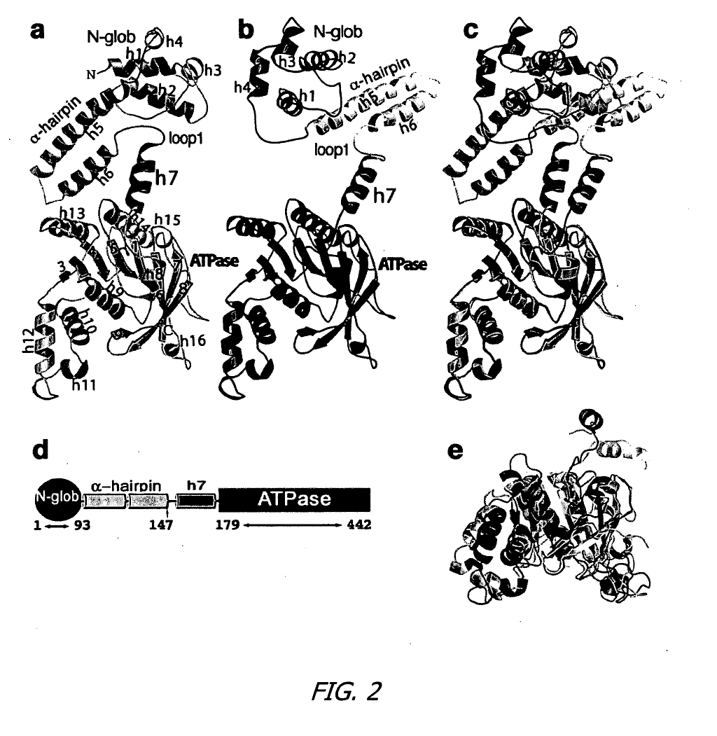

The full-length G40P monomer structure is composed of three domains: an N-terminal globular domain (residues 12-93), a “linker” region (residues 94-147) composed of two long α-helices, and a C-terminal RecA-like domain (residues 179-437)(FIG. 2a-d). The N-terminal globular domain (N-globe) consists of four α-helices (h1-h4, FIG. 2a), which is similar to the X-ray and NMR structures of the N-terminus of E. coli DnaB^{24,25}. The linker region folds into two consecutive α-helices (h5, h6) arranged in an anti-parallel fashion to form a hairpin-like structure α-hairpin) (FIGS. 2a-b). The RecA-like C-terminal domain (C-domain) consists of a nine-stranded β-sheet sandwiched by three α-helices on both sides. This β-sheet core is similar to that of T7 gp4 helicase domain^{26,27} with a superposition of 1.233 Å rmsd over 77 Cα-atoms from the β-sheet core (FIG. 2e). However, superposition over 253 Cα-atoms of G40P ATPase domains and T7 gp4 helicase domain has an rmsd of 2.825 Å, suggesting a much larger difference for the helical and loop regions outside the β-sheet core.

Cis- and Trans-Structures

One unique feature of the G40P hexamer is that the complex is composed of two drastically different monomer conformations, termed cis- and trans-structures (FIG. 2a vs. FIG. 2b). The cis-structure has the α-hairpin pointing to the same side (cis-side) as the C-domain (FIG. 2a). The N-globe in the cis-structure is projected away from the C-domain. The trans-structure has the α-hairpin pointing to the opposite side (trans-side) of the C-domain (FIG. 2b), which places the N-globe in a different position compared to that of the cis-structure. Another distinction is the connecting loop (loop1) between h7 and the α-hairpin U-shaped in the cis-structure (FIG. 2a), but nearly straight in the trans-structure (FIG. 2b). The differences of the cis and trans monomers are evident by superimposing the two C-domains (FIG. 2c). It appears that a large rotation of the α-hairpin relative to h7 would be needed to generate the marked positional switch of the α-hairpin and N-globe between the two conformers.

Architecture of the N-Terminal Tier

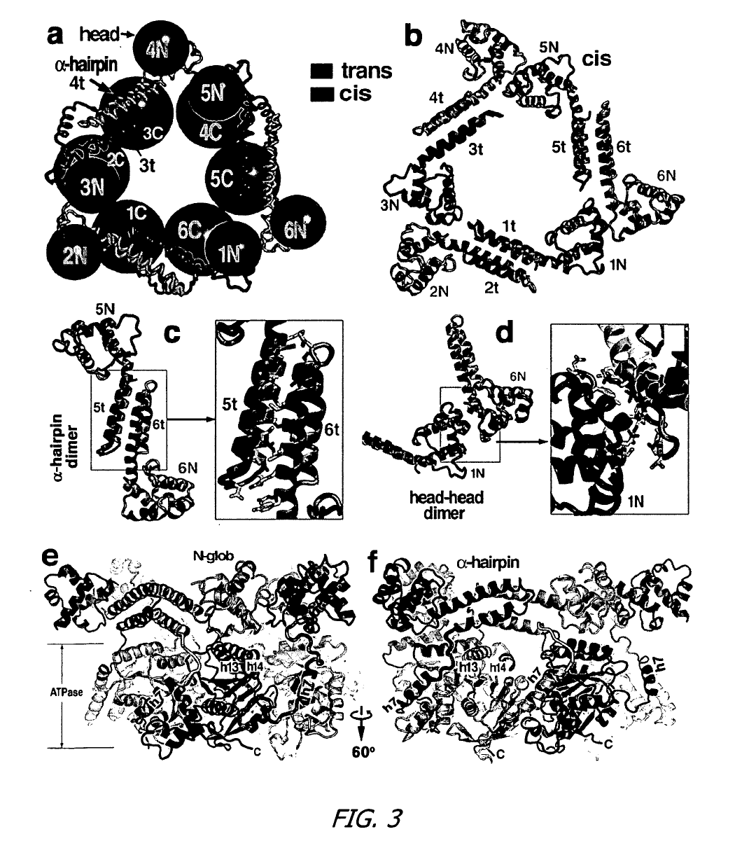

The cis and trans structures assemble in the hexameric ring in alternating arrangement (cis-monomers numbered 1, 3, 5, in purple, and trans-monomers 2, 4, 6, in blue, shown in FIG. 3a). The cis monomer 1 is positioned between two trans-monomers (2 and 6) to form the 2⇄1 and 1⇄6 interfaces. Thus, two distinct interfaces can be found within the N-tier, termed the hairpin-to-hairpin (FIG. 3b, 3c) and head-to-head dimers (FIG. 3b, 3d), both formed by paring a cis and a trans monomer. The α-hairpin dimer interface buries on average a surface area of 1,781 Å². The interface interactions are extensive and involve a total of 32 residues (FIG. 3c). In contrast to the extensive hairpin-to-hairpin interface, the head-to-head interaction has a relatively small interface burying on average 922 Å², with 19 residues making bonding contacts (FIG. 3d).

Hexamerization of the ATPase Domain

Because no strict symmetry exists along the hexameric channel, each of the six interfaces between ATPase domains within the C-tier is quite different. As a result, the surface area buried ranges from 2,243 Å² at the smallest interface to 3,122 Å² at the largest interface within a C-terminal ring (including the helix 7 and the entire ATPase domain, residues 158 to 436), suggesting plasticity in interactions between C-terminal ATPase domains. The h7 at the N-terminus of the ATPase domain also plays a role in holding the ATPase ring together (FIG. 3e-f). The h7 extends out like an invading arm to fit into a groove on the adjacent ATPase domain. Furthermore, this h7 arm projects its N-terminal α-hairpin and N-globe over the adjacent monomer (FIG. 3e-f, cyan monomer to red, or red monomer to yellow), which pack with the neighboring ATPase domain. Thus, h7 also acts like a bridge for a domain swap. Comparison of the six monomers in the hexamer structure in the region of h7 shows this hinged arm emanates from its own ATPase domain with different angles, largely due to the flexibility provided by two glycines (G173 and G177) on the loop connecting h7 to the ATPase domain. This loop's intrinsic flexibility allows h7 to grip the neighboring monomer when the interface area changes between adjacent ATPase domains, possibly facilitating conformational changes of the hexamer required for DNA unwinding.

Around the ATP binding pocket of the full-length G40P hexamer, crystallized in the absence of nucleotide, the critical Arg finger (R414) from the neighboring subunit is pointing away from the p-loop, which is similar to the empty site of T7 gp4 and SV40 large T helicases²⁶⁻²⁸. In contrast, in the G40P-Δ129 (FIGS. 6a, 6b) structure, crystallized as a complex with ATPγS, the residues involved in binding ATP, in particular the Arg finger (R414) together with K412, contact with the triphosphate groups of the nucleotide.

Besides the N—N and C—C intra-tier domain interactions, there are also inter-tier contacts, which are characterized by two types of N-to-C domain packing interactions. In one of these inter-tier contacts, an N-globe from a cis-monomer rests on the ATPase domain of an adjacent trans-monomer (FIG. 3e, cyan N-globe with red ATPase domain). The second N—C contact involves the α-hairpin of a trans-monomer and the ATPase domain from an adjacent cis-monomer (FIG. 3f, blue α-hairpin with cyan ATPase domain). These two N-to-C inter-tier contacts are comprised of mostly hydrophobic residues. Both of these packing interactions may be critical for proper inter-tier communication, as we found they have an unexpected role in helicase function and for the interplay with DnaG primase (discussed below).

N-Terminal Requirement for Helicase Activity and Primase Binding

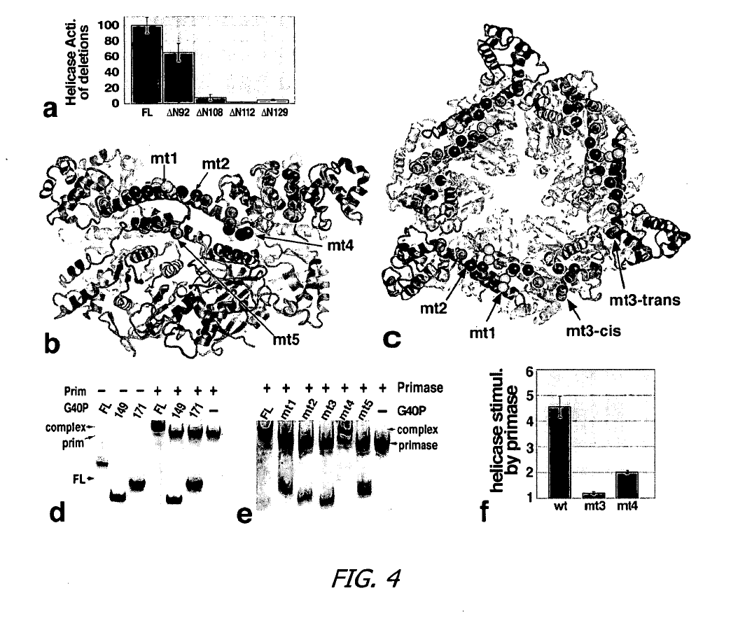

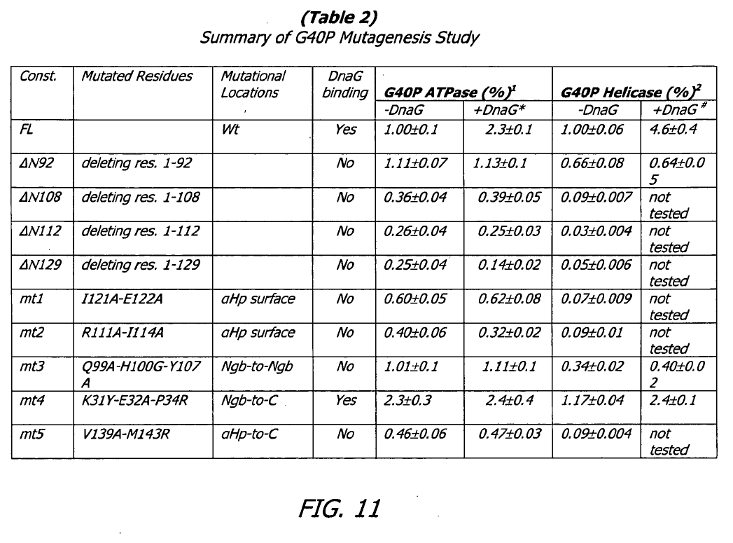

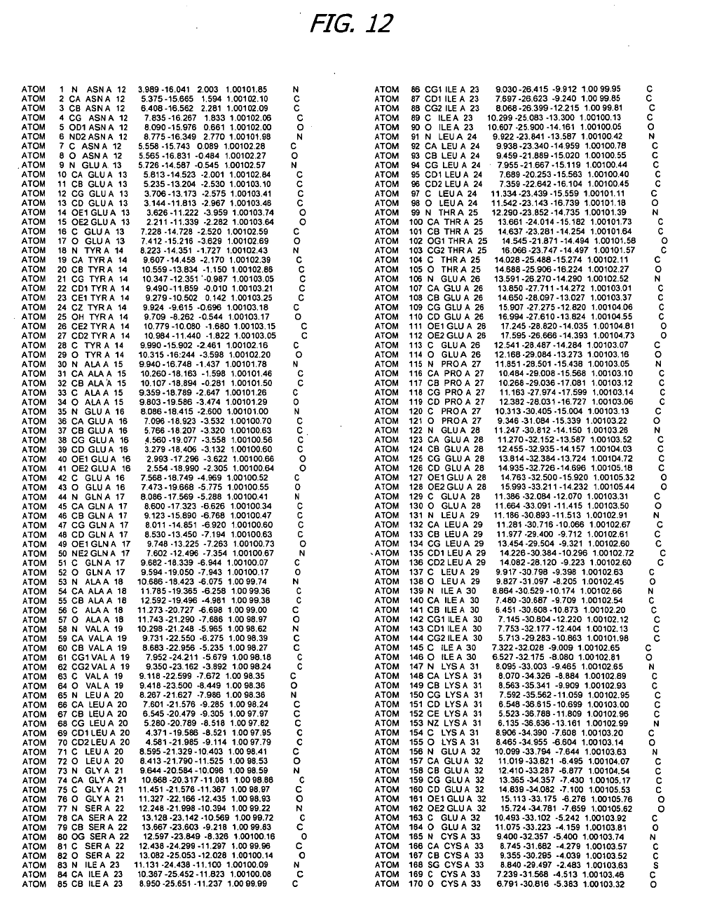

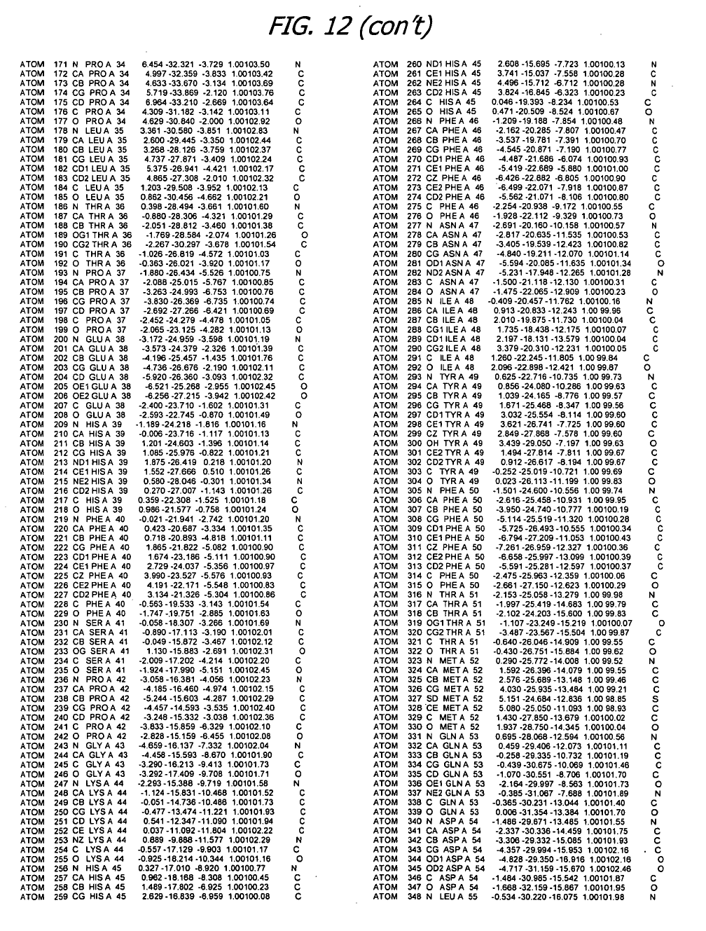

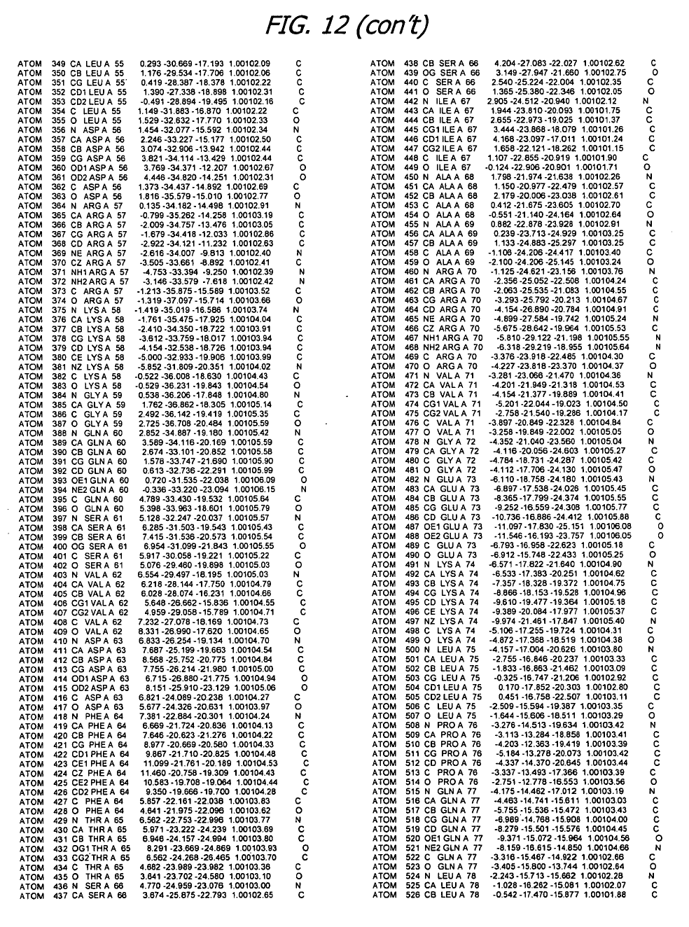

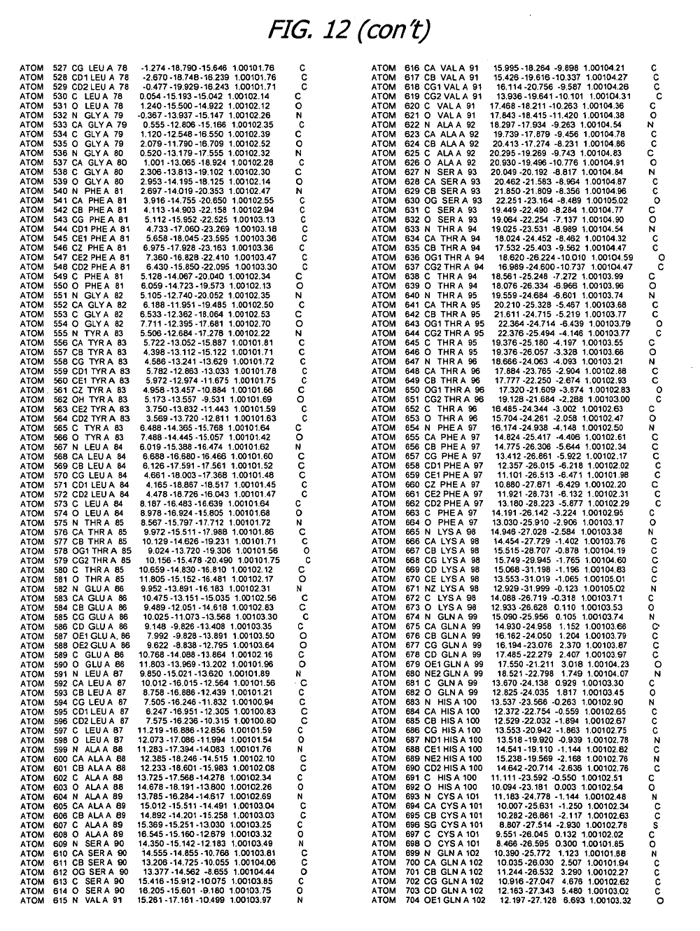

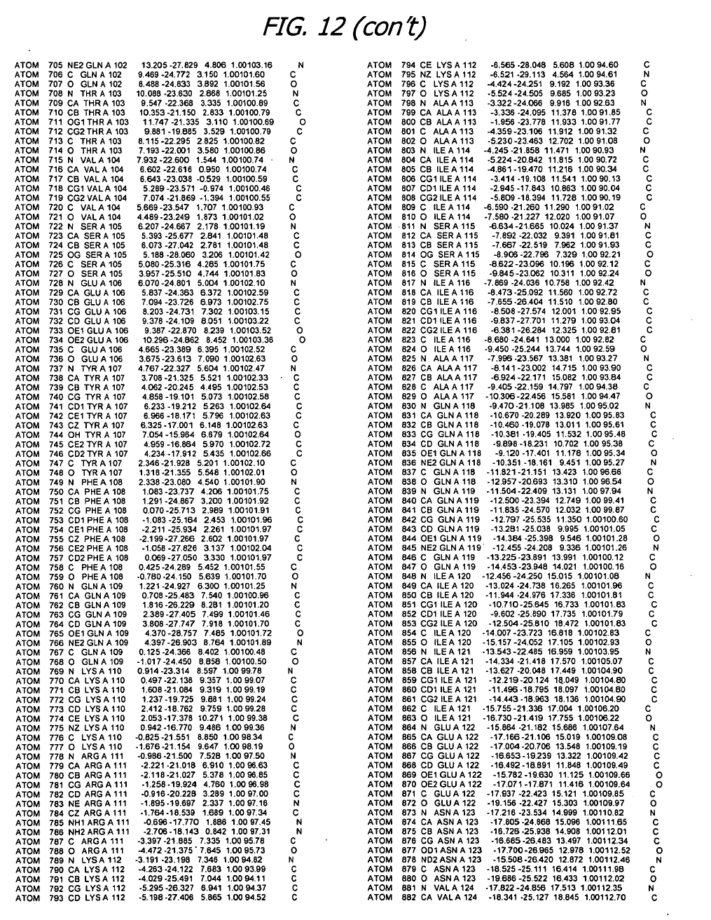

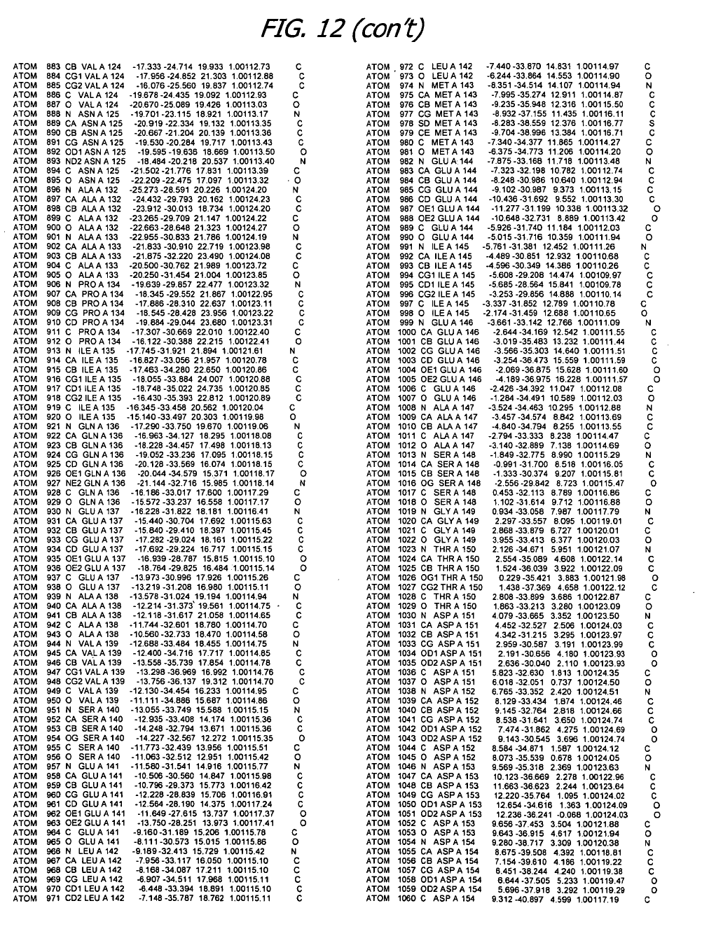

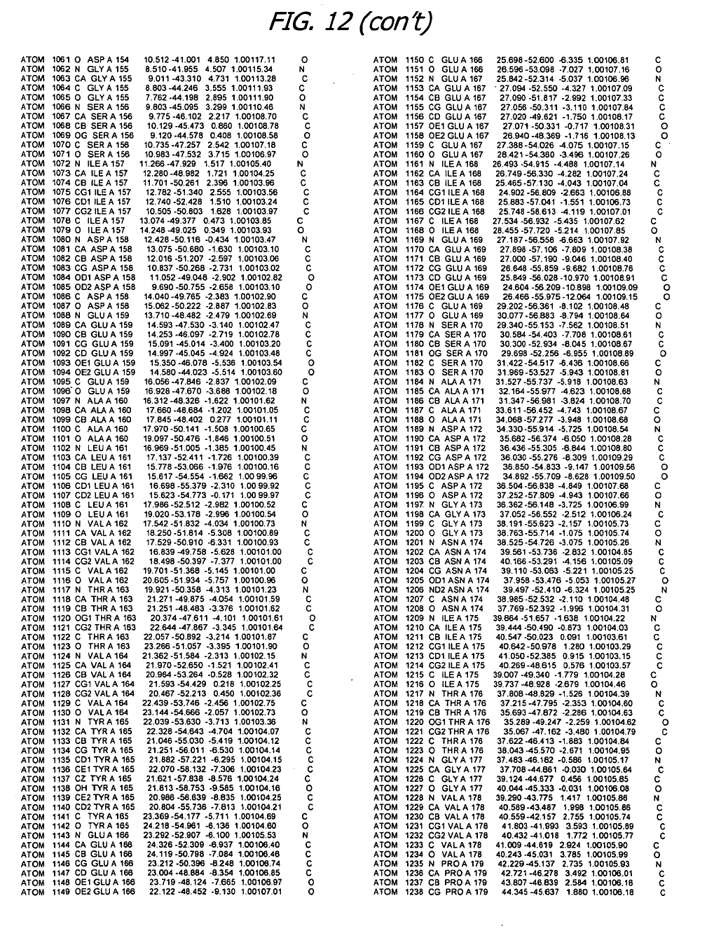

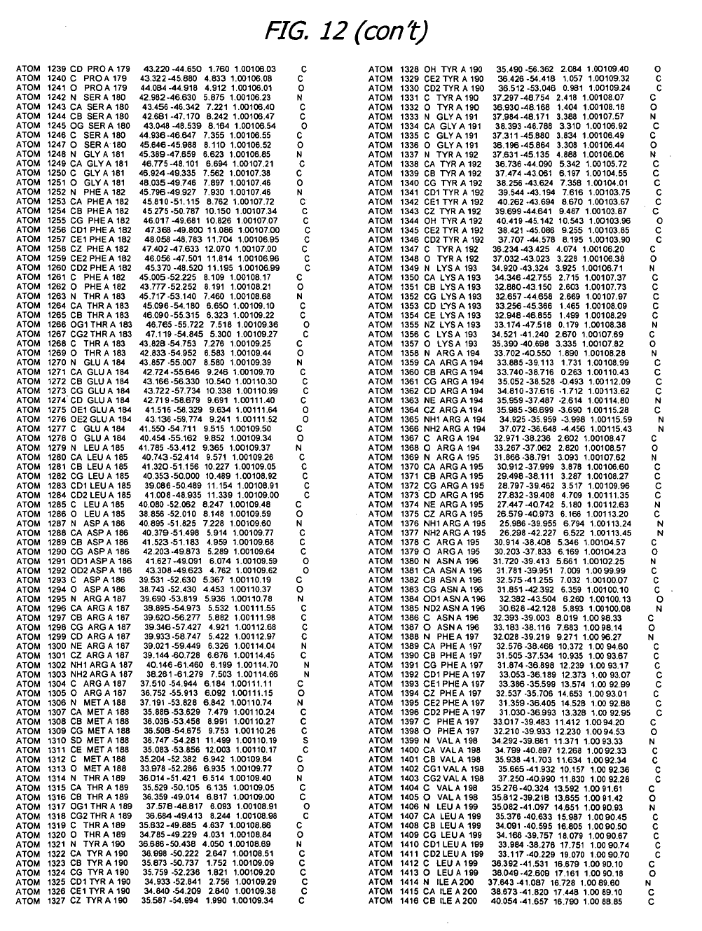

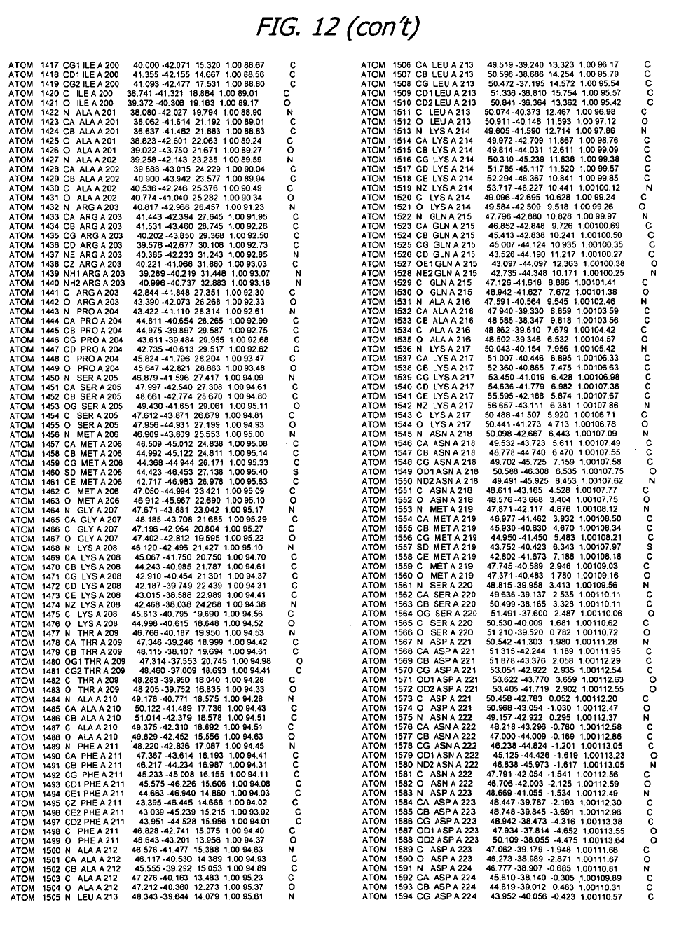

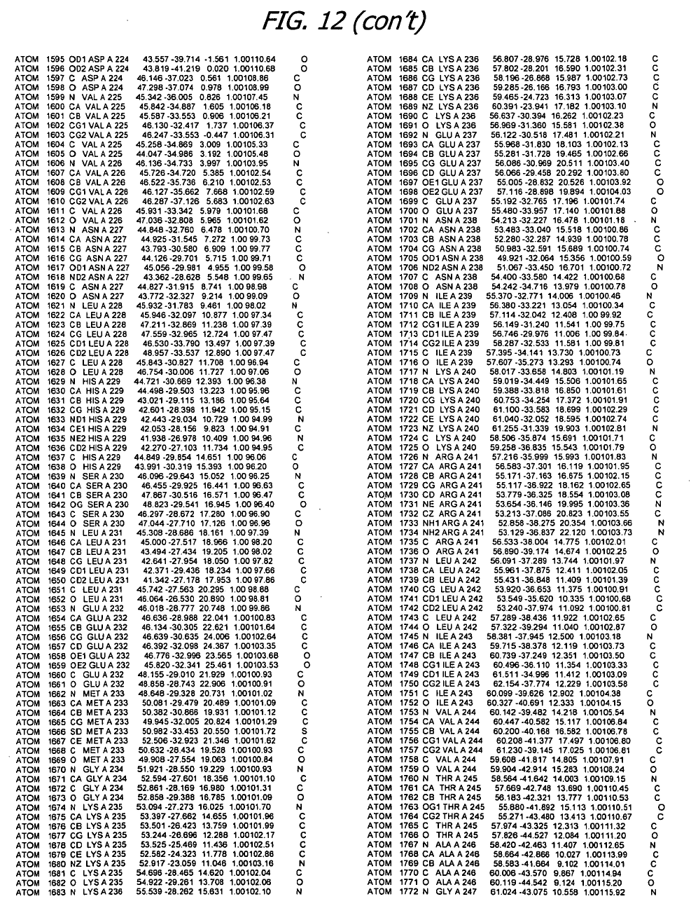

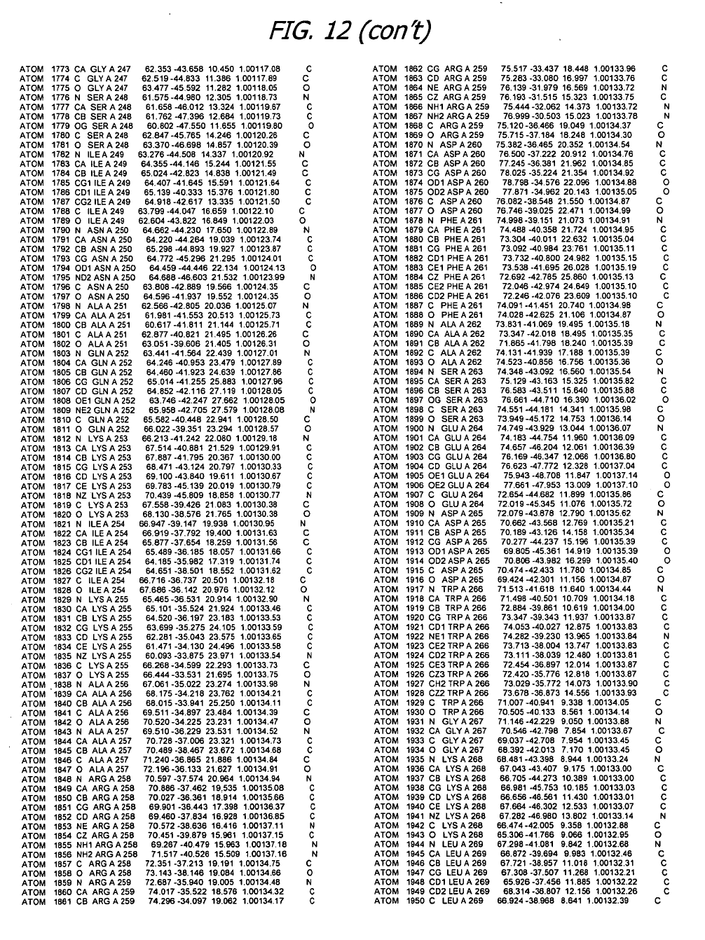

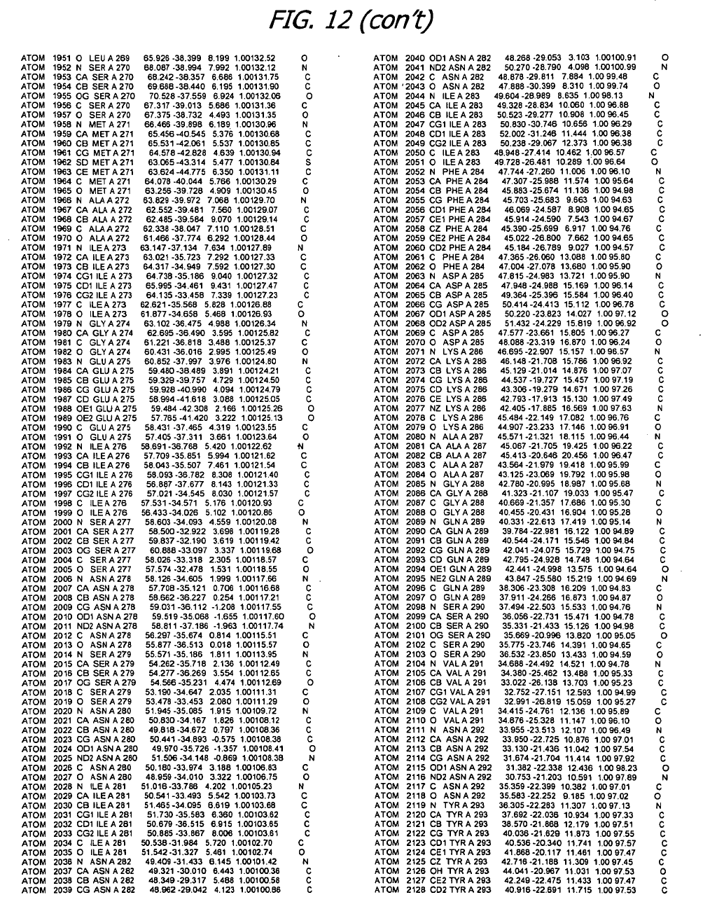

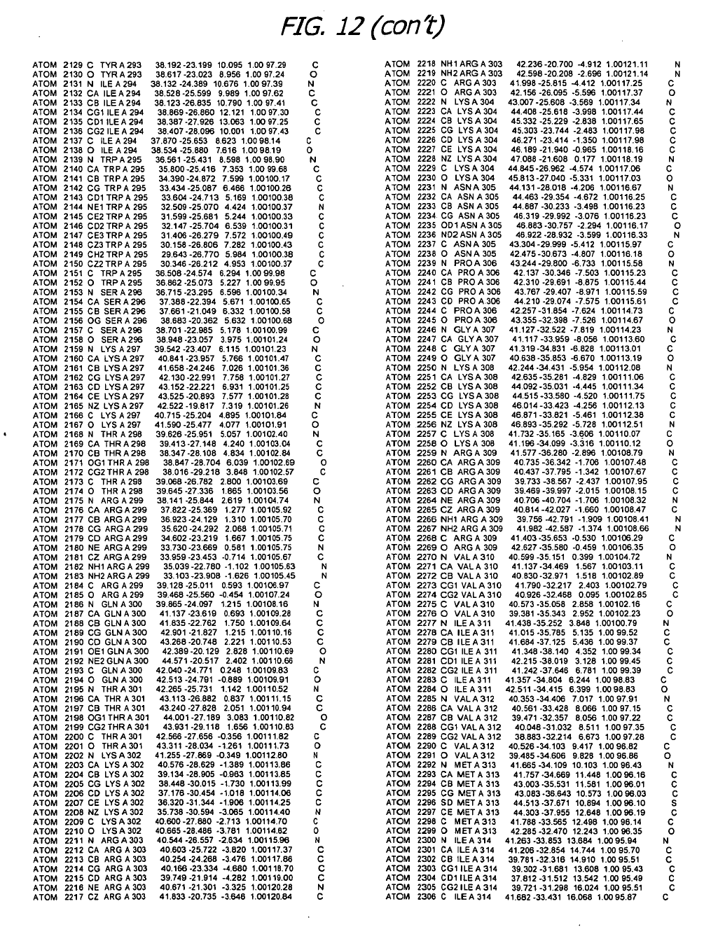

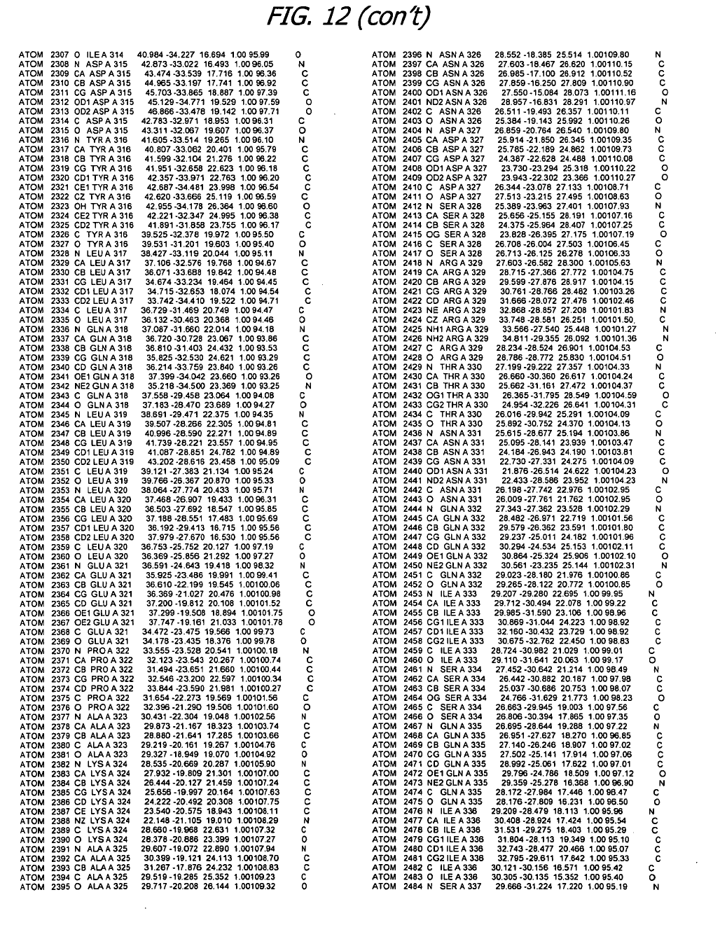

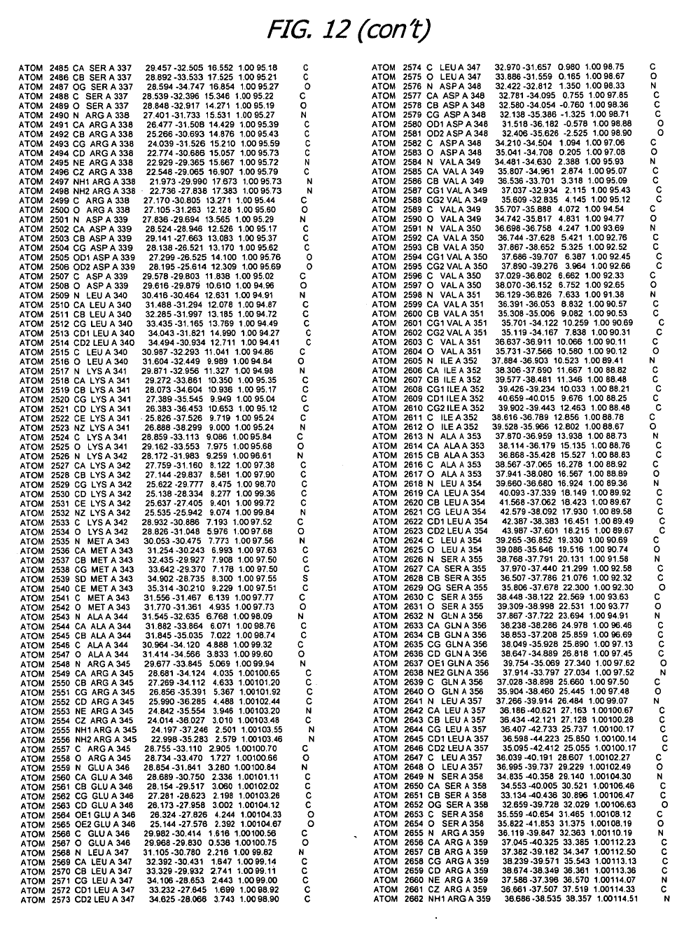

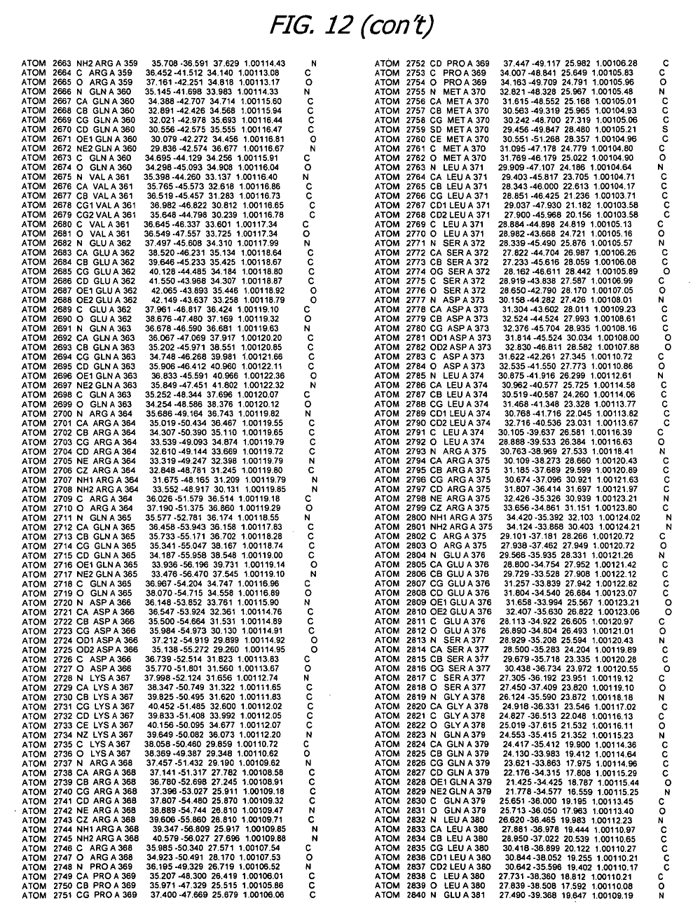

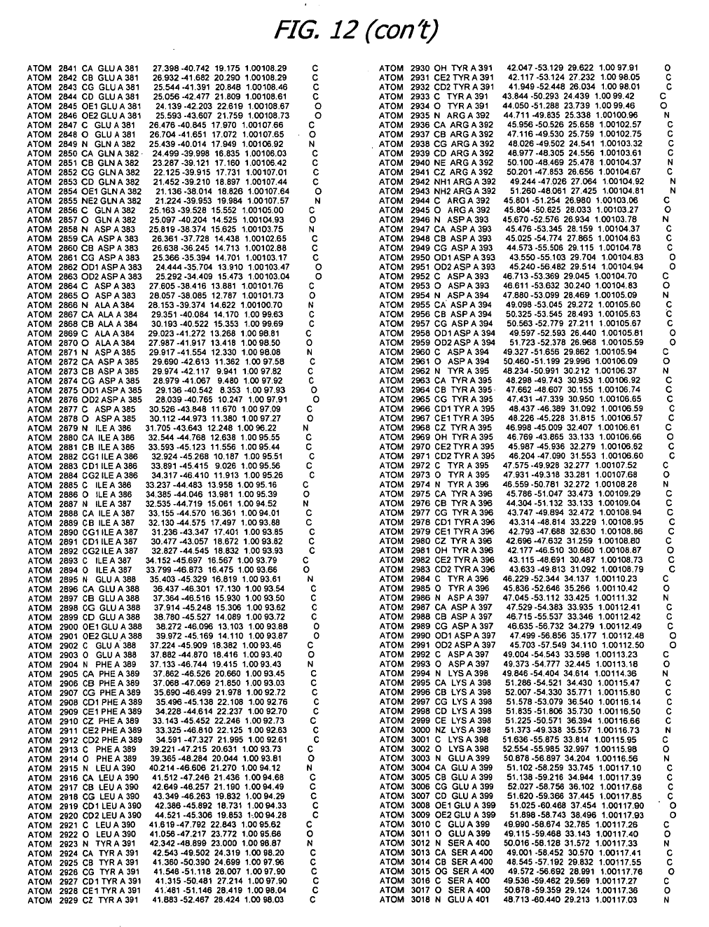

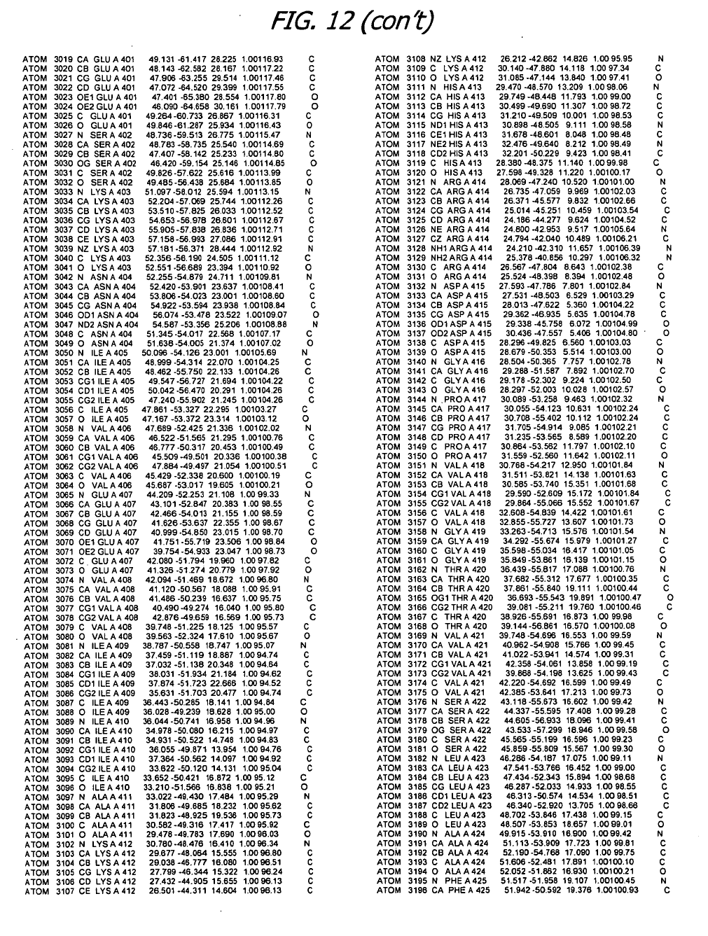

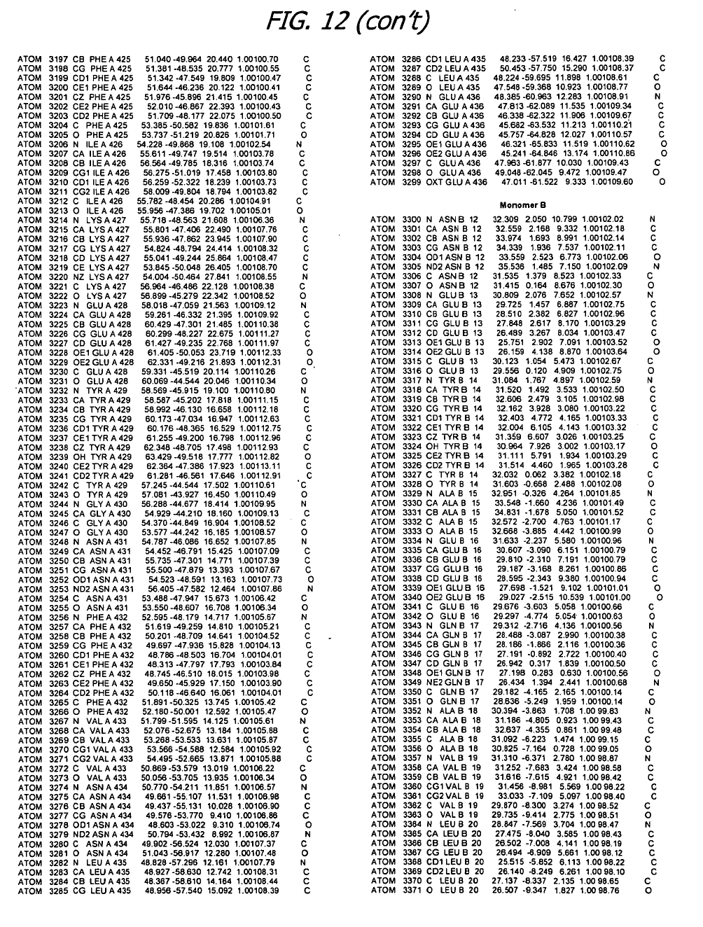

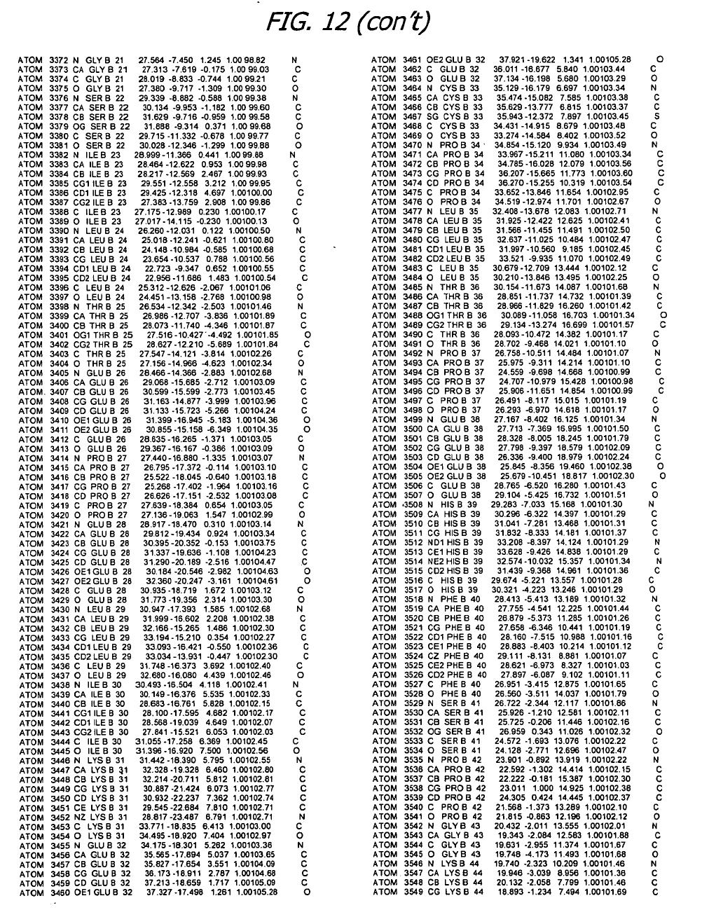

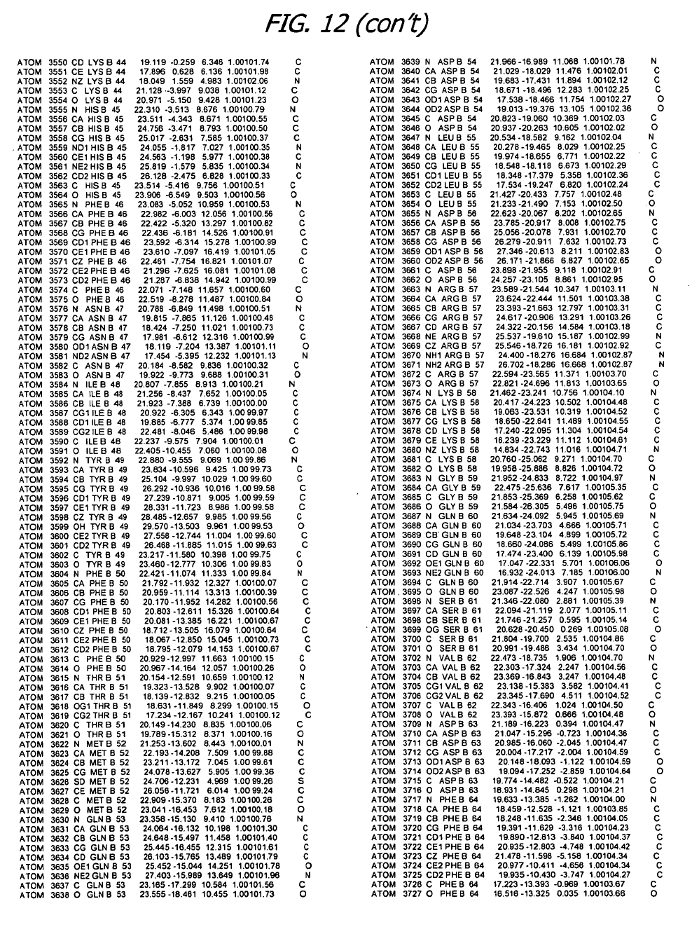

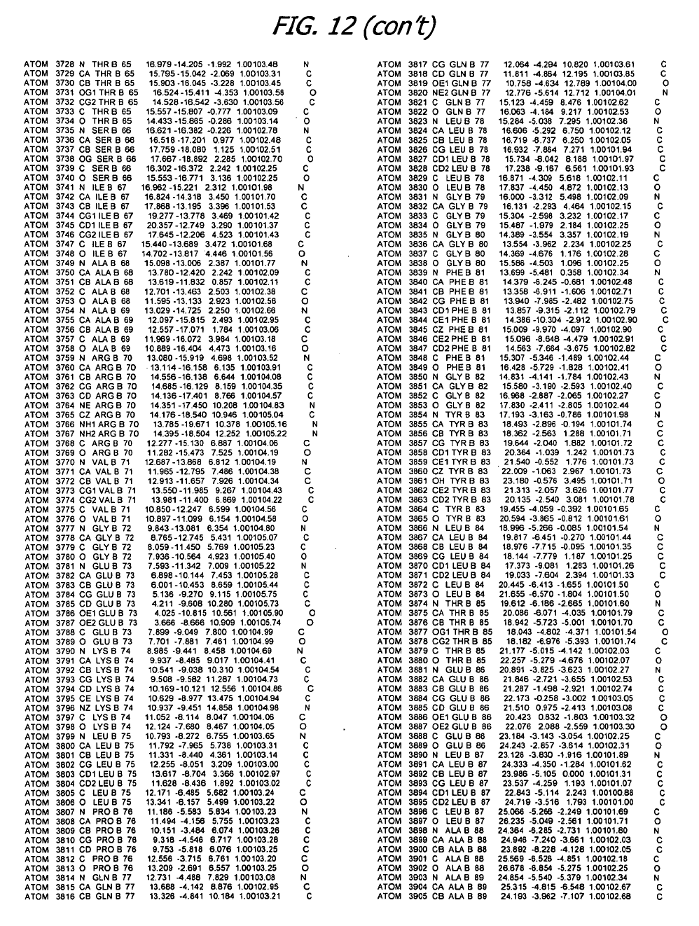

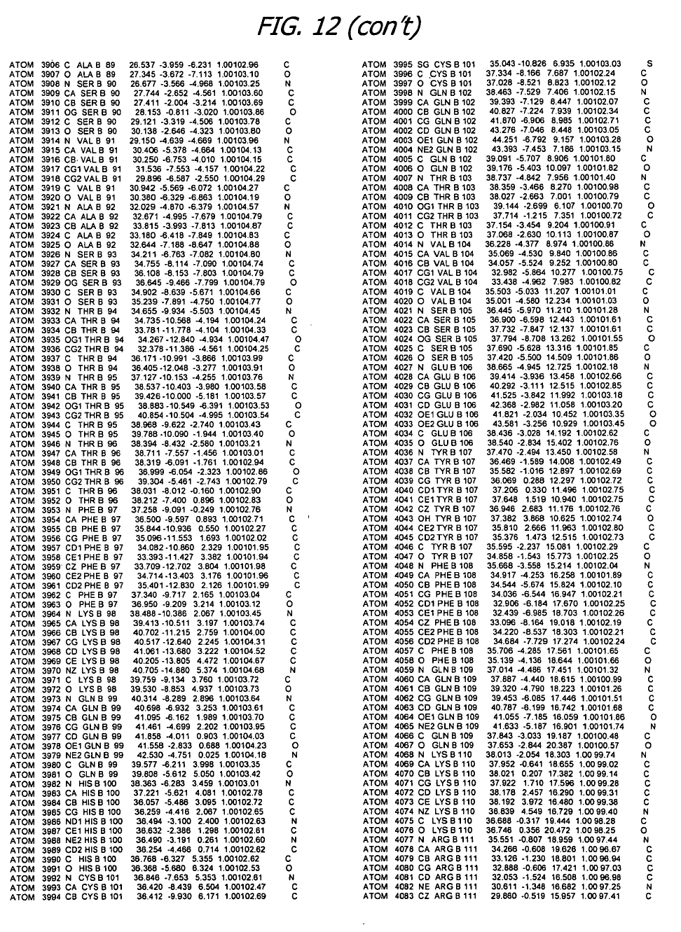

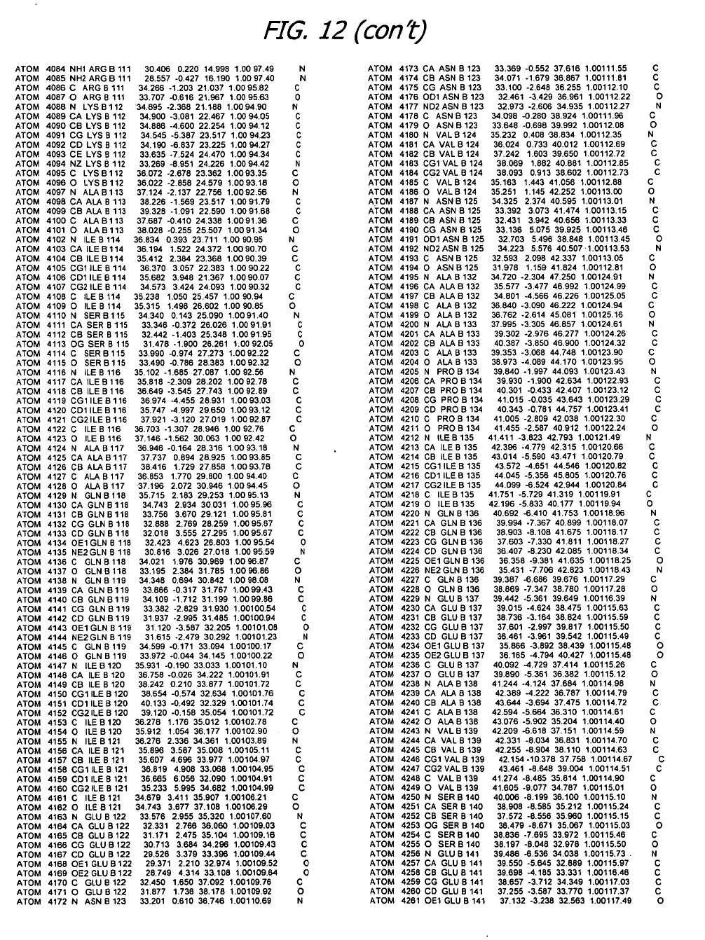

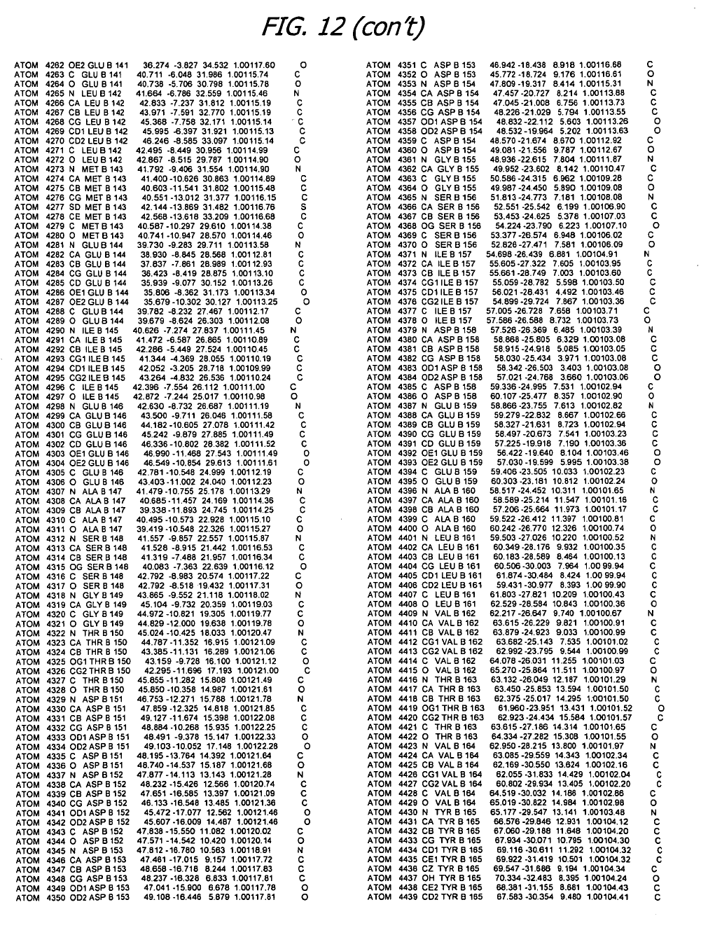

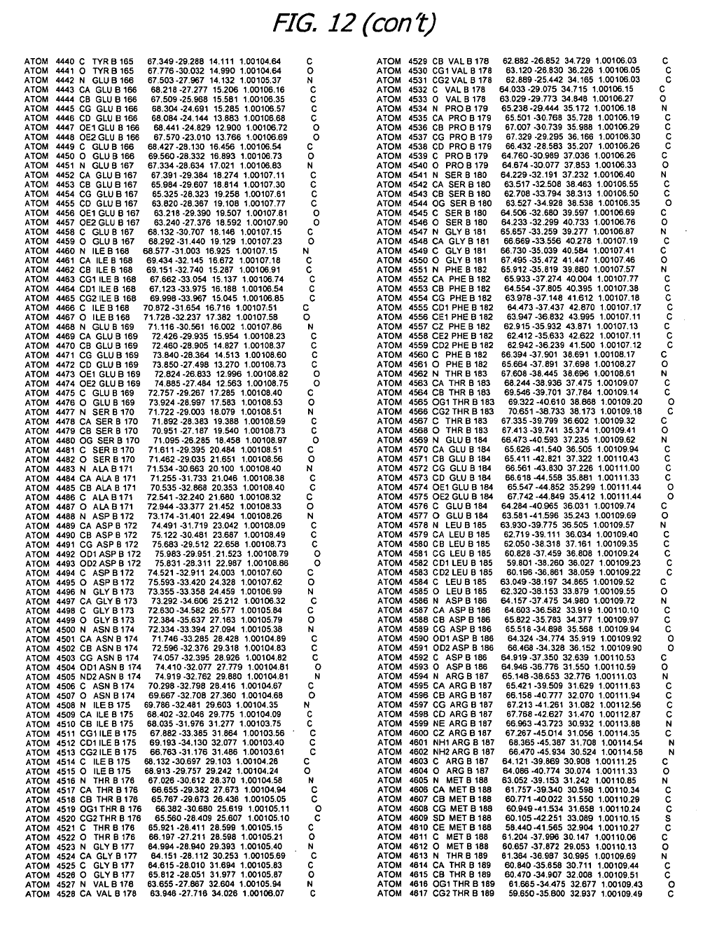

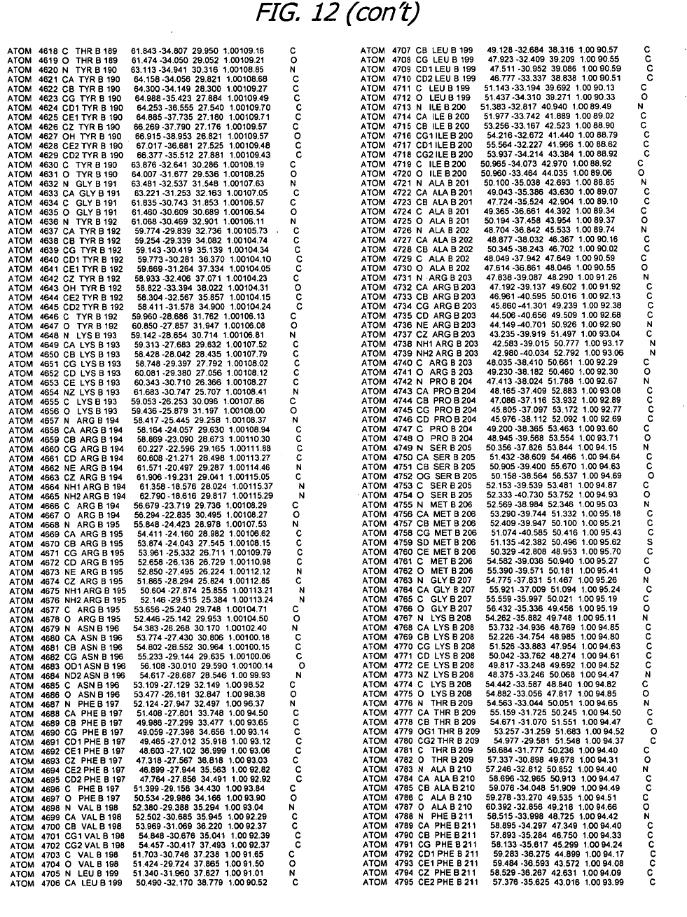

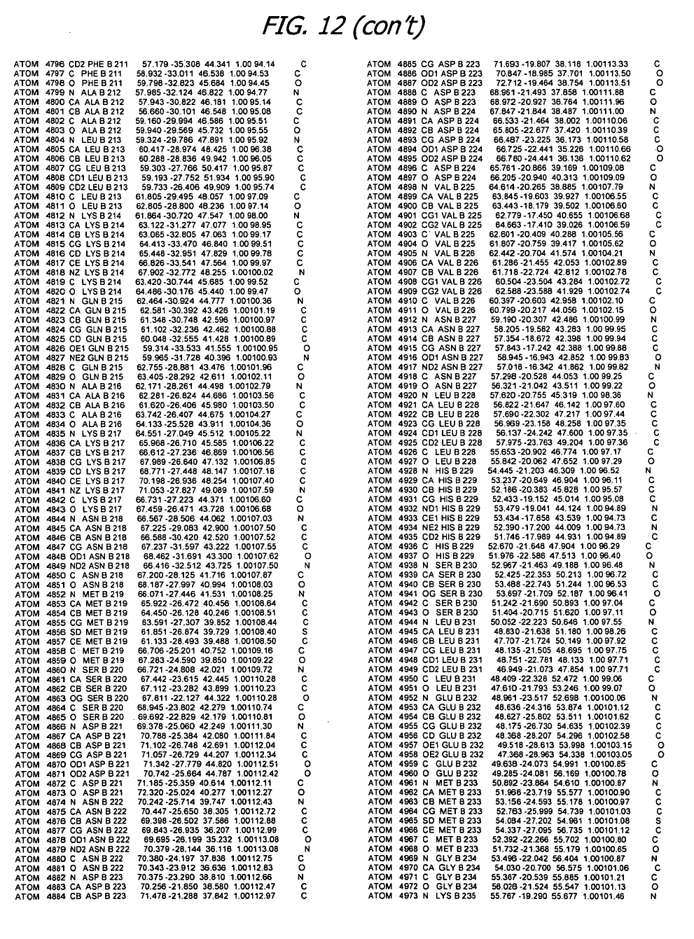

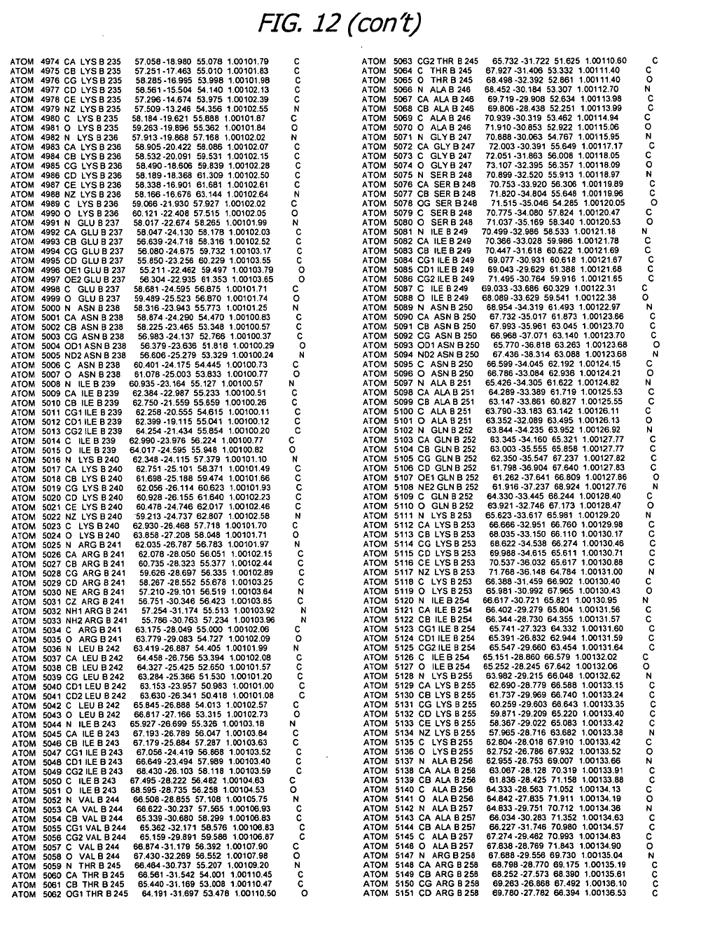

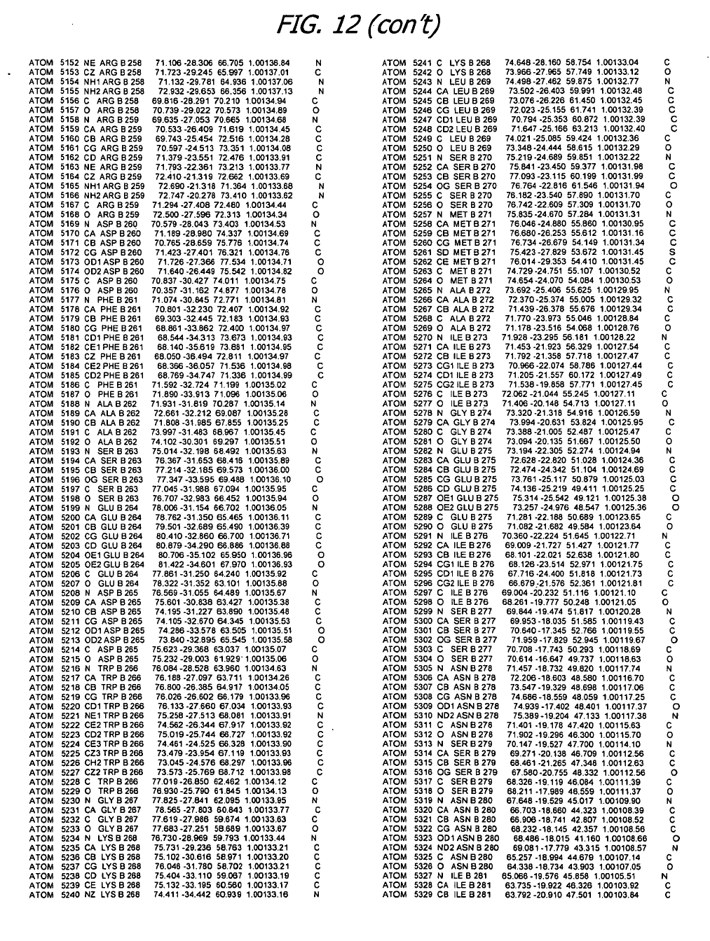

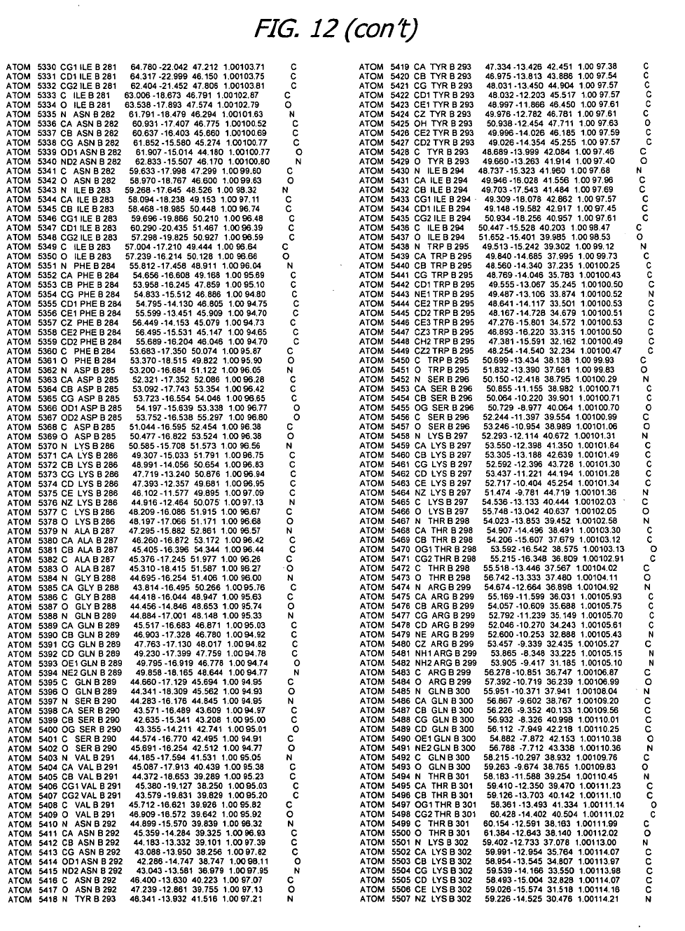

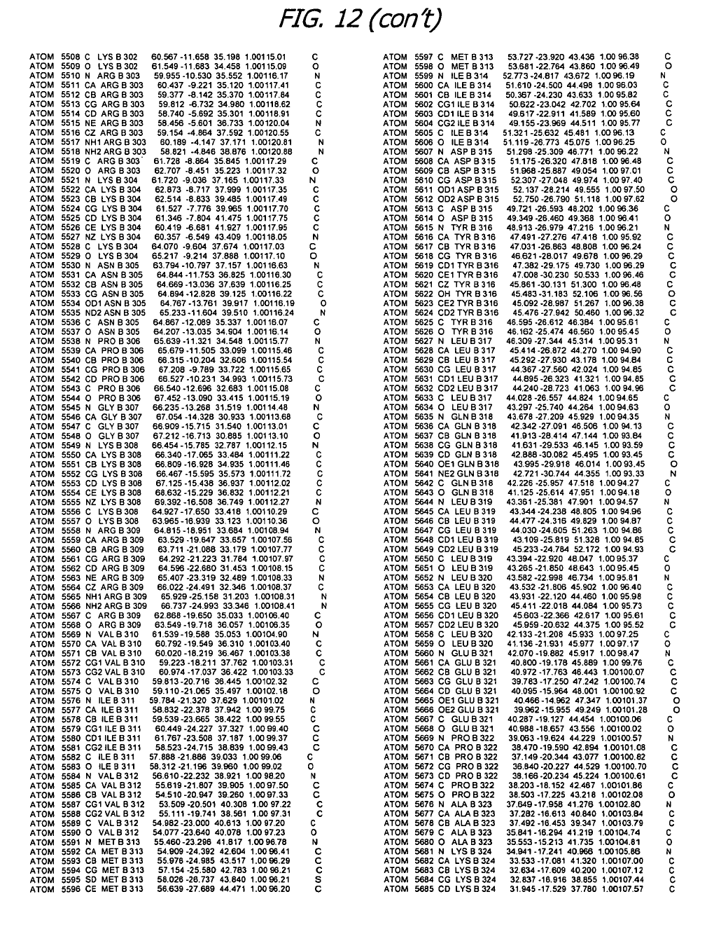

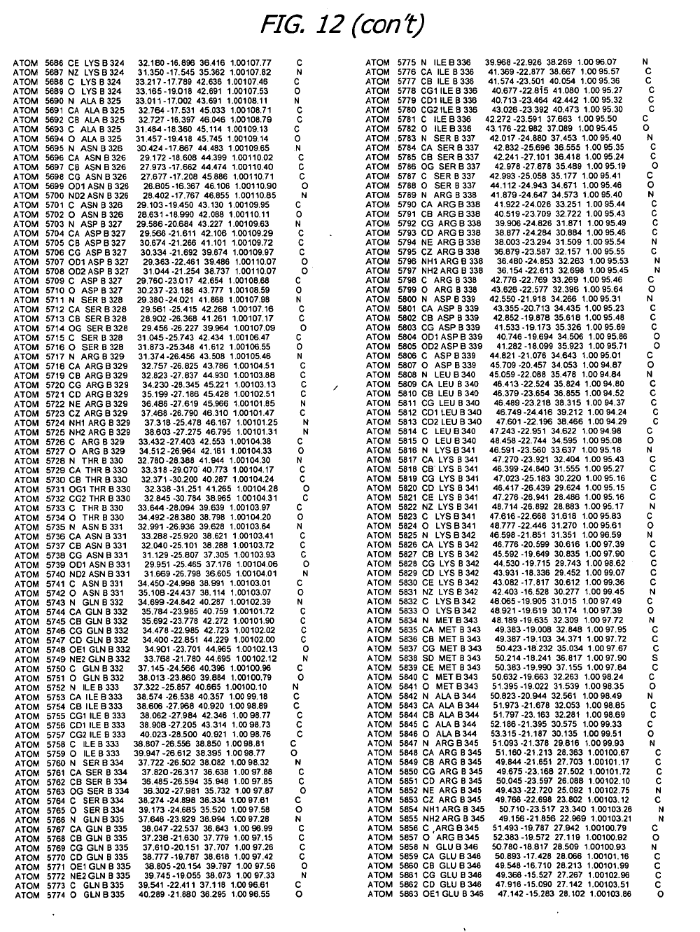

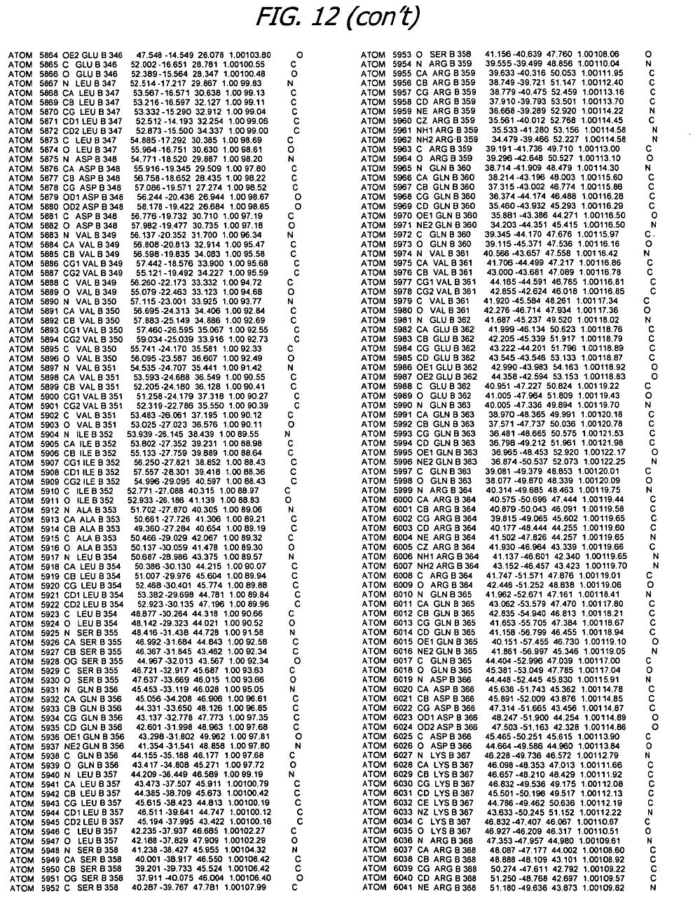

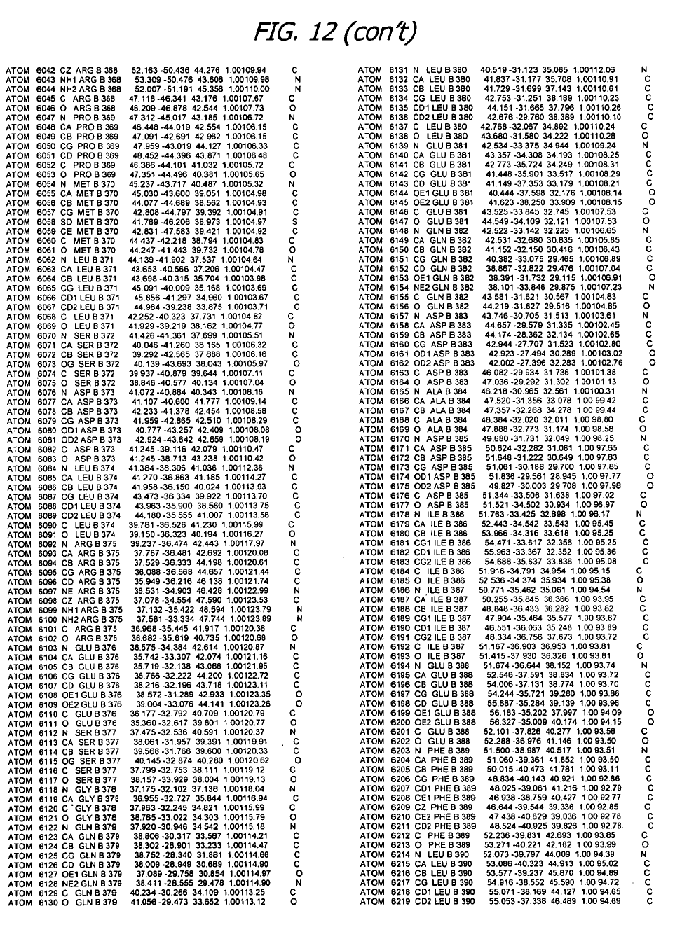

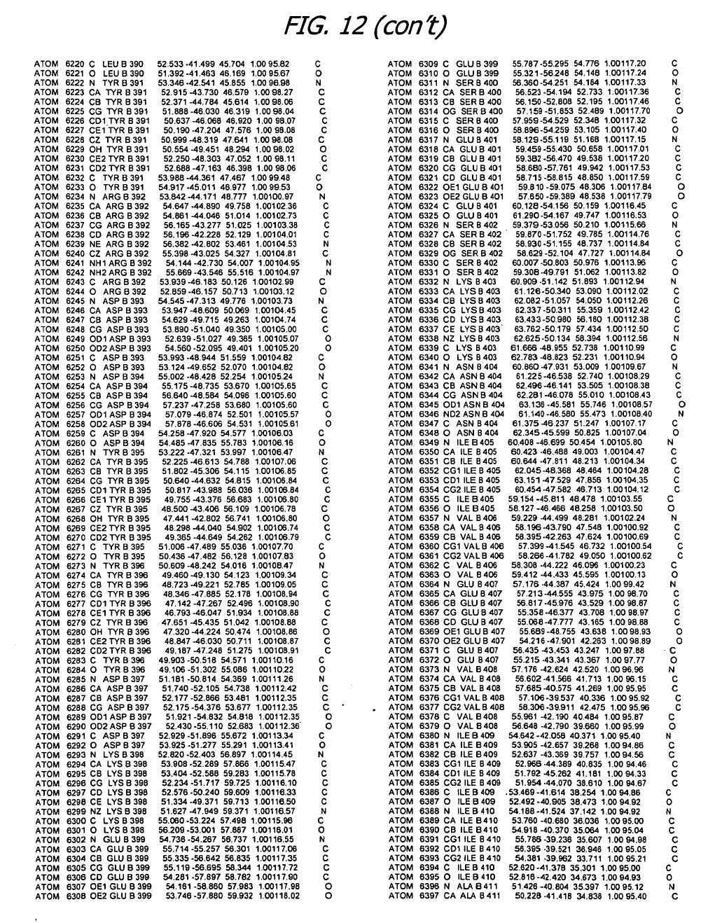

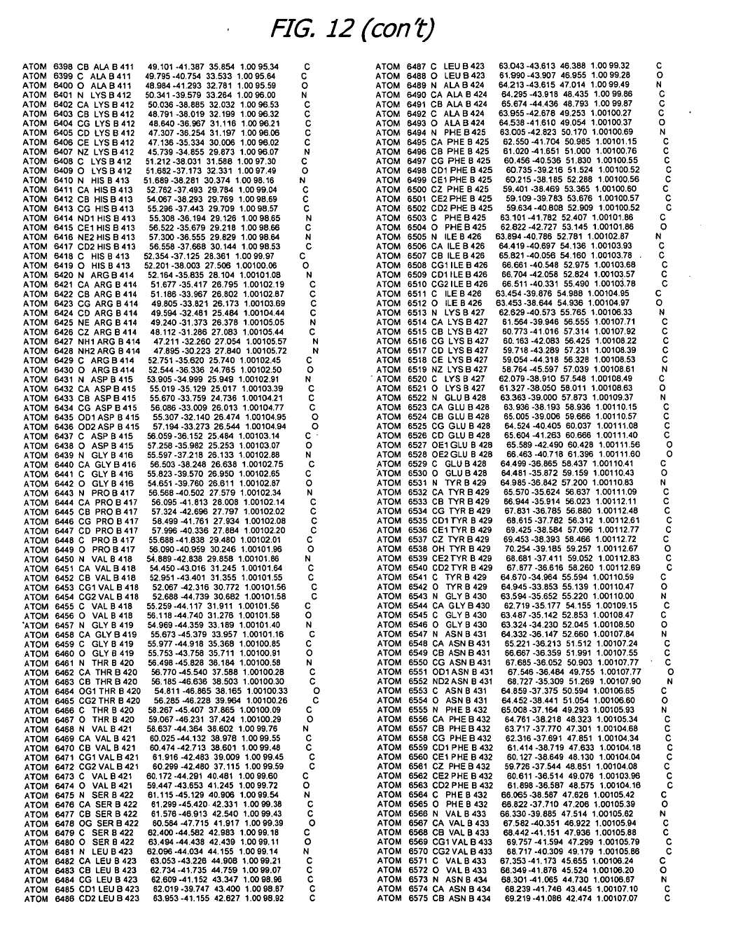

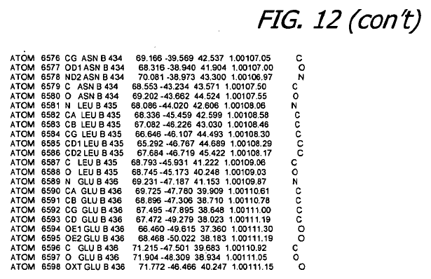

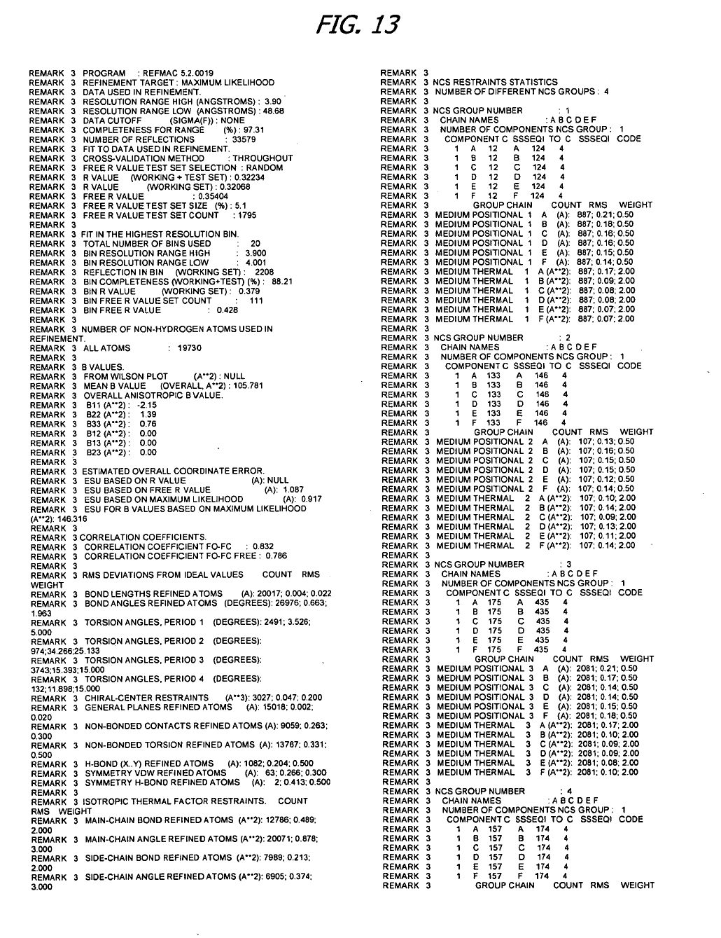

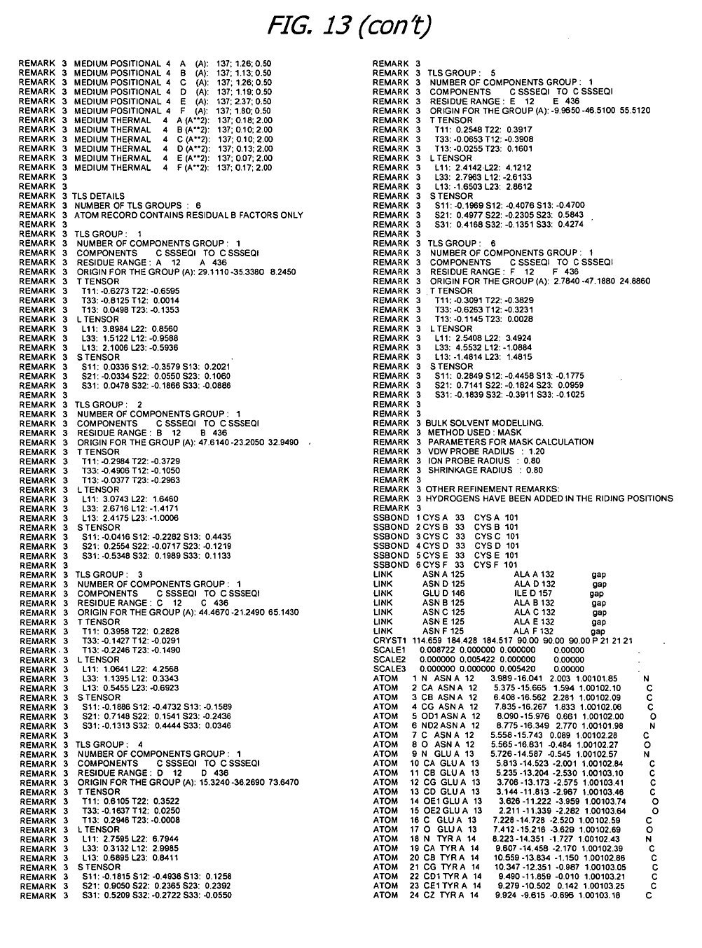

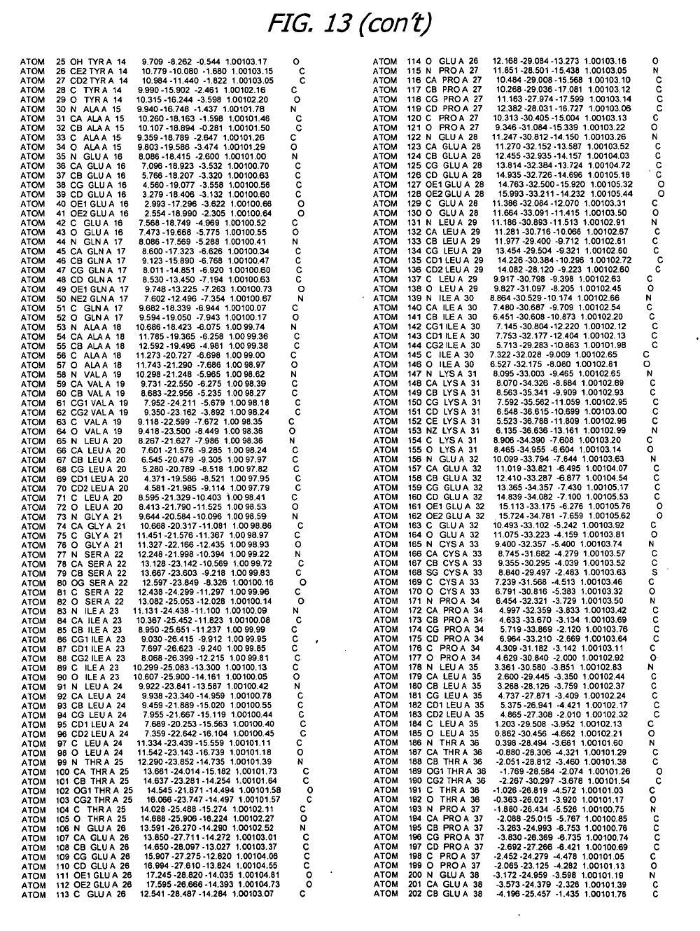

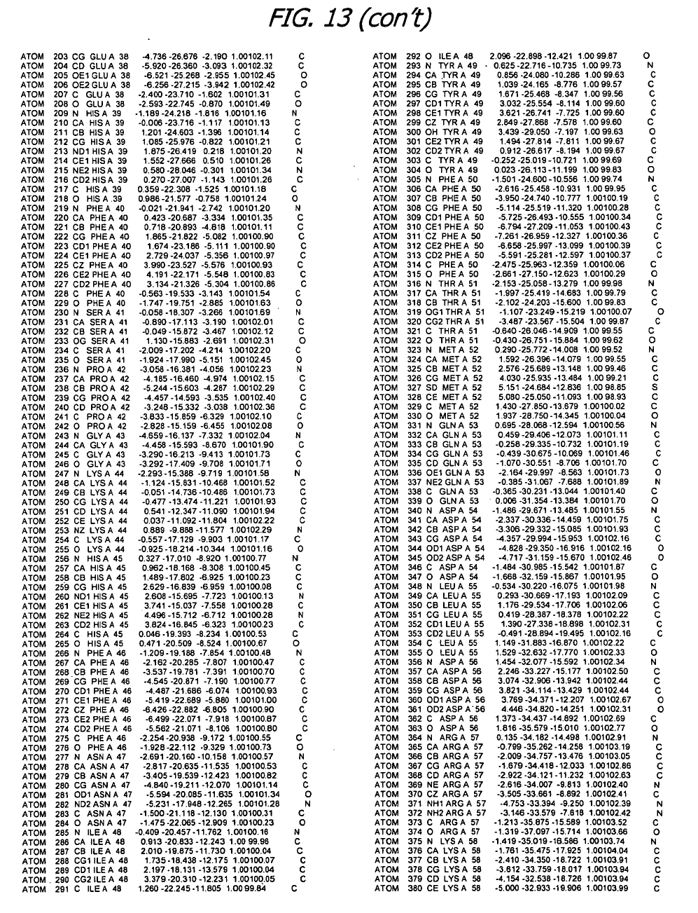

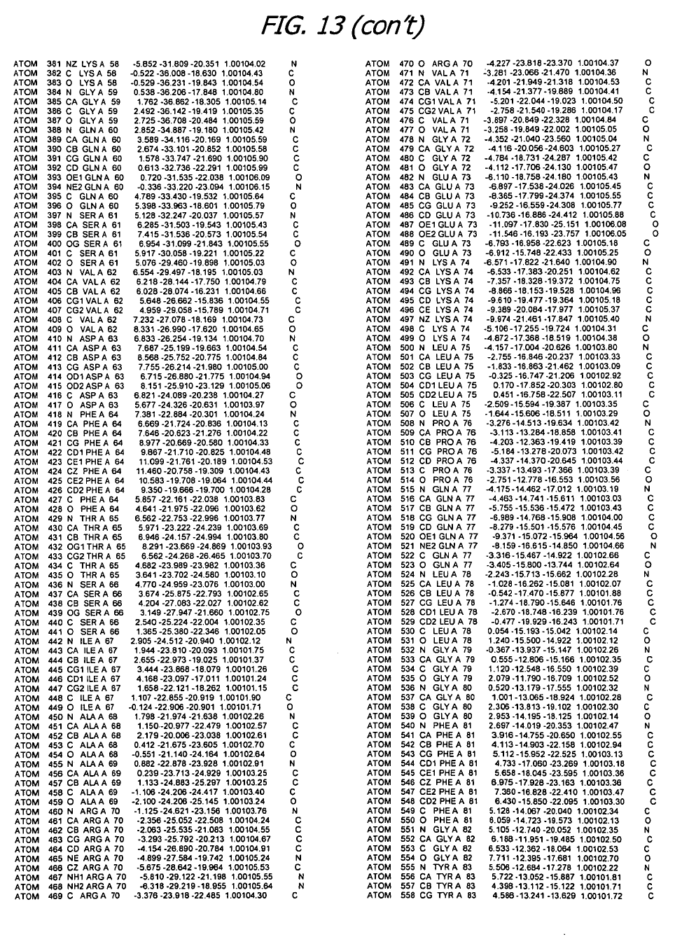

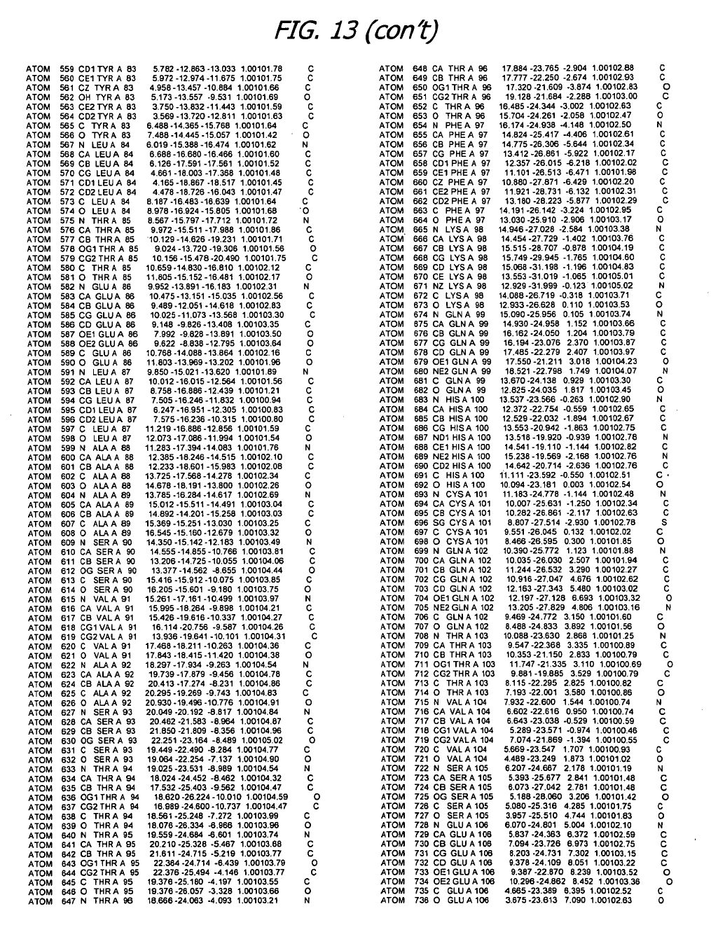

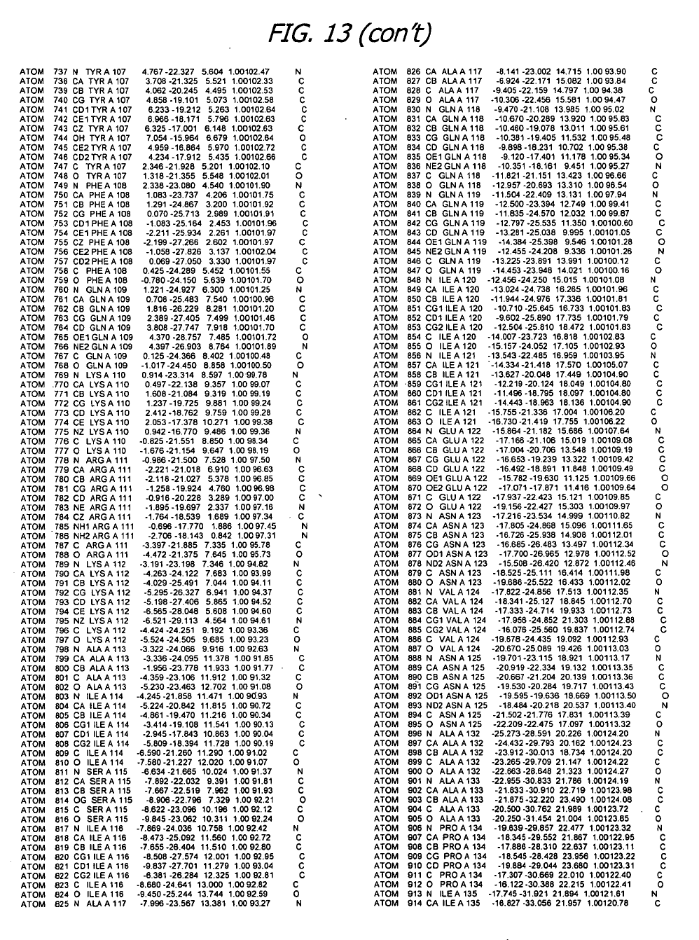

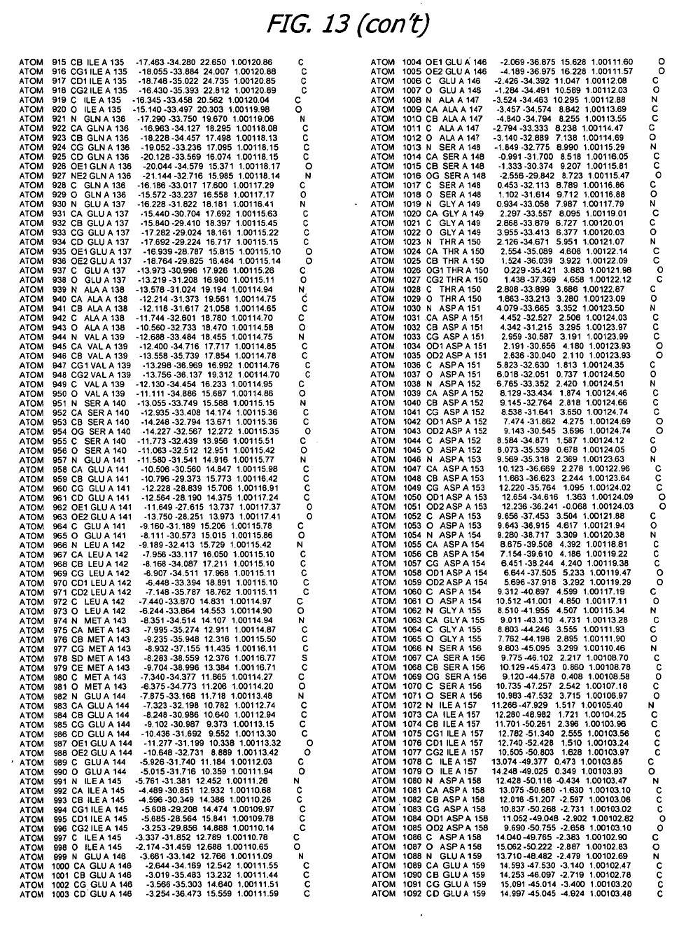

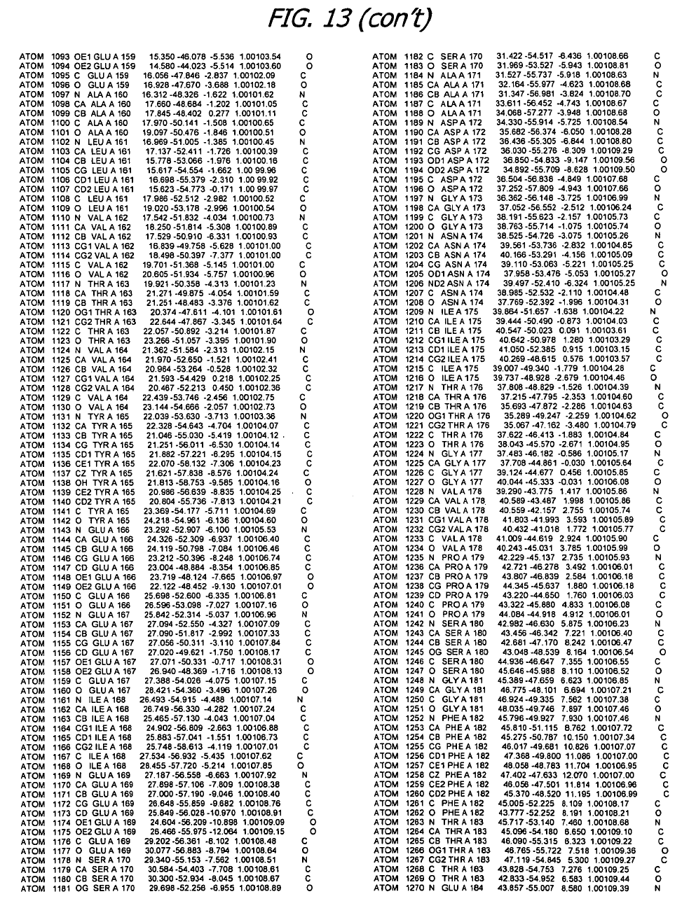

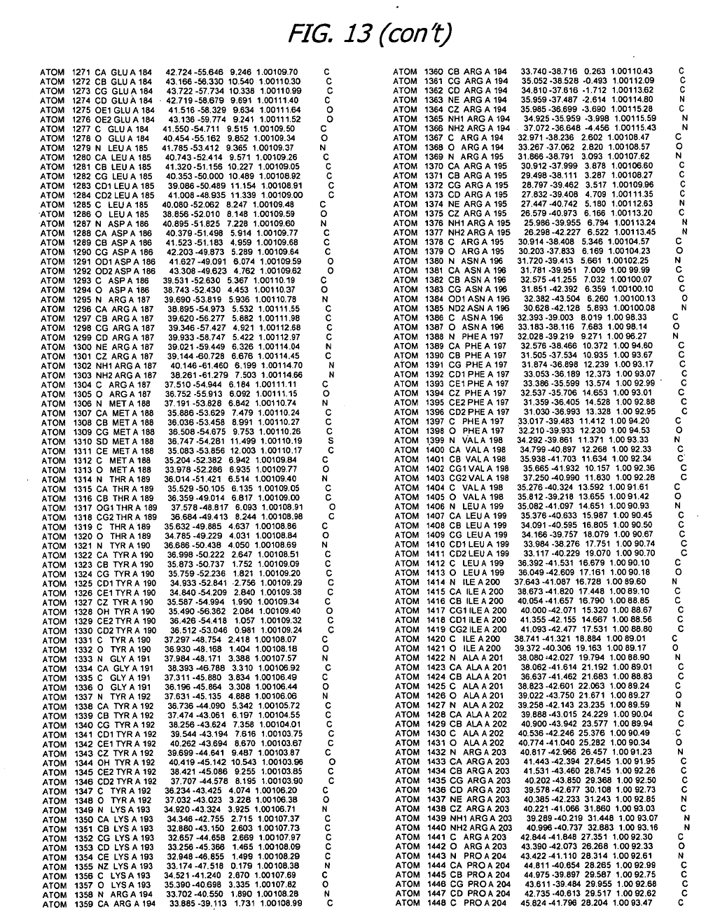

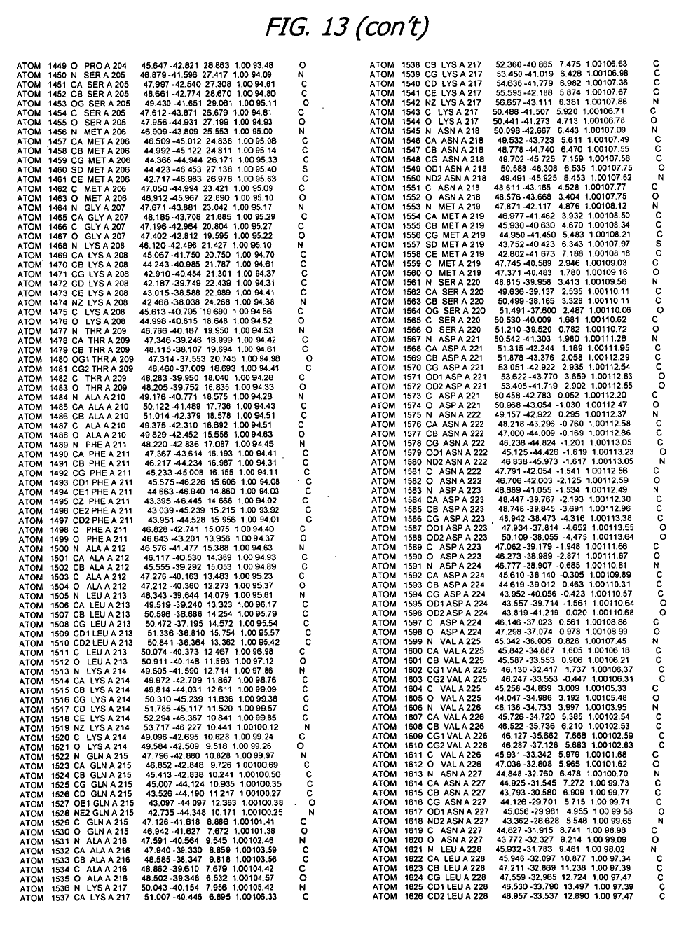

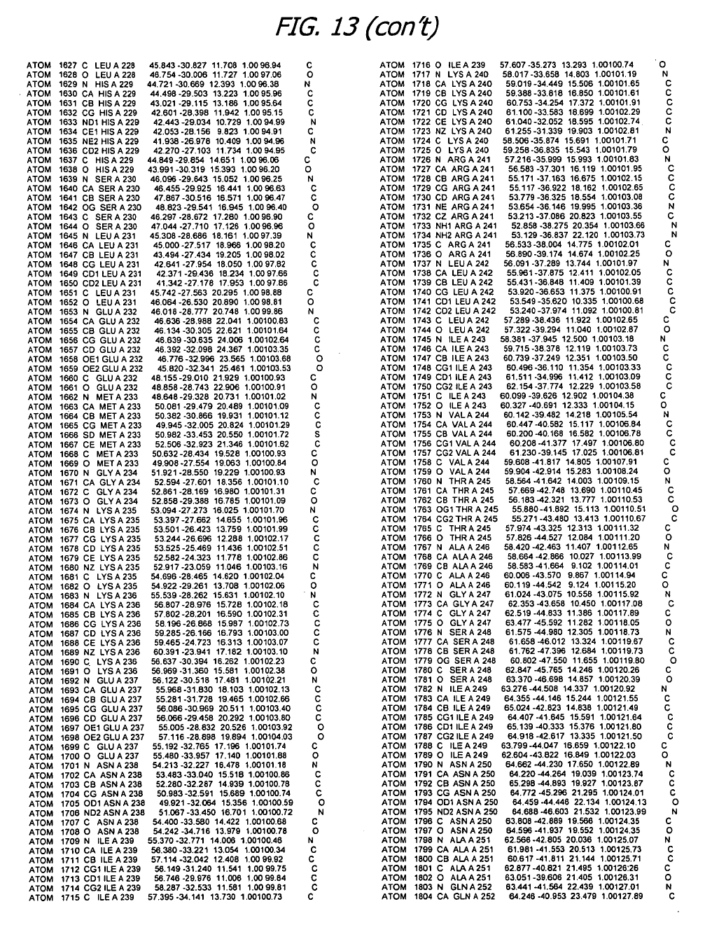

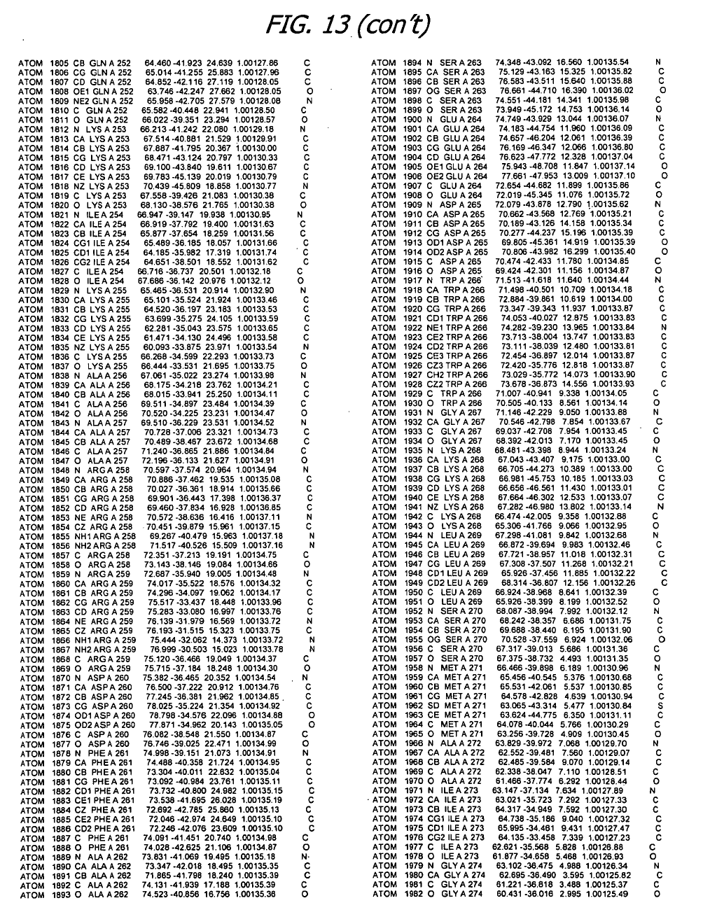

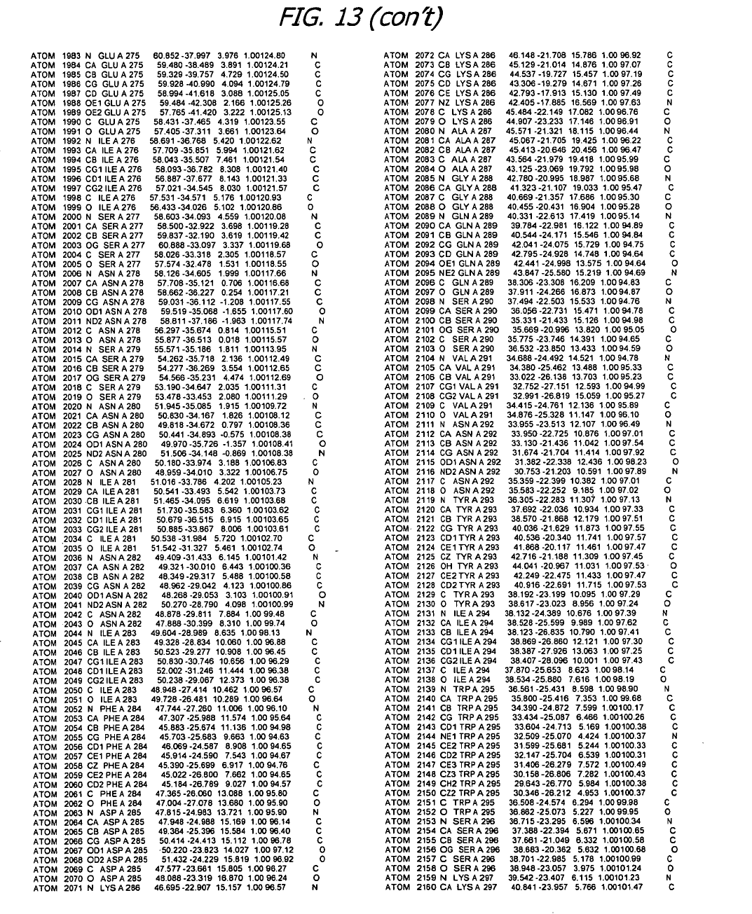

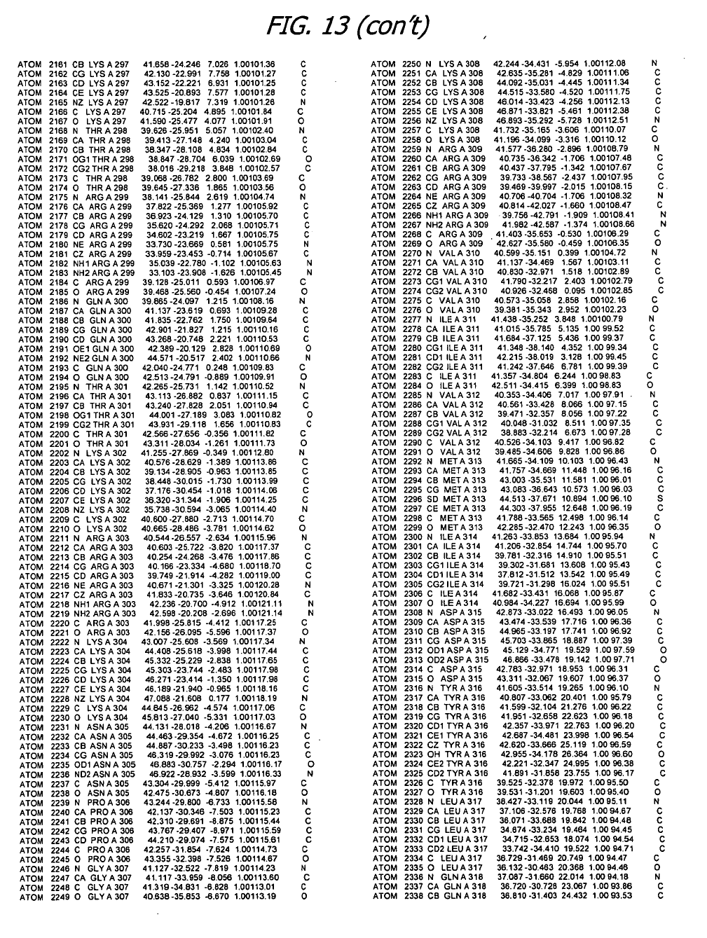

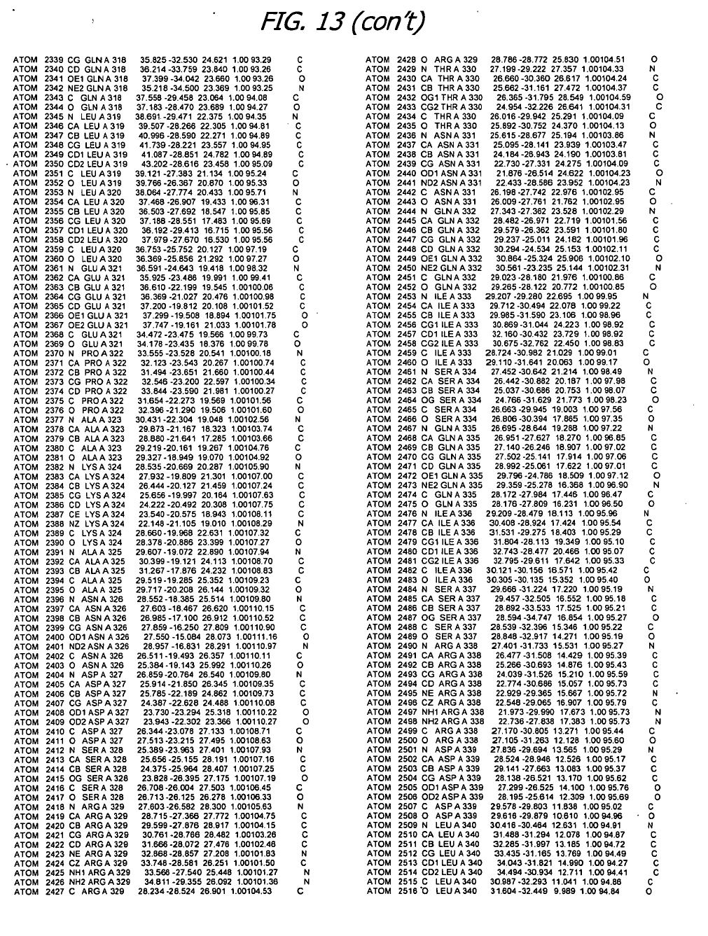

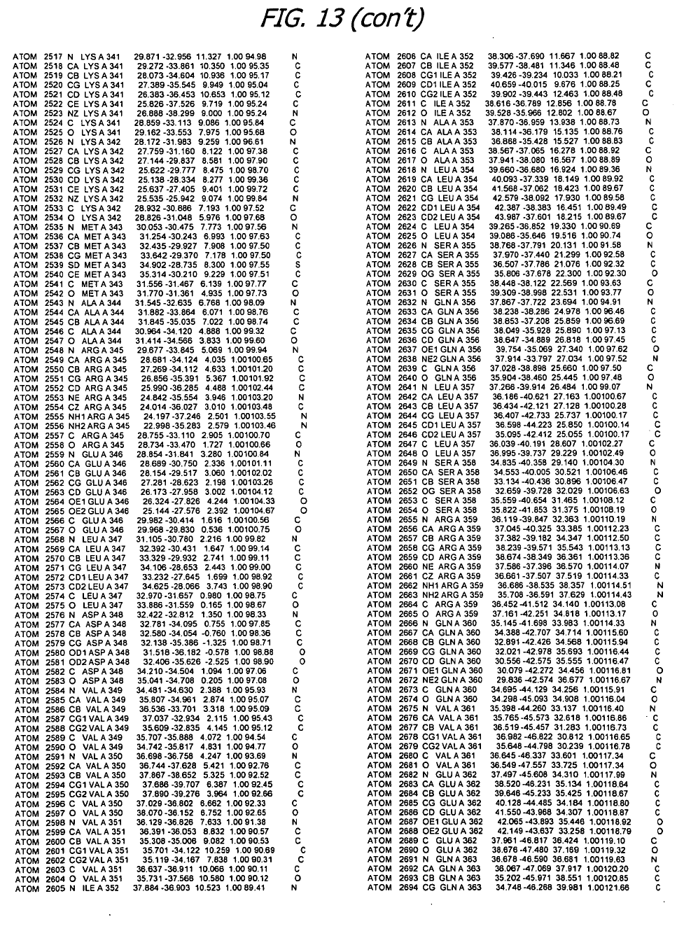

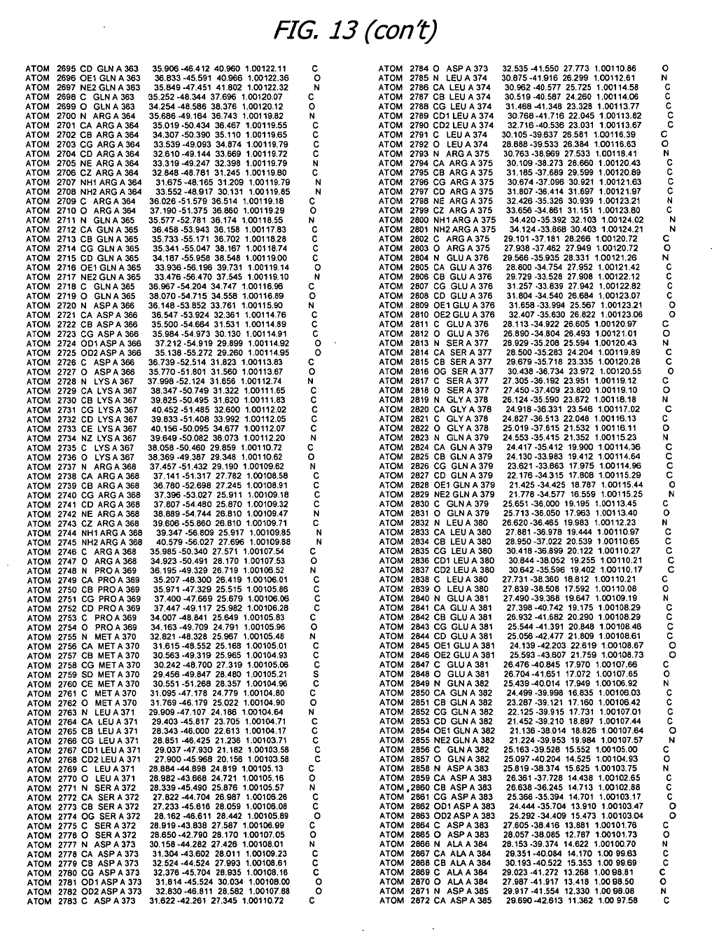

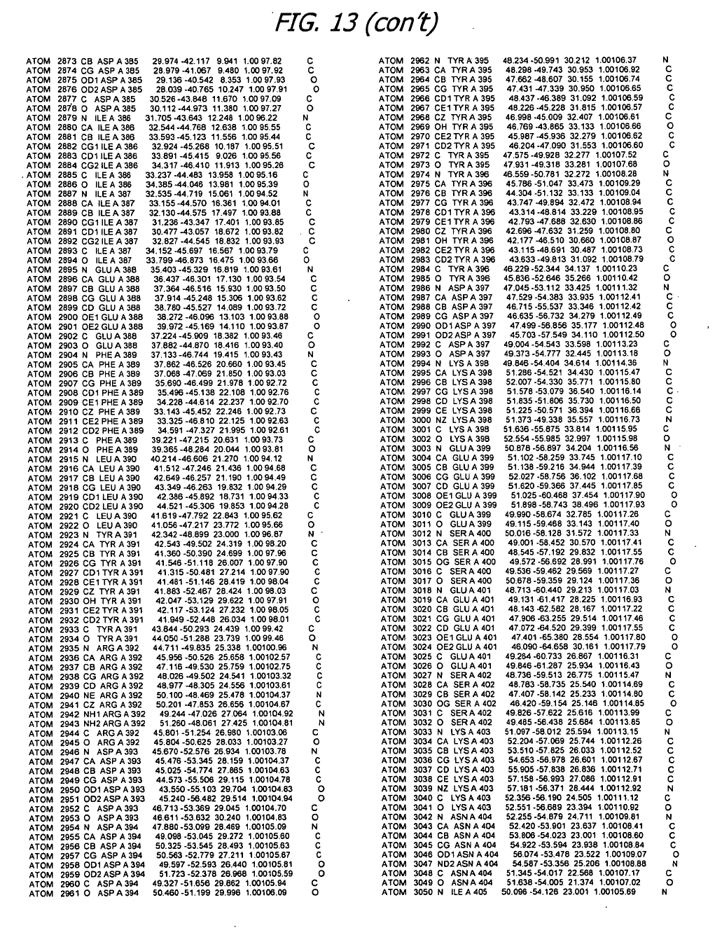

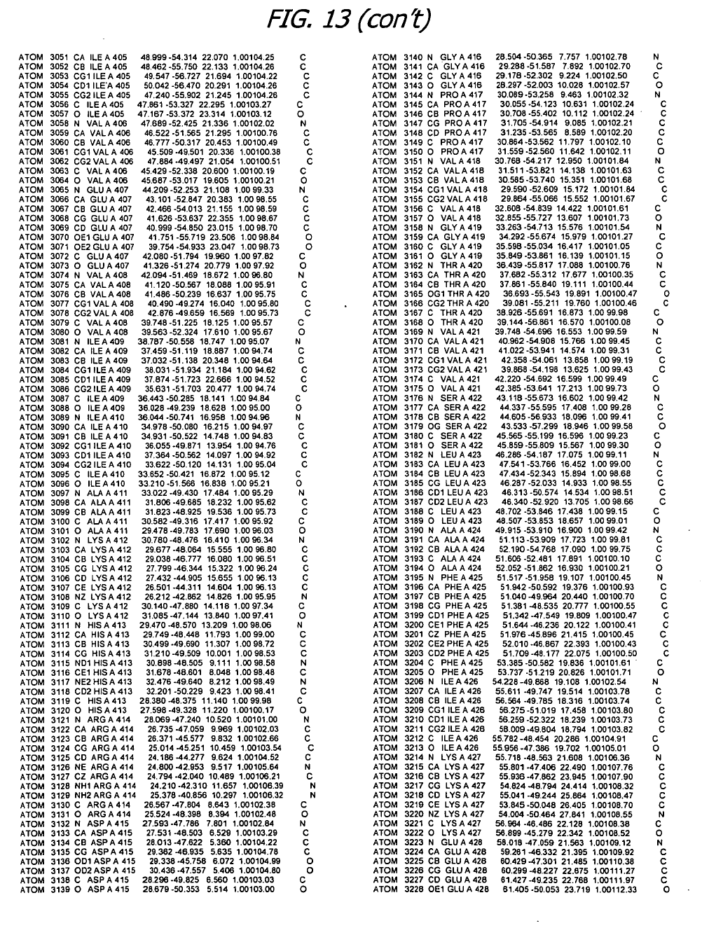

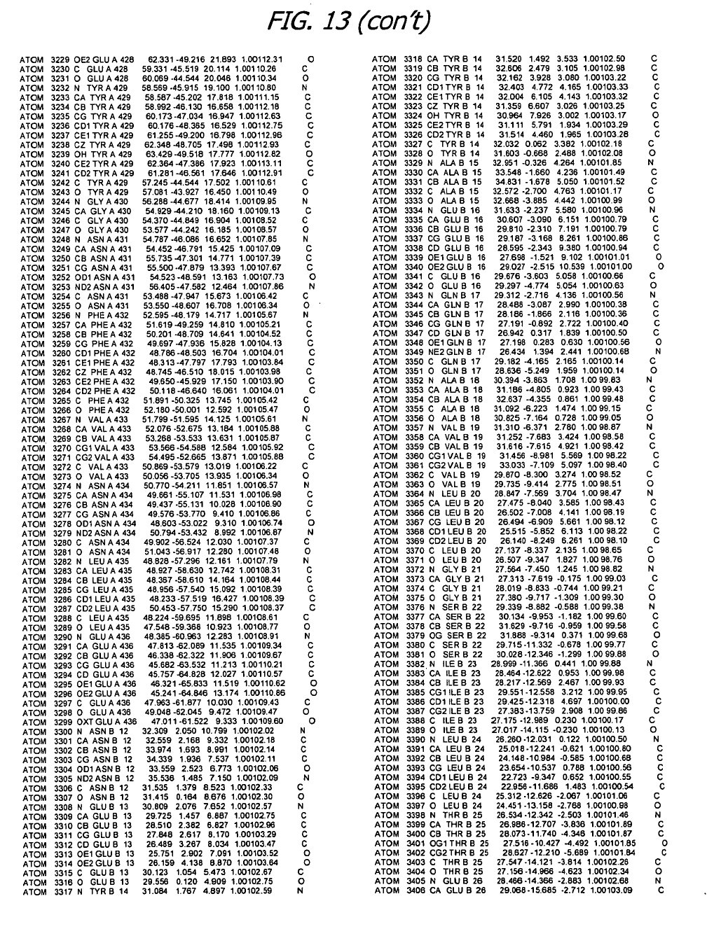

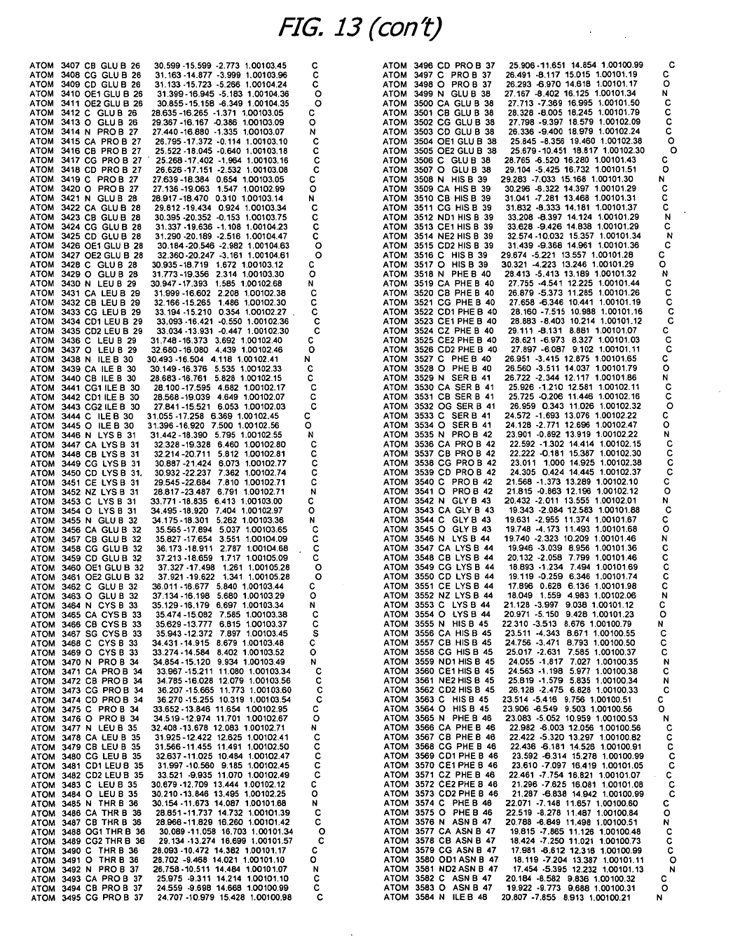

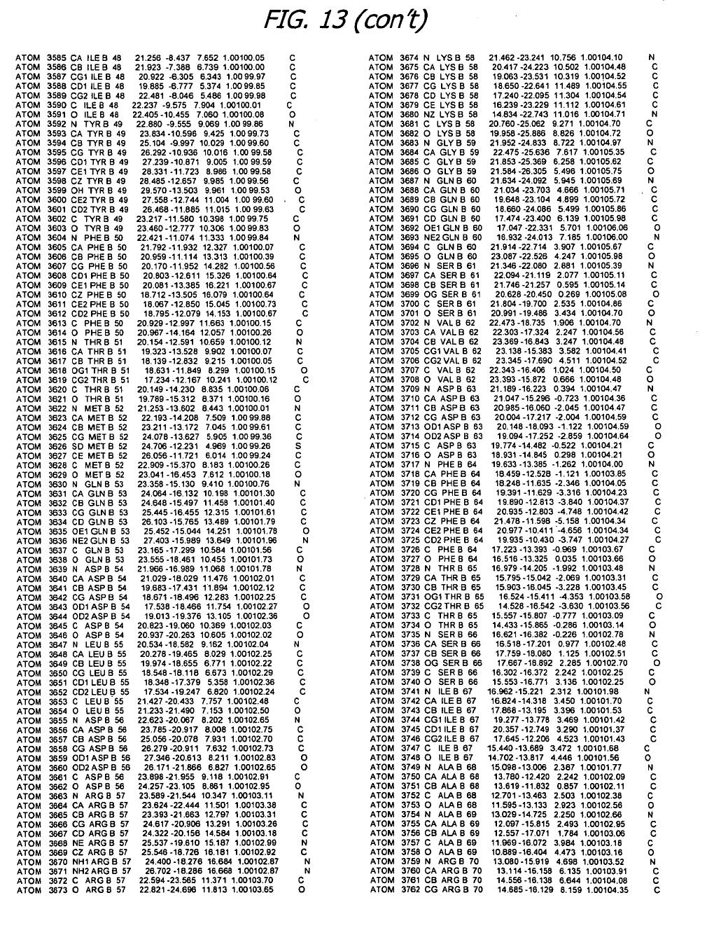

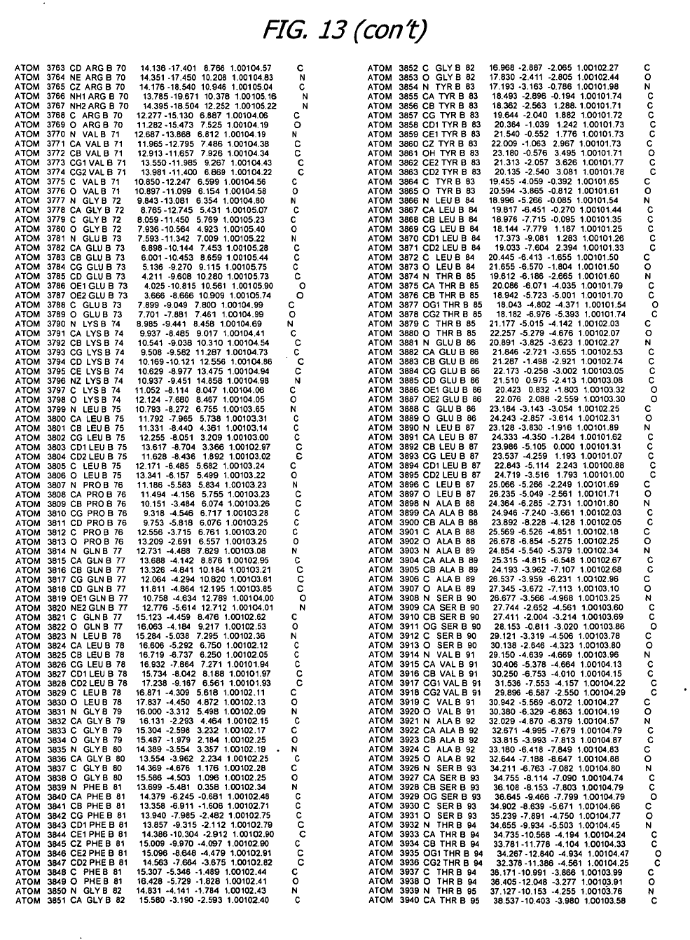

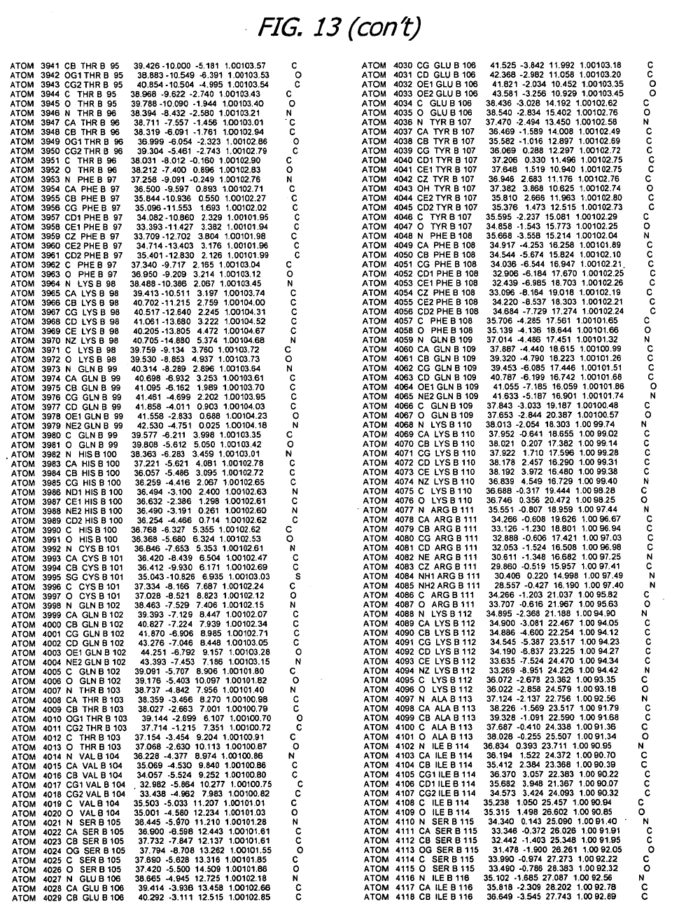

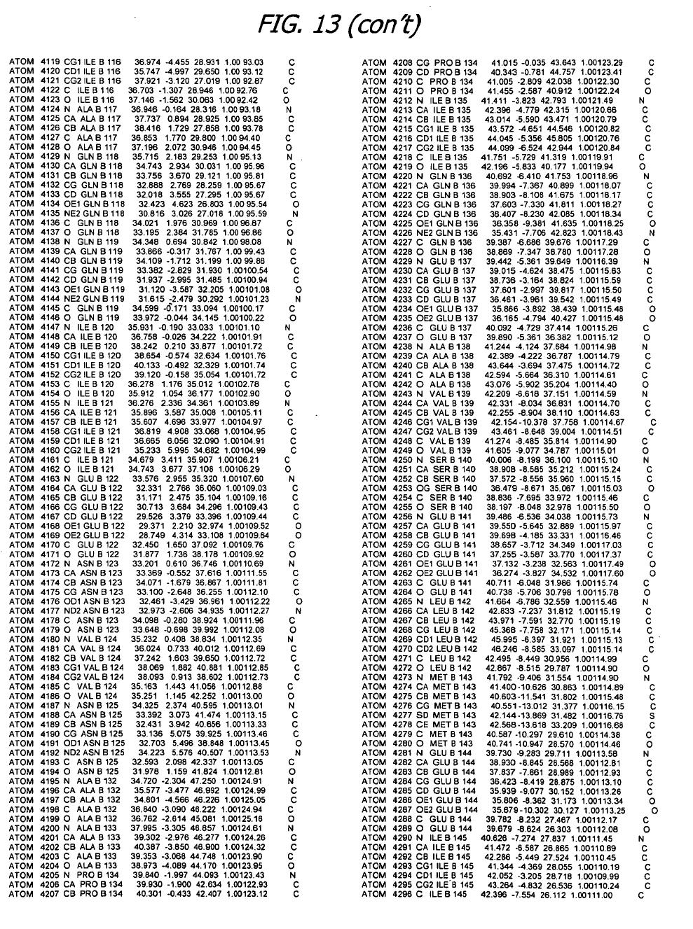

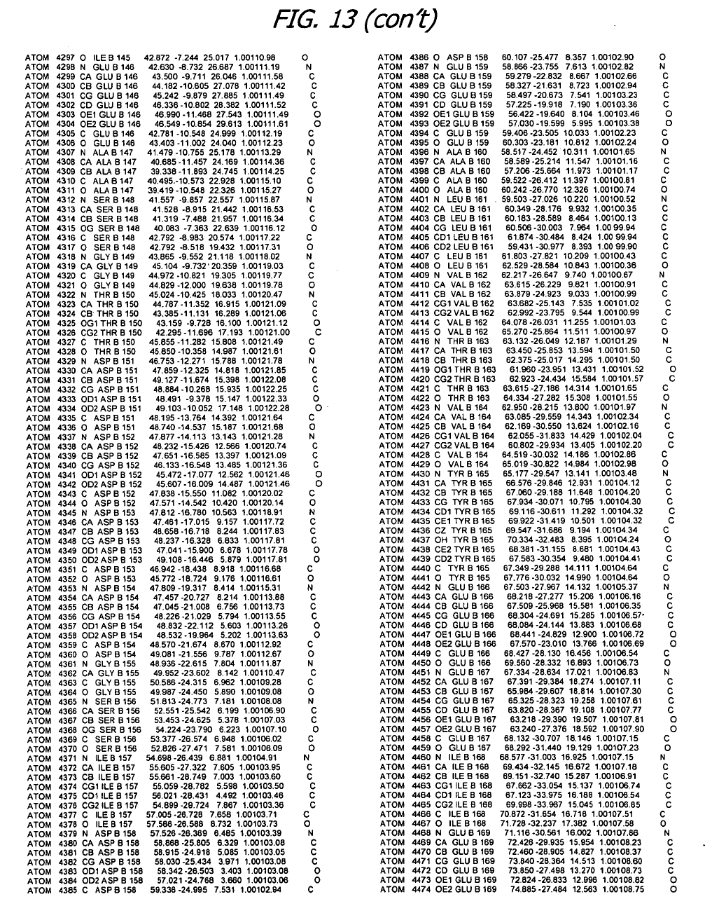

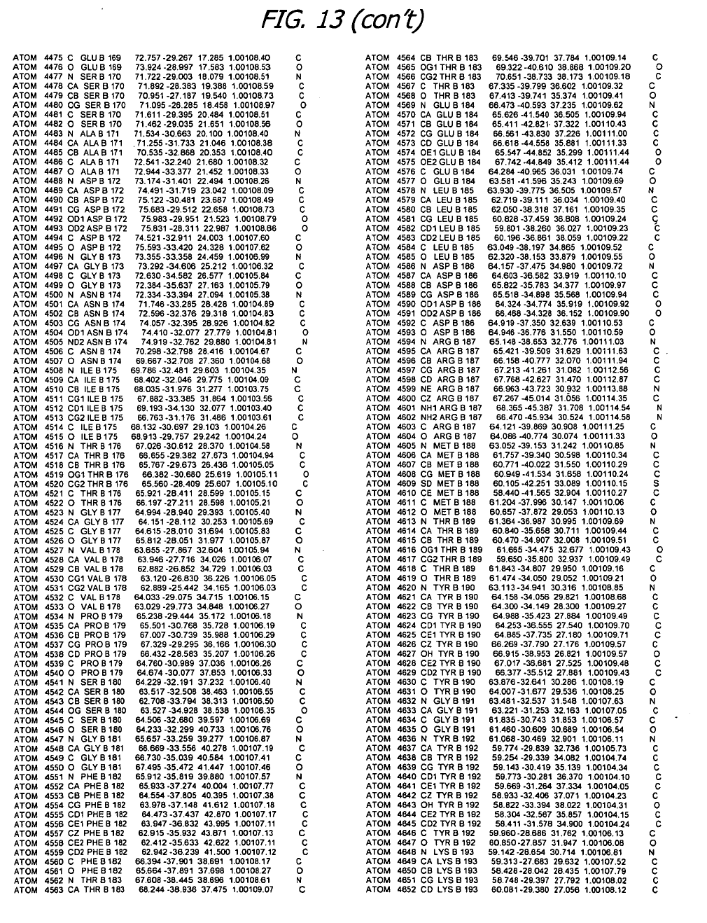

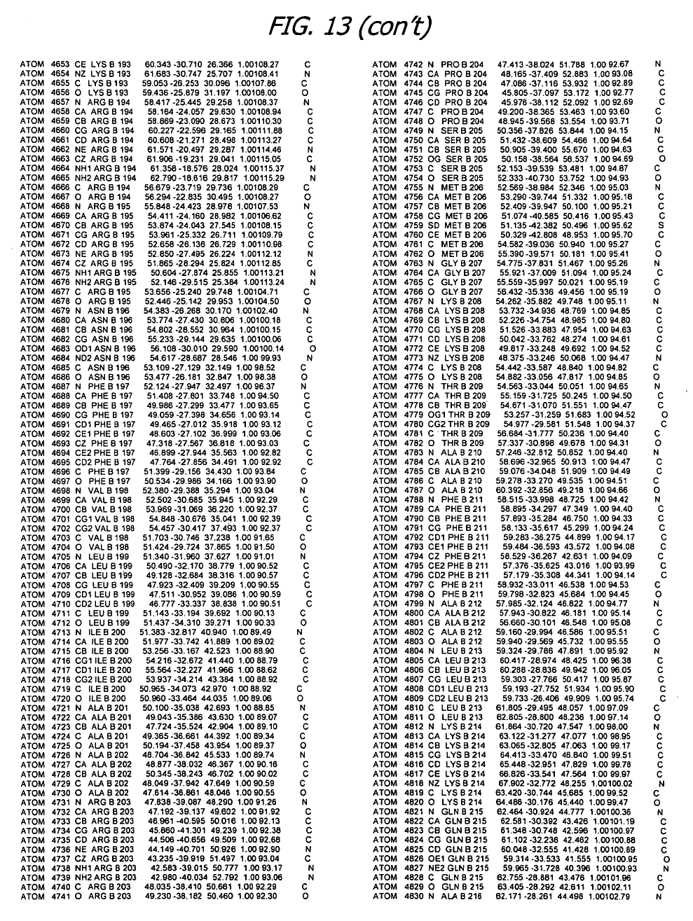

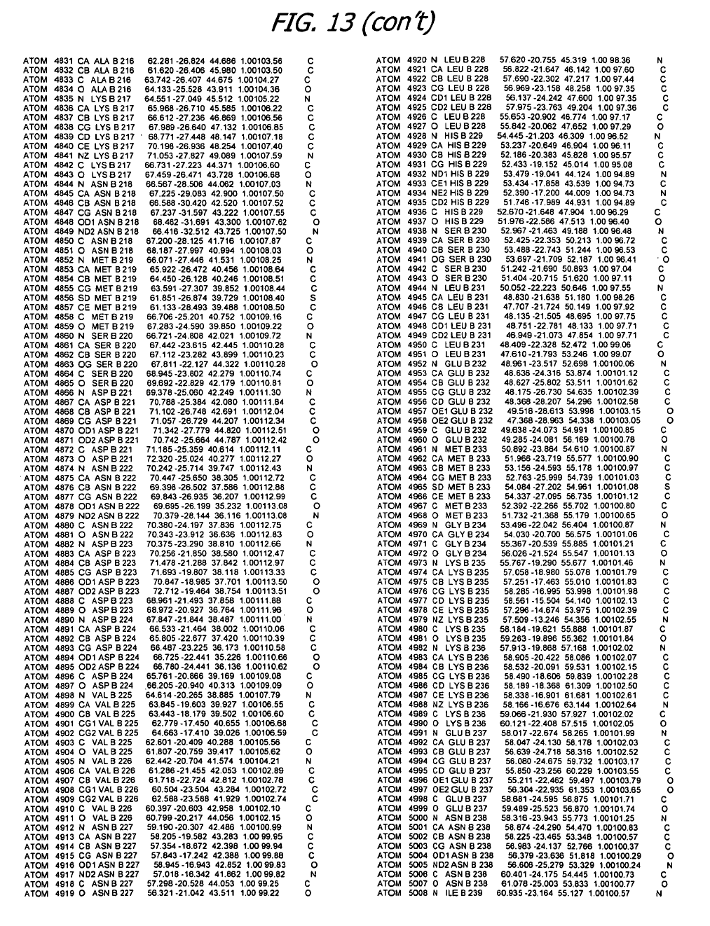

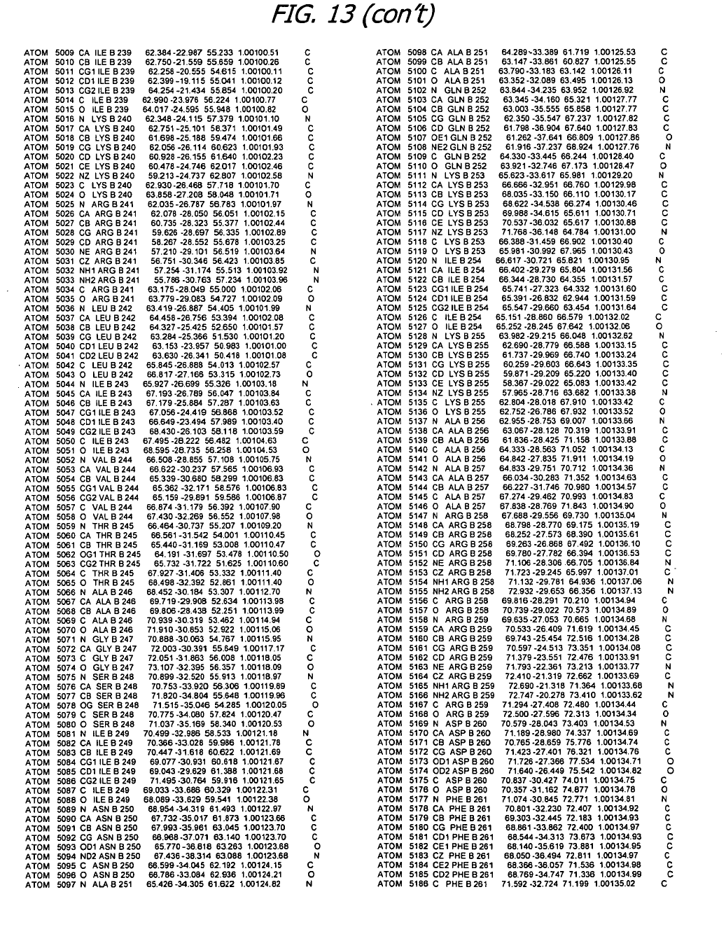

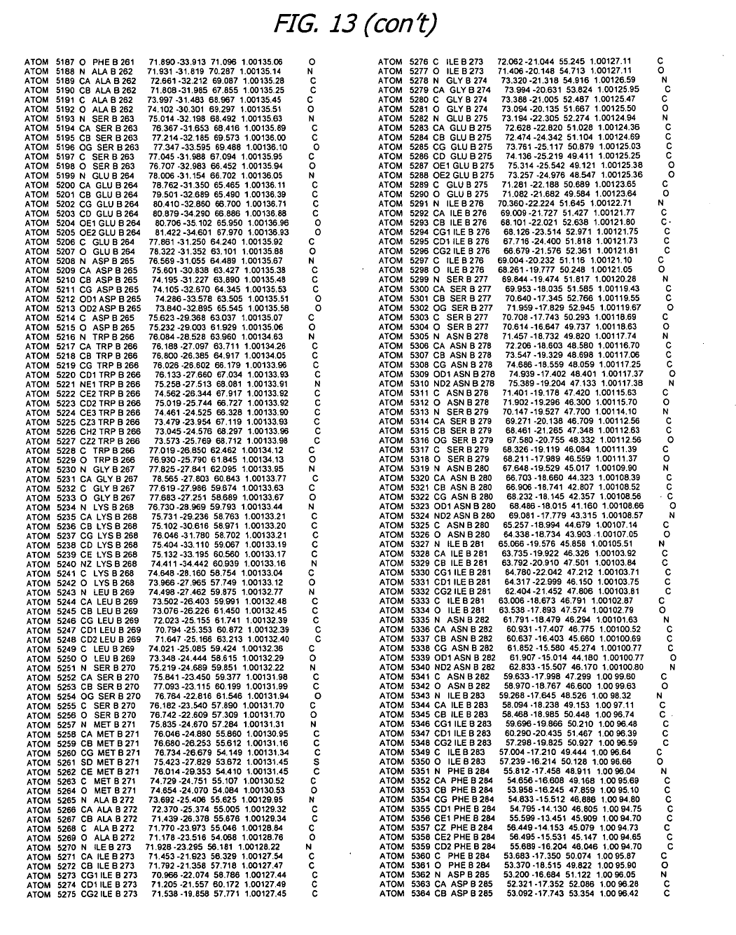

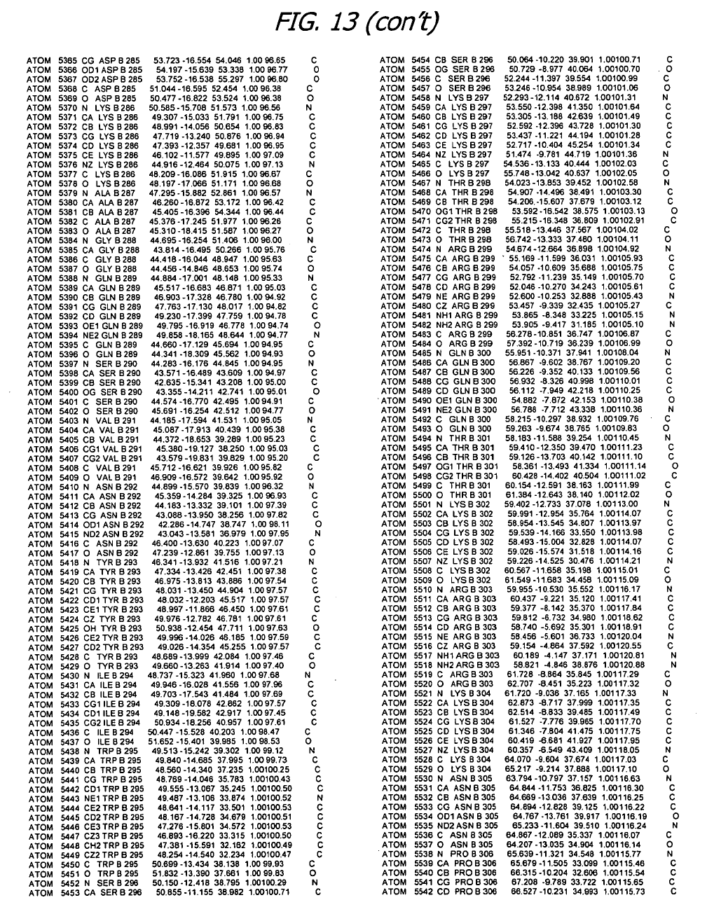

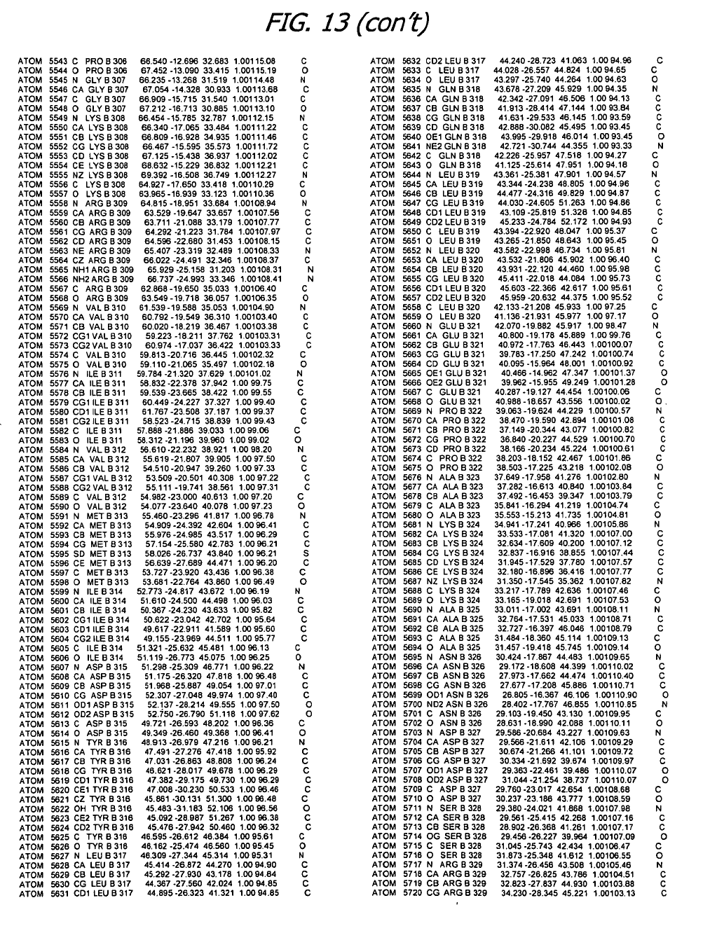

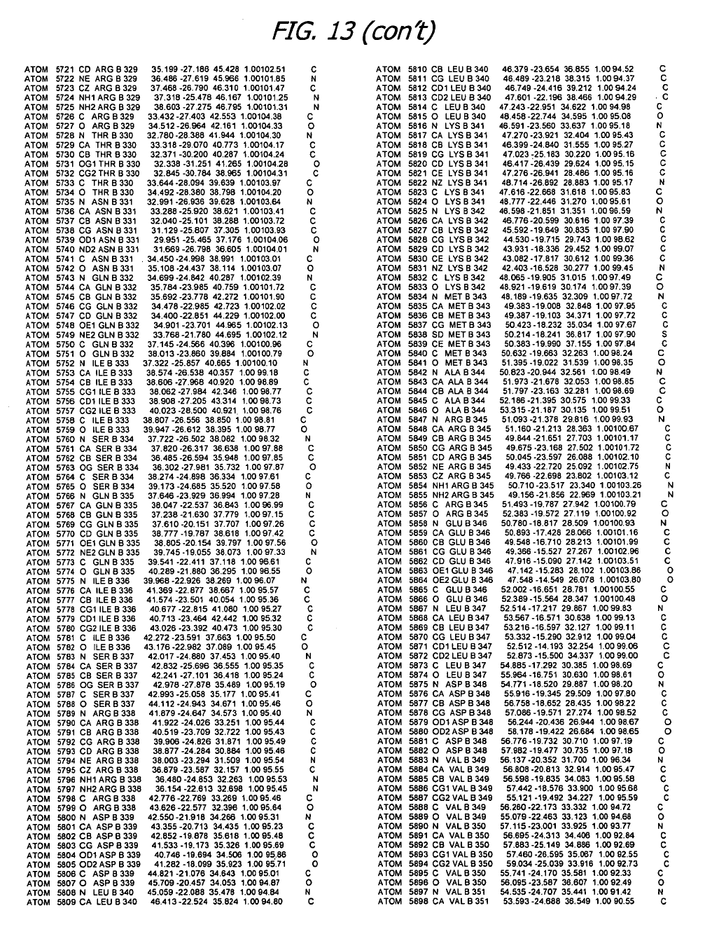

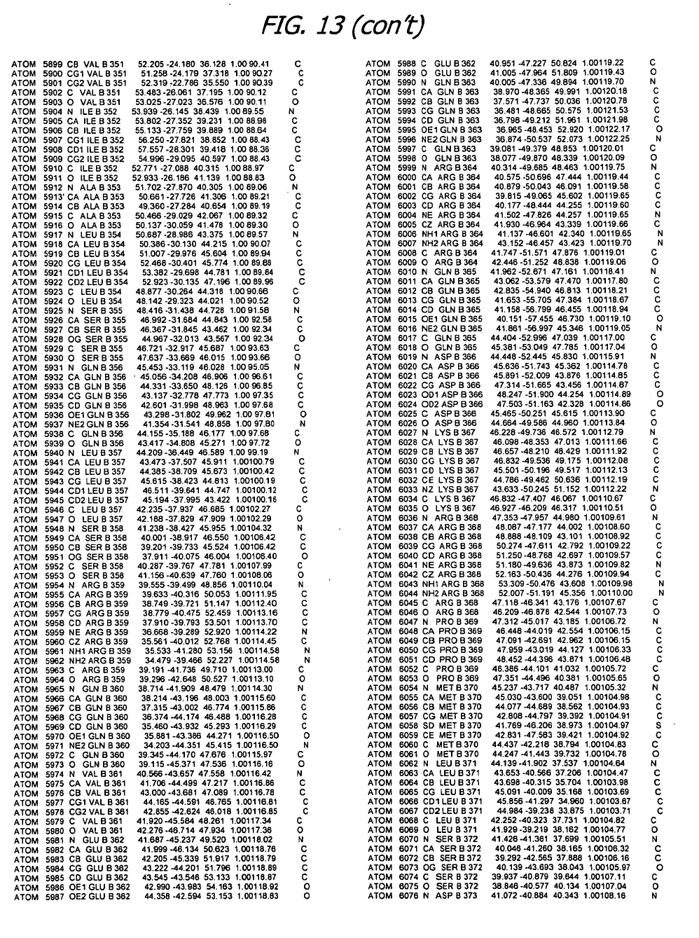

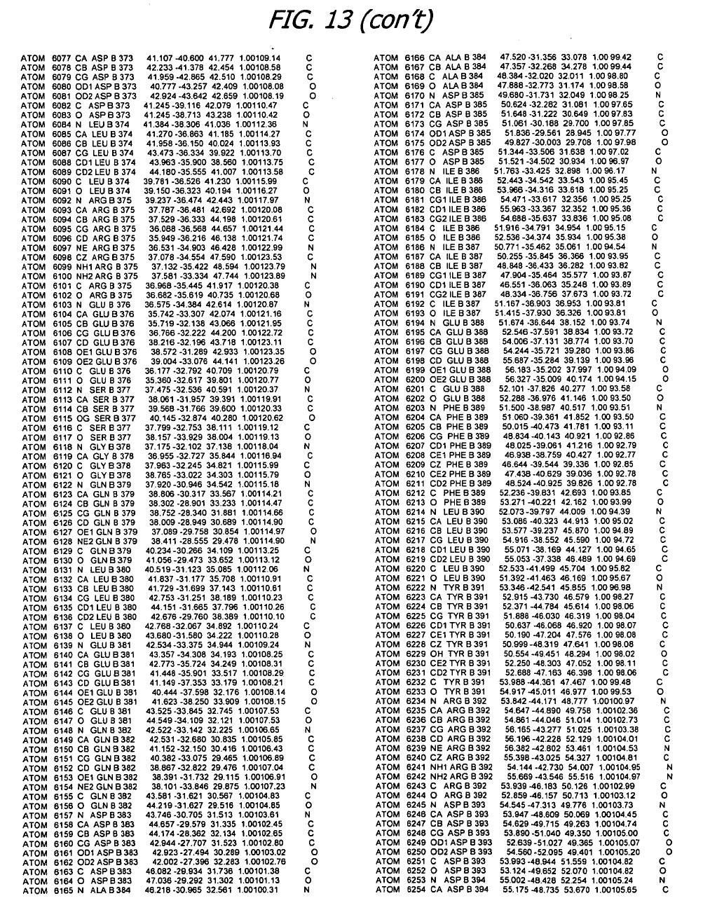

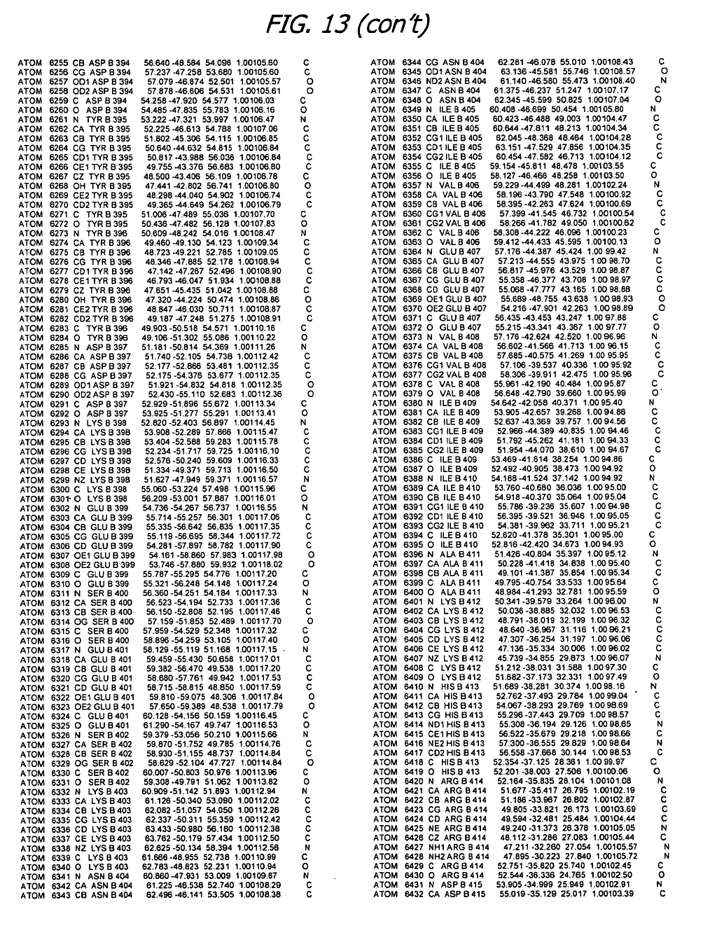

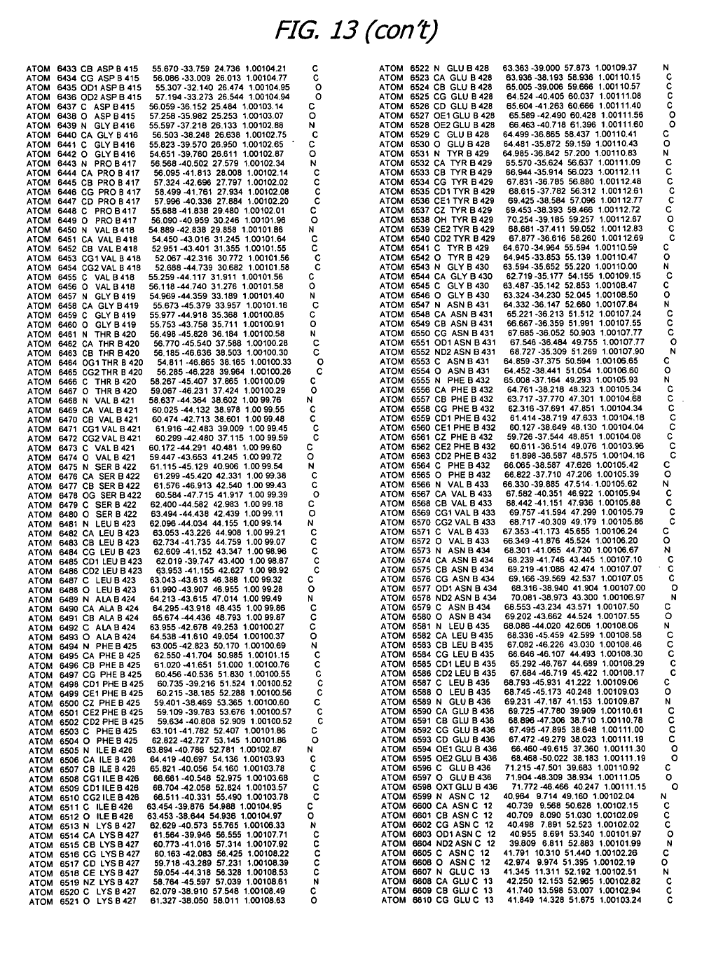

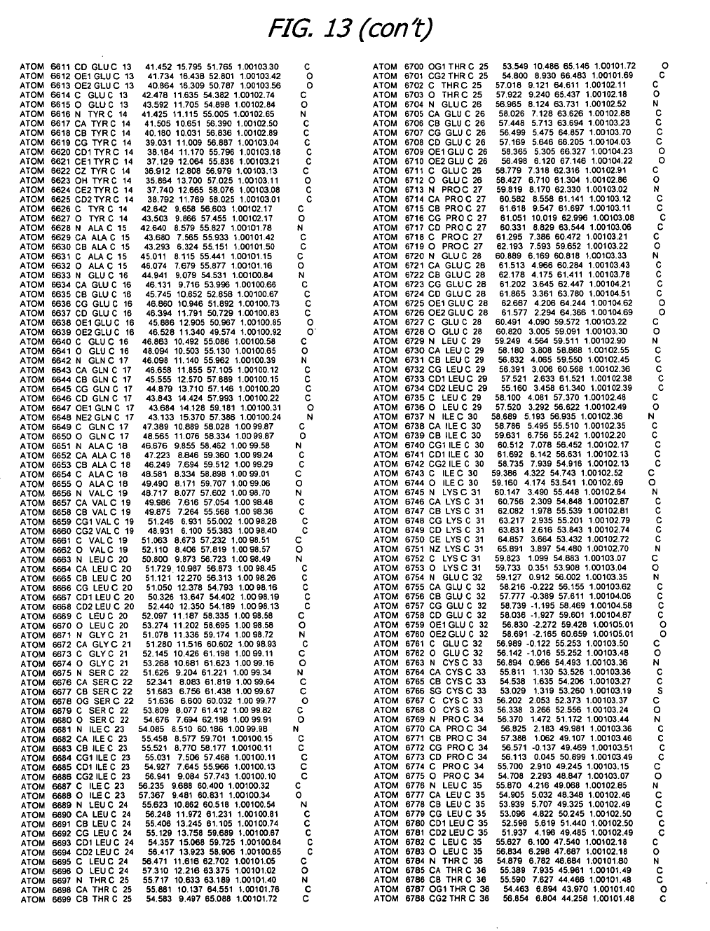

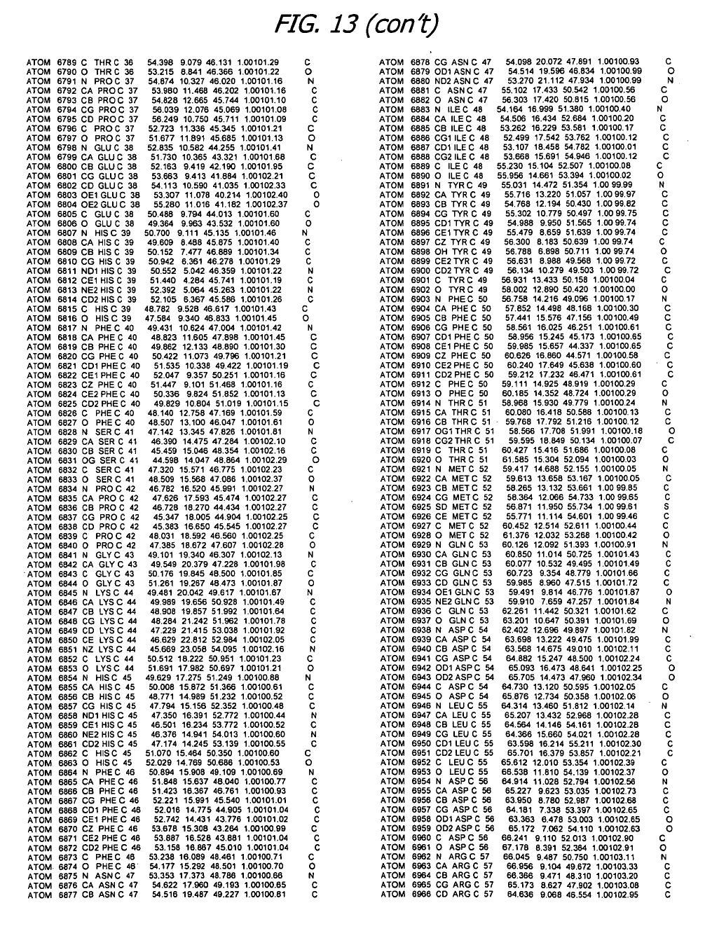

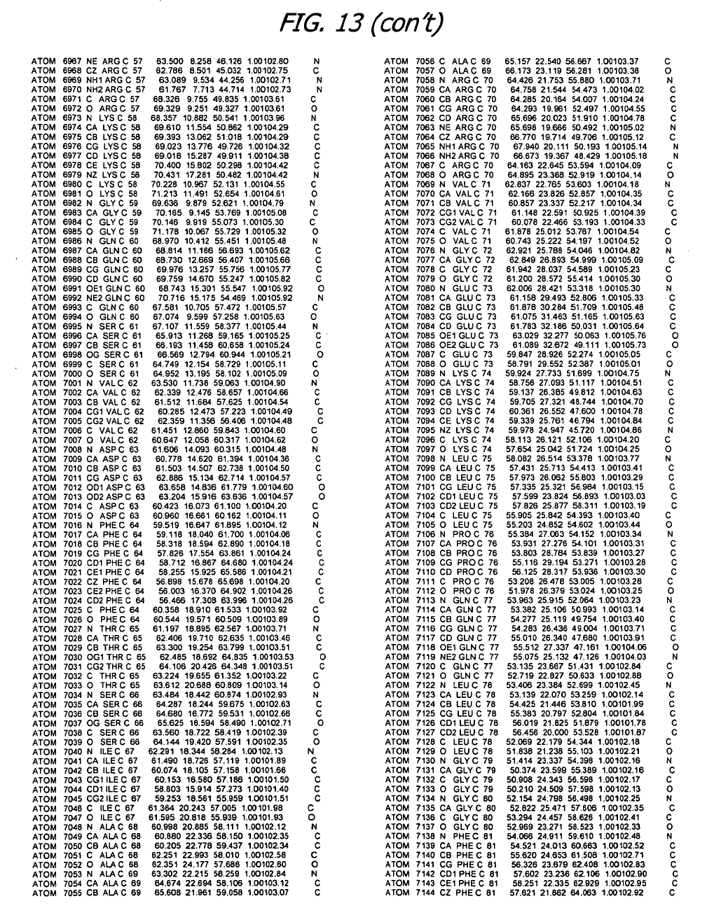

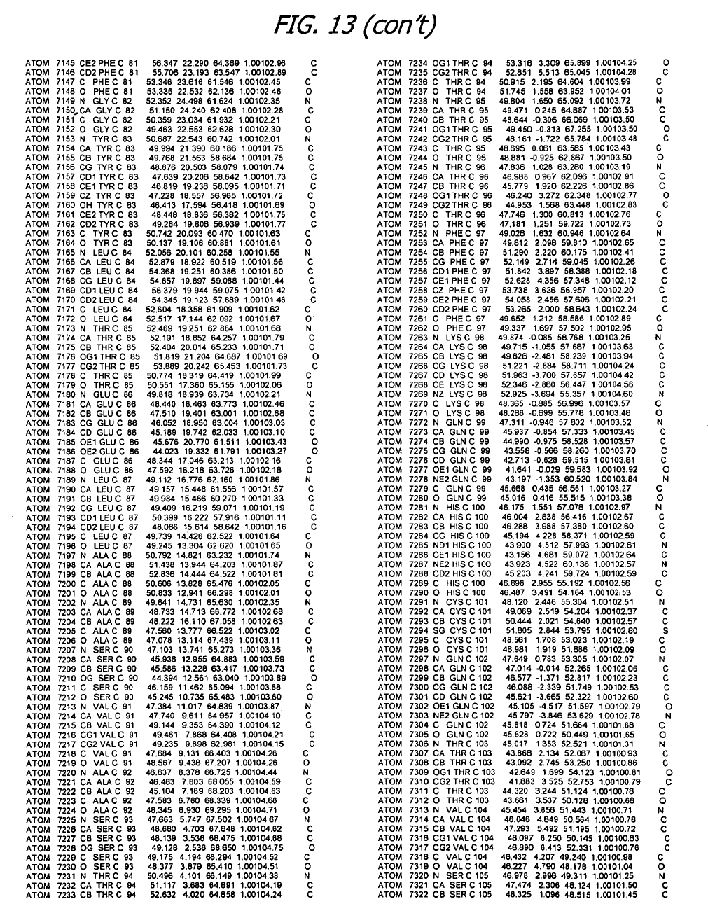

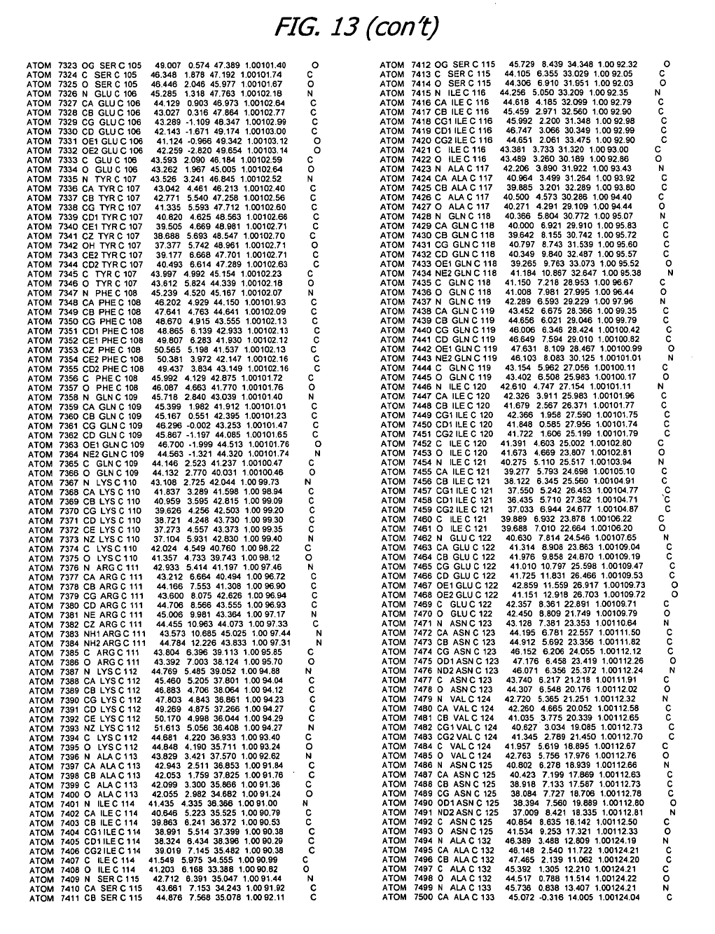

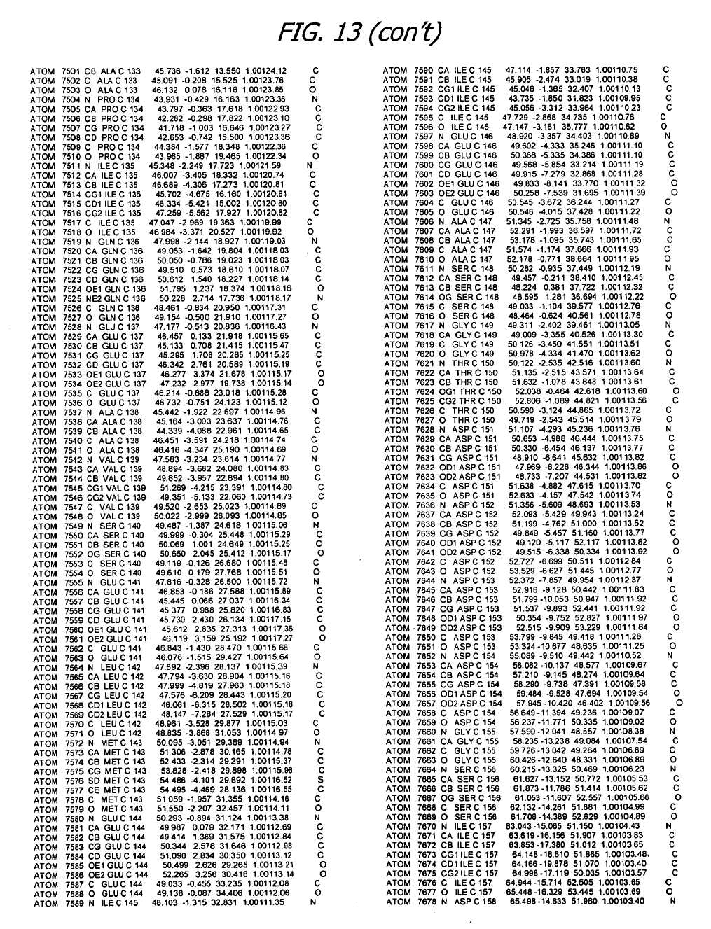

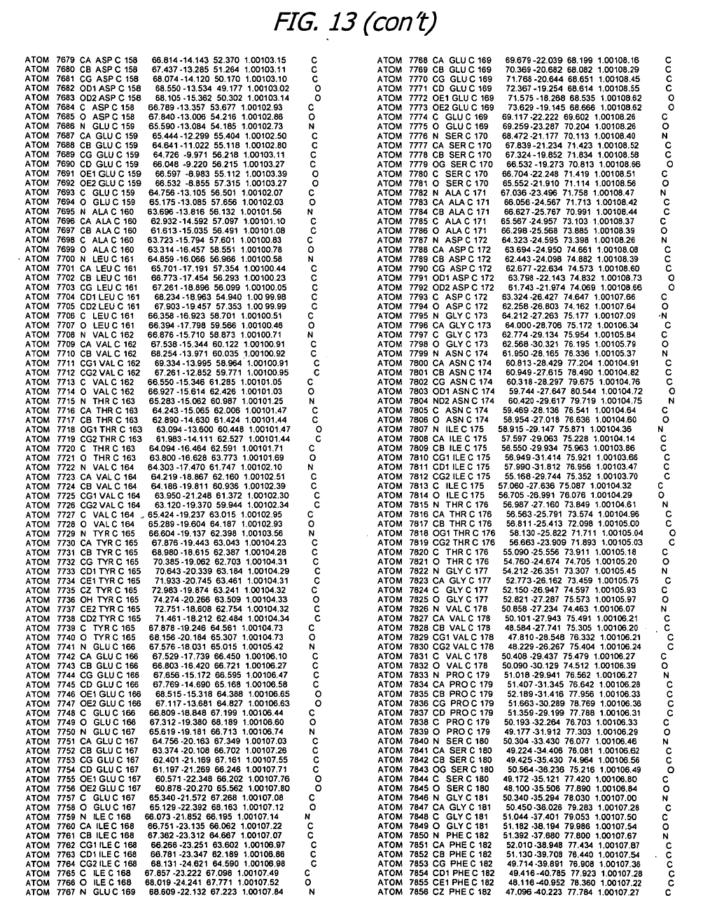

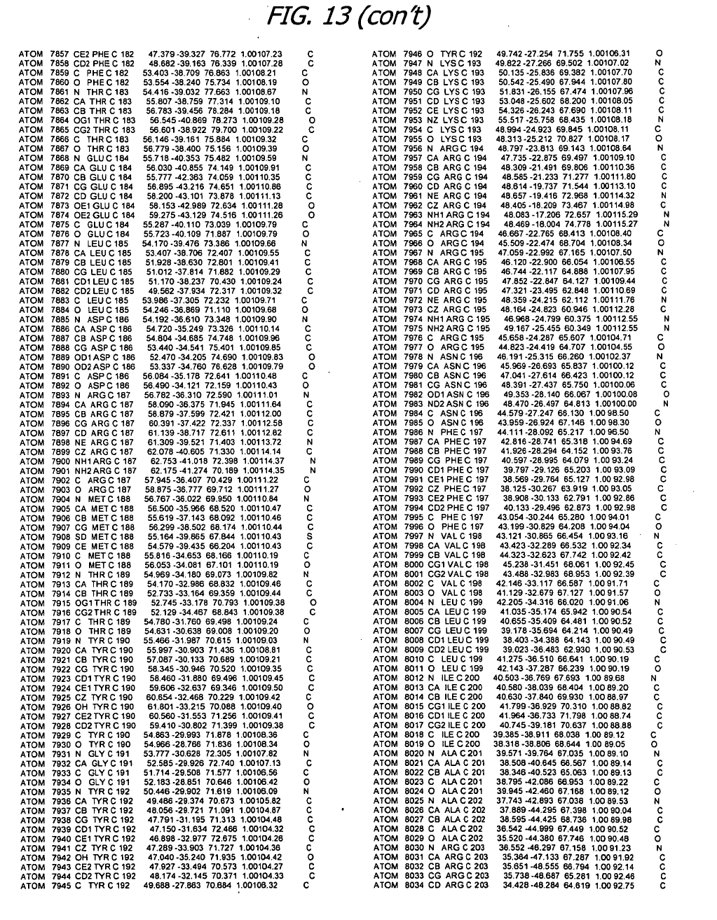

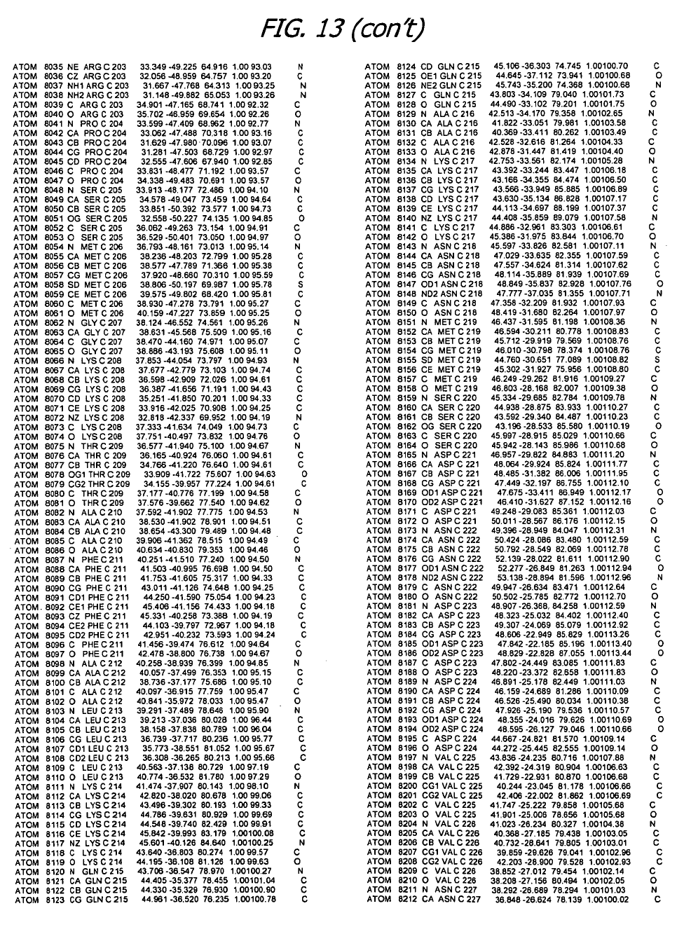

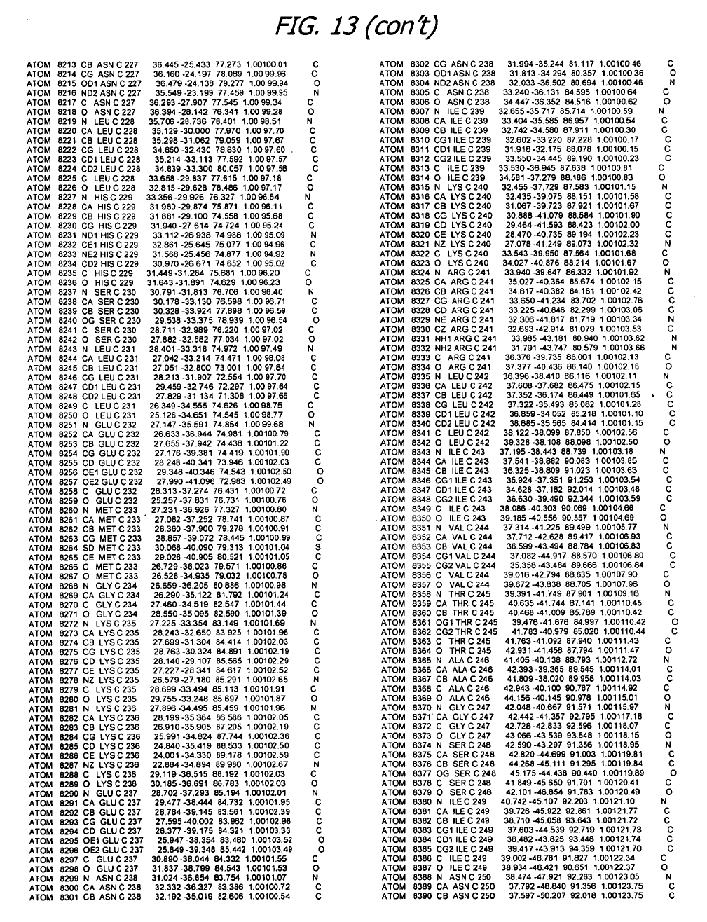

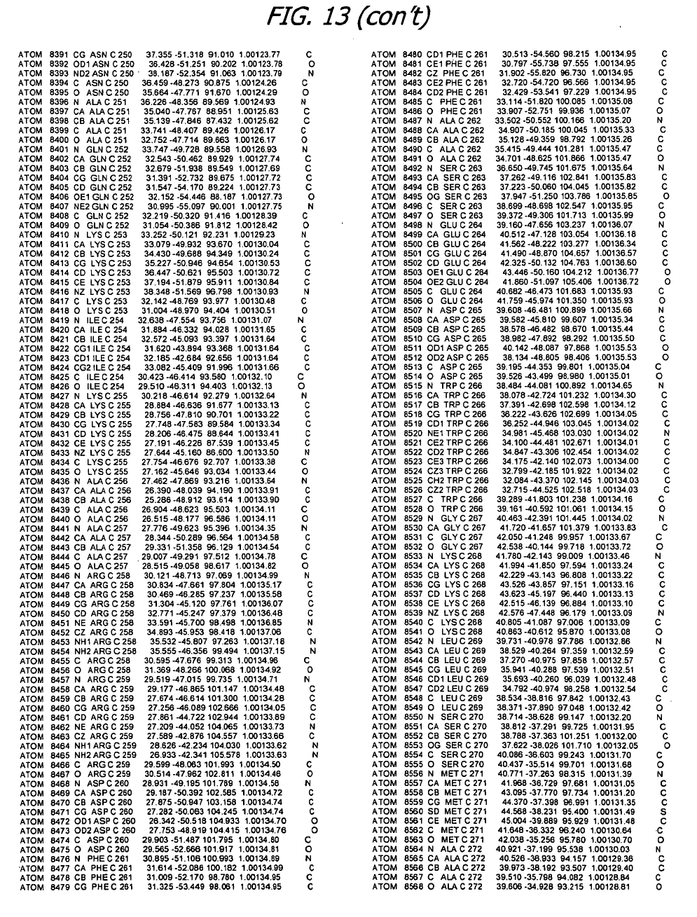

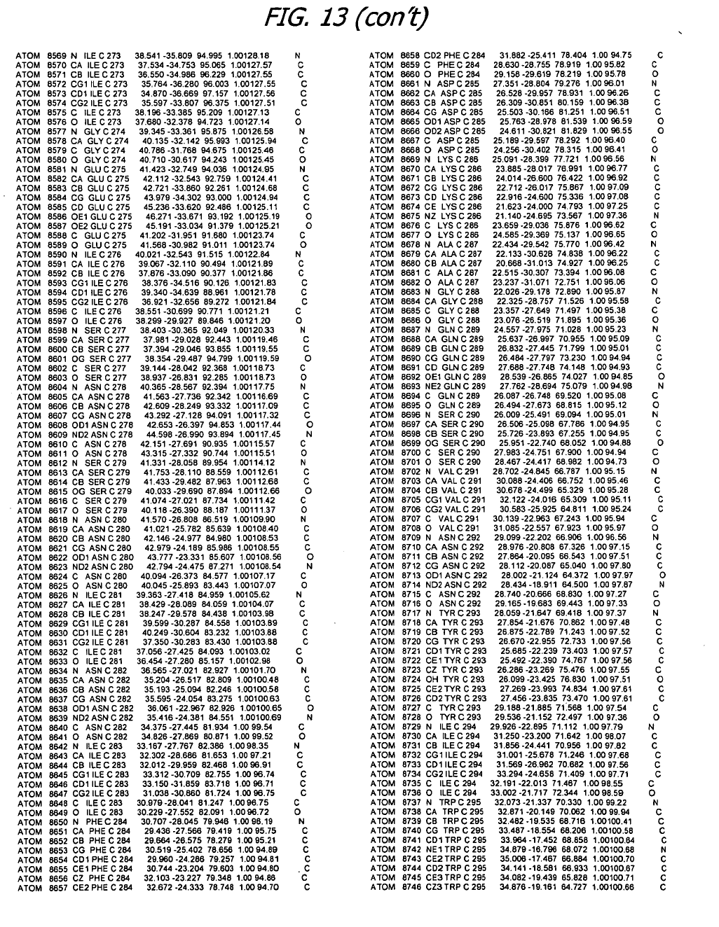

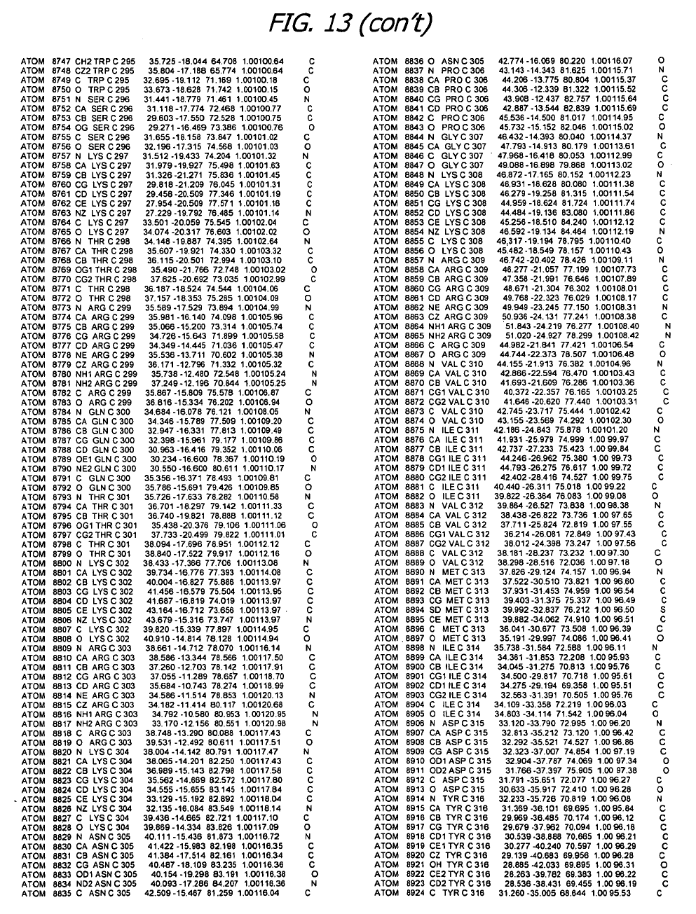

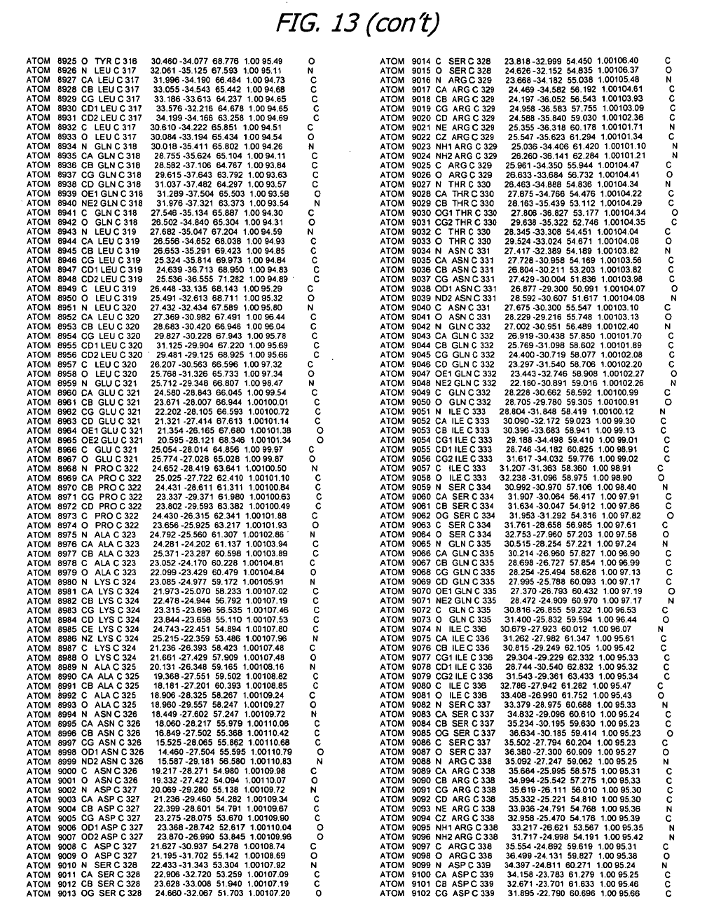

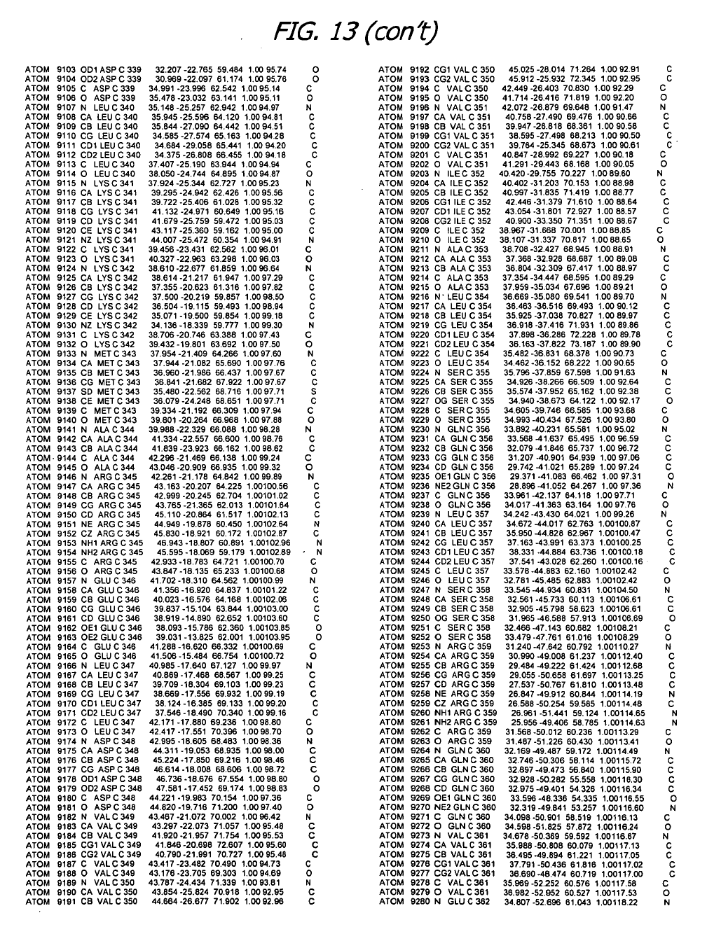

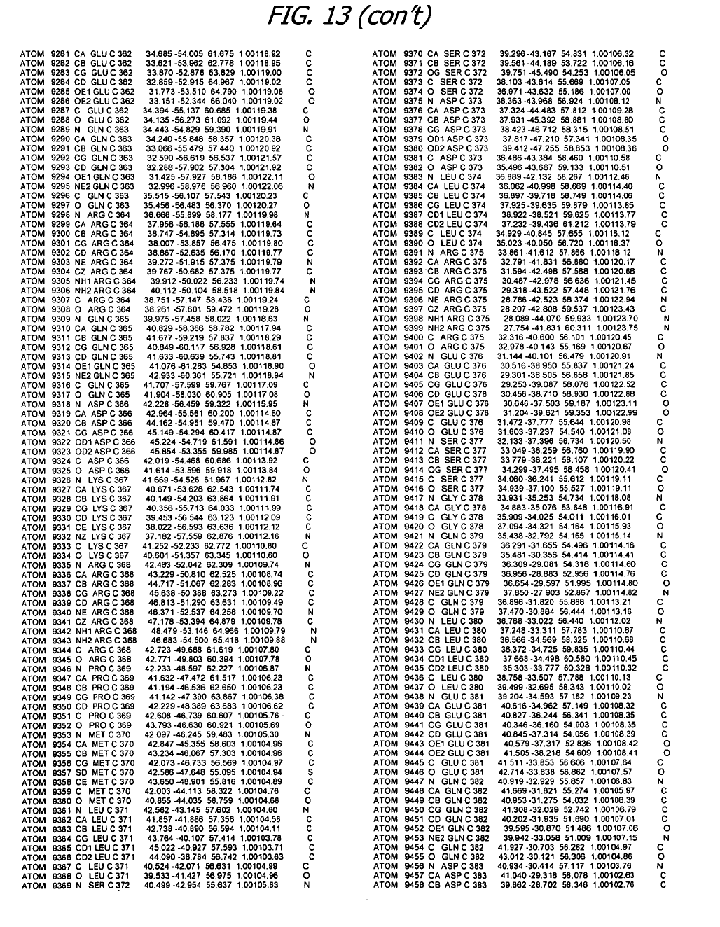

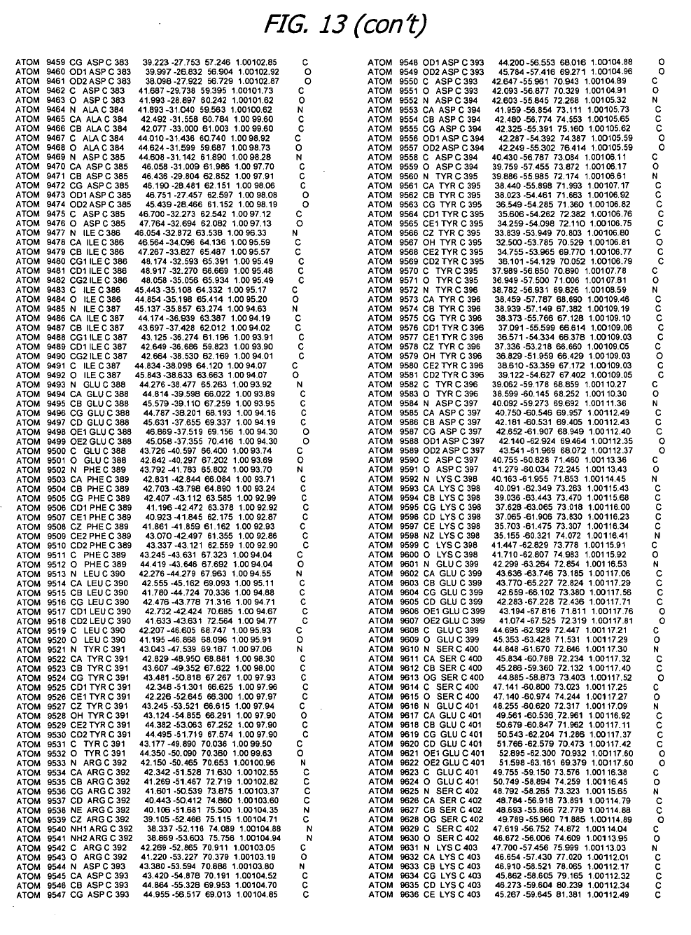

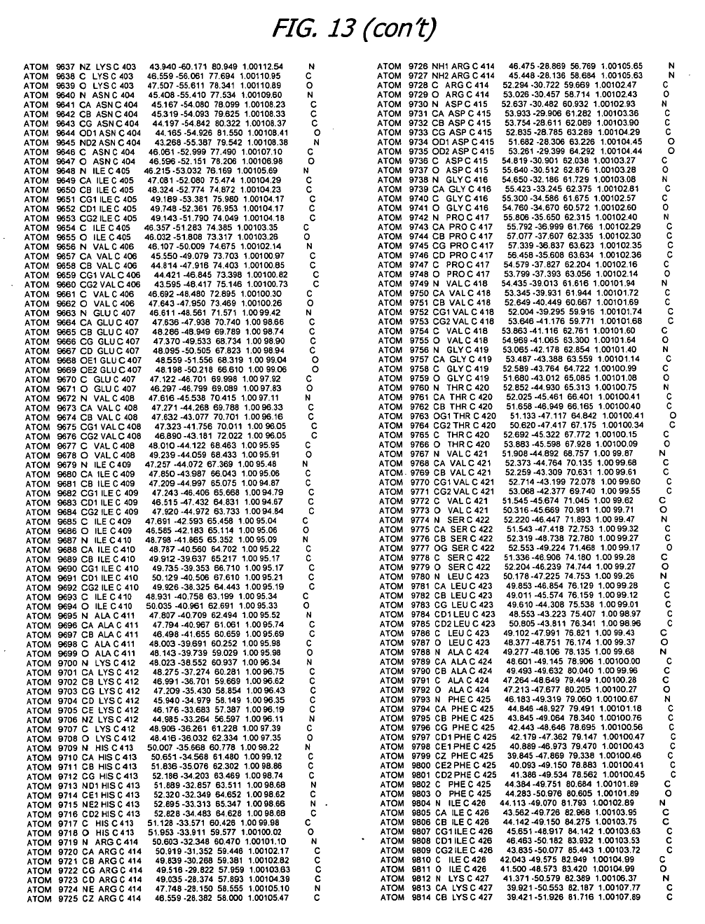

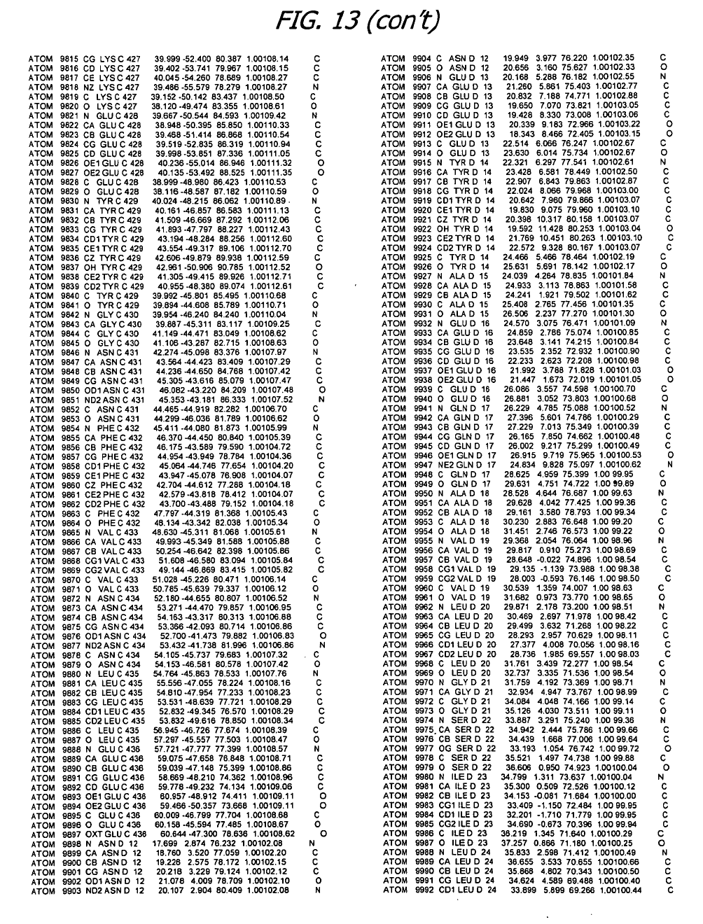

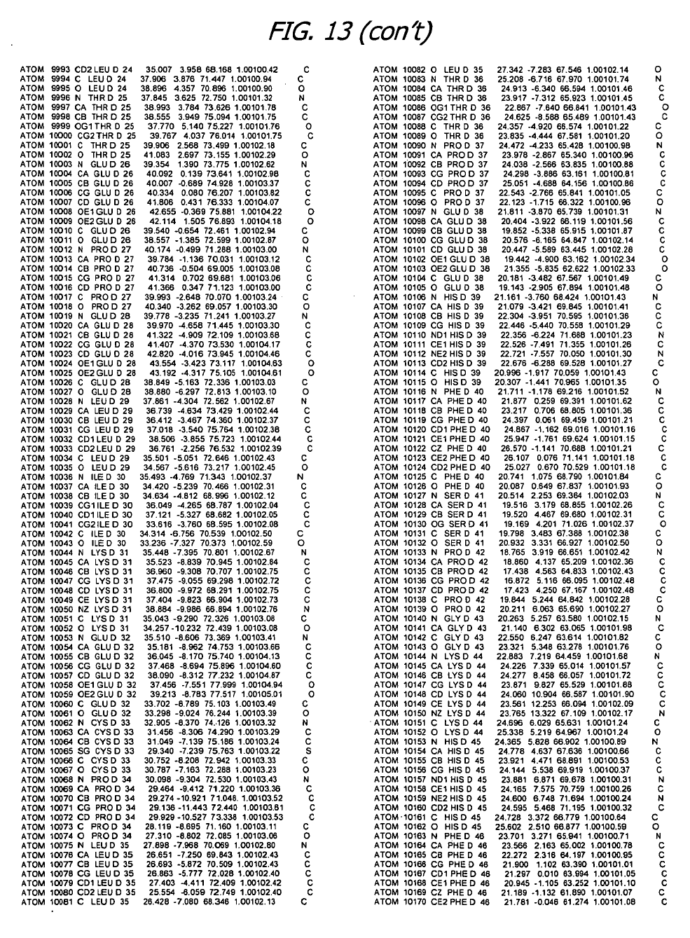

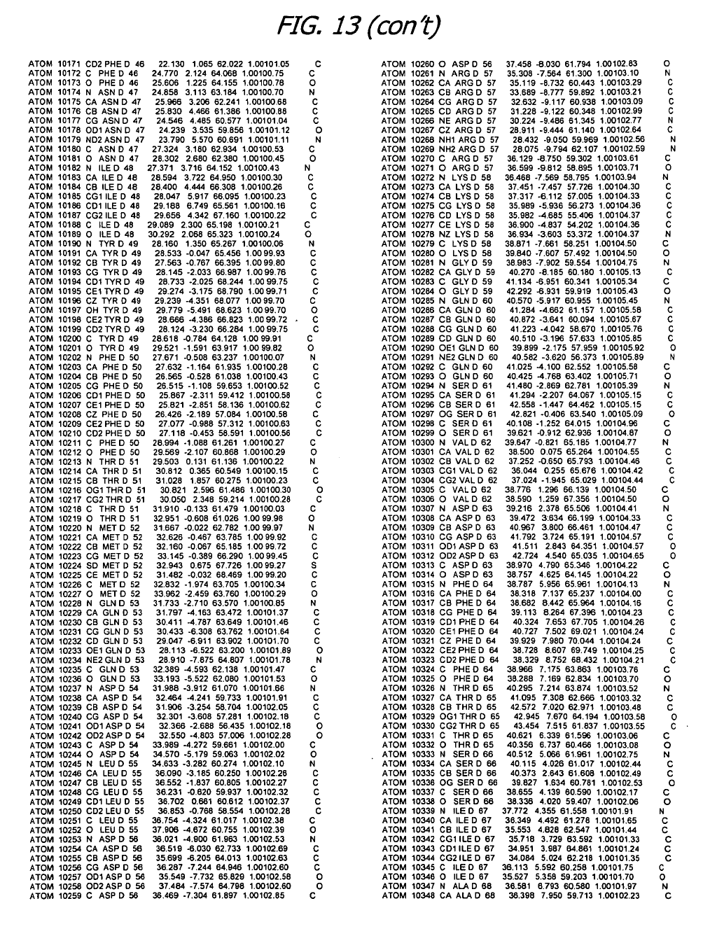

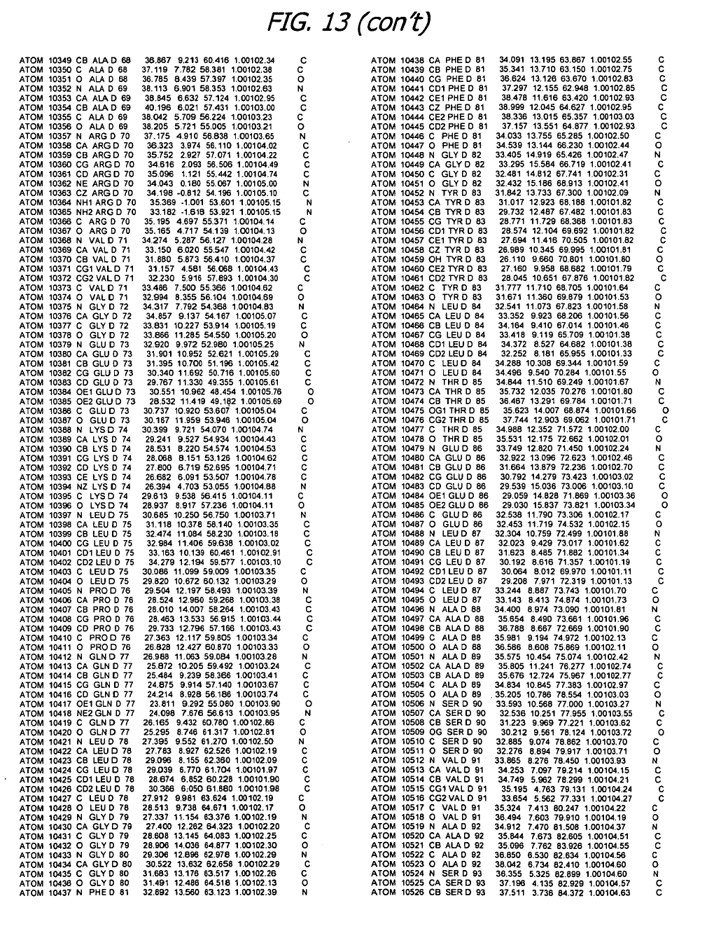

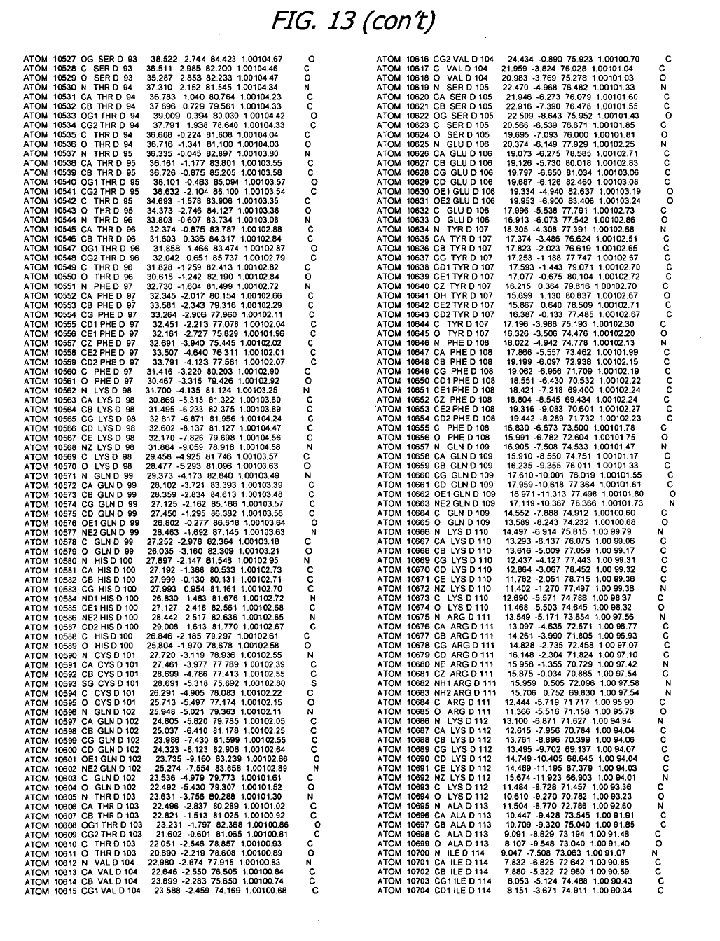

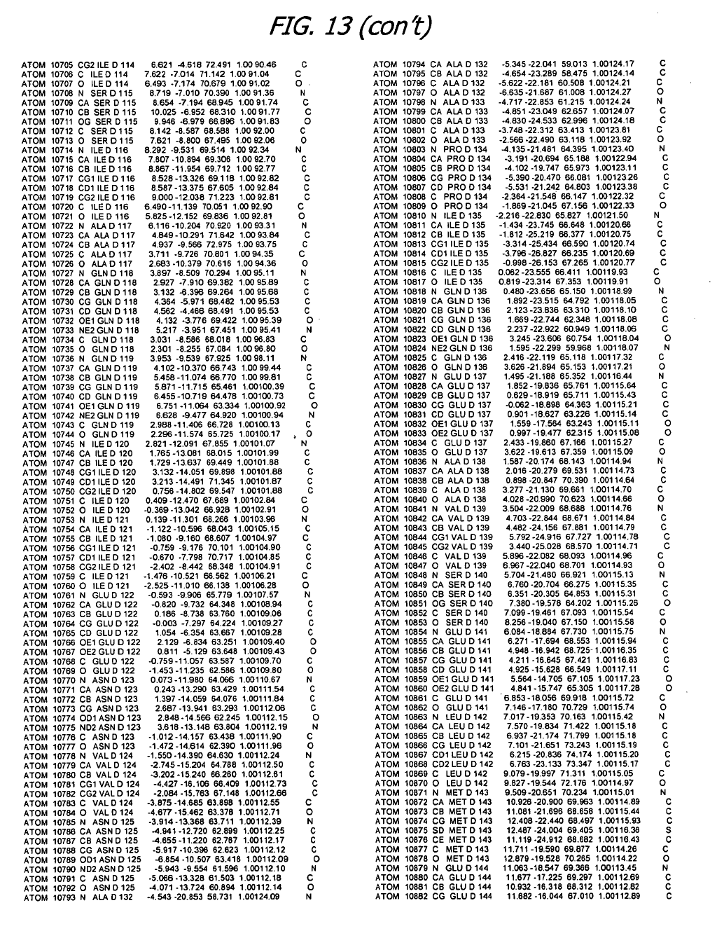

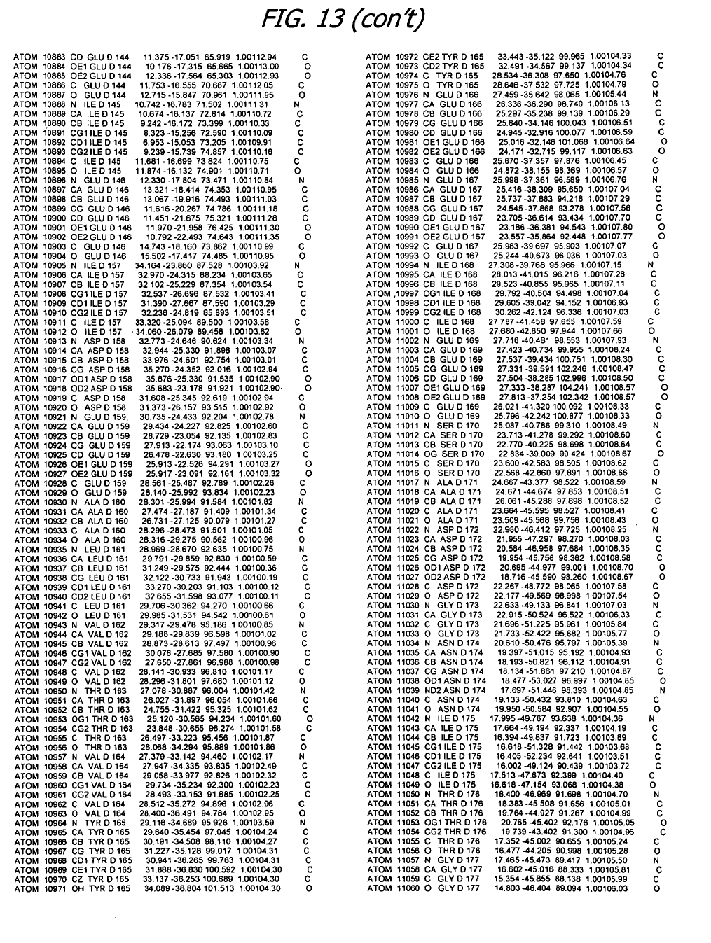

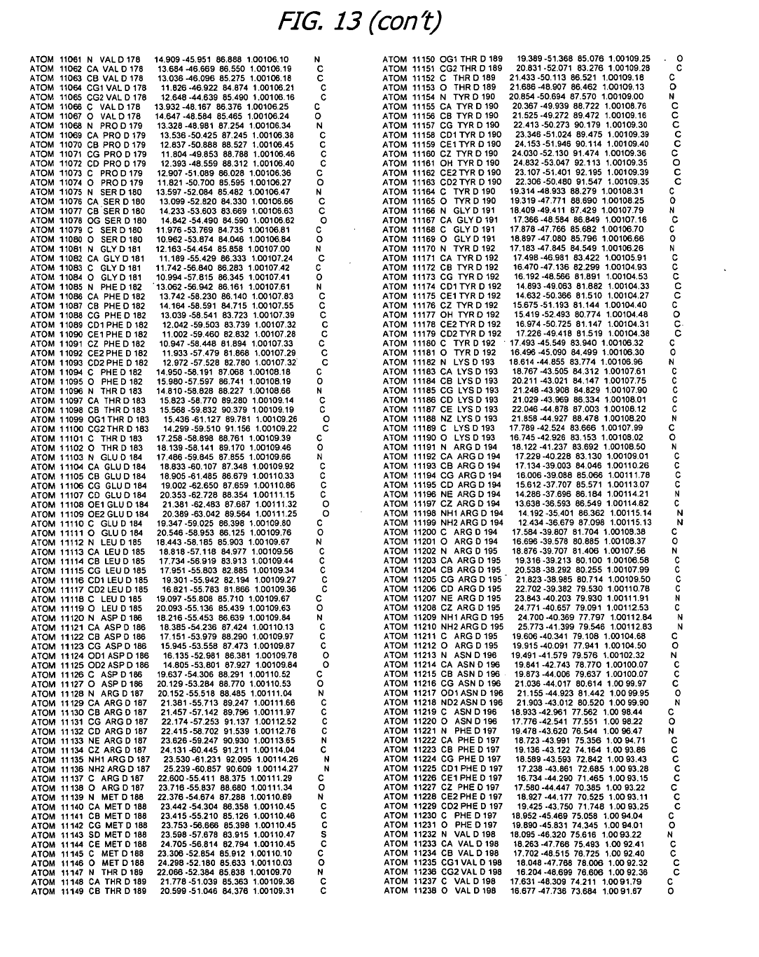

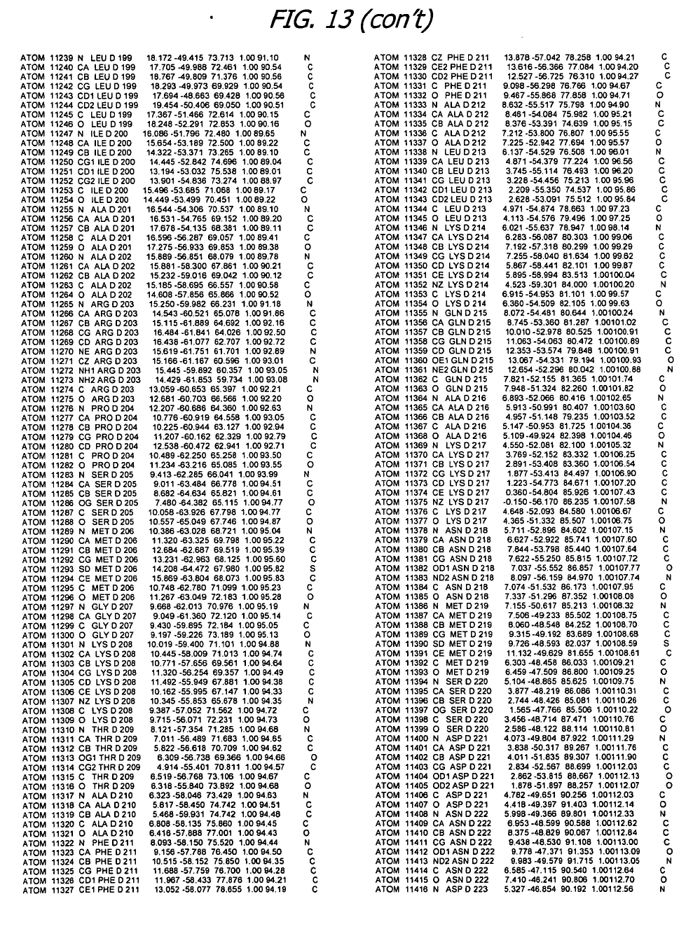

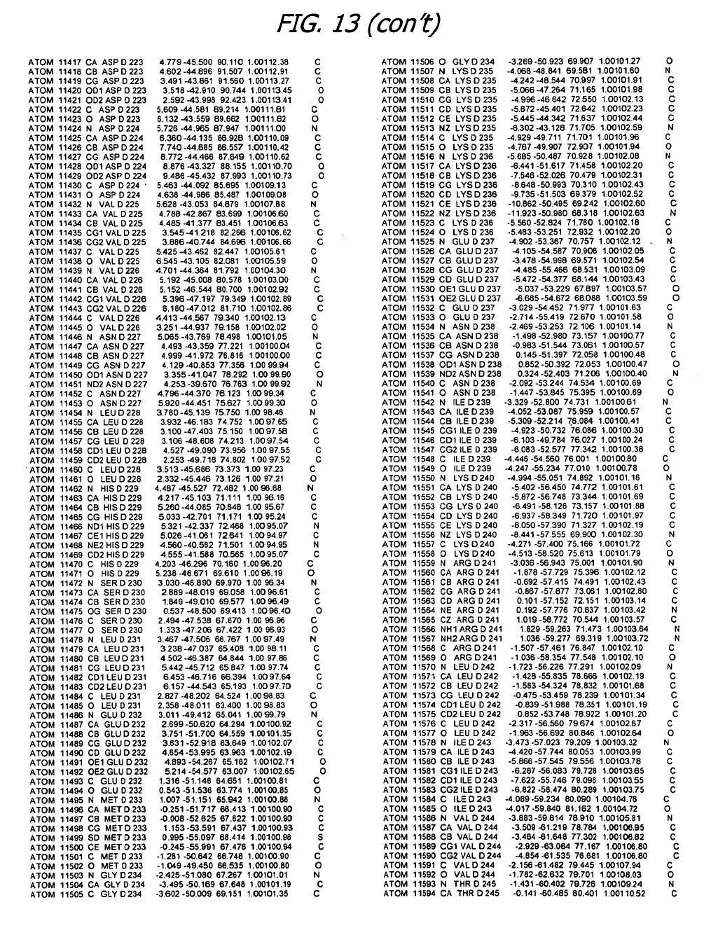

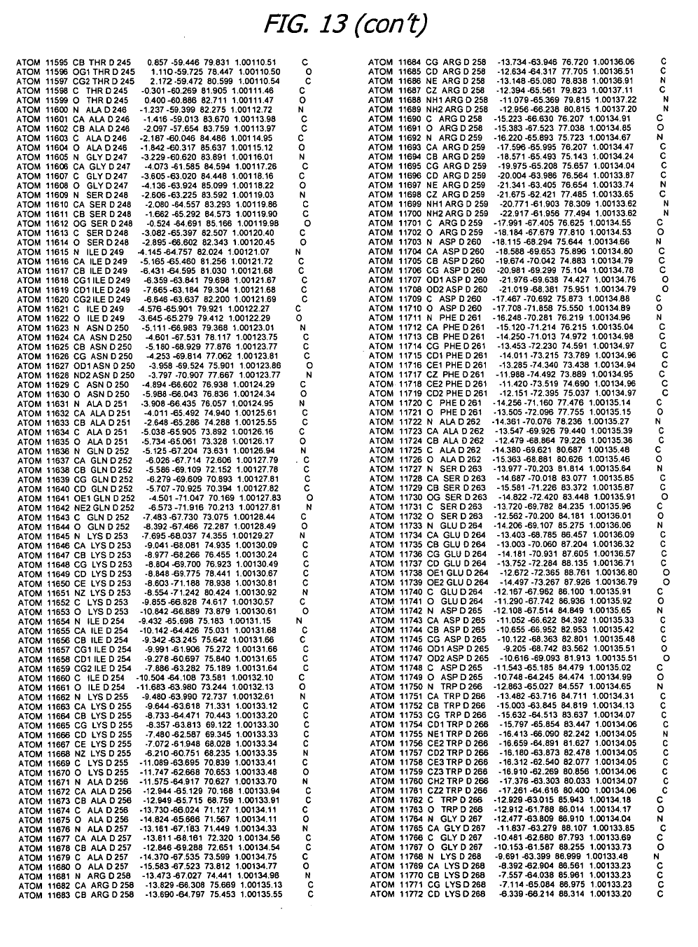

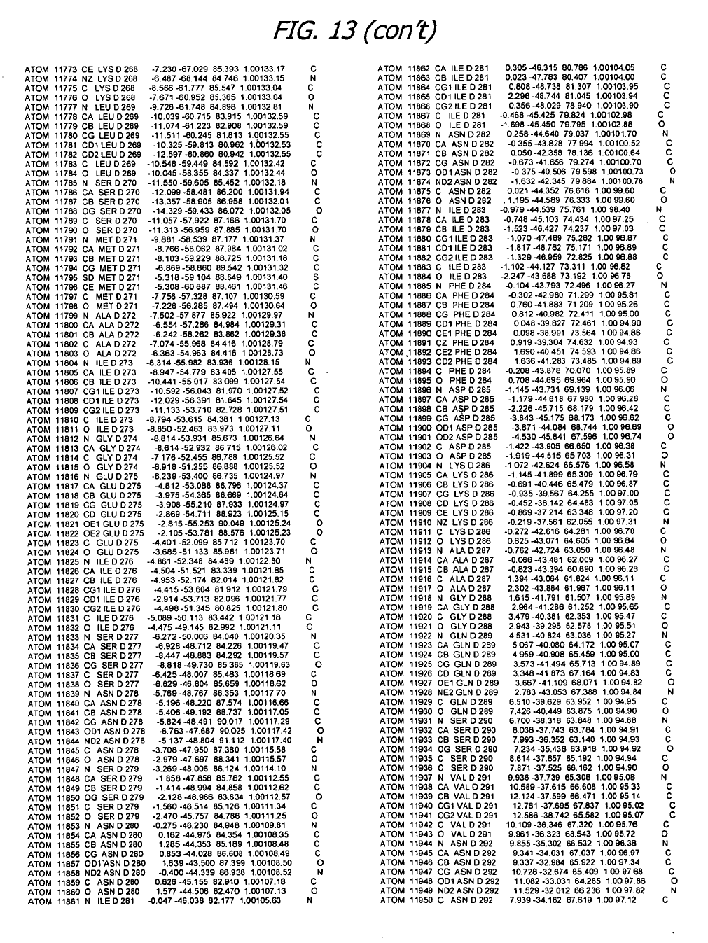

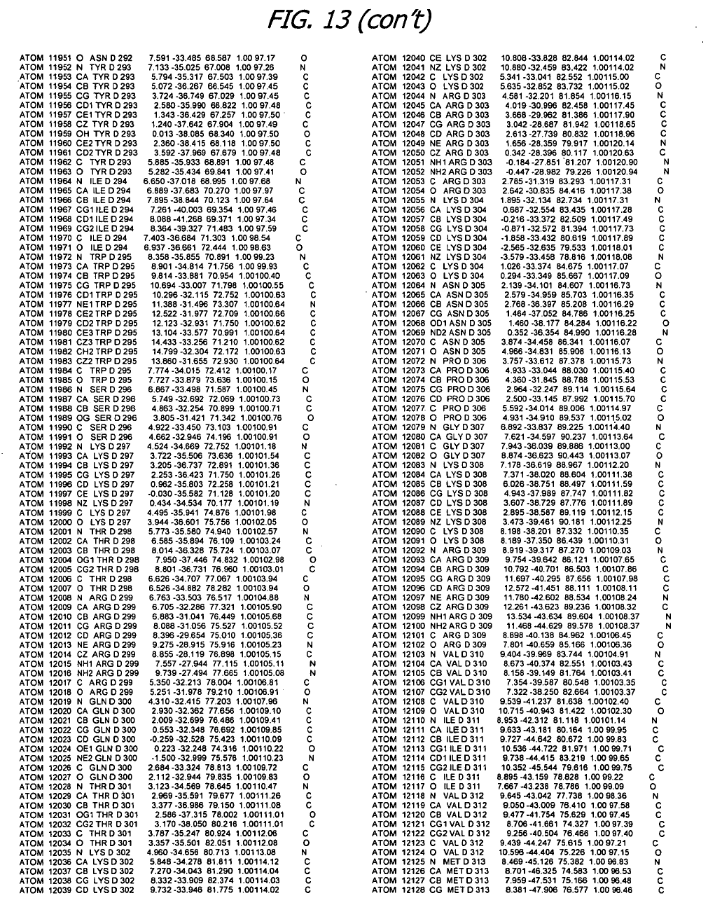

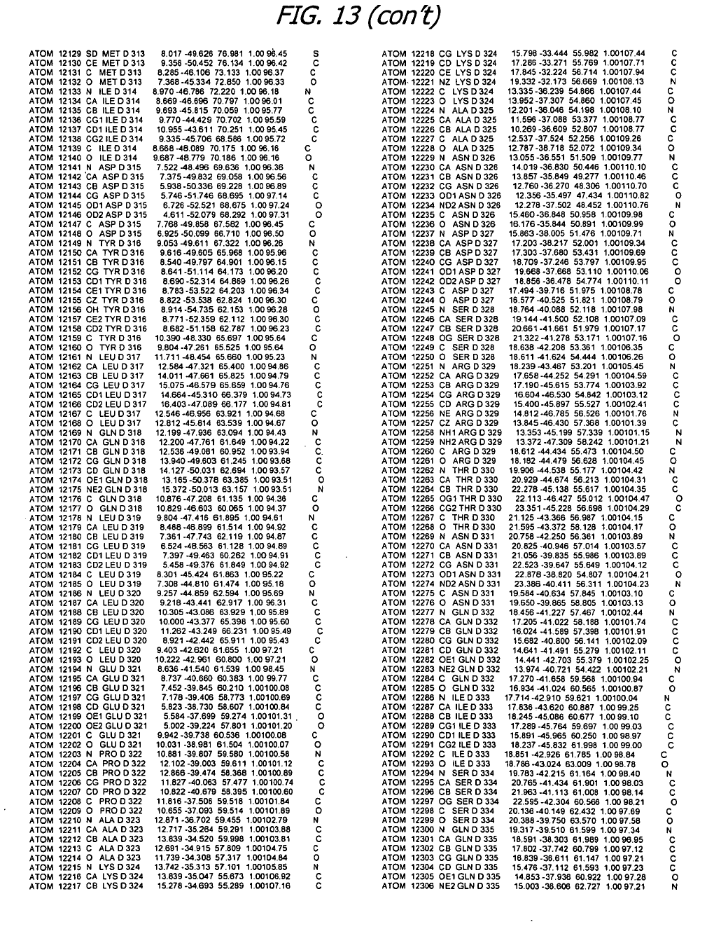

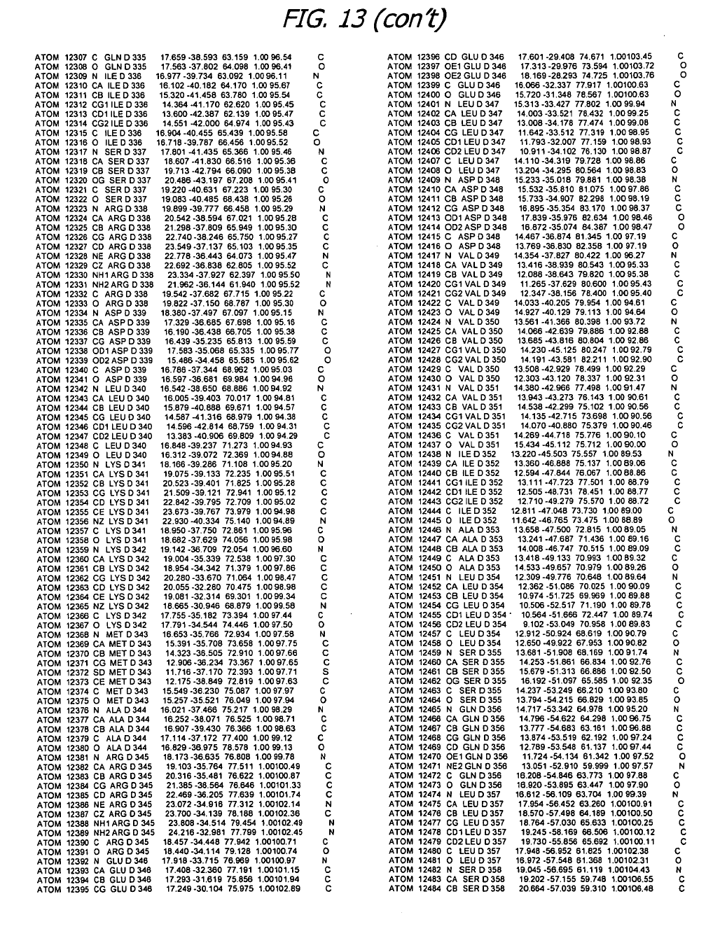

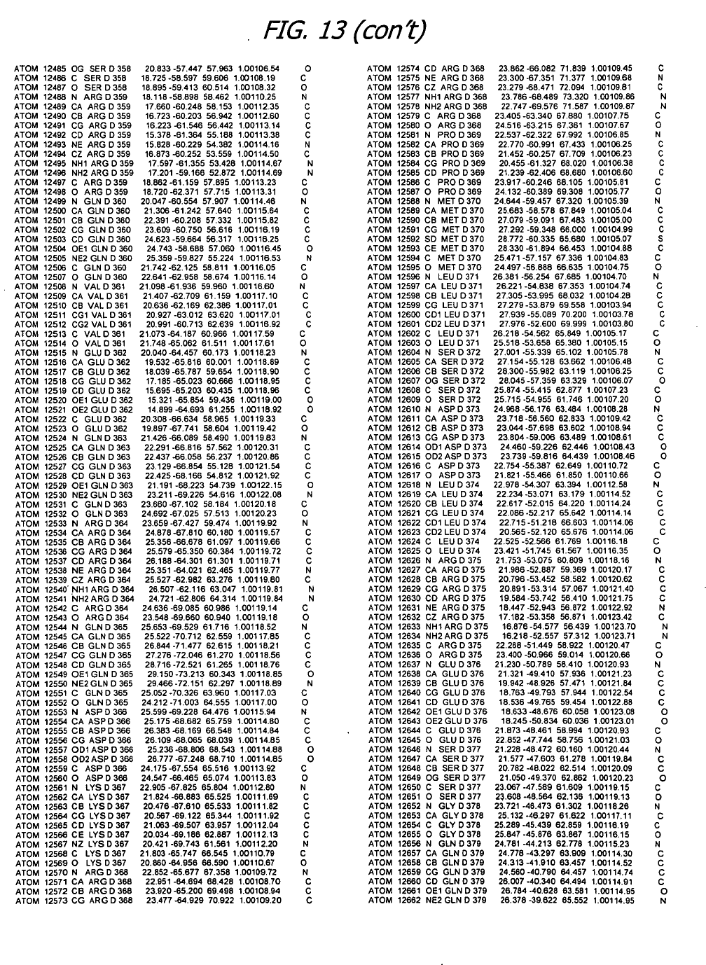

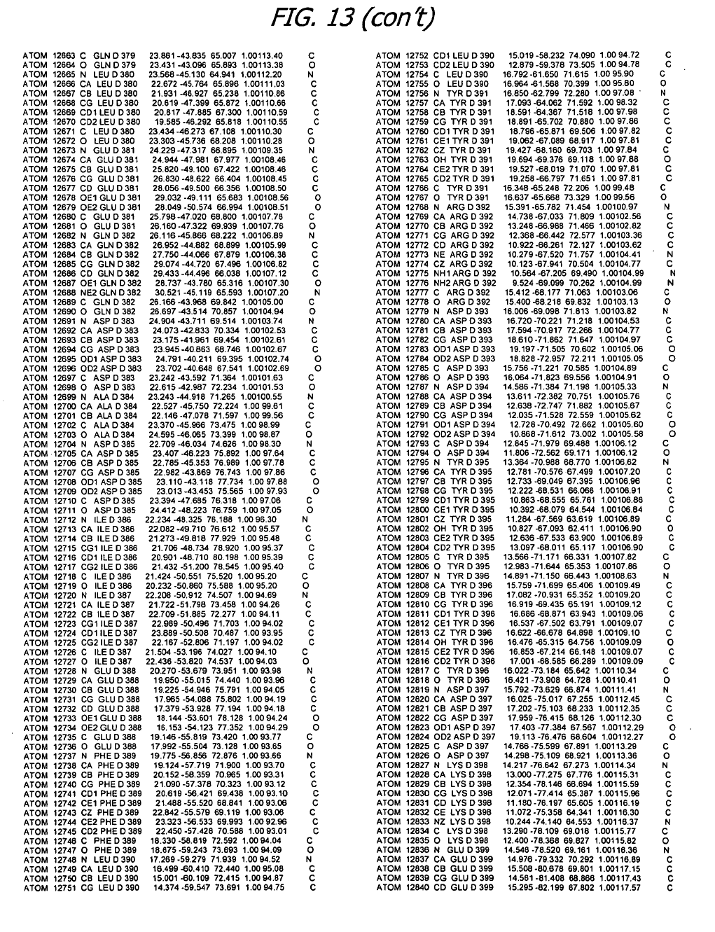

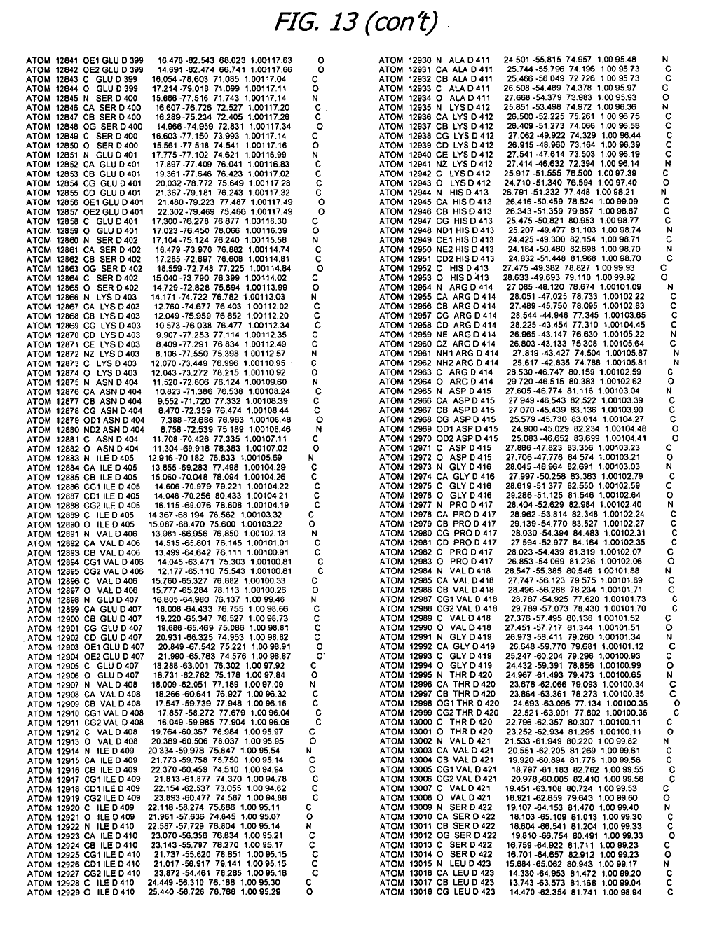

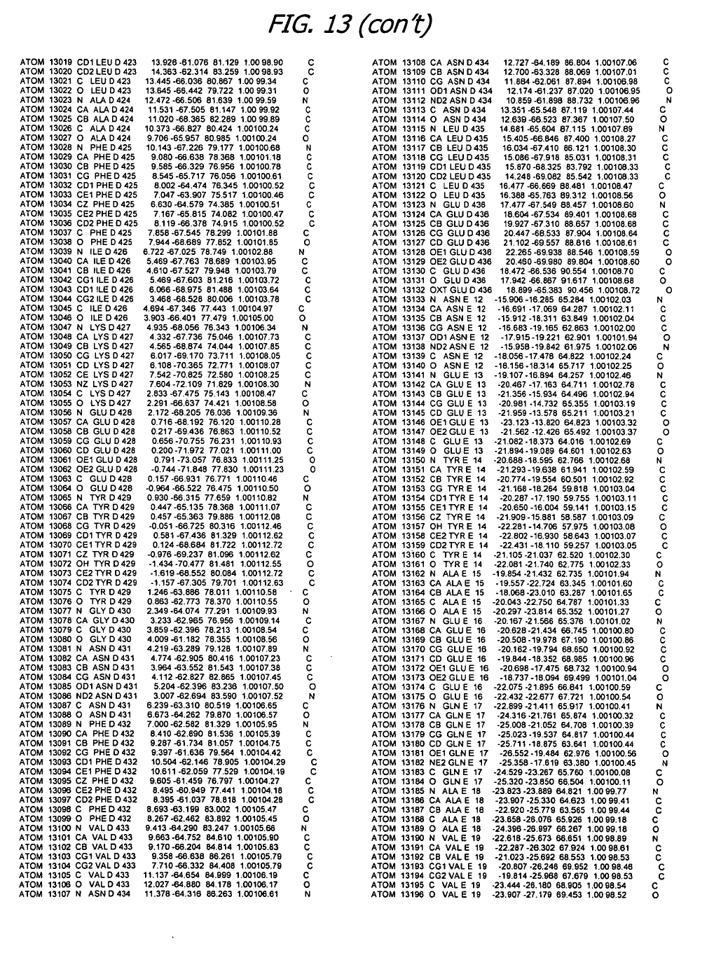

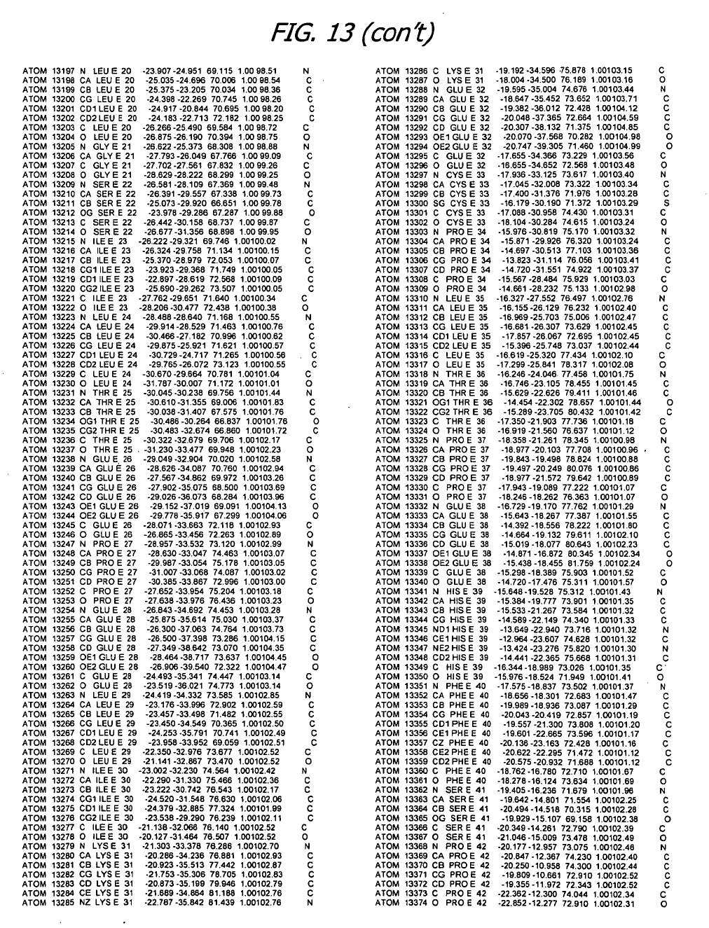

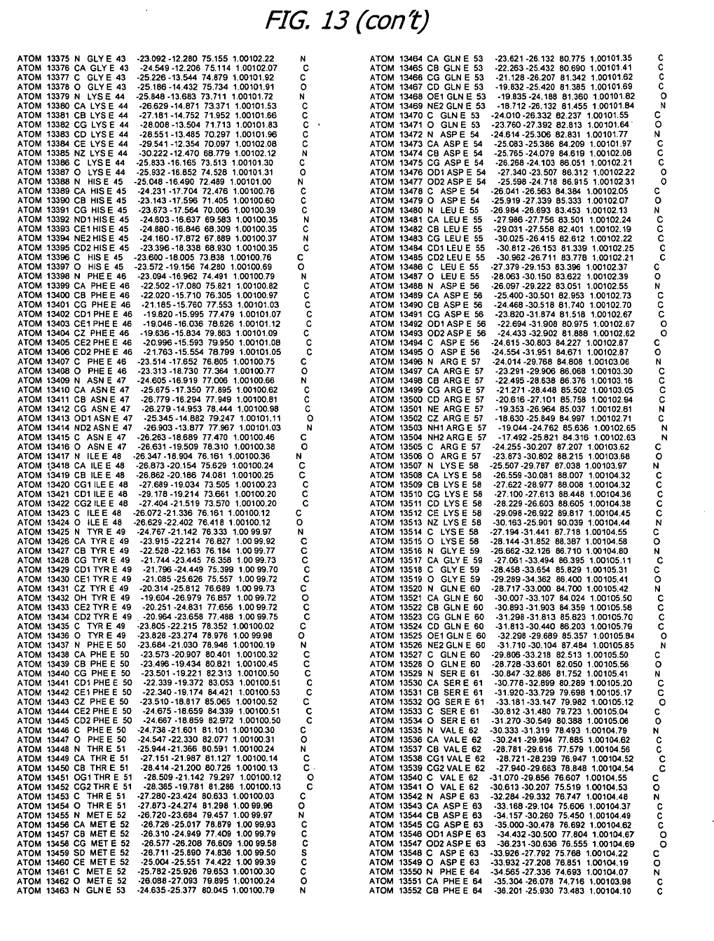

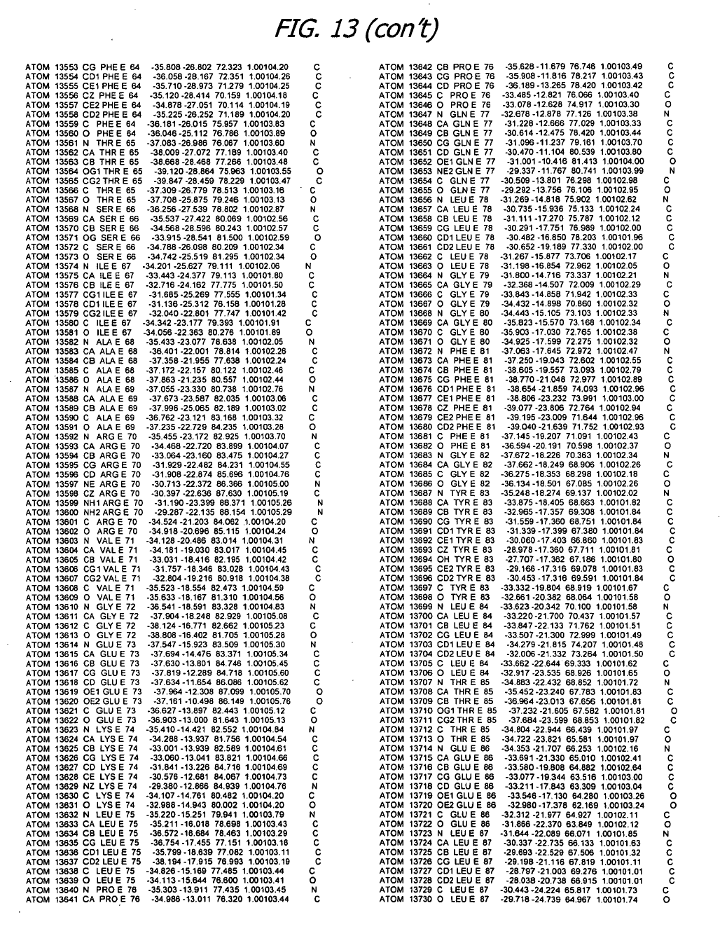

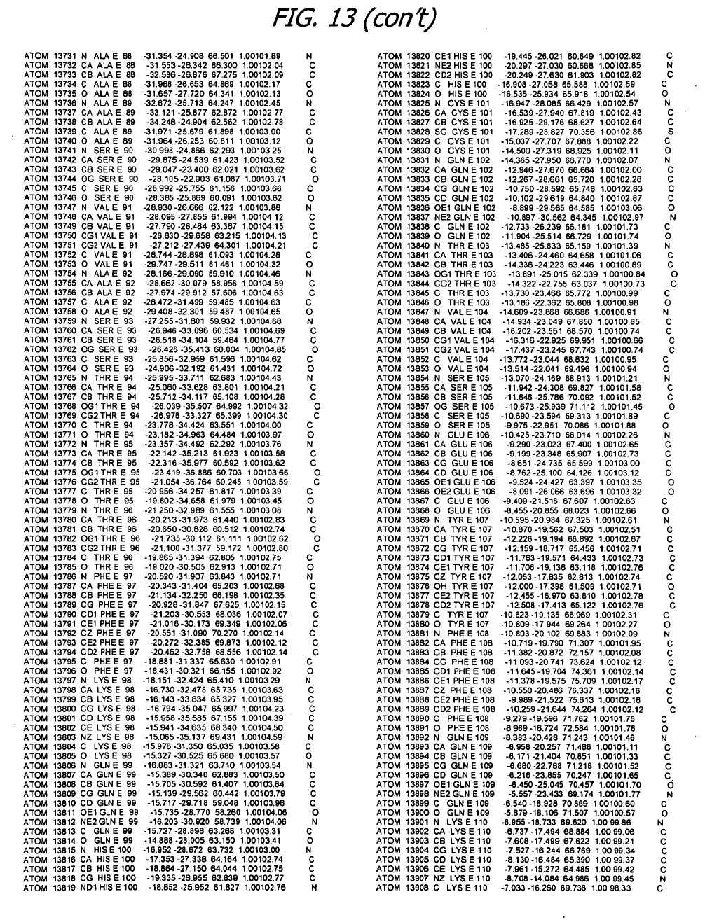

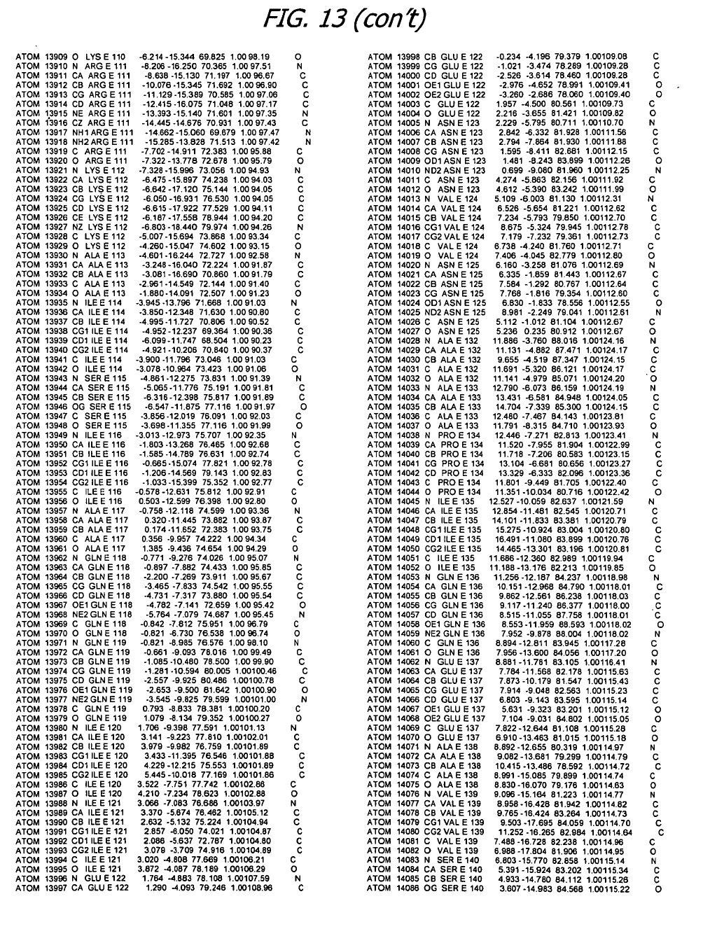

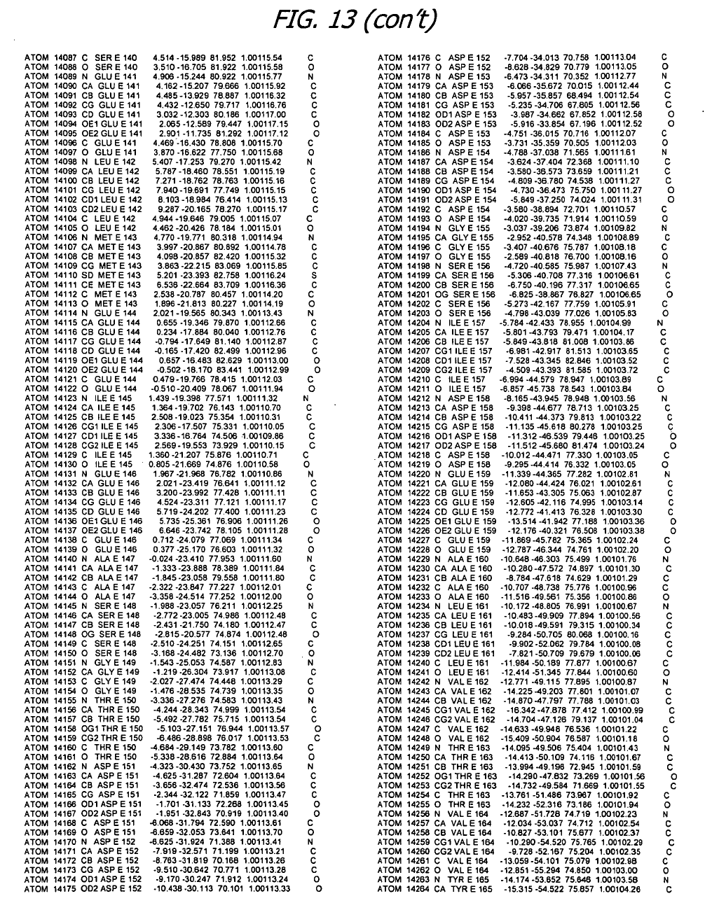

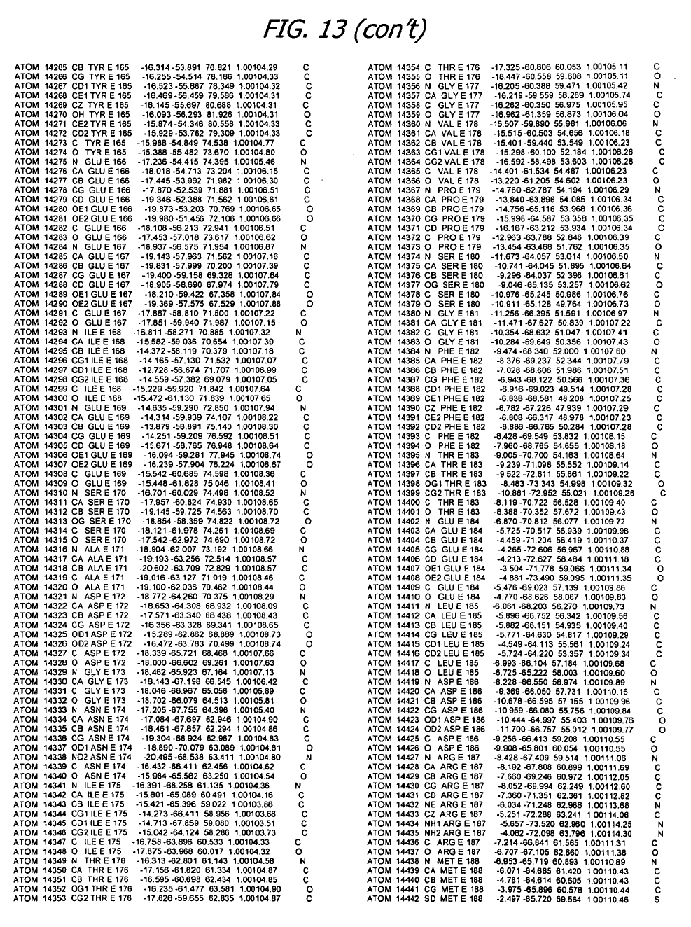

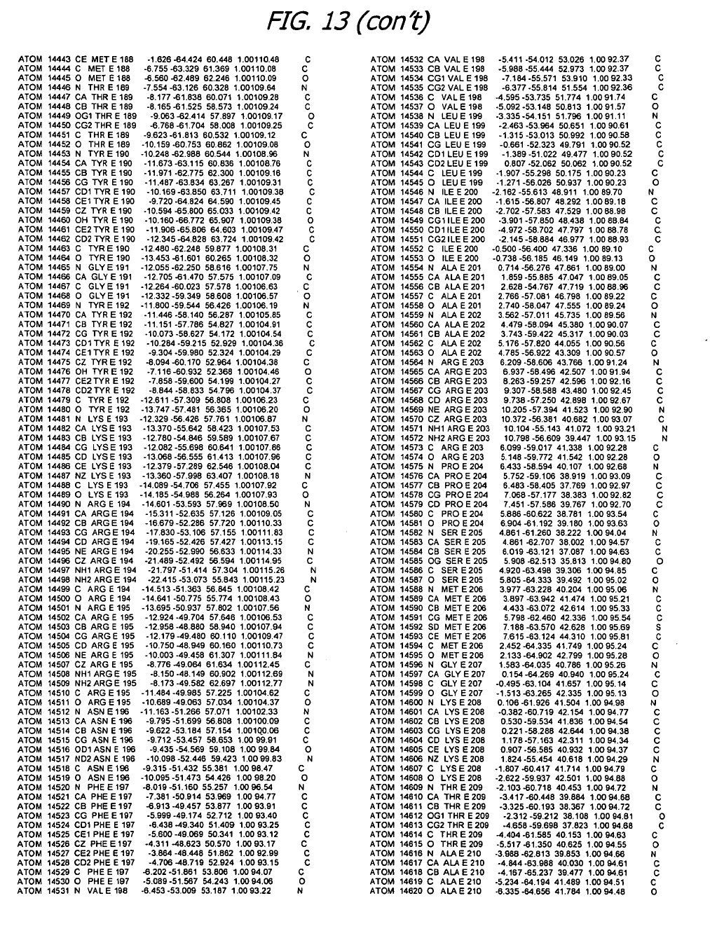

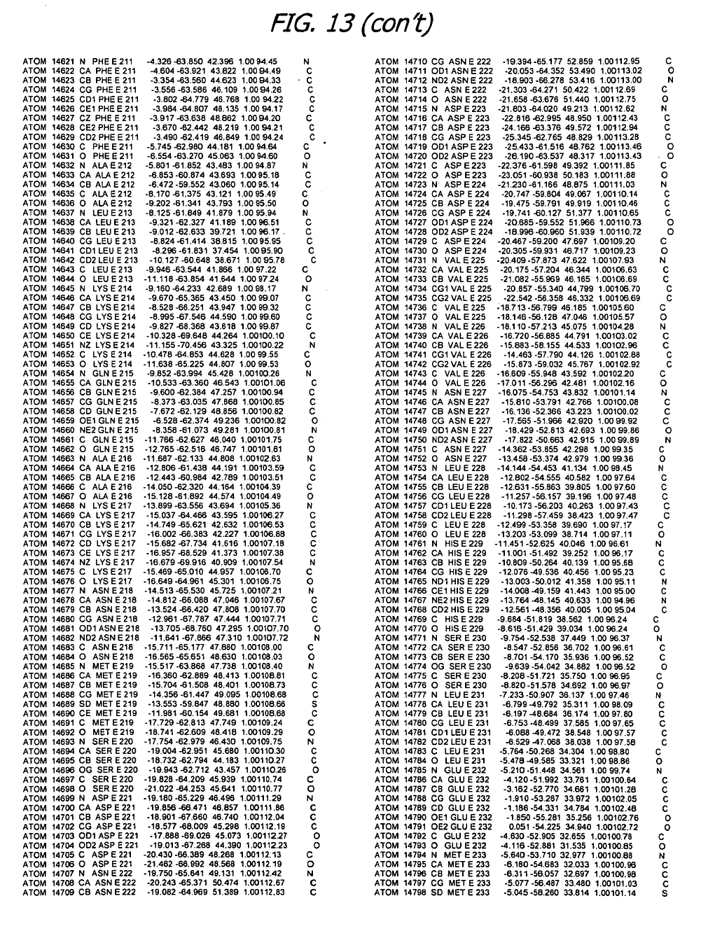

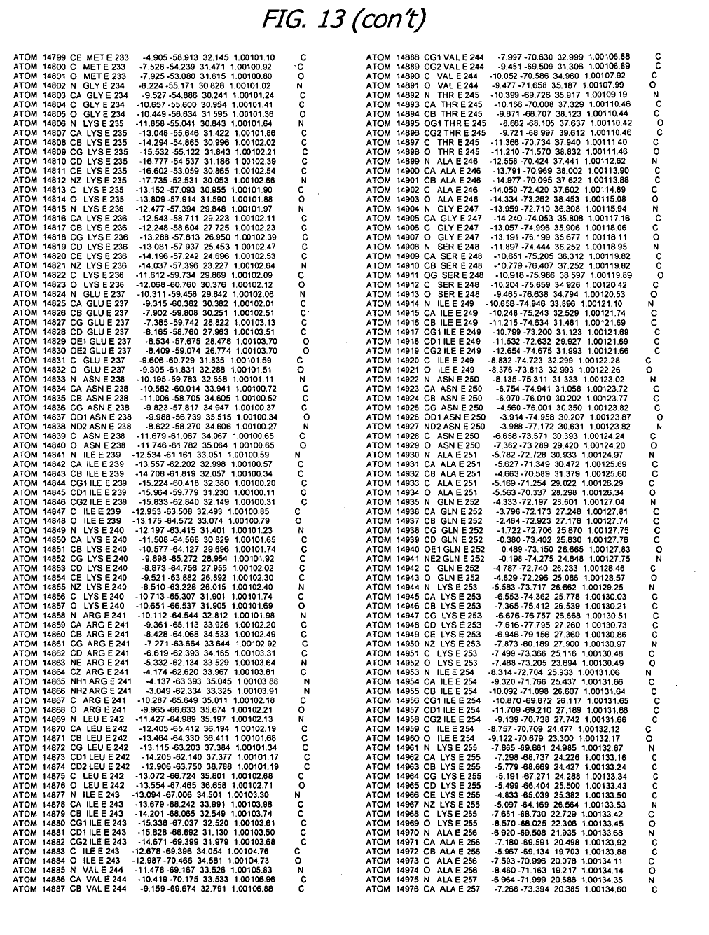

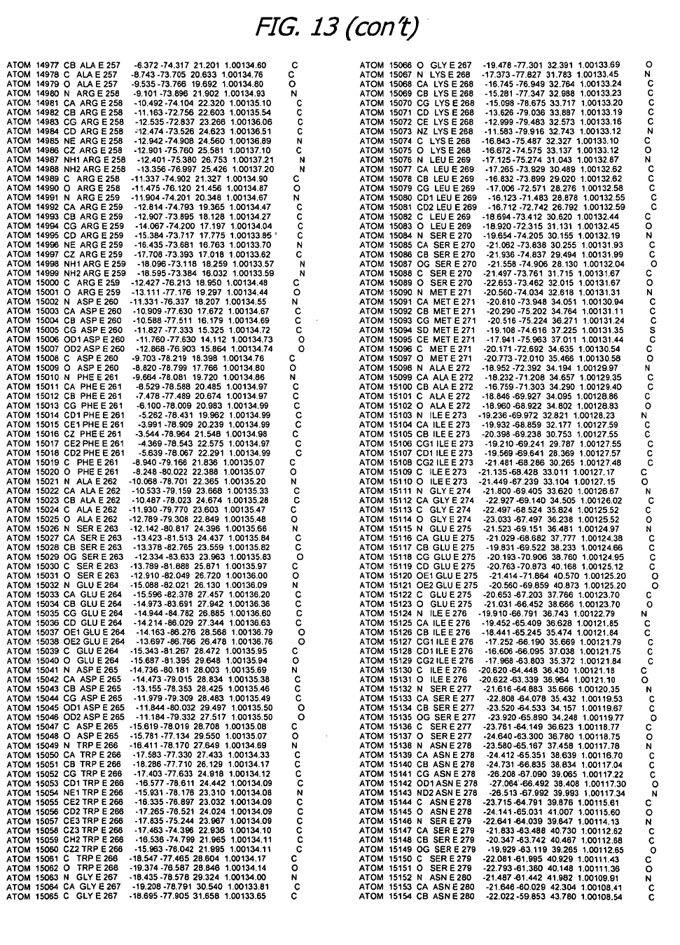

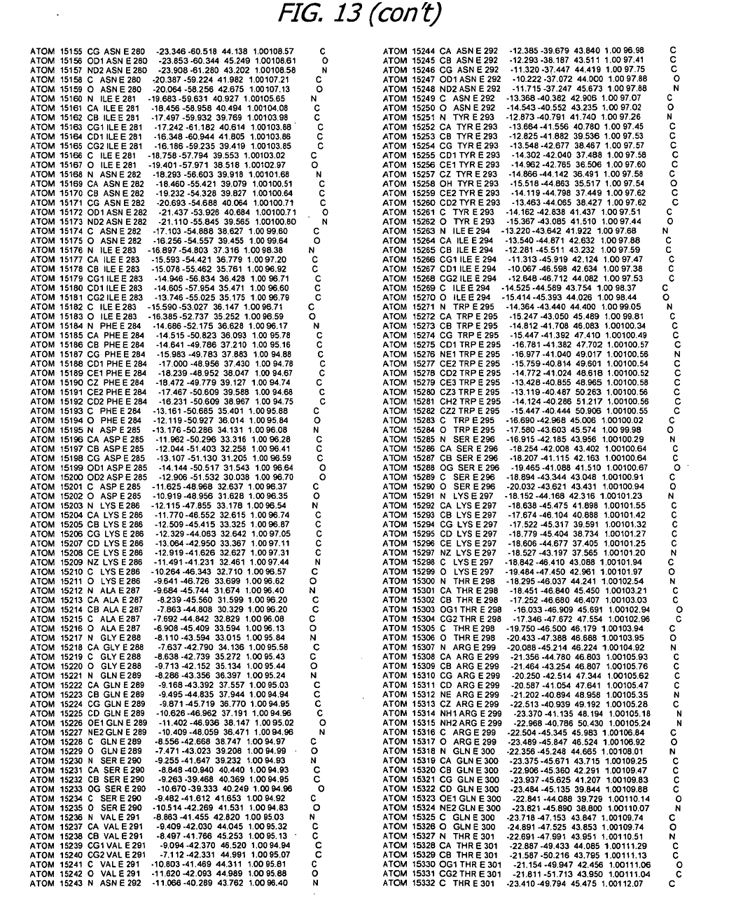

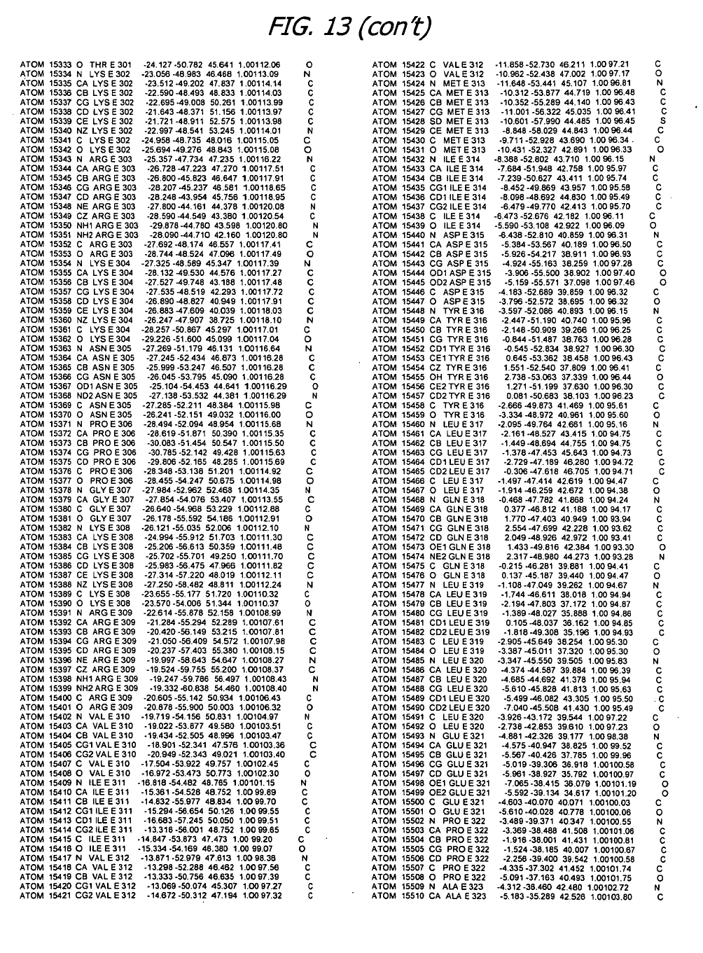

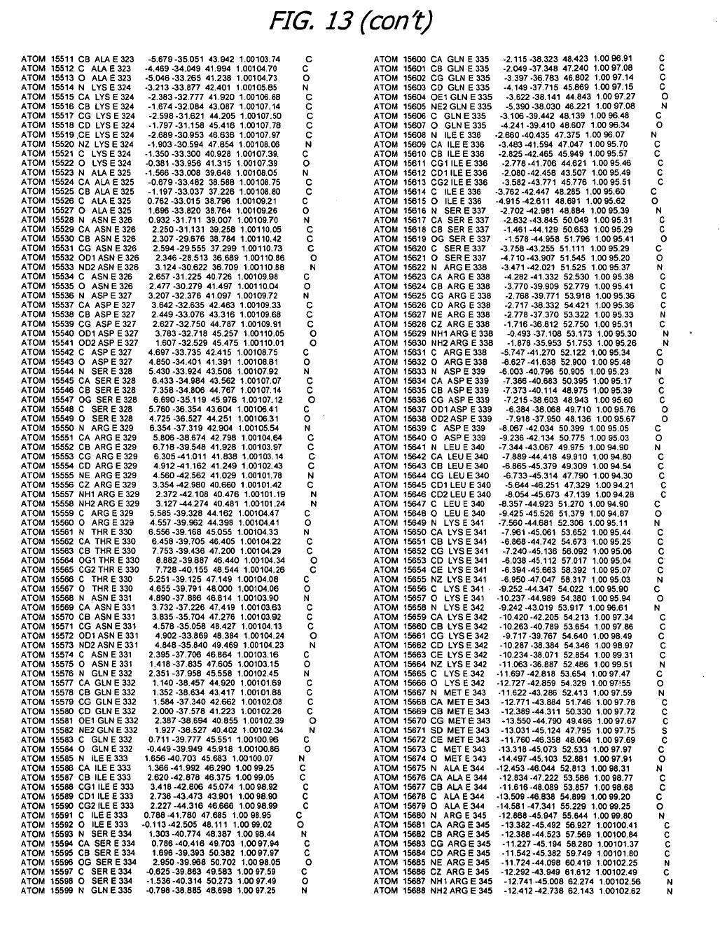

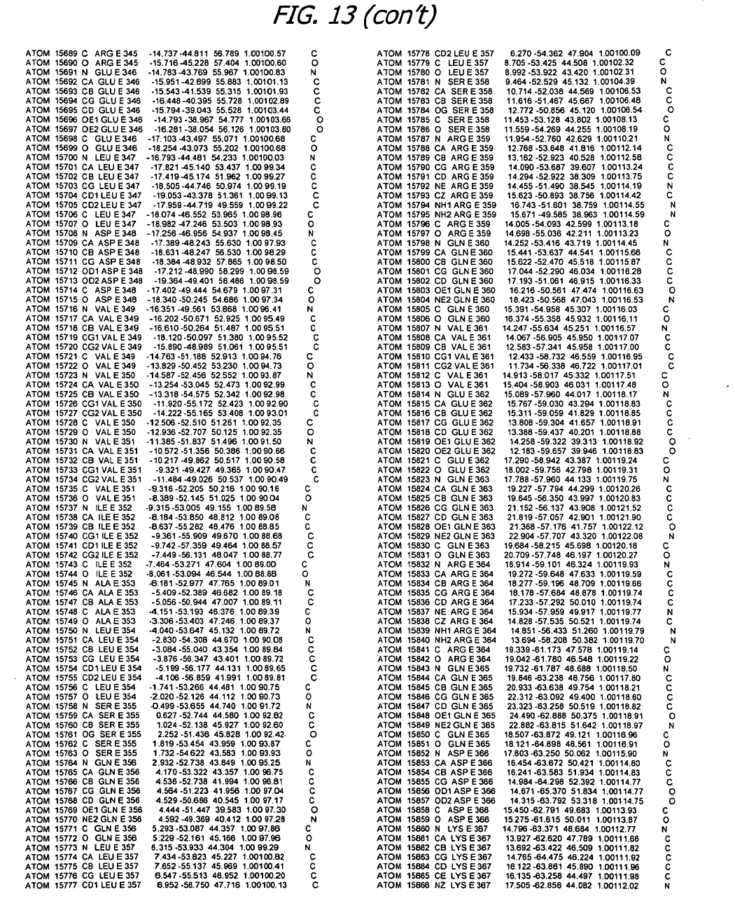

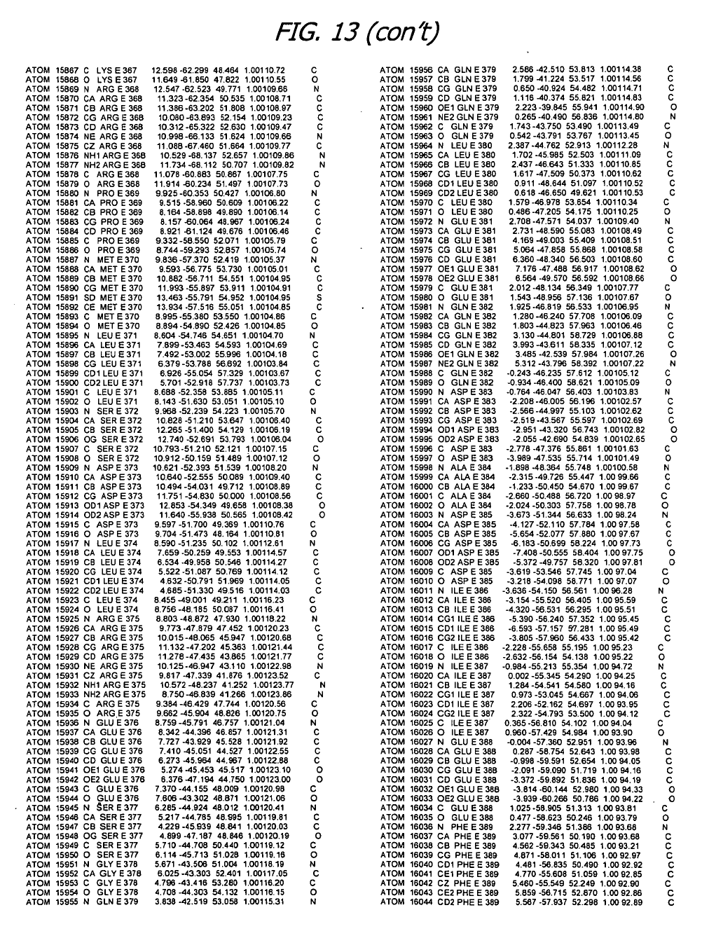

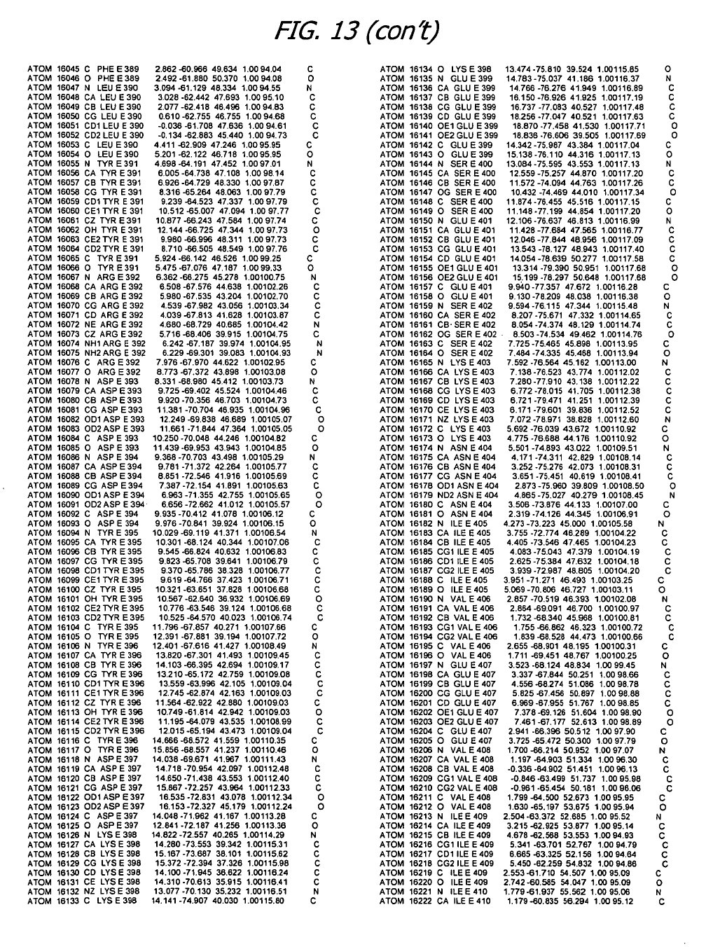

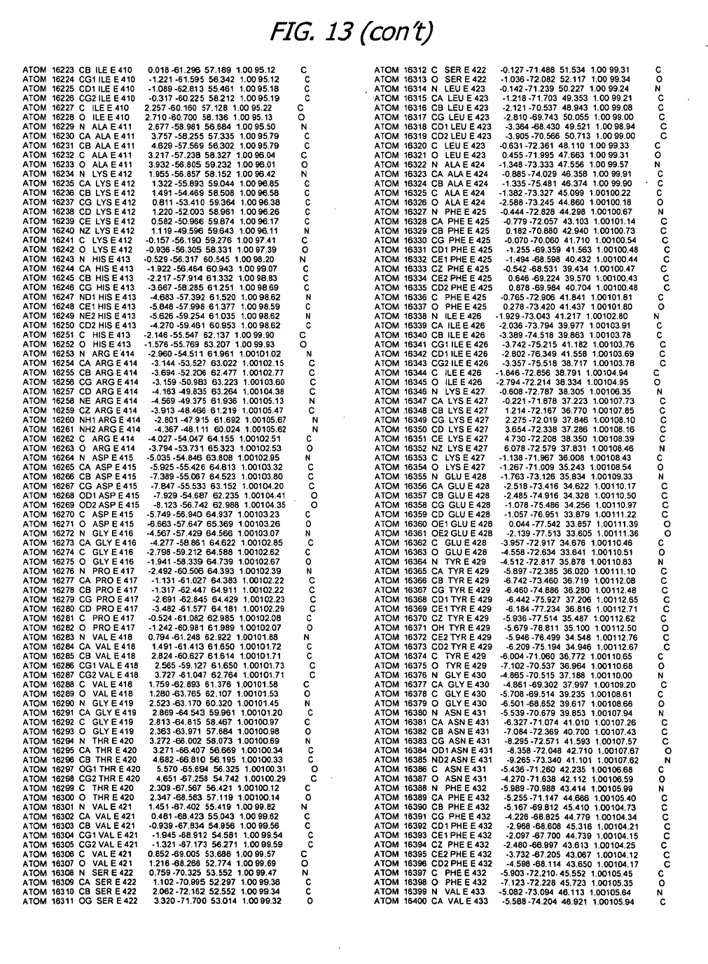

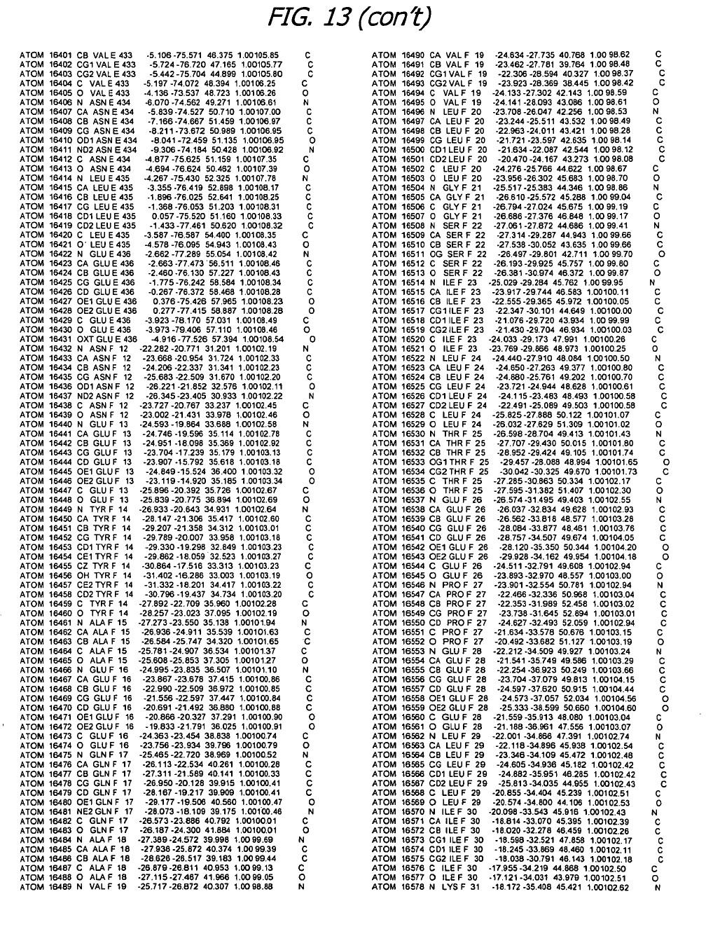

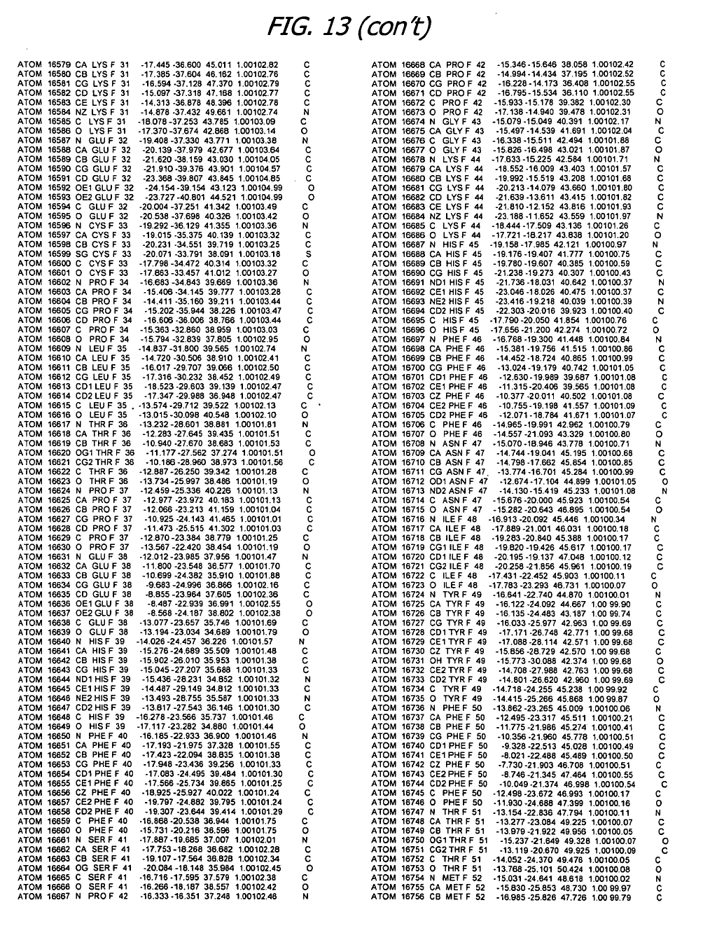

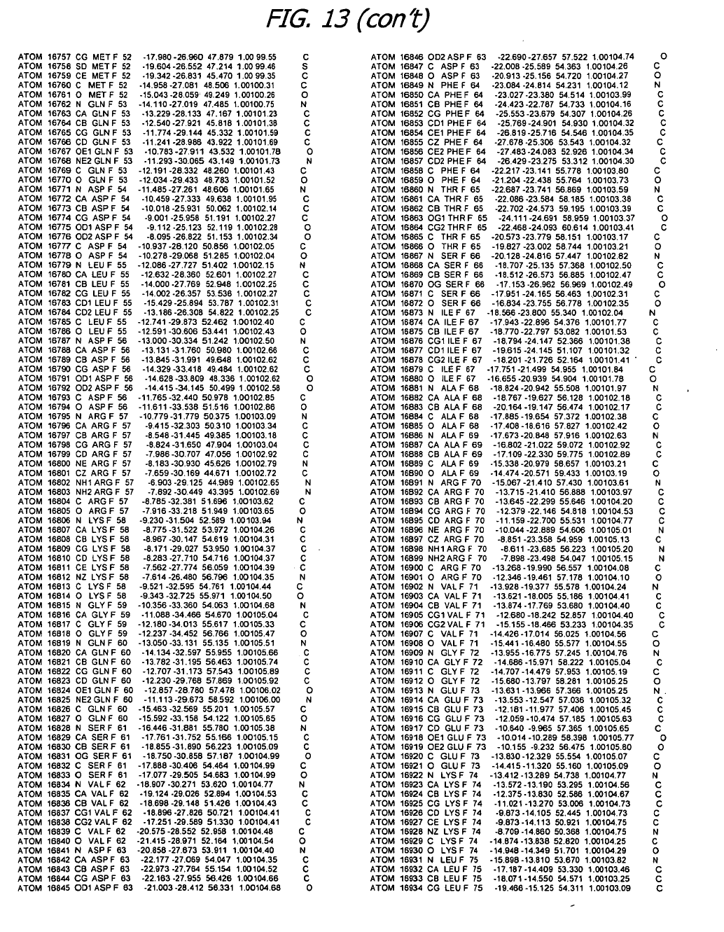

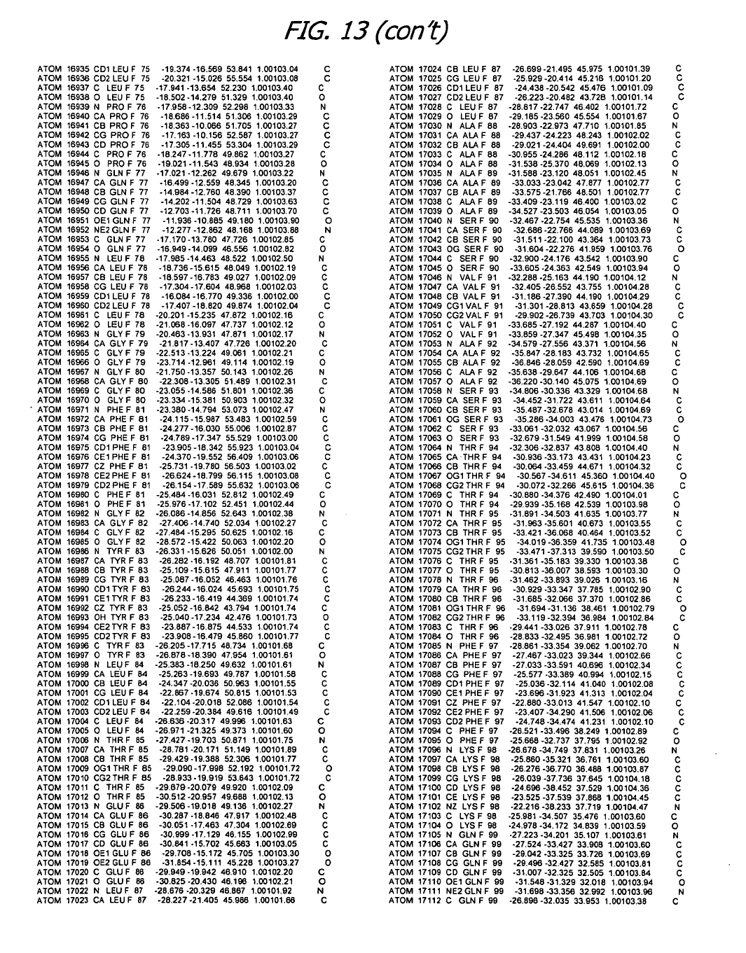

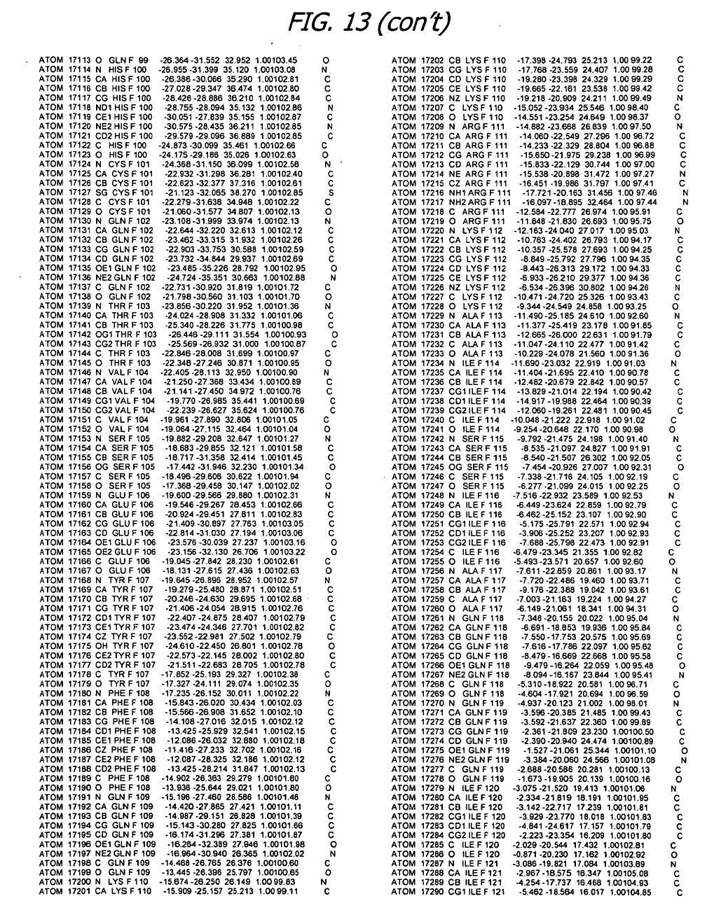

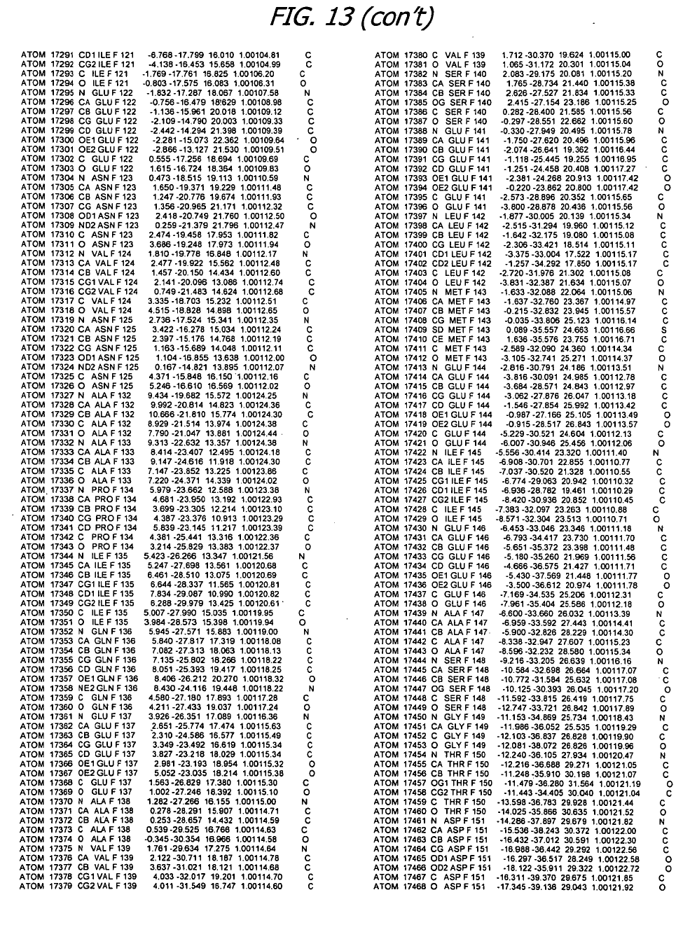

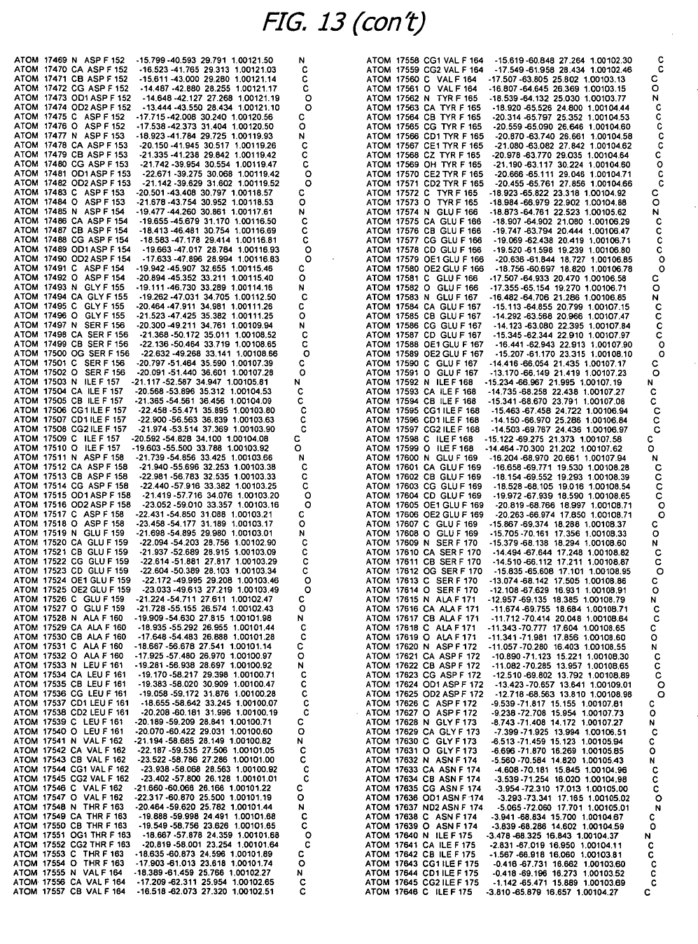

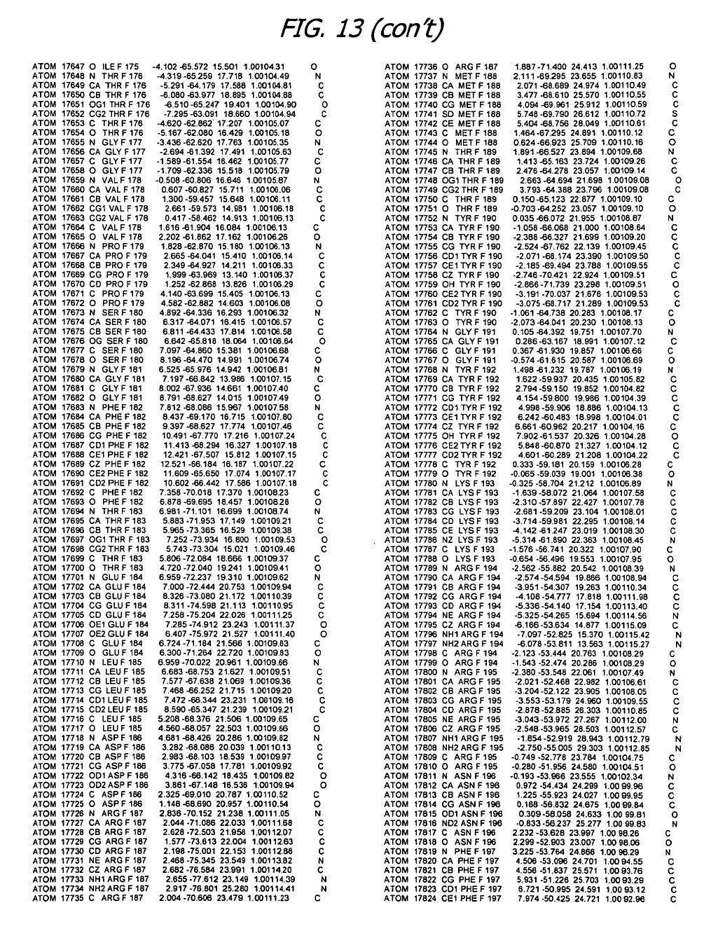

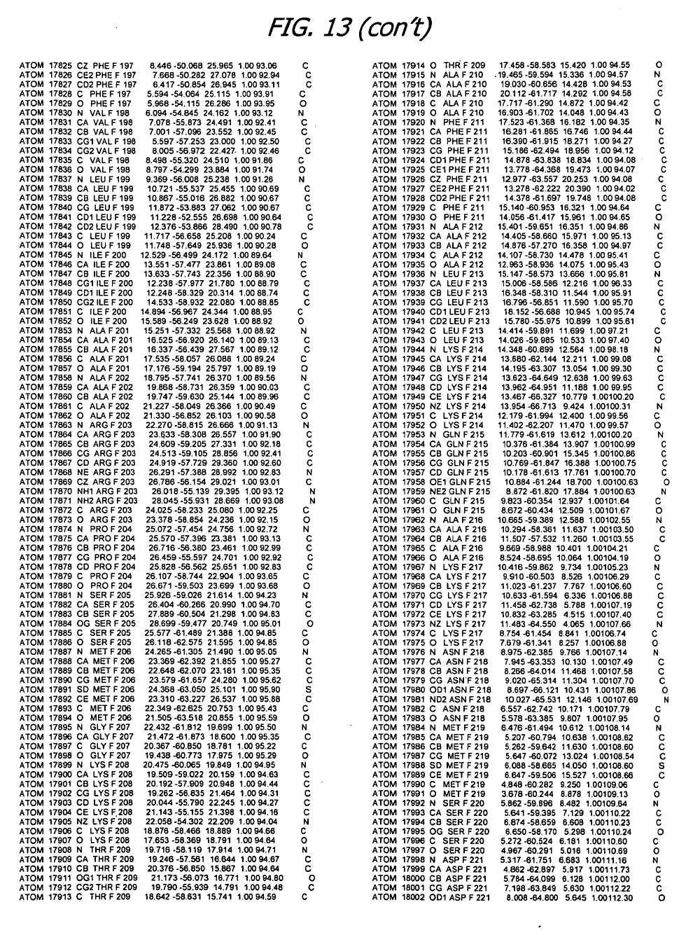

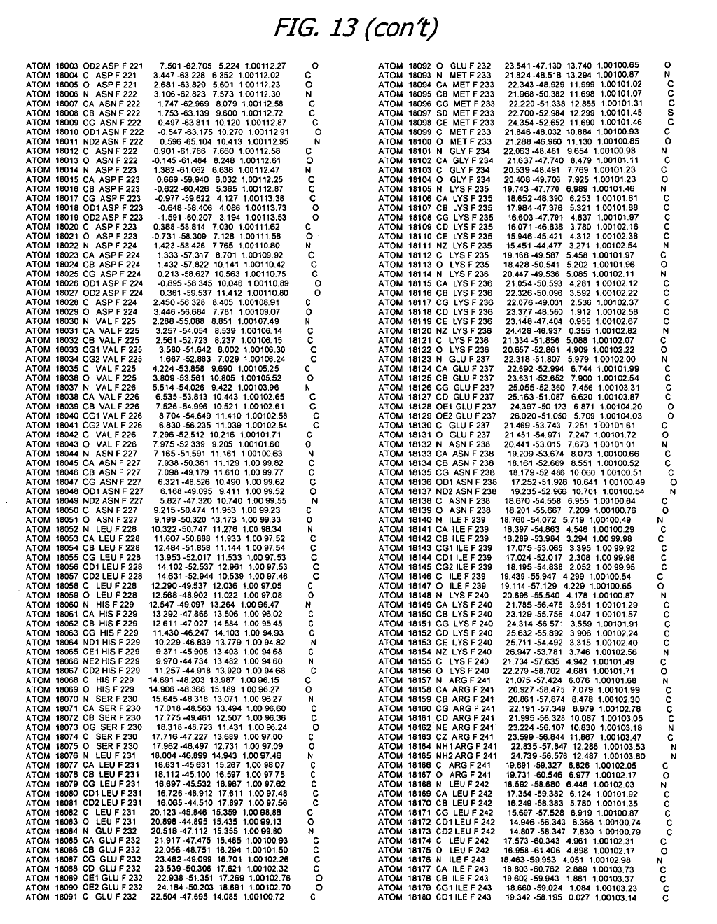

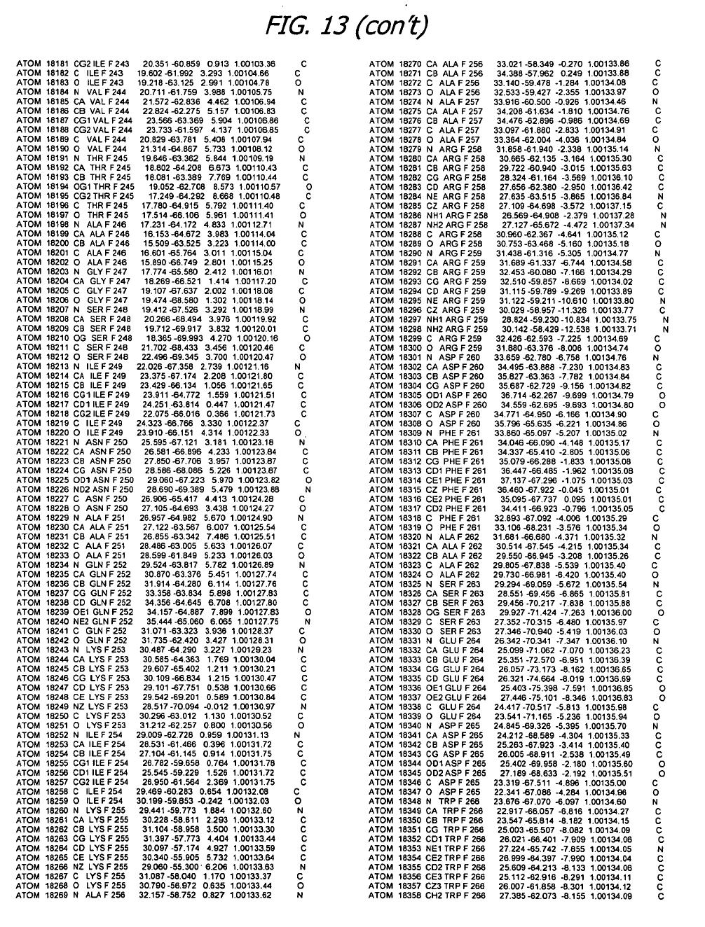

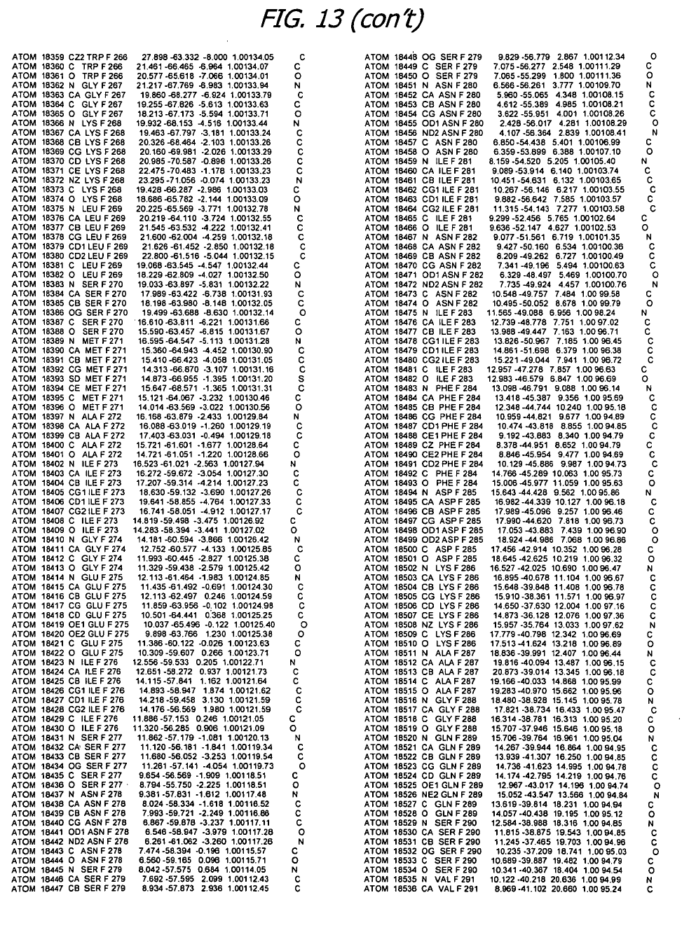

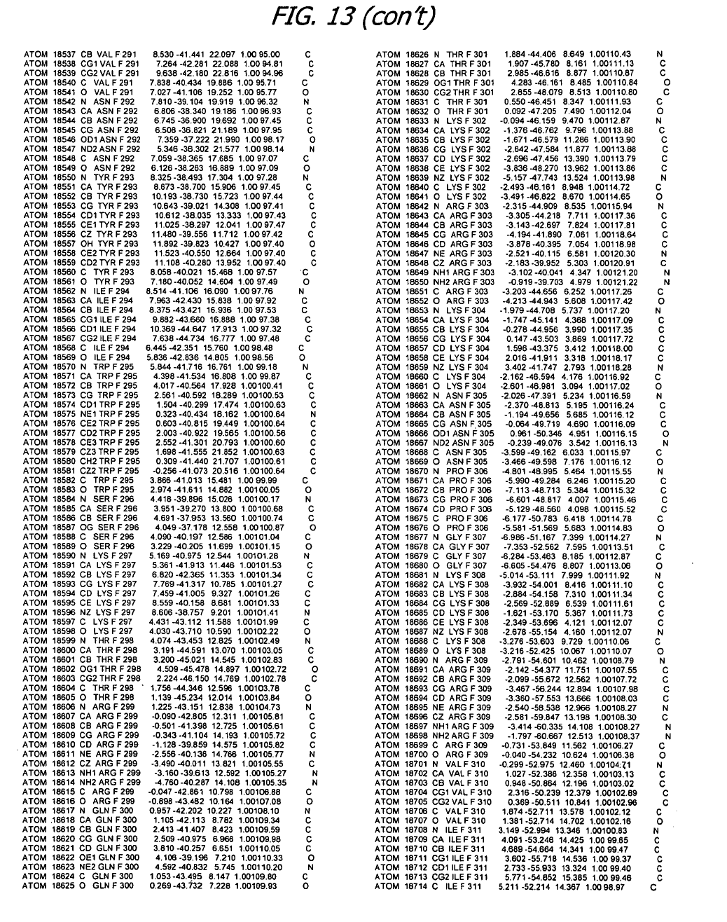

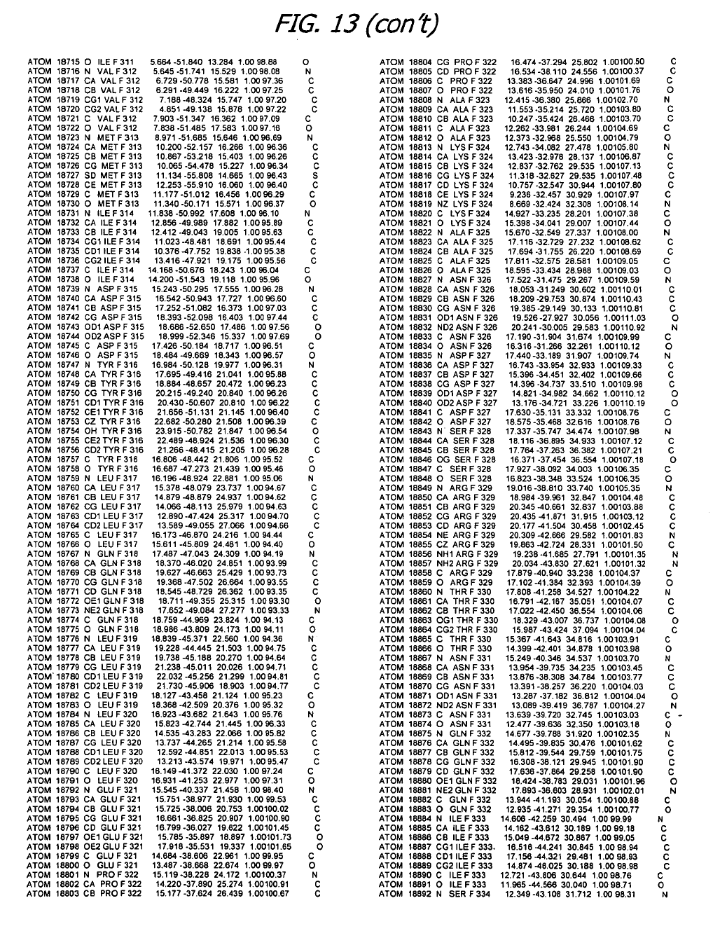

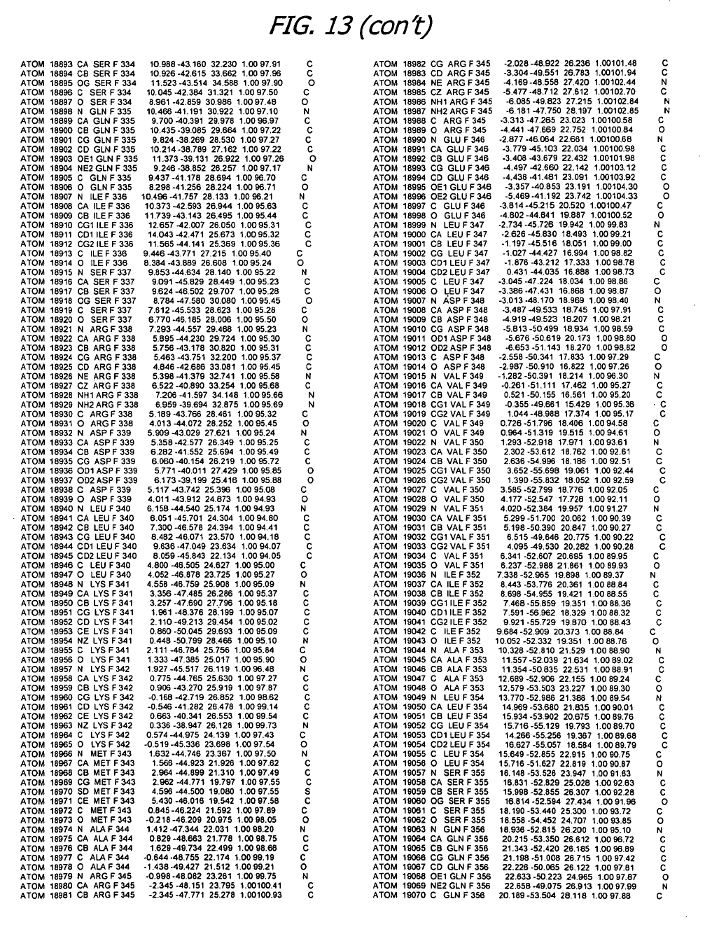

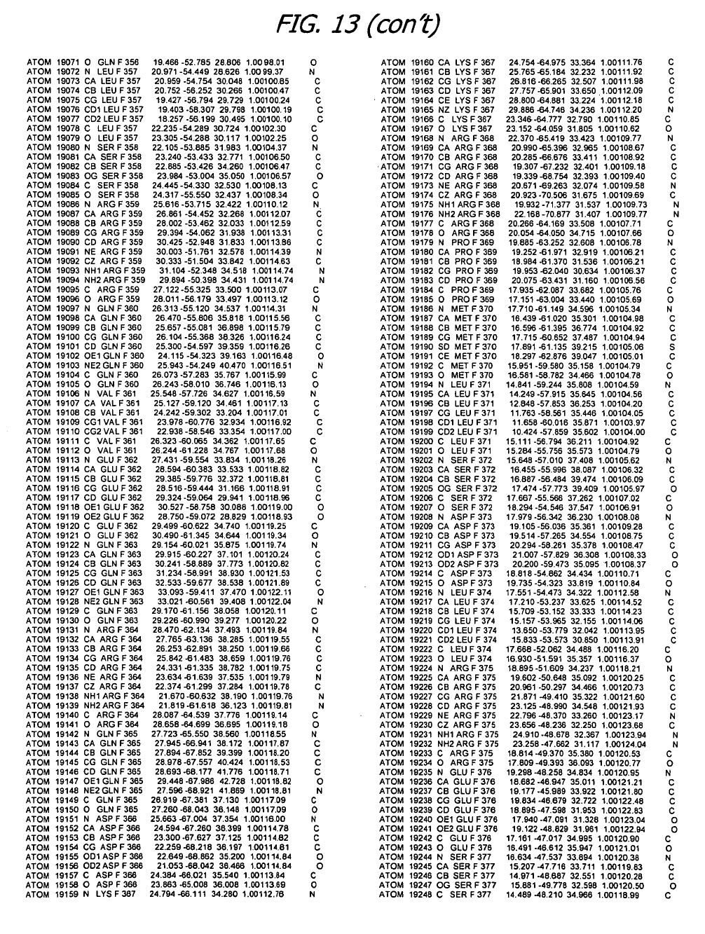

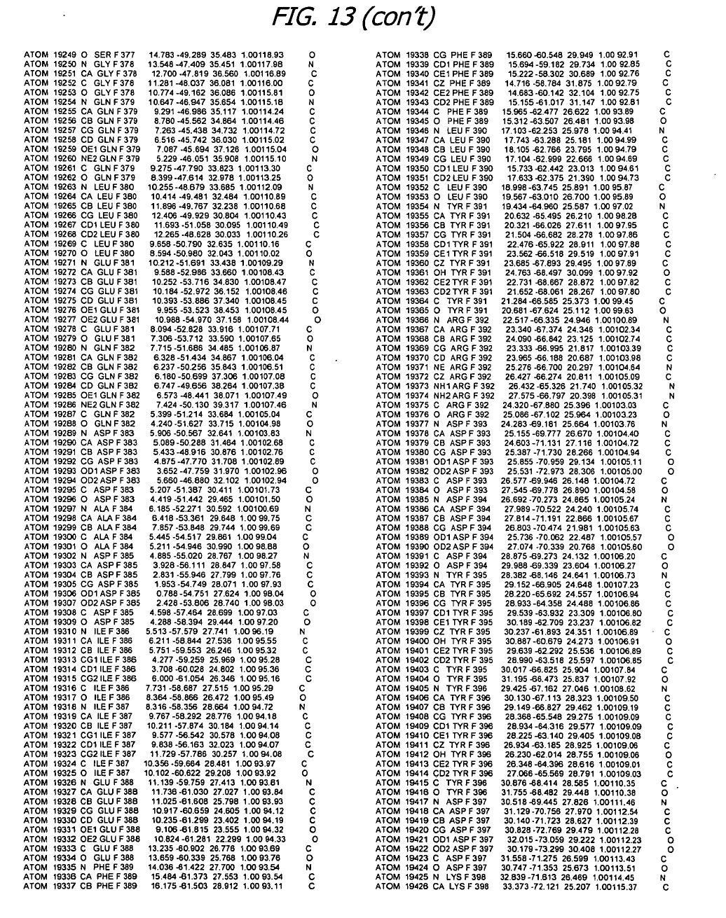

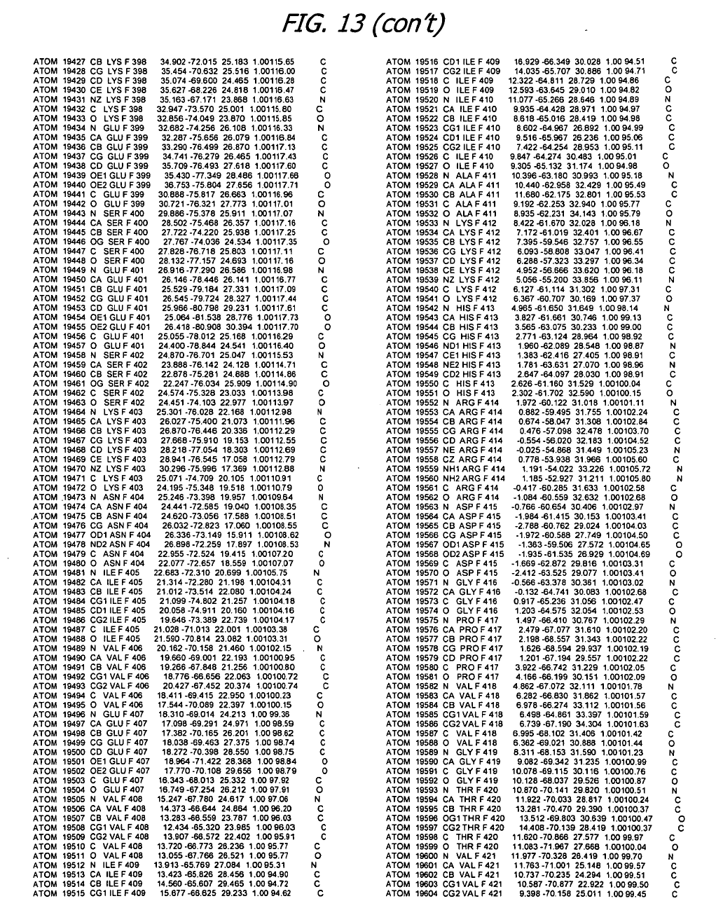

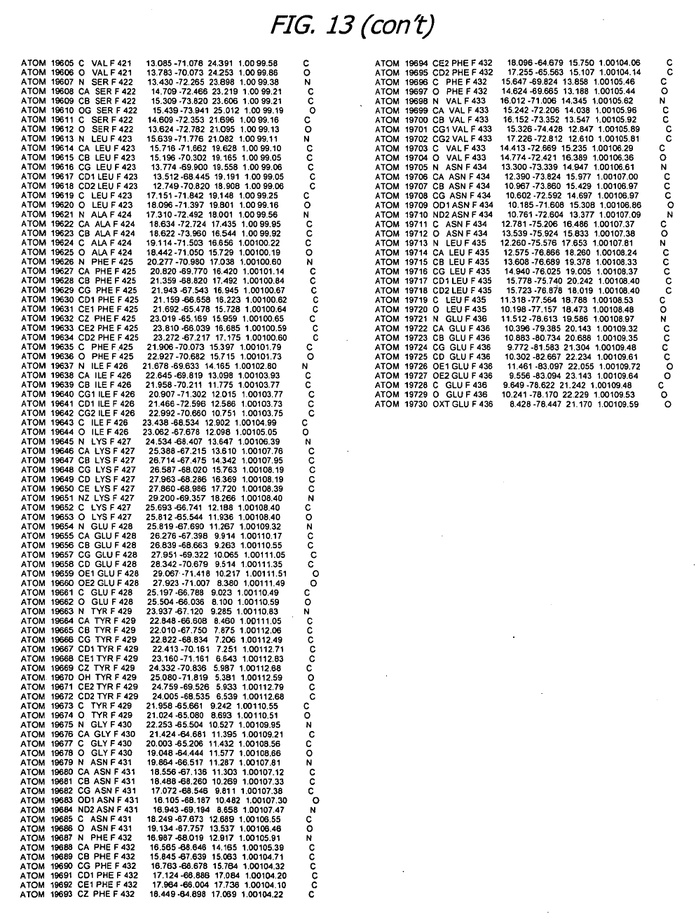

The role of N-terminal regions of DnaB homologs in helicase activity remains controversial. Therefore, we investigated the requirement of the N-globe and α-hairpin of G40P for helicase activity by N-terminal truncations (FIG. 4a). All the deletions (ΔN92, ΔN₁₀₈, ΔN112, ΔN129) and the full-length readily assembled into hexamers in gel filtration, even in the absence of ATP (data not shown). In helicase assays, the ΔN92 mutant that lacks the N-globe, but has an intact α-hairpin, retained ˜66% of the wt activity (FIG. 11). Other deletions (ΔN108, ΔN112, ΔN129) that lack an intact α-hairpin showed no detectable helicase activity. These results suggest that the α-hairpin structure of the “linker” region is critical for helicase activity. In contrast, the N-globe is not an essential component for helicase function, even though deleting the N-globe consistently resulted in reduced helicase activity.

G40P/DnaB replicative helicases bind DnaG primase at the replication fork, this interaction is important for coordinating DNA unwinding by the helicase with RNA primer synthesis by the primase. We investigated the requirement of N-terminal domain of G40P in primase binding using the N-terminal deletions. In contrast to the full-length G40P, all four N-deletions (ΔN92, ΔN108, ΔN112, ΔN129) were devoid of primase binding in a native gel-shift assay (FIG. 11), demonstrating the importance of the N-terminal domains containing at least the first 92 residues in DnaG binding. We next examined if the isolated N-terminal domain of G40P can bind primase. The two constructs containing only the N-terminal α-hairpin and N-globe domains (N149 and N171) had no detectable primase binding in native gel shift assays (FIGS. 4d and 11). Both constructs behaved like monomers in gel filtration (data not shown).

To identify residues on the N-terminal tier of G40P that participate in primase binding, we constructed three point mutations (mt1-mt3, FIG. 11). The location of mt1, mt2, and mt3 are shown in FIGS. 4b and 4c. These three mutants assembled into hexamers in gel filtration chromatography (data not shown). However, none of them showed any detectable primase binding (FIGS. 4e and 11), suggesting a role of these mutated residues in mediating the interaction with DnaG primase.

Inter-Tier Interactions are Required for Primase Stimulation of G40P

Primase binding to DnaB N-tier stimulates ATPase and helicase activity. In order to test the role of inter-tier interactions in the primase-mediated helicase stimulation, two mutants were designed to disrupt the interactions of the C-terminal helicase-tier with either the N-globe (mt4), or with the α-hairpin (mt5) of the N-tier (FIG. 4b). Both mutants assembled into stable hexamers. However, mt5 was unable to bind DnaG (FIGS. 4e and 11), possibly due to a disruption in the packing of the N-terminal α-hairpins with the helicase domain, disturbing the structural integrity of the N-tier that is important for primase-binding. Mt5 also lost helicase activity (FIG. 11), consistent with the essential role of the α-hairpin for helicase function. In contrast to mt5, mt4 possessed wt-level ATPase/helicase activities and bound primase (FIGS. 11, 4e and 4f). Interestingly, mt4 displayed much reduced primase-mediated stimulation of the ATPase and helicase activity of G40P (FIGS. 4f and 11).

Through the experiments discussed herein, the crystal structure of the full-length G40P that forms one complete hexamer in an asymmetric unit was determined. Two distinct monomer conformations, termed cis- and trans-structures, coming together alternatively to assemble into one hexamer with an unusual dual symmetry: a near 3-fold N-terminal tier and a pseudo 6-fold C-terminal tier, as disclosed herein, has been identified. The G40P structure guided mutagenesis has demonstrated the importance of the N-terminal domains for helicase function, and has mapped the DnaG-binding sites on to the N-terminal tier that is composed of the N-globe and α-hairpin structures, and has provided evidence to suggest a mechanism of how primase-binding affects DnaB helicase function.

This study clearly demonstrated the important role of the N-terminal domains for helicase function, as deleting the N-globe had significant reduction of helicase activity, and further deletion of a few residues into the α-hairpin region essentially caused a complete loss of helicase function (FIGS. 4a and 11). As these deletion mutants all retained significant level of ATPase activity (FIG. 11), these deletion results indicate that the structural integrity of the 3-fold N-tier has affected the helicase function much more than the ATPase activity.

The deletion studies revealed that the N-terminus comprising the N-globe or longer fragment plays an important role for primase binding. However, the isolated N-terminal fragments containing the N-globe and α-hairpin, which exist as a monomeric form and not in the 3-fold N-tier conformation, did not show any detectable primase binding. This suggests that primase may bind to the N-terminal domains only when they are assembled into the 3-fold N-tier, which may only occur in the context of the full-length hexamer.

Mutagenesis analysis of residues located on the surface of N-tier (mt1 and mt2, FIGS. 4b, 4c) suggests a potential role for these residues in mediating primase interaction either directly or indirectly. This result is consistent with published mutational and genetic studies in different organisms^{13,16,29,30}, in which the residues affected primase binding are mapped to the similar locations on the surface of the 3-fold N-tier of G40P (FIG. 7). In light of the structure by Bailey et al.³¹ in which one asu contains a DnaB dimer binding to one primase from Bacillus stereothermophilus (BH) through the two interacting N-globes of DnaB, the mutated residues on the α-hairpin surface of DnaB are not making direct contact with primase, suggesting that these residues disrupt primase binding indirectly. Alternatively, because of the reported difference in primase binding by DnaB from different organisms⁹, and the different structures shown for primase P16 fragment from BH and Escherichia coli (E. coli)^{32,33}, we can't rule out the possibility that more than one binding mode between DnaB and primase may exist.

The mutated residues of another mutant, mt3, are in two different environments depending if they are on the cis or trans monomer: these residues on the cis monomer are exposed (mt3-cis, FIG. 4b-c), and the residues in the trans monomer (mt3-trans, FIG. 4c) are at the interface with a cis N-globe to make the globe-globe interaction. This mutant was originally designed to disrupt the globe-globe interactions within the 3-fold N-tier to test the role of this interaction for primase binding. The recent publication by Bailey et al.³¹ suggests that not only this globe-globe interaction is important for primase binding, but also those exposed residues of mt3 on the cis monomer may be involved in direct contact with primase.

DnaB helicase can be stimulated by primase binding. If primase binds to G40P N-terminal tier that is distal to the C-terminal helicase tier, then primase-mediated stimulation of helicase should be channeled through contacts between the N-terminal tier and helicase domain. We showed that mutations of residues making contact between the N- and C-terminal tiers disrupted the primase-mediated stimulation of helicase activity, which provide evidence for the potential role of inter-tier interactions in channeling the primase stimulation effect from the N-tier to the C-terminal helicase tier.

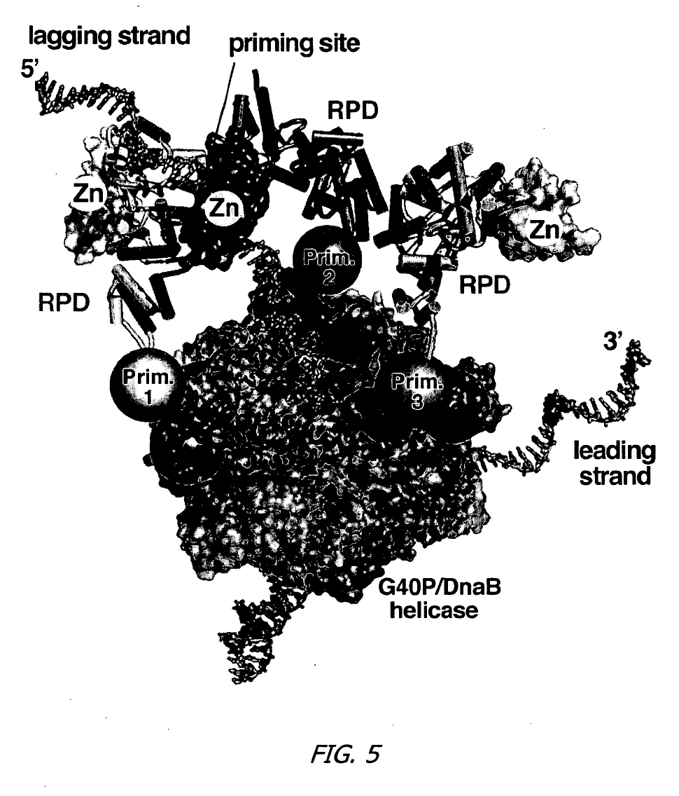

Structural and biochemical data indicate that one DnaB hexamer binds to three DnaG primase^{14,29,32,33,15,31,34}, which may also explain why the 3-fold N-tier is observed in hexameric helicases of only G40P/DnaB homologs. While primase binding can stimulate helicase function in the absence of active priming, it is conceivable that, when the primases start priming on the ssDNA produced by helicase action, the same primase binding can also exert structural constraints on the helicase to negatively regulate helicase function. This primase-imposed negative structural constraint becomes even more evident when considering that the priming direction goes oppositely as the unwinding direction of the helicase at the DNA replication fork, as shown in the model in FIG. 5. In addition, when two primases adjacently bound to the G40P/DnaB hexamer interact with each other to synthesize primer at the same ssDNA site^{35,36}, it is also expected to restrict the helicase conformational change and inhibit the helicase function.

In T7 replication, it is shown that leading strand synthesis pauses upon lagging strand priming at the replication fork³⁷, possibly due to the similar primase-imposed negative structural constraints on the helicase, which may be a potential mechanism for coordinating the leading and lagging strand synthesis. Thus, the primase-helicase interactions may stimulate G40P/DnaB helicase function when the bound primases idle, but suppress helicase function when the attached primases start priming.

Herein, the novel architecture and assembly mechanism for a DnaB-family helicase, G40P, and the structural requirements that determine interactions with another replication fork enzyme DnaG primase is disclosed. The G40P structure reveals a unique N-terminal 3-fold tier stacking on a classic quasi 6-fold hexameric ATPase ring. The double symmetries of the G40P hexamer are achieved through the alternating arrangement of three cis- and three trans-monomers. Structural and functional analyses of the interaction between G40P and DnaG indicate that the N-terminal tier and its structural integrity are essential for primase recognition and for primase-mediated stimulation, which provide a basis for the future understanding of how the helicase coordinates with other replication protein at DNA replication fork.

Methods

Protein Purification and Crystallization

The cDNAs encoding SPP1 helicase G40P and B. subtilis DnaG primase were PCR amplified and cloned into the E. coli expression vector PGEX-KG. All constructs were confirmed by sequencing of the entire open reading frame. For purification of recombinant proteins, E. coli cells were harvested by centrifugation; the cell pellet was suspended in 20 mM Tris-HCl (pH 8.0), 0.5 M NaCl, 1 mM DTT and lysed using a Microfluidics pressurized cell disrupter, followed by a brief sonication. After clarification by centrifugation, the GST-fusion protein was isolated using a glutathione affinity column at 4° C. The G40P protein was cleaved from the GST-fusion with thrombin, and purified using Resource-Q ion exchange column, followed by passage through a Superdex-200 gel filtration in 20 mM Tris-HCl (pH 8.0), 500 mM NaCl, 1 mM DTT. Proteins were concentrated to approximately 10 mg/ml for crystallization and biochemical assays.

Crystals of the two G40P constructs: full length (1-442) and ΔN129 (residues 130-442) were all obtained at 18° C. by hanging drop vapor diffusion method. The P2₁2₁2₁ crystals of the full-length G40P were grown in solutions containing 0.1 M Hepes (pH 7.5), 1-1.25 M MgAc, and 0.02-0.04% β-octylglucoside. Dehydration, by transferring crystals into slightly higher concentrations of mother liquor and incubating for 3-5 days over reservoir solution supplemented with 25% glycerol, improved the diffraction resolution from 6 Å to 3.9 Å. The P6₁ crystal form was obtained from ΔN129 protein in mother liquor containing 0.1 M sodium citrate (pH 5.6), 8-12% PEG-4000, 0.2 M ammonium acetate in the presence of 1 mM ATP-γ-S.

Data Collection and Structure Determination

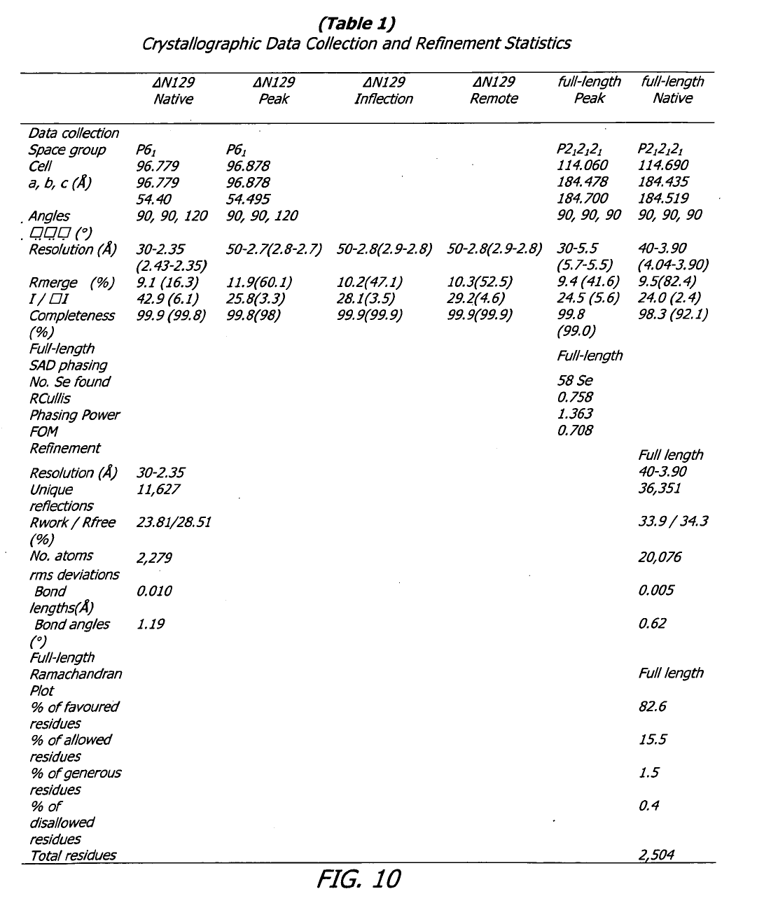

Native, Se-SAD, or Se-MAD data sets were collected at synchrotron beamlines using crystals frozen in liquid nitrogen. Diffraction data were processed with HKL2000³⁸ (FIG. 10). The structure of ΔN129 was determined with the program SOLVE³⁹ using a Se-MAD data set. A solvent flattening step with RESOLVE yielded an electron density map containing regions of well-featured α-helices, which allowed the initial model building with the program O⁴⁰. Higher resolution maps were obtained by combination of a two wavelength MAD dataset with a native dataset using a MIRAS phasing scheme in the program SHARP. Refinement with Refmac5 using the native data to 2.35 Å led to a final model with an Rfree of 28.51% and Rwork of 23.81% (FIG. 10).

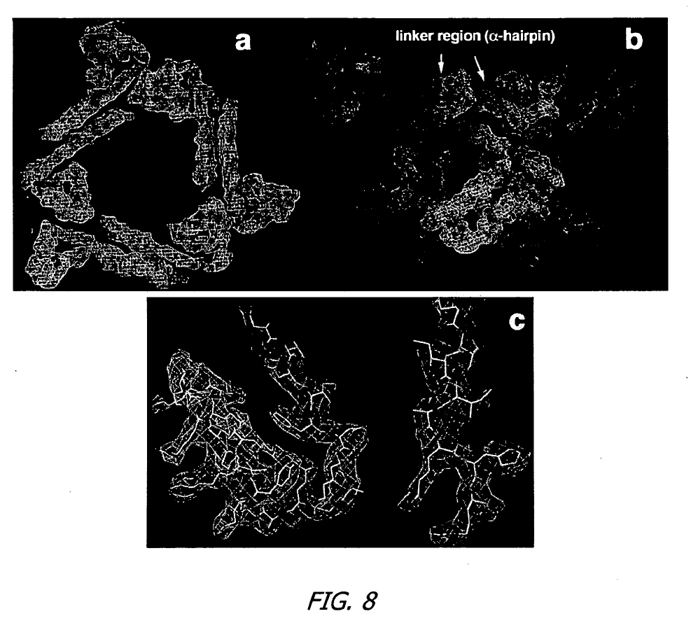

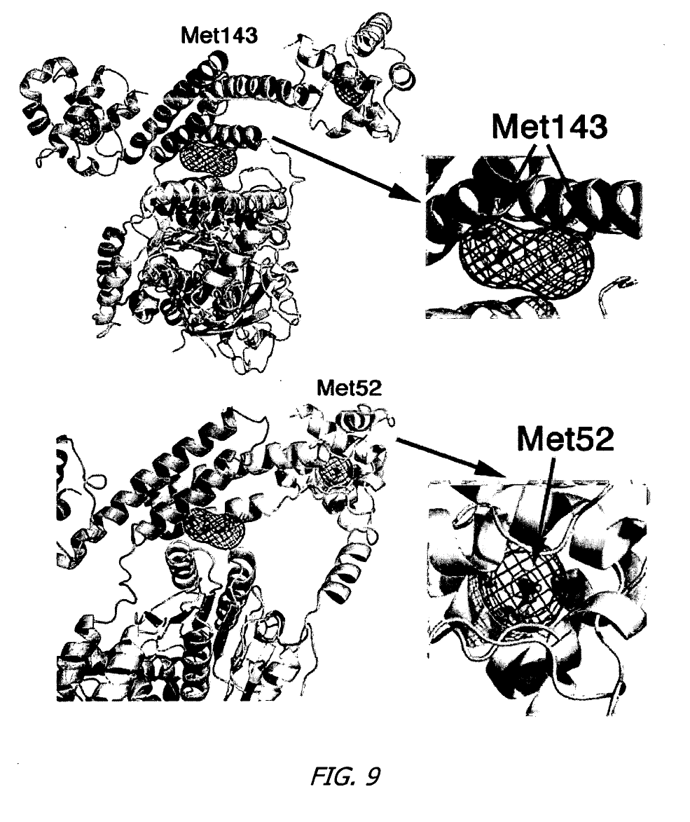

To determine the full-length G40P structure, 58 Se were located using SOLVE³⁹ from a SAD dataset in the resolution range of 30-5.5 Å. Heavy atom refinement and phasing were performed with SHARP⁴¹. The program RESOLVE automatically identified the initial 6-fold symmetry operators for NCS averaging and the resulting electron density map showed excellent main chain connectivity, which allowed the unambiguous docking of six copies of the ATPase domain structure from ΔN129 construct, as well as six copies of the homologous crystal structure of the N-terminal globular domain of E. coli DnaB (PDB 1B79), by phased translation searches 42 as well as manual fitting using O. Subsequent 2-domain (N-terminal and C-terminal domains) 6-fold NCS averaging and phase extension to 4.5 Å using DM⁴³ in CCP4 improved the density map that revealed the missing parts with well connected main-chain density throughout the molecule, including the α-hairpin (FIG. 8). The phases were further improved via a phase combination of the anomalous experimental phases with the hexameric model phases using SHARP⁴¹, which produced a contiguous density for the entire G40P hexamer.

The program MOLREP⁴⁴ placed the hexameric G40P model into the 3.90 Å native data for torsional simulated annealing and minimization refinement using CNS program. At this point, the electron density maps allowed the building of all the missing side chains, and the 58 Se sites, along with well featured side-chain density, were helpful for checking the registry of the polypeptide (FIGS. 7 and 8). NCS restraints were applied throughout the refinement process in CNS as well as in TLS 45 refinement with REFMAC5⁴⁶. Four different NCS groups were used: group one, the six N-terminal domain (6-fold); group two, the six C-terminal domains (6-fold); group three, the three cis α-hairpin (3-fold); and group 4, the three trans α-hairpins (3-fold). Geometry restrained refinement yielded Rfree and Rwork of 34.3% and 33.9% respectively (FIG. 10). The final model has been validated by comparing it with the experimental map and by calculating simulated-annealing omit maps⁴⁷. The maps calculated with sharpened data by applying a B factor of −90 produced well-featured side chain electron density.

Helicase Assay

The substrate for the helicase assay was prepared by annealing a ³²P-labeled ssDNA (a 60-base oligonucleotide) to the circular M13mp18 ssDNA. This oligonucleotide has 35 nucleotides annealed to the M13 DNA, leaving a 25-nucleotide 5′ overhang. The substrate DNA was incubated with various amounts of different G40P mutant proteins in the presence or absence of primase at 37 degrees C. for 30 minutes in a buffer containing 20 mM Tris-HCl (pH 7.5), 5 mM ATP, 10 mM MgCl₂, 1 mM DTT, and 50 mM NaCl. The reaction was terminated by adding a stop solution containing 100 mM EDTA, 0.5% SDS, and 50% glycerol. Samples were analyzed on a 12% native polyacrylamide gel in 1 M Tris/borate/EDTA running buffer. The unwinding of the substrate DNA was detected by autoradiography.

ATPase Assay

15 μL reactions containing 20 mM Tris-HCL (pH 7.5), 10 mM MgCl₂, 1 mM DTT, 0.1 mg/mL BSA, 1 μCi [α-³²P]ATP (Amersham, ˜3000 Ci/mmol), plus 100 μM cold ATP, and various amounts of G40P or G40P with varying amounts of primase to be tested were assembled on ice. Reactions were incubated at 37 degrees C. for 30 minutes and were stopped by addition of 10 mM EDTA and by being placed on ice. 5 uL from each reaction was placed onto a prewashed PEI-cellulose TLC plate (SelectoScientific), dried, and run for two hours in 2 M acetic acid and 0.5 M LiCl. Plates were then dried, autoradiographed using phosphorimaging plates, and quantified.

Native Gel Shift Assay

Interactions between G40P and B. subtilis DnaG primase were examined using a native gel shift assay. 10 ug of various G40P constructs were mixed with 10 ug primase in a buffer containing 25 mM TrisHCl pH 8.0, 50 mM NaCl, 5 mM MgCl₂, and 1 mM ATP-γ-S and incubated for 30 minutes on ice. The protein mixtures were then analyzed by 6% polyacrymamide native gel electrophoresis at 150 voltage for one hour at 4 degrees C. The gel was stained by Coomassie blue for detection.

Another study reporting the full-length DnaB hexamer structure from Bacillus stearothermophilus bound with the P16 fragment of DnaG by Bailey et al. was recently published³¹.

According to the present disclosure, G40P is a protein that is characterized by the amino acid sequence represented in Tables 1 and 2 above. According to the present disclosure, general reference to G40P protein is a protein that, at a minimum, contains any portion of the N globe and C domains of G40P and DnaB like helicases, and includes other biologically active fragments of G40P proteins. A homologue of a G40P protein includes proteins which differ from a naturally occurring G40P in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). Preferably, a G40P homologue has an amino acid sequence that is at least about 70% identical to the amino acid sequence of a naturally occurring G40P, and more preferably, at least about 75%, and more preferably, at least about 80%, and more preferably, at least about 85%, and more preferably, at least about 90%, and more preferably, at least about 95% identical to the amino acid sequence of a naturally occurring G40P. Preferred three-dimensional structural homologues of a G40P are described in detail below. According to the present disclosure, a G40P homologue preferably has, at a minimum, the ability to bind to a naturally occurring ligand of G40P (e.g., dsDNA, ssDNA, ATP, primase (including any additional fragments with G40P-binding ability). Such homologues include fragments of a full length G40P (e.g., the N globe or the C domain) and can be referred to herein as a G40P ligand-binding fragment. In one embodiment, a G40P homologue has the biological activity of a naturally occurring G40P. Reference to a G40P protein can also generally refer to G40P in complex with a ligand.

In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue or mimetic (discussed below), may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein. As used herein, a protein that has “G40P biological activity” or that is referred to as a G40P refers to a protein that has an activity that can include any one, and preferably more than one, of the following characteristics: (a) binds to a natural ligand of G40P (e.g., dsDNA, ssDNA, ATP or primase or other G40P-binding fragments); (b) mediates interactions between the natural ligands and other proteins.

An isolated protein (e.g., an isolated G40P protein), according to the present disclosure, is a protein that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated protein, and particularly, an isolated G40P protein and/or other G40P-binding fragment, is produced recombinantly. According to the present disclosure, a G40P-binding fragment can include any portion of the ligand that contains at least a portion of the ligand that is sufficient to bind to G40P, and can include, but is not limited to, portions of the ligand, an isolated segment or a portion thereof. The terms “fragment”, “segment” and “portion” can be used interchangeably herein with regard to referencing a part of a protein.

Proteins of the present disclosure are preferably retrieved, obtained, and/or used in “substantially pure” form. As used herein, “substantially pure” refers to a purity that allows for the effective use of the protein in vitro, ex vivo or in vivo according to the present disclosure. For a protein to be useful in an in vitro, ex vivo or in vivo method according to the present disclosure, it is substantially free of contaminants, other proteins and/or chemicals that might interfere or that would interfere with its use in a method disclosed by the present disclosure, or that at least would be undesirable for inclusion with the protein when it is used in a method disclosed by the present disclosure. For example, for a G40P protein, such methods include crystallization of the protein, use of a portion of the protein as a drug delivery vehicle, agonist/antagonist identification assays, and all other methods disclosed herein. Preferably, a “substantially pure” protein, as referenced herein, is a protein that can be produced by any method (i.e., by direct purification from a natural source, recombinantly, or synthetically), and that has been purified from other protein components such that the protein comprises at least about 80% weight/weight of the total protein in a given composition (e.g., the protein is about 80% of the protein in a solution/composition/buffer), and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, and more preferably at least about 99%, weight/weight of the total protein in a given composition.

As used herein, a “structure” of a protein refers to the components and the manner of arrangement of the components to constitute the protein. The “three dimensional structure” or “tertiary structure” of the protein refers to the arrangement of the components of the protein in three dimensions. Such term is well known to those of skill in the art. It is also to be noted that the terms “tertiary” and “three dimensional” can be used interchangeably.

The present disclosure provides the atomic coordinates that define the three dimensional structure of a G40P truncated monomers and full-length monomer. More specifically, Tables 1 and 2 provide the atomic coordinates for G40P truncated monomer and the full-length hexamer.

A G40P-ligand complex, refers to the complex (e.g., interaction, binding), that forms between G40P and any of its ligands (e.g., dsDNA, ssDNA, ATP or primase) in the absence of a compound that interferes with the interaction between the G40P and its ligand(s). A complex is naturally formed between at least one full length G40P and a full-length ligand, but according to the present disclosure, a G40P-ligand can also include complexes that minimally contain: (1) a G40P fragment and/or G40P domain; and (2) a G40P-contacting portion of a ligand of G40P.

One embodiment of the present disclosure includes a G40P protein in crystalline form. The present disclosure specifically exemplifies a portion of G40P comprising the full-length protein. As used herein, the terms “crystalline G40P” and “G40P crystal” both refer to crystallized G40P protein and are intended to be used interchangeably. Preferably, a crystalline G40P is produced using the crystal formation method described herein, in particular according to the method disclosed in Example 1. A G40P crystal of the present disclosure can comprise any crystal structure and preferably crystallizes as an orthorhombic crystal lattice. A suitable crystalline G40P of the present disclosure includes a monomer or a dimer, hexamer, or a multimer of G40P protein. One preferred crystalline G40P comprises between one and six G40P proteins in an asymmetric unit. A more preferred crystalline G40P comprises a hexamer of G40P proteins. Preferably, a composition of the present disclosure includes G40P protein molecules arranged in a crystalline manner in a space group P212121 so as to form a unit cell of dimensions a=115 Å, b=185 Å, c=185 Å. A preferred crystal of the present disclosure provides X-ray diffraction data for determination of atomic coordinates of the G40P protein to a resolution of about 4.0 Å, and preferably to about 3.0 Å, and more preferably to about 2.0 Å.

One embodiment of the present disclosure includes a method for producing crystals of G40P, comprising combining G40P protein with another liquor and inducing crystal formation to produce the G40P crystals. By way of example, crystals of the two G40P constructs: full length (1-442) and ΔN129 (residues 130-442) were all obtained at 18 degrees C. by hanging drop vapor diffusion method. The P2₁2₁2₁ crystals of the full-length G40P were grown in solutions containing 0.1 M Hepes (pH 7.5), 1-1.25 M MgAc, and 0.02-0.04% β-octylglucoside. Dehydration, by transferring crystals into slightly higher concentrations of mother liquor and incubating for 3-5 days over reservoir solution supplemented with 25% glycerol, improved the diffraction resolution from 6 Å to 3.9 Å. The P6₁ crystal form was obtained from ΔN129 protein in mother liquor containing 0.1 M sodium citrate (pH 5.6), 8-12% PEG-4000, 0.2 M ammonium acetate in the presence of 1 mM ATP-γ-S. Supersaturated solutions of G40P can be induced to crystallize by several methods including, but not limited to, vapor diffusion, liquid diffusion, batch crystallization, constant temperature and temperature induction or a combination thereof. Preferably, supersaturated solutions of G40P are induced to crystallize by hanging drop vapor diffusion. In a vapor diffusion method, G40P is combined with a mother liquor of the present disclosure that will cause the G40P solution to become supersaturated and form G40P crystals at a constant temperature. Vapor diffusion is preferably performed under a controlled temperature and, by way of example, can be performed at 18 degrees C.

One embodiment of the present disclosure includes a representation, or model, of the three dimensional structure of a G40P protein, such as a computer model. A computer model of the present disclosure can be produced using any suitable software program, including, but not limited to, MOLSCRIPT 2.0 (Avatar Software AB, Heleneborgsgatan 21C, SE-11731 Stockholm, Sweden), the graphical display program 0 (Jones et. al., Acta Crystallography, vol. A47, p. 110, 1991), the graphical display program GRASP, or the graphical display program INSIGHT. Suitable computer hardware useful for producing an image of the present disclosure is known to those of skill in the art (e.g., a Silicon Graphics Workstation).

A representation, or model, of the three dimensional structure of the G40P structure for which a crystal has been produced can also be determined using techniques which include molecular replacement or SIR/MIR (single/multiple isomorphous replacement). Methods of molecular replacement are generally known by those of skill in the art (generally described in Brunger, Meth. Enzym., vol. 276, pp. 558-580, 1997; Navaza and Saludjian, Meth. Enzym., vol. 276, pp. 581-594, 1997; Tong and Rossmann, Meth. Enzym., vol. 276, pp. 594-611, 1997; and Bentley, Meth. Enzym., vol. 276, pp. 611-619, 1997, each of which are incorporated by this reference herein in their entirety) and are performed in a software program including, for example, AmoRe (CCP4, Acta Cryst. D50, 760-763 (1994) or XPLOR. Briefly, X-ray diffraction data is collected from the crystal of a crystallized target structure. The X-ray diffraction data is transformed to calculate a Patterson function. The Patterson function of the crystallized target structure is compared with a Patterson function calculated from a known structure (referred to herein as a search structure). The Patterson function of the crystallized target structure is rotated on the search structure Patterson function to determine the correct orientation of the crystallized target structure in the crystal. The translation function is then calculated to determine the location of the target structure with respect to the crystal axes. Once the crystallized target structure has been correctly positioned in the unit cell, initial phases for the experimental data can be calculated. These phases are necessary for calculation of an electron density map from which structural differences can be observed and for refinement of the structure. Preferably, the structural features (e.g., amino acid sequence, conserved di-sulphide bonds, and β-strands or β-sheets) of the search molecule are related to the crystallized target structure.

As used herein, the term “model” refers to a representation in a tangible medium of the three-dimensional structure of a protein, polypeptide or peptide. For example, a model can be a representation of the three dimensional structure in an electronic file, on a computer screen, on a piece of paper (i.e., on a two dimensional medium), and/or as a ball-and-stick figure. Physical three-dimensional models are tangible and include, but are not limited to, stick models and space-filling models. The phrase “imaging the model on a computer screen” refers to the ability to express (or represent) and manipulate the model on a computer screen using appropriate computer hardware and software technology known to those skilled in the art. Such technology is available from a variety of sources including, for example, Evans and Sutherland, Salt Lake City, Utah, and Biosym Technologies, San Diego, Calif. The phrase “providing a picture of the model” refers to the ability to generate a “hard copy” of the model. Hard copies include both motion and still pictures. Computer screen images and pictures of the model can be visualized in a number of formats including space-filling representations, a carbon traces, ribbon diagrams and electron density maps.

Preferably, a three dimensional structure of a G40P protein provided by the present disclosure includes: (a) a structure defined by atomic coordinates of a three dimensional structure of a crystalline G40P; (b) a structure defined by atomic coordinates selected from the group consisting of: (i) atomic coordinates selected from Tables 1 and 2 above; and, (ii) atomic coordinates that define a three dimensional structure, wherein at least 50% of the structure has an average root-mean-square deviation (RMSD) from backbone atoms in secondary structure elements in at least one domain of a three dimensional structure represented by the atomic coordinates of (1) of equal to or less than about 1.0 Å; and/or (c) a structure defined by atomic coordinates derived from G40P protein molecules arranged in a crystalline manner in a space group P212121 so as to form a unit cell of dimensions a=115 Å, b=185 Å, c=185 Å.

The present inventors have provided the atomic coordinates that define the three dimensional structure of a crystalline G40P. Using the guidance provided herein, one of skill in the art will be able to reproduce such a crystalline structure and define atomic coordinates of such a structure. Example 1 demonstrates the production of a G40P arranged in a crystalline manner in a space group P212121 so as to form a unit cell of dimensions a=115 Å, b=185 Å, c=185 Å. The atomic coordinates determined from this crystal structure are represented in Table 2.

In one embodiment, a three dimensional structure of a G40P protein provided by the present disclosure includes a structure represented by atomic coordinates that define a three dimensional structure, wherein at least 50% of the structure has an average root-mean-square deviation (RMSD) from backbone atoms in secondary structure elements in at least one domain of a three dimensional structure represented by the atomic coordinates of Tables 1 or 2 of equal to or less than about 1.0 Å. Such a structure can be referred to as a structural homologue of the G40P structures defined by Tables 1 and 2. Preferably, at least 50% of the structure has an average root-mean-square deviation (RMSD) from backbone atoms in secondary structure elements in at least one domain of a three dimensional structure represented by the atomic coordinates of Tables 1 and 2 of equal to or less than about 0.7 Å, equal to or less than about 0.5 Å, and most preferably, equal to or less than about 0.3 Å. In a more preferred embodiment, a three dimensional structure of a G40P protein provided by the present disclosure includes a structure defined by atomic coordinates that define a three dimensional structure, wherein at least about 75% of such structure has the recited average root-mean-square deviation (RMSD) value, and more preferably, at least about 90% of such structure has the recited average root-mean-square deviation (RMSD) value, and most preferably, about 100% of such structure has the recited average root-mean-square deviation (RMSD) value.

In one embodiment, RMSD of a structural homologue of G40P can be extended to include atoms of amino acid side chains. As used herein, the phrase “common amino acid side chains” refers to amino acid side chains that are common to both the structural homologue and to the structure that is actually represented by such atomic coordinates. Preferably, at least 50% of the structure has an average root-mean-square deviation (RMSD) from common amino acid side chains in at least one domain of a three dimensional structure represented by the atomic coordinates of Tables 1 and 2 of equal to or less than about 1.0 Å equal to or less than about 0.7 Å, equal to or less than about 0.5 Å, and most preferably, equal to or less than about 0.3 Å. In a more preferred embodiment, a three dimensional structure of a G40P protein provided by the present disclosure includes a structure defined by atomic coordinates that define a three dimensional structure, wherein at least about 75% of such structure has the recited average root-mean-square deviation (RMSD) value, and more preferably, at least about 90% of such structure has the recited average root-mean-square deviation (RMSD) value, and most preferably, about 100% of such structure has the recited average root-mean-square deviation (RMSD) value.

One embodiment of the present disclosure relates to a method of structure-based identification of compounds which potentially bind to G40P, comprising: (a) providing a three dimensional structure of a G40P; and (b) identifying a candidate compound for binding to G40P by performing structure based drug design with the structure of (a) to identify a compound structure that binds to the three dimensional structure of G40P. The three dimensional structure of G40P is selected from the group of: (i) a structure defined by atomic coordinates of a three dimensional structure of a crystalline G40P; (ii) a structure defined by atomic coordinates selected from the group consisting of: (1) atomic coordinates represented in a table selected from the group consisting of G40P (Tables 1 and 2); (2) atomic coordinates that define a three dimensional structure, wherein at least 50% of the structure has an average root-mean-square deviation (RMSD) from backbone atoms in secondary structure elements in at least one domain of a three dimensional structure represented by the atomic coordinates of (1) of equal to or less than about 1.0 Å; and (iii) a structure defined by atomic coordinates derived from G40P protein molecules arranged in a crystalline manner in a space group P212121 so as to form a unit cell of dimensions a=115 Å, b=185 Å, c=185 Å.

The structures used to perform the above-described method have been described in detail above and in the Examples Section. According to the present disclosure, the phrase “providing a three dimensional structure of G40P” is defined as any means of providing, supplying, accessing, displaying, retrieving, or otherwise making available the three dimensional structure of G40P. For example, the step of providing can include, but is not limited to, accessing the atomic coordinates for the structure from a database; importing the atomic coordinates for the structure into a computer or other database; displaying the atomic coordinates and/or a model of the structure in any manner, such as on a computer, on paper, etc.; and determining the three dimensional structure of G40P de novo using the guidance provided herein.

The second step of the method of structure based identification of compounds of the present disclosure includes identifying a candidate compound for binding to G40P by performing structure based drug design with the structure of (a) to identify a compound structure that binds to the three dimensional structure of G40P. Therefore, identification and/or design of compounds that mimic, enhance, disrupt or compete with the interactions of G40P with its ligands are highly desirable. Such compounds can be designed using structure based drug design. Until the discovery of the three-dimensional structure of the present disclosure, the only information available for the development of therapeutic compounds based on the G40P protein was based on the primary sequence of the G40P protein. Structure based drug design refers to the prediction of a conformation of a peptide, polypeptide, protein, or conformational interaction between a peptide or polypeptide, and a compound, using the three dimensional structure of the peptide, polypeptide or protein. Typically, structure based drug design is performed with a computer. For example, generally, for a protein to effectively interact with (e.g., bind to) a compound, it is necessary that the three dimensional structure of the compound assume a compatible conformation that allows the compound to bind to the protein in such a manner that a desired result is obtained upon binding.

Knowledge of the three-dimensional structure of the protein enables a skilled artisan to design a compound having such compatible conformation, or to select such a compound from available libraries of compounds. For example, knowledge of the three dimensional structure of G40P enables one of skill in the art to design a compound that binds to G40P, is stable and results in, for example, inhibition of a biological response. In addition, for example, knowledge of the three-dimensional structure of G40P enables a skilled artisan to design a substrate analog of G40P.

Suitable structures and models useful for structure based drug design are disclosed herein. Preferred target structures to use in a method of structure based drug design include any representations of structures produced by any modeling method disclosed herein, including molecular replacement and fold recognition related methods.

According to the present disclosure, the step of designing a compound for testing in a method of structure based identification of the present disclosure can include creating a new chemical compound or searching databases of libraries of known compounds (e.g., a compound listed in a computational screening database containing three dimensional structures of known compounds). Designing can also be performed by simulating chemical compounds having substitute moieties at certain structural features. The step of designing can include selecting a chemical compound based on a known function of the compound. A preferred step of designing comprises computational screening of one or more databases of compounds in which the three dimensional structure of the compound is known and is interacted (e.g., docked, aligned, matched, interfaced) with the three dimensional structure of a G40P by computer (e.g. as described by Humblet and Dunbar, Animal Reports in Medicinal Chemistry, vol. 28, pp. 275-283, 1993, M Venuti, ed., Academic Press). Methods to synthesize suitable chemical compounds are known to those of skill in the art and depend upon the structure of the chemical being synthesized. Methods to evaluate the bioactivity of the synthesized compound depend upon the bioactivity of the compound (e.g., inhibitory or stimulatory) and are disclosed herein.

Various other methods of structure-based drug design are disclosed in Maulik et al., 1997, Molecular Biotechnology: Therapeutic Applications and Strategies, Wiley-Liss, Inc., which is incorporated herein by reference in its entirety. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

In the present method of structure based drug design, it is not necessary to align a candidate chemical compound (i.e., a chemical compound being analyzed in, for example, a computational screening method of the present disclosure) to each residue in a target site (target sites will be discussed in detail below). Suitable candidate chemical compounds can align to a subset of residues described for a target site. Preferably, a candidate chemical compound comprises a conformation that promotes the formation of covalent or noncovalent crosslinking between the target site and the candidate chemical compound. Preferably, a candidate chemical compound binds to a surface adjacent to a target site to provide an additional site of interaction in a complex. When designing an antagonist (i.e., a chemical compound that inhibits the binding of a ligand to G40P by blocking a binding site or interface), for example, the antagonist should bind with sufficient affinity to the binding site or to substantially prohibit a ligand (i.e., a molecule that specifically binds to the target site) from binding to a target area. It will be appreciated by one of skill in the art that it is not necessary that the complementarity between a candidate chemical compound and a target site extend over all residues specified here in order to inhibit or promote binding of a ligand.

In general, the design of a chemical compound possessing stereochemical complementarity can be accomplished by techniques that optimize, chemically or geometrically, the “fit” between a chemical compound and a target site. Such techniques are disclosed by, for example, Sheridan and Venkataraghavan, Acc. Chem. Res., vol. 20, p. 322, 1987: Goodford, J. Med. Chem., vol. 27, p. 557, 1984; Beddell, Chem. Soc Reviews, vol. 279, 1985; Hol, Angew. Chem., vol. 25, p. 767, 1986; and Verlinde and Hol, Structure, vol. 2, p. 577, 1994, each of which are incorporated by this reference herein in their entirety.

One embodiment of the present disclosure for structure based drug design comprises identifying a chemical compound that complements the shape of a G40P, including a portion of G40P. Such method is referred to herein as a “geometric approach”. In a geometric approach, the number of internal degrees of freedom (and the corresponding local minima in the molecular conformation space) is reduced by considering only the geometric (hard-sphere) interactions of two rigid bodies, where one body (the active site) contains pockets” or “grooves” that form binding sites for the second body (the complementing molecule, such as a ligand).

The geometric approach is described by Kuntz et al., J. Mol. Biol., vol. 161, p. 269, 1982, which is incorporated by this reference herein in its entirety. The algorithm for chemical compound design can be implemented using the software program DOCK Package, Version 1.0 (available from the Regents of the University of California). Pursuant to the Kuntz algorithm, the shape of the cavity or groove on the surface of a structure (e.g., G40P) at a binding site or interface is defined as a series of overlapping spheres of different radii. One or more extant databases of crystallographic data (e.g., the Cambridge Structural Database System maintained by University Chemical Laboratory, Cambridge University, Lensfield Road, Cambridge CB2 1EW, U.K.) or the Protein Data Bank maintained by Brookhaven National Laboratory, is then searched for chemical compounds that approximate the shape thus defined.

Chemical compounds identified by the geometric approach can be modified to satisfy criteria associated with chemical complementarity, such as hydrogen bonding, ionic interactions or Van der Waals interactions.

Another embodiment of the present disclosure for structure-based identification of compounds comprises determining the interaction of chemical groups (“probes”) with an active site at sample positions within and around a binding site or interface, resulting in an array of energy values from which three-dimensional contour surfaces at selected energy levels can be generated. This method is referred to herein as a “chemical-probe approach.” The chemical-probe approach to the design of a chemical compound of the present disclosure is described by, for example, Goodford, J Med. Chem., vol. 28, p. 849, 1985, which is incorporated by this reference herein in its entirety, and is implemented using an appropriate software package, including for example, GRID (available from Molecular Discovery Ltd., Oxford 0X2 9LL, U.K.). The chemical prerequisites for a site-complementing molecule can be identified at the outset, by probing the active site of a G40P, for example, (as represented by the atomic coordinates shown in Tables 1 and 2 above) with different chemical probes, e.g., water, a methyl group, an amine nitrogen, a carboxyl oxygen and/or a hydroxyl. Preferred sites for interaction between an active site and a probe are determined. Putative complementary chemical compounds can be generated using the resulting three-dimensional pattern of such sites.

According to the present disclosure, suitable candidate compounds to test using the method of the present disclosure include proteins, peptides or other organic molecules, and inorganic molecules. Suitable organic molecules include small organic molecules. Peptides refer to small molecular weight compounds yielding two or more amino acids upon hydrolysis. A polypeptide is comprised of two or more peptides. As used herein, a protein is comprised of one or more polypeptides. Preferred therapeutic compounds to design include peptides composed of “L” and/or “D” amino acids that are configured as normal or retroinverso peptides, peptidomimetic compounds, small organic molecules, or homo- or hetero-polymers thereof, in linear or branched configurations.

Preferably, a compound that is identified by the method of the present disclosure originates from a compound having chemical and/or stereochemical complementarity with G40P. Such complementarity is characteristic of a compound that matches the surface of the protein either in shape or in distribution of chemical groups and binds to G40P to promote or inhibit G40P ligand binding in a cell expressing G40P upon the binding of the compound to G40P. More preferably, a compound that binds to a ligand binding site of G40P associates with an affinity of at least about 10-6 M, and more preferably with an affinity of at least about 10-7 M, and more preferably with an affinity of at least about 10-8 M.

Preferably, five general sites of the G40P are targets for structure based drug design (i.e., target sites), although other sites may become apparent to those of skill in the art. The three preferred sites include: (1) the interfaces between G40P monomers; (2) the interfaces between the N-globe, alpha-helix and C-terminal domains of G40P; and (3) the ATPase binding pocket, (4) the primase binding sites, (5) DNA binding sites. Combinations of any of these general sites are also suitable target sites.

The following discussion provides specific detail on compound identification (e.g., drug design) using target sites of G40P based on its three-dimensional structure. It is to be understood, however, that one of skill in the art, using the description of the G40P structure provided herein, will be able to identify compounds that are potential candidates for inhibiting, stimulating or enhancing the interaction of G40P with its other ligands.

A candidate compound for binding to a G40P protein, including to one of the preferred target sites described above, is identified by one or more of the methods of structure-based identification discussed above. As used herein, a “candidate compound” refers to a compound that is selected by a method of structure-based identification described herein as having a potential for binding to a G40P protein (or its ligand) on the basis of a predicted conformational interaction between the candidate compound and the target site of the G40P protein. The ability of the candidate compound to actually bind to a G40P protein can be determined using techniques known in the art, as discussed in some detail below. A “putative compound” is a compound with an unknown regulatory activity, at least with respect to the ability of such a compound to bind to and/or regulate G40P as described herein. Therefore, a library of putative compounds can be screened using structure based identification methods as discussed herein, and from the putative compounds, one or more candidate compounds for binding to G40P can be identified. Alternatively, a candidate compound for binding to G40P can be designed de novo using structure based drug design, also as discussed above. Candidate compounds can be selected based on their predicted ability to inhibit the binding of G40P to its ligand, to stabilize (e.g., enhance) the binding of G40P to its ligand, to bind to and activate G40P, to bind to and inhibit the activation of G40P, to bind to and activate a ligand of G40P, to bind to and inhibit the activation of a ligand of G40P, to disrupt the oligomerization of G40P monomers, or to stabilize the oligomerization of G40P monomers.

Accordingly, in one aspect of the present disclosure, the method of structure-based identification of compounds that potentially bind to G40P proteins or to a complex of G40P and its ligand further includes steps which confirm whether or not a candidate compound has the predicted properties with respect to its effect on G40P (or a ligand of G40P). In one embodiment, the candidate compound is predicted to be an inhibitor of the binding of G40P to its ligand, and the method further includes: (c) contacting the candidate compound identified in step (b) with G40P or a fragment thereof and a G40P ligand or a fragment thereof under conditions in which a G40P-G40P ligand complex can form in the absence of the candidate compound; and (d) measuring the binding affinity of the G40P or fragment thereof to the G40P ligand or fragment thereof. A candidate inhibitor compound is selected as a compound that inhibits the binding of G40P to its ligand when there is a decrease in the binding affinity of the G40P or fragment thereof for the G40P ligand or fragment thereof, as compared to in the absence of the candidate inhibitor compound.

In another embodiment, the candidate compound is predicted to be a stabilizer of the binding of G40P to its ligand, and the method further comprises: (c) contacting the candidate compound identified in step (b) with a G40P-G40P ligand complex, wherein the G40P-G40P ligand complex comprises G40P or a fragment thereof and a G40P ligand, or a fragment thereof; (d) measuring the stability of the G40P-G40P ligand complex of (i) A candidate stabilizer compound is selected as a compound that stabilizes the G40P-G40P ligand complex when there is an increase in the stability of the complex as compared to in the absence of the candidate stabilizer compound.

In another embodiment, the candidate compound is predicted to bind to and activate G40P (i.e., an agonist), and the method further comprises: (c) contacting the candidate compound identified in step (b) with G40P or a ligand-binding fragment thereof, under conditions wherein in the absence of the compound, G40P is not activated; and, (d) measuring the ability of the candidate compound to bind to G40P to activate G40P. A candidate agonist compound is selected as a compound that binds to G40P and activates G40P as compared to in the absence of the candidate agonist compound. A similar embodiment includes the identification of candidate compounds that bind to target sites on the G40P ligand which are now known as a result of the present inventors' work, and the determination of the ability of the candidate compound to bind to and activate the ligand of G40P (e.g., by mimicking the structure of G40P).

In another embodiment, the candidate compound is predicted to bind to and inhibit G40P (i.e., an antagonist), and the method further comprises: (c) contacting the candidate compound identified in step (b) with G40P or a ligand-binding fragment thereof, wherein in the absence of the compound, G40P is not activated; and, (d) measuring the ability of the candidate compound to bind to G40P and activate G40P. A candidate antagonist compound is selected as a compound that binds to G40P but does not activate and, in some embodiments, inhibits any constitutive activation, of the G40P. A similar embodiment includes the identification of candidate compounds that bind to target sites on the G40P ligand which are now known as a result of the present inventors' work, and the determination of the ability of the candidate compound to bind to but not activate the ligand of G40P.

In another embodiment, the candidate compound is predicted to bind to G40P and to disrupt the oligomerization of G40P monomers, and the method further comprises: (c) contacting the candidate compound identified in step (b) with at least two G40P monomers or ligand-binding fragments thereof, in the presence and in the absence of a G40P ligand or fragment thereof; and, (d) measuring the ability of the candidate compound to bind to G40P, the ability of the G40P monomers to oligomerize, and/or the ability of the G40P ligand to activate G40P. A candidate compound for the disruption of G40P oligomerization is selected as a compound that binds to G40P but inhibits the oligomerization of G40P and in some embodiments, inhibits the activation of G40P by its ligand. Similarly, a candidate compound for stabilizing the oligomerization of G40P is a compound that binds to G40P, prolongs the oligomerization of G40P as compared to in the absence of the candidate compound, and in some embodiments, enhances or prolongs the activation of G40P by its ligand.

In one embodiment, the conditions under which a G40P according to the present disclosure is contacted with a candidate compound, such as by mixing, are conditions in which the protein is not bound to a natural ligand if essentially no candidate compound is present. For example, such conditions include normal culture conditions in the absence of a stimulatory compound (a stimulatory compound being, e.g., the natural ligand for the receptor (e.g., DNA, ATP, or primase). In this embodiment, the candidate compound is then contacted with the G40P. In this embodiment, the step of detecting is designed to indicate whether the candidate compound binds to G40P, and in some embodiments, whether the candidate compound activates G40P.

In an alternate embodiment, the conditions under which G40P according to the present disclosure is contacted with a candidate compound, such as by mixing, are conditions in which the protein is normally bound by a ligand or additionally stimulated (activated) if essentially no candidate compound is present. Such conditions can include, for example, contact of G40P with a stimulator molecule (a stimulatory compound being, e.g., the natural ligand for G40P or other equivalent stimulus) which binds to G40P and causes G40P to become activated. In this embodiment, the candidate compound can be contacted with G40P prior to the contact of G40P with the stimulatory compound (e.g., to determine whether the candidate compound blocks or otherwise inhibits the binding and/or stimulation of G40P by the stimulatory compound), or after contact of G40P with the stimulatory compound (e.g., to determine whether the candidate compound downregulates, or reduces the activation of G40P).

In accordance with the present disclosure, a cell-based assay is conducted under conditions which are effective to screen for candidate compounds useful in the method of the present disclosure. Effective conditions include, but are not limited to, appropriate media, temperature, pH and oxygen conditions that permit the growth of the cell that expresses the receptor. An appropriate, or effective, medium refers to any medium in which a cell that naturally or recombinantly expresses a G40P, when cultured, is capable of cell growth and expression of G40P. Such a medium is typically a solid or liquid medium comprising growth factors and assimilable carbon, nitrogen and phosphate sources, as well as appropriate salts, minerals, metals and other nutrients, such as vitamins. Culturing is carried out at a temperature, pH and oxygen content appropriate for the cell. Such culturing conditions are within the expertise of one of ordinary skill in the art.

Cells that are useful in the cell-based assays of the present disclosure include any cell that expresses a G40P and particularly, other proteins that are associated with G40P. Such cells include bacterial cells. Additionally, certain cells may be induced to express G40P recombinantly. Therefore, cells that express G40P can include cells that naturally express G40P, recombinantly express G40P, or which can be induced to express G40P. Cells useful in some embodiments can also include cells that express a natural ligand of G40P, such as bacterial cells.

The assay of the present disclosure can also be a non-cell based assay. In this embodiment, the candidate compound can be directly contacted with isolated G40P or fragment of G40P, and the ability of the candidate compound to bind to G40P or can be evaluated by a binding assay. The assay can, if desired, additionally include the step of further analyzing whether candidate compounds which bind to a portion of G40P are capable of increasing or decreasing the activity of G40P. Such further steps can be performed by cell-based assay, as described above, or by non-cell-based assay.

Alternatively, soluble G40P may be recombinantly expressed and utilized in non-cell based assays to identify compounds that bind to G40P. Recombinantly expressed G40P polypeptides or fusion proteins containing one or more extracellular domains of G40P can be used in the non-cell based screening assays. In non-cell based assays the recombinantly expressed G40P is attached to a solid substrate by means well known to those in the art. For example, G40P and/or cell lysates containing such proteins can be immobilized on a substrate such as: artificial membranes, organic supports, biopolymer supports and inorganic supports. The protein can be immobilized on the solid support by a variety of methods including adsorption, cross-linking (including covalent bonding), and entrapment. Adsorption can be through van del Waal's forces, hydrogen bonding, ionic bonding, or hydrophobic binding. Exemplary solid supports for adsorption immobilization include polymeric adsorbents and ion-exchange resins. Solid supports can be in any suitable form, including in a bead form, plate form, or well form. The test compounds are then assayed for their ability to bind to G40P.

Another embodiment of the present disclosure relates to a therapeutic composition that, when administered to an animal, inhibits or prevents replication of harmful bacterial in the animal. The therapeutic composition comprises a compound that inhibits the activity of G40P, the compound being identified by the method comprising: (a) providing a three dimensional structure of G40P as previously described herein; (b) identifying a candidate compound for binding to G40P by performing structure based drug design with the structure of (a) to identify a compound structure that binds to the three dimensional structure of G40P; (c) synthesizing the candidate compound; and (d) selecting candidate compounds that inhibit the biological activity of G40P. Preferably, the compounds inhibit the formation of a complex between G40P and a G40P ligand, such ligand including, but not limited to, dsDNA, ssDNA, primase and ATP. In a more preferred embodiment, the compound inhibits the activity of G40P.

Methods of identifying candidate compounds and selecting compounds that bind to and activate or inhibit G40P have been previously described herein. Candidate compounds can be synthesized using techniques known in the art, and depending on the type of compound. Synthesis techniques for the production of non-protein compounds, including organic and inorganic compounds are well known in the art.

For smaller peptides, chemical synthesis methods are preferred. For example, such methods include well-known chemical procedures, such as solution or solid-phase peptide synthesis, or semi-synthesis in solution beginning with protein fragments coupled through conventional solution methods. Such methods are well known in the art and may be found in general texts and articles in the area such as: Merrifield, 1997, Methods Enzymol. 289:3-13; Wade et al., 1993, Australas Biotechnol. 3(6):332-336; Wong et al., 1991, Experientia 47(11-12):1123-1129; Carey et al., 1991, Ciba Found Symp. 158:187-203; Plaue et al., 1990, Biologicals 18(3): 147-157; Bodanszky, 1985, Int. J. Pept. Protein Res. 25(5):449-474; H. Dugas and C. Penney, BIOORGANIC CHEMISTRY, (1981) at pages 54-92, all of which are incorporated herein by reference in their entirety. For example, peptides may be synthesized by solid-phase methodology utilizing a commercially available peptide synthesizer and synthesis cycles supplied by the manufacturer. One skilled in the art recognizes that the solid phase synthesis could also be accomplished using the FMOC strategy and a TFA/scavenger cleavage mixture.

If larger quantities of a protein are desired, or if the protein is a larger polypeptide, the protein can be produced using recombinant DNA technology. A protein can be produced recombinantly by culturing a cell capable of expressing the protein (i.e., by expressing a recombinant nucleic acid molecule encoding the protein) under conditions effective to produce the protein, and recovering the protein. Effective culture conditions include, but are not limited to, effective media, bioreactor, temperature, pH and oxygen conditions that permit protein production. An effective medium refers to any medium in which a cell is cultured to produce the protein. Such medium typically comprises an aqueous medium having assimilable carbon, nitrogen and phosphate sources, and appropriate salts, minerals, metals and other nutrients, such as vitamins. Recombinant cells (i.e., cells expressing a nucleic acid molecule encoding the desired protein) can be cultured in conventional fermentation bioreactors, shake flasks, test tubes, microtiter dishes, and Petri plates. Culturing can be carried out at a temperature, pH and oxygen content appropriate for a recombinant cell. Such culturing conditions are within the expertise of one of ordinary skill in the art. Such techniques are well known in the art and are described, for example, in Sambrook et al., 1988, Molecular Cloning: A Laboratory Manual, Cold Spring Harbor Press, Cold Spring Harbor Laboratory, Cold Spring Harbor, N.Y. or Current Protocols in Molecular Biology (1989) and supplements.

As discussed above, a composition, and particularly a therapeutic composition, of the present disclosure generally includes the therapeutic compound (e.g., the compound identified by the structure based identification method) and a carrier, and preferably, a pharmaceutically acceptable carrier. Pharmaceutically acceptable carriers and preferred methods of administration of therapeutic compositions of the present disclosure have been described in detail above with regard to the administration of an inhibitor compound to a patient. Such carriers and administration protocols are applicable to this embodiment.

Another embodiment of the present disclosure relates to a computer for producing a three-dimensional model of a molecule or molecular structure, wherein the molecule or molecular structure comprises a three dimensional structure defined by atomic coordinates of GP40, or a three-dimensional model of a homologue of the molecule or molecular structure, wherein the homologue comprises a three dimensional structure that has an average root-mean-square deviation (RMSD) of equal to or less than about 1.0 Å for the backbone atoms in secondary structure elements in the GP40 protein, wherein the computer comprises: a) a computer-readable medium encoded with the atomic coordinates of the GP40 protein to create an electronic file; b) a working memory for storing a graphical display software program for processing the electronic file; c) a processor coupled to the working memory and to the computer-readable medium which is capable of representing the electronic file as the three dimensional model; and, d) a display coupled to the processor for visualizing the three dimensional model; wherein the three dimensional structure of the GP40 protein is displayed on the computer.

While the G40P structure, related DnaB helicase structures, their uses and related methods have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures.

### REFERENCES

1. Schaeffer, P. M., Headlam, M. J. & Dixon, N. E. Protein—protein interactions in the eubacterial replisome. IUBMB Life. 57, 5-12. (2005).
2. Corn, J. E. & Berger, J. M. Regulation of bacterial priming and daughter strand synthesis through helicase-primase interactions. Nucleic Acids Res 34, 4082-8 (2006).
3. Wickner, S. & Hurwitz, J. Interaction of Escherichia coli dnaB and dnaC(D) gene products in vitro. Proc Natl Acad Sci USA. 72, 921-5. (1975).
4. Erzberger, J. P., Mott, M. L. & Berger, J. M. Structural basis for ATP-dependent DnaA assembly and replication-origin remodeling. Nat Struct Mol. Biol. 13, 676-83. Epub 2006 Jul. 9. (2006).
5. Clarey, M. G. et al. Nucleotide-dependent conformational changes in the DnaA-like core of the origin recognition complex. Nat Struct Mol. Biol. 13, 684-90. Epub 2006 Jul. 9. (2006).
6. Arai, K. & Kornberg, A. A general priming system employing only dnaB protein and primase for DNA replication. Proc Natl Acad Sci USA. 76, 4308-12. (1979).
7. Tougu, K., Peng, H. & Marians, K. J. Identification of a domain of Escherichia coli primase required for functional interaction with the DnaB helicase at the replication fork. J Biol Chem 269, 4675-82 (1994).
8. Johnson, S. K., Bhattacharyya, S. & Griep, M. A. DnaB helicase stimulates primer synthesis activity on short oligonucleotide templates. Biochemistry 39, 736-44 (2000).
9. Soultanas, P. The bacterial helicase-primase interaction: a common structural/functional module. Structure 13, 839-44 (2005).
10. Glover, B. P. & McHenry, C. S. The DNA polymerase III holoenzyme: an asymmetric dimeric replicative complex with leading and lagging strand polymerases. Cell. 105, 925-34. (2001).
11. Benkovic, S. J., Valentine, A. M. & Salinas, F. Replisome-mediated DNA replication. Annu Rev Biochem 70, 181-208 (2001).
12. Patel, S. S. & Picha, K. M. Structure and function of hexameric helicases. Annu Rev Biochem 69, 651-97 (2000).
13. Lu, Y. B., Ratnakar, P. V., Mohanty, B. K. & Bastia, D. Direct physical interaction between DnaG primase and DnaB helicase of Escherichia coli is necessary for optimal synthesis of primer RNA. Proc Natl Acad Sci USA 93, 12902-7 (1996).
14. Tougu, K. & Marians, K. J. The extreme C terminus of primase is required for interaction with DnaB at the replication fork. J Biol Chem 271, 21391-7 (1996).
15. Bird, L. E., Pan, H., Soultanas, P. & Wigley, D. B. Mapping protein-protein interactions within a stable complex of DNA primase and DnaB helicase from Bacillus stearothermophilus. Biochemistry 39, 171-82 (2000).
16. Thirlway, J. & Soultanas, P. In the Bacillus stearothermophilus DnaB-DnaG complex, the activities of the two proteins are modulated by distinct but overlapping networks of residues. J Bacteriol 188, 1534-9 (2006).
17. Ayora, S., Langer, U. & Alonso, J. C. Bacillus subtilis DnaG primase stabilises the bacteriophage SPP1 G40P helicase-ssDNA complex. FEBS Lett 439, 59-62 (1998).
18. Pedre, X., Weise, F., Chai, S., Luder, G. & Alonso, J. C. Analysis of cis and trans acting elements required for the initiation of DNA replication in the Bacillus subtilis bacteriophage SPP1. J Mol Biol 236, 1324-40 (1994).
19. Yu, X., Jezewska, M. J., Bujalowski, W. & Egelman, E. H. The hexameric E. coli DnaB helicase can exist in different Quaternary states. J Mol Biol 259, 7-14 (1996).
20. San Martin, M. C., Stamford, N. P., Dammerova, N., Dixon, N. E. & Carazo, J. M. A structural model for the Escherichia coli DnaB helicase based on electron microscopy data. J Struct Biol 114, 167-76 (1995).
21. San Martin, C. et al. Three-dimensional reconstructions from cryoelectron microscopy images reveal an intimate complex between helicase DnaB and its loading partner DnaC. Structure 6, 501-9 (1998).
22. Yang, S. et al. Flexibility of the rings: structural asymmetry in the DnaB hexameric helicase. J Mol Biol 321, 839-49 (2002).
23. Nunez-Ramirez, R. et al. Quaternary polymorphism of replicative helicase G40P: structural mapping and domain rearrangement. J Mol Biol 357, 1063-76 (2006).
24. Fass, D., Bogden, C. E. & Berger, J. M. Crystal structure of the N-terminal domain of the DnaB hexameric helicase. Structure 7, 691-8 (1999).
25. Weigelt, J., Brown, S. E., Miles, C. S., Dixon, N. E. & Otting, G. NMR structure of the N-terminal domain of E. coli DnaB helicase: implications for structure rearrangements in the helicase hexamer. Structure. 7, 681-90. (1999).
26. Sawaya, M. R., Guo, S., Tabor, S., Richardson, C. C. & Ellenberger, T. Crystal structure of the helicase domain from the replicative helicase-primase of bacteriophage T7. Cell 99, 167-77 (1999).
27. Singleton, M. R., Sawaya, M. R., Ellenberger, T. & Wigley, D. B. Crystal structure of T7 gene 4 ring helicase indicates a mechanism for sequential hydrolysis of nucleotides. Cell 101, 589-600 (2000).
28. Gai, D., Zhao, R., Li, D., Finkielstein, C. V. & Chen, X. S. Mechanisms of conformational change for a replicative hexameric helicase of SV40 large tumor antigen. Cell 119, 47-60 (2004).
29. Thirlway, J. et al. DnaG interacts with a linker region that joins the N- and C-domains of DnaB and induces the formation of 3-fold symmetric rings. Nucleic Acids Res 32, 2977-86 (2004).
30. Kaito, C., Kurokawa, K., Hossain, M. S., Akimitsu, N. & Sekimizu, K. Isolation and characterization of temperature-sensitive mutants of the Staphylococcus aureus dnaC gene. FEMS Microbiol Lett 210, 157-64 (2002).
31. Bailey, S., Eliason, W. K. & Steitz, T. A. Structure of hexameric DnaB helicase and its complex with a domain of DnaG primase. Science 318, 459-63 (2007).
32. Oakley, A. J. et al. Crystal and solution structures of the helicase-binding domain of Escherichia coli primase. J Biol Chem 280, 11495-504 (2005).
33. Syson, K., Thirlway, J., Hounslow, A. M., Soultanas, P. & Waltho, J. P. Solution structure of the helicase-interaction domain of the primase DnaG: a model for helicase activation. Structure. 13, 609-16. (2005).
34. Mitkova, A. V., Khopde, S. M. & Biswas, S. B. Mechanism and stoichiometry of interaction of DnaG primase with DnaB helicase of Escherichia coli in RNA primer synthesis. J Biol Chem 278, 52253-61 (2003).
35. Corn, J. E., Pease, P. J., Hura, G. L. & Berger, J. M. Crosstalk between primase subunits can act to regulate primer synthesis in trans. Mol Cell. 20, 391-401. (2005).
36. Kato, M., Ito, T., Wagner, G., Richardson, C. C. & Ellenberger, T. Modular architecture of the bacteriophage T7 primase couples RNA primer synthesis to DNA synthesis. Mol Cell. 11, 1349-60. (2003).
37. Lee, J. B. et al. DNA primase acts as a molecular brake in DNA replication. Nature. 439, 621-4. (2006).
38. Otwinowski, Z. & Minor, W. in Methods in Enzymology 307-326 (1997).
39. Terwilliger, T. C. & Berendzen, J. Automated MAD and MIR structure solution. Acta Crystallogr D Biol Crystallogr 55, 849-61 (1999).
40. Jones, T. A., Zou, J. Y., Cowan, S. W. & Kjeldgaard, M. Improved methods for building protein models in electron density maps and the location of errors in these models. Acta Crystallogr A 47 (Pt 2), 110-9 (1991).
41. Vonrhein, C., Blanc, E., Roversi, P. & Bricogne, G. Automated Structure Solution With autoSHARP. Methods Mol Biol 364, 215-30 (2006).
42. Strokopytov, B. V. et al. Phased translation function revisited: structure solution of the cofilin-homology domain from yeast actin-binding protein 1 using six-dimensional searches. Acta Crystallogr D Biol Crystallogr 61, 285-93 (2005).
43. Cowtan, K. ‘dm’: An automated procedure for phase improvement by density modification. Joint CCP4 and ESF-EACBM Newsletter on Protein Crystallography 31, 34-38 (1994).
44. Vagin, A. A. & Teplyakov, A. MOLREP: an Automated Program for Molecular Replacement. J. Appl. Cryst. 30, 1022-1025 (1997).
45. Winn, M. D., Murshudov, G. N. & Papiz, M. Z. Macromolecular TLS refinement in REFMAC at moderate resolutions. Methods Enzymol 374, 300-21 (2003).
46. Murshudov, G. N., Vagin, A. A. & Dodson, E. J. Refinement of macromolecular structures by the maximum-likelihood method. Acta Crystallogr D Biol Crystallogr 53, 240-55 (1997).
47. Brunger, A. T. et al. Crystallography & NMR system: A new software suite for macromolecular structure determination. Acta Crystallogr D Biol Crystallogr 54, 905-21 (1998).
48. Keck, J. L., Roche, D. D., Lynch, A. S. & Berger, J. M. Structure of the RNA polymerase domain of E. coli primase. Science. 287, 2482-6. (2000).

1. A method for identifying a compound that binds to any fragment of a G40P protein, the method comprising:
(a), obtaining the three dimensional structure of the G40P hexamer whose sequence consists of SEQ ID NO:1; and
(b) identifying or designing one or more compounds that bind, mimic, enhance, disrupt, or compete with the G40P protein whose sequence consists of SEQ ID NO:1 or interactions of the G40P protein with its ligands based on the three dimensional structure of the G40P hexamer whose sequence consists of SEQ ID NO:1.

2. The method of claim 1, further comprising contacting one or more compounds identified in step (b) with the protein whose sequence consists of SEQ ID NO:1.

3. The method of claim 2, further comprising measuring an activity of the protein whose sequence consists of SEQ ID NO:1, when the protein is contacted with the one or more compounds.

4. The method of claim 3, further comprising comparing activities of the protein whose sequence consists of SEQ ID NO:1, when the protein is in the presence of and in the absence of the one or more compounds.

5. The method of claim 1, further comprising contacting one or more compounds identified in step (b) with a cell that expresses a protein whose sequence consists of SEQ ID NO:1 and detecting whether a phenotype of the cell changes when the one or more compounds are present.

6. The method of claim 1, wherein a therapeutically effective amount of the one or more compounds is effective at treating one or more strains of bacteria that cause Tubercle bacillus in a mammal.

7. The method of claim 1, wherein a therapeutically effective amount of the one or more compounds is effective at treating one or more strains of bacteria that cause Listeria monocytogenes in a mammal.

8. The method of claim 1, wherein a therapeutically effective amount of the one or more compounds is effective at treating one or more strains of bacteria that cause Streptococcus pneumoniae in a mammal.

9. A method for identifying a compound that binds to any fragment of a G40P protein, the method comprising:
(a), obtaining the three dimensional structure of the G40P monomer whose sequence consists of SEQ ID NO:2; and
(b) identifying or designing one or more compounds that bind, mimic, enhance, disrupt, or compete with the G40P protein whose sequence consists of SEQ ID NO:2 or interactions of the G40P protein with its ligands based on the three dimensional structure of the G40P monomer whose sequence consists of SEQ ID NO:2.

10. The method according to claim 9, further comprising contacting one or more compounds identified in step (b) with the protein whose sequence consists of SEQ ID NO:2.

11. The method according to claim 10, further comprising measuring an activity of the protein whose sequence consists of SEQ ID NO:2, when the protein is contacted with the one or more compounds.

12. The method according to claim 11, further comprising comparing activities of the protein whose sequence consists of SEQ ID NO:2, when the protein is in the presence of and in the absence of the one or more compounds.

13. The method according to claim 12, further comprising contacting one or more compounds identified in step (b) with a cell that expresses a protein whose sequence consists of SEQ ID NO:2; and detecting whether a phenotype of the cell changes when the one or more compounds are present.

14. The method of claim 9, wherein a therapeutically effective amount of the one or more compounds is effective at treating one or more strains of bacteria that cause Tubercle bacillus in a mammal.

15. The method of claim 9, wherein a therapeutically effective amount of the one or more compounds is effective at treating one or more strains of bacteria that cause Listeria monocytogenes in a mammal.

16. The method of claim 9, wherein a therapeutically effective amount of the one or more compounds is effective at treating one or more strains of bacteria that cause Streptococcus pneumoniae in a mammal.

17. A method for identifying a compound that binds to any fragment of a DnaB-like helicase protein that bears similarity with a root-mean-square deviation (RMSD) of 2.0 with at least one of the N-globe, alpha-hairpin and the C-terminal ATPase domains the method comprising:
(a), obtaining the three dimensional structure of the DnaB-like helicase protein that bears similarity with a root-mean-square deviation (RMSD) of 2.0 with at least one of the N-globe, alpha-hairpin and the C-terminal ATPase domains whose sequence consists of SEQ ID NO:1 or SEQ ID NO:2; and
(b) identifying or designing one or more compounds that bind, mimic, enhance, disrupt, or compete with the DnaB-like helicase protein that bears similarity with a root-mean-square deviation (RMSD) of 2.0 with at least one of the N-globe, alpha-hairpin and the C-terminal ATPase domains.

18. The method according to claim 17, further comprising measuring an activity of the protein of any DnaB-like helicase that bears similarity with a root-mean-square deviation (RMSD) of 2.0 with at least one of the N-globe, alpha-hairpin and the C-terminal ATPase domains whose sequence consists of SEQ ID NO:1 or SEQ ID NO:2, when the protein is contacted with the one or more compounds.

19. The method according to claim 18, further comprising comparing activities of the protein of any DnaB-like helicase that bears similarity with a root-mean-square deviation (RMSD) of 2.0 with at least one of the N-globe, alpha-hairpin and the C-terminal ATPase domains whose sequence consists of SEQ ID NO:1 or SEQ ID NO:2, when the protein is in the presence of and in the absence of the one or more compounds.

20. The method according to claim 19, further comprising contacting one or more compounds identified in step (b) with a cell that expresses a protein of any DnaB-like helicase that bears similarity with a root-mean-square deviation (RMSD) of 2.0 with at least one of the N-globe, alpha-hairpin and the C-terminal ATPase domains whose sequence consists of SEQ ID NO:1 or SEQ ID NO:2; and detecting whether a phenotype of the cell changes when the one or more compounds are present.


SEQUENCE LISTING (2 sequences)

SEQ ID NO: 1 - protein, 2652 aa, Bacillus subtilis
MNPEEKTVMLYNEYAEQAVLGSILTEPELIKECPLTPEHFSPGKHFNIYFTMQDLDRKGQSVDFTSIAARVGEKLPQLGGFGYLTELAASVASTTTFKQHCQTVSEYFQKRKAISIAQQIIENVNESDDGPVKPIQEAVSELMEIEASGTDDDDGSIDEALVTVYEEIESADGNITGVPSGFTELDRMTYGYKRRNFVLIAARPSMGKTAFALKQAKNMSDNDDVVNLHSLEMGKKENIKRLIVTAGSINAQKIKAARRDFASEDWGKLSMAIGEISNSNINIFDKAGQSVNYIWSKTRQTKRKNPGKRVIVMIDYLQLLEPAKANDSRTNQISQISRDLKKMARELDVVVIALSQLSRQVEQRQDKRPMLSDLRESGQLEQDADIIEFLYRDDYYDKESESKNIVEVIIAKHRDGPVGTVSLAFIKEYGNFVNLERRFDDRMNPEEKTVMLYNEYAEQAVLGSILTEPELIKECPLTPEHFSPGKHFNIYFTMQDLDRKGQSVDFTSIAARVGEKLPQLGGFGYLTELAASVASTTTFKQHCQTVSEYFQKRKAISIAQQIIENVNESDDGPVKPIQEAVSELMEIEASGTDDDDGSIDEALVTVYEEIESADGNITGVPSGFTELDRMTYGYKRRNFVLIAARPSMGKTAFALKQAKNMSDNDDVVNLHSLEMGKKENIKRLIVTAGSINAQKIKAARRDFASEDWGKLSMAIGEISNSNINIFDKAGQSVNYIWSKTRQTKRKNPGKRVIVMIDYLQLLEPAKANDSRTNQISQISRDLKKMARELDVVVIALSQLSRQVEQRQDKRPMLSDLRESGQLEQDADIIEFLYRDDYYDKESESKNIVEVIIAKHRDGPVGTVSLAFIKEYGNFVNLERRFDDRMNPEEKTVMLYNEYAEQAVLGSILTEPELIKECPLTPEHFSPGKHFNIYFTMQDLDRKGQSVDFTSIAARVGEKLPQLGGFGYLTELAASVASTTTFKQHCQTVSEYFQKRKAISIAQQIIENVNESDDGPVKPIQEAVSELMEIEASGTDDDDGSIDEALVTVYEEIESADGNITGVPSGFTELDRMTYGYKRRNFVLIAARPSMGKTAFALKQAKNMSDNDDVVNLHSLEMGKKENIKRLIVTAGSINAQKIKAARRDFASEDWGKLSMAIGEISNSNINIFDKAGQSVNYIWSKTRQTKRKNPGKRVIVMIDYLQLLEPAKANDSRTNQISQISRDLKKMARELDVVVIALSQLSRQVEQRQDKRPMLSDLRESGQLEQDADIIEFLYRDDYYDKESESKNIVEVIIAKHRDGPVGTVSLAFIKEYGNFVNLERRFDDRMNPEEKTVMLYNEYAEQAVLGSILTEPELIKECPLTPEHFSPGKHFNIYFTMQDLDRKGQSVDFTSIAARVGEKLPQLGGFGYLTELAASVASTTTFKQHCQTVSEYFQKRKAISIAQQIIENVNESDDGPVKPIQEAVSELMEIEASGTDDDDGSIDEALVTVYEEIESADGNITGVPSGFTELDRMTYGYKRRNFVLIAARPSMGKTAFALKQAKNMSDNDDVVNLHSLEMGKKENIKRLIVTAGSINAQKIKAARRDFASEDWGKLSMAIGEISNSNINIFDKAGQSVNYIWSKTRQTKRKNPGKRVIVMIDYLQLLEPAKANDSRTNQISQISRDLKKMARELDVVVIALSQLSRQVEQRQDKRPMLSDLRESGQLEQDADIIEFLYRDDYYDKESESKNIVEVIIAKHRDGPVGTVSLAFIKEYGNFVNLERRFDDRMNPEEKTVMLYNEYAEQAVLGSILTEPELIKECPLTPEHFSPGKHFNIYFTMQDLDRKGQSVDFTSIAARVGEKLPQLGGFGYLTELAASVASTTTFKQHCQTVSEYFQKRKAISIAQQIIENVNESDDGPVKPIQEAVSELMEIEASGTDDDDGSIDEALVTVYEEIESADGNITGVPSGFTELDRMTYGYKRRNFVLIAARPSMGKTAFALKQAKNMSDNDDVVNLHSLEMGKKENIKRLIVTAGSINAQKIKAARRDFASEDWGKLSMAIGEISNSNINIFDKAGQSVNYIWSKTRQTKRKNPGKRVIVMIDYLQLLEPAKANDSRTNQISQISRDLKKMARELDVVVIALSQLSRQVEQRQDKRPMLSDLRESGQLEQDADIIEFLYRDDYYDKESESKNIVEVIIAKHRDGPVGTVSLAFIKEYGNFVNLERRFDDRMNPEEKTVMLYNEYAEQAVLGSILTEPELIKECPLTPEHFSPGKHFNIYFTMQDLDRKGQSVDFTSIAARVGEKLPQLGGFGYLTELAASVASTTTFKQHCQTVSEYFQKRKAISIAQQIIENVNESDDGPVKPIQEAVSELMEIEASGTDDDDGSIDEALVTVYEEIESADGNITGVPSGFTELDRMTYGYKRRNFVLIAARPSMGKTAFALKQAKNMSDNDDVVNLHSLEMGKKENIKRLIVTAGSINAQKIKAARRDFASEDWGKLSMAIGEISNSNINIFDKAGQSVNYIWSKTRQTKRKNPGKRVIVMIDYLQLLEPAKANDSRTNQISQISRDLKKMARELDVVVIALSQLSRQVEQRQDKRPMLSDLRESGQLEQDADIIEFLYRDDYYDKESESKNIVEVIIAKHRDGPVGTVSLAFIKEYGNFVNLERRFDDR

SEQ ID NO: 2 - protein, 312 aa, Bacillus subtilis
PVKPIQEAVSELMEIEASGTDDDDGSIDEALVTVYEEIESADGNITGVPSGFTELDRMTYGYKRRNFVLIAARPSMGKTAFALKQAKNMSDNDDVVNLHSLEMGKKENIKRLIVTAGSINAQKIKAARRDFASEDWGKLSMAIGEISNSNINIFDKAGQSVNYIWSKTRQTKRKNPGKRVIVMIDYLQLLEPAKANDSRTNQISQISRDLKKMARELDVVVIALSQLSRQVEQRQDKRPMLSDLRESGQLEQDADIIEFLYRDDYYDKESESKNIVEVIIAKHRDGPVGTVSLAFIKEYGNFVNLERRFDDR